(19) United States
(12) Patent Application Publication
(10) Pub. No.: US 2024/0228499 A1
(43) Pub. Date: Jul. 11, 2024

(54) MASP-2 INHIBITORS AND METHODS OF USE

(71) Applicant: Omeros Corporation, Seattle, WA (US)

(72) Inventors: Neil S. Cutshall, Snohomish, WA (US); Jennifer Lynn Gage, Kenmore, WA (US); Do Yeon Kwon, Seattle, WA (US); Thomas L. Little, Seattle, WA (US); Markus Metz, Bellevue, WA (US); Peter Kurt Nollert von Specht, Bainbridge Island, WA (US); Jennifer Tsoung, Seattle, WA (US); Sudheer Babu Vaddela, Bellevue, WA (US); Connor Weidle, Seattle, WA (US)

(73) Assignee: Omeros Corporation, Seattle, WA (US)

(21) Appl. No.: 18/523,642

(22) Filed: Nov. 29, 2023

Related U.S. Application Data
(60) Provisional application No. 63/385,597, filed on Nov. 30, 2022.
(60) Provisional application No. 63/588,653, filed on Oct. 6, 2023.

(51) Int. Cl. C07D 487/04; A61K 31/519; A61K 31/5377; C07D 519/00

(52) U.S. Cl. CPC C07D 487/04; A61K 31/519; A61K 31/5377; C07D 519/00

(57) ABSTRACT

The disclosure provides synthetic compositions useful as inhibitors of mannan-binding lectin-associated serine protease-2 (MASP-2), including compositions that selectively inhibit MASP-2 over thrombin, as well as methods for the manufacture and use thereof.

### CROSS-REFERENCE(S) TO RELATED APPLICATION(S)

This patent application claims the benefit of U.S. Provisional Patent Application Ser. No. 63/385,597, filed on Nov. 30, 2022, and the benefit of U.S. Provisional Patent Application Ser. No. 63/588,653, filed on Oct. 6, 2023, the disclosures of which are hereby incorporated by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 4278-P6US_Seq_List.xml. The XML file is 2,415 bytes; was created on May 17, 2023; and is being submitted electronically via Patent Center with the filing of the specification.

### FIELD OF THE INVENTION

The disclosure provides synthetic compositions useful as inhibitors of mannan-binding lectin-associated serine protease-2 (MASP-2), including compositions that selectively inhibit MASP-2 over thrombin, as well as methods for the manufacture and use thereof.

### BACKGROUND

The complement system plays a role in the inflammatory response and becomes activated upon tissue damage or microbial infection. Complement activation must be tightly regulated to ensure selective targeting of invading microorganisms and to avoid self-inflicted damage (Ricklin et al., Nat. Immunol. 11:785-797, 2010).

Currently, it is widely accepted that the complement system is activated through three distinct pathways: the classical pathway, the lectin pathway, and the alternative pathway. The classical pathway is usually triggered by a complex composed of host antibodies bound to a foreign particle (i.e., an antigen), and generally requires prior exposure to an antigen for the generation of a specific antibody response. Since activation of the classical pathway depends on a prior adaptive immune response by the host, the classical pathway is part of the acquired immune system response. In contrast, both the lectin and alternative pathways are independent of adaptive immunity, and are instead part of the innate immune system response.

Mannan-binding lectin-associated serine protease-2 (MASP-2) has been shown to be required for the function of the lectin pathway, one of the principal complement activation pathways (Vorup-Jensen et al., J. Immunol 165:2093-2100, 2000; Ambrus et al., J Immunol. 170:1374-1382, 2003; Schwaeble et al., PNAS 108:7523-7528, 2011).

Inhibition of MASP-2 appears to not interfere with the antibody-dependent classical complement activation pathway. As described in U.S. Pat. No. 9,011,860 (assigned to Omeros Corporation), which is hereby incorporated by reference in its entirety, a fully human monoclonal antibody targeting human MASP-2 has been generated, which binds to human MASP-2 with high affinity and blocks the lectin pathway complement activity.

MASP-2-dependent complement activation has been implicated as contributing to the pathogenesis of numerous acute and chronic disease states, including certain conditions caused by severe acute respiratory syndrome coronavirus 2 (SARS-COV-2) infection.

Therefore, a need exists for compounds suitable for administration and treatment of subjects suffering from MASP-2-associated diseases and disorders, including diseases that are not suitably or efficiently treated with large molecule biologic inhibitors.

### SUMMARY

In some aspects, provided herein is a compound having Structure (I):

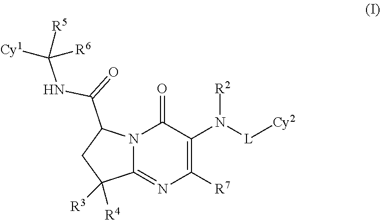

or a stereoisomer, tautomer, or pharmaceutically acceptable salt thereof, wherein:
Cy¹ is a substituted aryl or a substituted or unsubstituted 5-10-membered heteroaryl, or
Cy¹, together with one of R⁵ or R⁶ and the carbon to which they are attached, forms a substituted or unsubstituted C₃-C₆ cycloalkyl fused to a substituted or unsubstituted 5-10-membered heteroaryl, or a substituted or unsubstituted phenyl;
Cy² is a substituted aryl, a substituted or unsubstituted C₃-C₆ cycloalkyl, a substituted or unsubstituted 5-10-membered heteroaryl, or is hydrogen;
R² is hydrogen, a substituted or unsubstituted C₁-C₃ alkyl, or a substituted or unsubstituted C₃-C₆ cycloalkyl;
R³ and R⁴ are each independently hydrogen, halogen, substituted or unsubstituted C₁-C₃ alkyl, or substituted or unsubstituted C₃-C₆ cycloalkyl, or R³ and R⁴, together with the carbon to which they are attached, form a substituted or unsubstituted C₃-C₆ cycloalkyl or a substituted or unsubstituted C₅-C₆ cycloalkenyl;
R⁵ and R⁶ are each independently hydrogen, C₁-C₃ alkyl, alkoxy, haloalkyl, hydroxyalkyl, haloalkoxy, or C₃-C₆ cycloalkyl;
R⁷ is selected from the group consisting of hydrogen, halogen, substituted or unsubstituted C₁-C₃ alkyl, and substituted or unsubstituted C₃-C₆ cycloalkyl;
L is hydrogen or —(CR^{8a}R^{8b})ₙ—, wherein each —(CR^{8a}R^{8b})— is independently the same or different;
R^{8a} and R^{8b} are each independently hydrogen, substituted or unsubstituted linear or branched C₁-C₃ alkyl, or R^{8a} and R^{8b}, together with the carbon to which they are attached, form a substituted or unsubstituted C₃-C₆ cycloalkyl; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms in Structure (I) is optionally replaced with a deuterium atom; and
provided that the compound of Structure (I) does not have the structure:

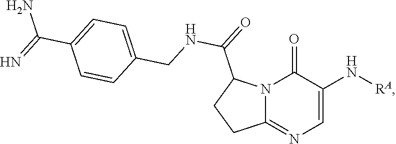

wherein R^{A} is benzyl, phenethyl, or 3-CF₃-benzyl.

In other aspects, provided herein is a compound having Structure (II):

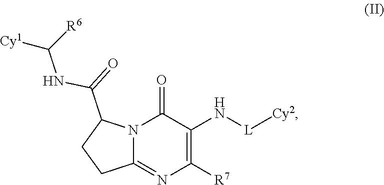

or a stereoisomer, tautomer, or pharmaceutically acceptable salt thereof.

In yet other aspects, provided herein is a compound having Structure (III):

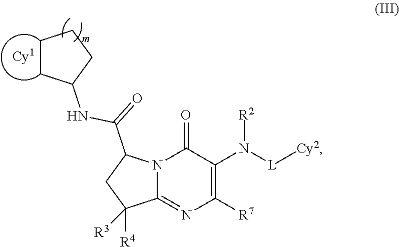

or a stereoisomer, tautomer, or pharmaceutically acceptable salt thereof, wherein:
Cy¹ is a substituted or unsubstituted aryl, or a substituted or unsubstituted 5-10-membered heteroaryl;
Cy² is a substituted aryl, a substituted or unsubstituted C₃-C₆ cycloalkyl, or a substituted or unsubstituted 5-10-membered heteroaryl;
R² is hydrogen, a substituted or unsubstituted C₁-C₃ alkyl, or a substituted or unsubstituted C₃-C₆ cycloalkyl;
R³ and R⁴ are each independently hydrogen, halogen, substituted or unsubstituted C₁-C₃ alkyl, or substituted or unsubstituted cycloalkyl, or R³ and R⁴, together with the carbon to which they are attached, form a substituted or unsubstituted C₃-C₆ cycloalkyl or a substituted or unsubstituted 5-6-membered cycloalkenyl;
R⁷ is selected from the group consisting of hydrogen, halogen, substituted or unsubstituted C₁-C₃ alkyl, and substituted or unsubstituted C₃-C₆ cycloalkyl;
L is absent or —(CR^{8a}R^{8b})ₙ—, wherein each —(CR^{8a}R^{8b})— is independently the same or different;
R^{8a} and R^{8b} are each independently hydrogen, substituted or unsubstituted C₁-C₃ alkyl, or R^{8a} and R^{8b}, together with the carbon to which they are attached, form a substituted or unsubstituted C₃-C₆ cycloalkyl;
m is 1 or 2; and
n is 0, 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms is optionally replaced with a deuterium atom.

In some embodiments, provided herein is a pharmaceutical composition, comprising a compound of any one of Structures (I), (II), or (III), or a stereoisomer, tautomer, or pharmaceutically acceptable salt thereof, and a pharmaceutically acceptable carrier or excipient.

In some embodiments, provided herein is a method for inhibiting MASP-2 in a subject, comprising administering to the subject a compound of Structures (I), (II), or (III), or a stereoisomer, tautomer, or pharmaceutically acceptable salt thereof, in an amount effective to inhibit MASP-2.

In some embodiments, provided herein is a method for treating a disease or disorder treatable by inhibiting MASP-2, comprising administering to a subject in need thereof a therapeutically effective amount of a compound of Structures (I), (II), or (III), or a stereoisomer, tautomer, or pharmaceutically acceptable salt thereof.

In some embodiments, provided herein is a method of inhibiting MASP-2 dependent complement activation in a subject.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the subject matter of the present disclosure, suitable methods and materials are described below. In addition, the materials, methods, and examples are illustrative only and not intended to be limiting.

### I. Definitions

In certain embodiments herein, reference is made to features and aspects of the disclosure, including method steps. All possible combinations of such features and aspects within the embodiments of the disclosure are included, at least to the extent that such combinations are non-contradictory. For example, if an embodiment presents aspects A, B, and C, it is understood that this also discloses embodiments including both aspects A and B, both aspects B and C, and both aspects A and C, as well as an embodiment with aspects A, B, and C.

The terms “a,” “an,” or “the” not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art.

The terms “about” and “approximately” refer to an acceptable degree of error for the quantity measured given the nature or precision of the measurements. Typical, exemplary degrees of error are within +20 percent (%); preferably, within +10%; and more preferably, within +5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written support for a claim limitation of, e.g., “0.98X.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11 mg/kg” is equivalent to “about 7, about 9, or about 11 mg/kg.”

The term “essentially” refers to completely or nearly completely. For example, an essentially pure composition is a composition which is about 100% pure, at least about 99% pure, at least about 98% pure, or at least about 97% pure.

The term “or” refers to an alternative and should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The group “A or B” is equivalent to the group “selected from the group consisting of A and B.”

The linking term “comprising” or “comprise” is not closed. For example, “a composition comprising A” must include at least the component A, but it may also include one or more other components (e.g., B; B and C; B, C, and D; and the like). The term “comprising” therefore should in general be construed as not excluding additional ingredients. For example, a claim to “a composition comprising A” would cover compositions that include A and B; A, B, and C; A, B, C, and D; A, B, C, D, and E; and the like.

The term “MASP-2” refers to mannan-binding lectin-associated serine protease-2. For example, human MASP-2 protein can have UniProt accession code O00187 (SEQ ID NO:1). The Serine Protease Domain (‘B-chain’=Mannan-binding lectin serine protease 2 B chain, based on UniProtKB—O00187 (MASP-2_HUMAN)) includes residues 445 to 686 (or consists of residues 445 to 686).

The term “MASP-2-dependent complement activation” refers to MASP-2-dependent activation of the lectin pathway, which occurs under physiological conditions (i.e., in the presence of Ca⁺⁺) leading to the formation of the lectin pathway C3 convertase C4b2a and upon accumulation of the C3 cleavage product C3b subsequently to the C5 convertase C4b2a(C3b)n.

The term “MASP-2-dependent complement-associated disease or disorder” refers to a disease or disorder that is associated with MASP-2-dependent complement activation.

The term “MASP-2-associated disease or disorder” refers to a disease or disorder that is associated with activation or activity of MASP-2, including MASP-2-dependent complement-associated disease or disorders, and wherein inhibition of MASP-2 is, or is expected to be, therapeutically beneficial.

Typically, the active site of serine proteases, such as MASP-2, is shaped as a cleft where the substrate or inhibitor binds.

The term “lectin pathway” refers to complement activation that occurs via the specific binding of serum and non-serum carbohydrate-binding proteins including mannan-binding lectin (MBL), CL-11, and the ficolins (e.g., H-ficolin, M-ficolin, or L-ficolin).

The term “a subject” includes all mammals, including without limitation, humans, non-human primates, dogs, cats, horses, sheep, goats, cows, rabbits, pigs, and rodents.

“Mammal” includes humans; domestic animals such as laboratory animals and household pets (e.g., cats, dogs, swine, cattle, sheep, goats, horses, rabbits); and non-domestic animals such as wildlife, and the like.

As used herein, the terms “disease,” “condition,” and “disorder” may be used interchangeably or may be different in that the particular malady, condition, disorder, or syndrome may not have a known causative agent (so that etiology has not yet been worked out) and it is therefore not yet recognized as a disease but only as an undesirable condition, disorder, or syndrome, or is a disruption of normal processes or functions, wherein a more or less specific set of symptoms has been identified by a clinician or researcher. In some embodiments, a disease is a pathological condition of an organ, a body part, or a system, resulting from various causes such as infection, genetic defect, or environmental stress that is characterized by an identifiable group of symptoms.

“Therapeutically effective amount” or “effective amount” refers to the amount of a compound of the disclosure that, when administered to a mammal (e.g., a human), is sufficient to effect treatment as defined herein, reduction in symptoms, or cure, of a disease or condition in the mammal, preferably a human. The amount of a compound of the disclosure which constitutes a “therapeutically effective amount” will vary depending on the compound; the condition and its severity; the manner of administration; and the age, weight, and genetics of the mammal to be treated, but can be determined routinely by one of ordinary skill in the art having regard to his own knowledge and to this disclosure.

The term “agent” refers to a compound or mixture of compounds that, when added to a composition, tends to produce an effect on the composition's properties. For example, a composition comprising a thickening agent is likely to be more viscous than an otherwise identical comparative composition that lacks the thickening agent.

A “synthetic” compound means a compound that is not naturally occurring or that has been synthesized by humans. Reference to a compound herein may be understood to include reference to synthetic compounds, unless the context indicates otherwise.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

At various places in the present specification, certain features of the compounds are disclosed in groups or in ranges. It is specifically intended that such a disclosure include each and every individual sub-combination of the members of such groups and ranges. For example, the terms “C₁₋₆ alkyl” and “C₁-C₆ alkyl” are specifically intended to individually disclose (without limitation) methyl, ethyl, C₃ alkyl, C₄ alkyl, C₅ alkyl, and C₆ alkyl, including all linear and branched compositions (e.g., n-butyl, sec-butyl, and tert-butyl for C₄ alkyl).

The term “substituted” means that an atom or group of atoms formally replaces hydrogen as a “substituent” attached to another group. The term “substituted” means that at least one hydrogen atom is replaced with a non-hydrogen substituent. Additionally, a compound can be substituted with a hydrogen, and hydrogen can be a substituent. The term “substituted,” unless otherwise indicated, refers to any level of substitution, e.g., mono-, di-, tri-, tetra-, penta-, or higher substitution, where such substitution is permitted. The substituents are independently selected, and substitution may be at any chemically accessible position. It is to be understood that substitution at a given atom is limited by valency. A single divalent substituent, e.g., oxo, can replace two hydrogen atoms.

The phrase “optionally substituted” means substituted or unsubstituted.

The terms “Cₙ₋ₘ” and “Cₙ-Cₘ,” where n and m are integers, indicates a group that contains from n to m carbon atoms, includes both linear and branched configurations, and does not exclude substituents. Examples include C₁₋₄, C₁₋₆, and the like. The term is intended to expressly disclose every member in the range, i.e., Cₙ, Cₙ₊₁, Cₙ₊₂ . . . Cₘ₋₂, Cₘ₋₁, Cₘ. For example, C₁₋₆ is intended to disclose C₁, C₂, C₃, C₄, C₅, and C₆. As used herein, “Cₙ₋ₘ” means the same as “Cₙ-Cₘ.”

The term “n-membered,” where n is an integer (e.g., 6-membered), typically describes the number of ring-forming atoms in a moiety where the number of ring-forming atoms is n. The term “n-m membered” wherein n and m are integers (e.g., 6-10 membered) describes a range wherein the number of ring forming atoms is from n to m. For example, piperidinyl is an example of a 6-membered heterocyclyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

“Alkyl” refers to a straight or branched hydrocarbon group consisting solely of carbon and hydrogen atoms, containing no unsaturation, having from one to twelve carbon atoms, preferably one to eight carbon atoms, more preferably one to six carbon atoms, and which is attached to the molecule by a single bond. The alkyl group may optionally contain one or more heteroatoms, wherein a carbon atom of the alkyl group is replaced with a heteroatom selected from oxygen, nitrogen or sulfur. An alkyl group is an alkane with one C—H bond replaced by the point of attachment of the alkyl group to the remainder of the molecule. Alkyl groups can be linear or branched. For example, representative alkyl groups can be methyl, ethyl, n-propyl, 1-methylethyl (iso-propyl), n-butyl, 1,1-dimethylethyl (t-butyl), sec-butyl, isobutyl, n-pentyl, 3-methylhexyl, 2-methylhexyl, and the like. In another example, C₁-C₃ alkyl means methyl, ethyl, n-propyl, and isopropyl. In certain specific embodiments, an alkyl group may be optionally substituted.

“Alkenyl” refers to a straight or branched hydrocarbon group consisting solely of carbon and hydrogen atoms, comprising one or more carbon-carbon double bonds, having from two to twelve carbon atoms, preferably two to eight carbon atoms, and which is attached to the molecule by a single bond. An alkenyl group is an alkene with one C—H bond replaced by the point of attachment of the alkenyl group to the remainder of the molecule. For example, representative alkenyl groups can be ethenyl, prop-1-enyl, but-1-enyl, pent-1-enyl, penta-1,4-dienyl, and the like. In certain embodiments, an alkenyl group may be optionally substituted.

“Alkynyl” refers to a straight-chain or branched hydrocarbon group consisting solely of carbon and hydrogen atoms, comprising one or more carbon-carbon triple bonds, having from two to twelve carbon atoms, preferably two to eight carbon atoms, and which is attached to the molecule by a single bond. An alkynyl group is an alkyne with one C—H bond replaced by the point of attachment of the alkynyl group to the remainder of the molecule. The term “Cₙ₋ₘ alkynyl” and “Cₙ-Cₘ alkynyl” refer to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms. In certain embodiments, an alkynyl group may be optionally substituted.

“Alkylene” or “alkylene chain” refers to a straight or branched divalent hydrocarbon chain linking the molecule to another group, or linking two parts of the molecule, and consists solely of carbon and hydrogen, contains no unsaturation, and has from one to twelve carbon atoms, e.g., methylene, ethylene, propylene, n-butylene, and the like. The alkylene chain may optionally contain one or more heteroatoms, wherein a carbon atom of the alkylene chain is replaced with a heteroatom selected from oxygen, nitrogen or sulfur. The alkylene chain is attached to the molecule through a single bond and to the other group through a different single bond, or is attached to two parts of the molecule through a single bond at each point of attachment. In some embodiments, an alkylene group may be optionally substituted with one or more substituents.

The term “heteroalkyl” refers to a 3- to 18-membered non-aromatic and non-cyclic alkyl group which comprises two to twelve carbon atoms and one to six heteroatoms selected from the group consisting of nitrogen, oxygen, and sulfur. The nitrogen, carbon, or sulfur atoms in the heteroalkyl group may be optionally oxidized; the nitrogen atom may be optionally quaternized; and the heteroalkyl group may be saturated or unsaturated (e.g., the heteroalkyl comprises one or more double bond, and which can alternatively be termed a “heteroalkenyl”).

The term “hydroxyalkyl” refers to an alkyl group as defined herein, and in which one or more of the hydrogen atoms has been replaced by a hydroxy group (i.e., —OH). The term “Cₙ₋ₘ hydroxyalkyl” refers to a Cₙ₋ₘ alkyl group having n to m carbon atoms and at least one hydroxy group. In some embodiments, the hydroxyalkyl group comprises one hydroxy group. In some embodiments, the hydroxyalkyl group comprises two or more hydroxy groups (e.g., a “dihydroxyalkyl”), wherein the hydroxy groups are bound to the same or different carbon atom(s). In certain aspects, the hydroxyalkyl group has 1, 2, 3, 4, 5, 6, or more hydroxy groups. Examples may include, but are not limited to, hydroxymethyl, 2-hydroxyethyl, and 1-hydroxyethyl.

“Alkoxy” refers to a group having the following formula “—O-alkyl,” wherein the alkyl group is as defined herein. Example alkoxy groups include methoxy, ethoxy, propoxy (e.g., n-propoxy and isopropoxy), 1-butoxy, and the like. In some embodiments, the alkyl group has 1 to 6, 1 to 4, or 1 to 3 carbon atoms. Unless indicated otherwise, alkoxy groups are optionally substituted with one or more substituents, which can be the same or can be different.

“Aminylalkyl” refers to an alkyl group as defined above, and in which one or more hydrogen atoms have been replaced by an aminyl group, or amino group (i.e., —NRR′ wherein R and R′ are each independently hydrogen, alkyl, alkenyl, or alkynyl, as defined herein). In some embodiments, the aminylalkyl comprises one aminyl group. In some embodiments, the aminyl group is —NH₂.

“Aryl” refers to a hydrocarbon ring system comprising hydrogen, 6 to 18 carbon atoms, and at least one aromatic ring. For purposes of this disclosure, the aryl group may be a monocyclic, bicyclic, tricyclic or tetracyclic ring system, which may include fused or bridged ring systems. Aryl groups include, but are not limited to, aryl groups derived from phenyl, benzene, naphthalene, anthracene, aceanthrylene, acenaphthylene, acephenanthrylene, azulene, chrysene, fluoranthene, fluorene, as-indacene, s-indacene, indane, indene, phenalene, phenanthrene, pleiadene, pyrene, and triphenylene. In some embodiments, an aryl group may be unsubstituted or substituted. In embodiments wherein the aryl can be substituted, the aryl is substituted with one or more substituents, one substituent, two substituents, three substituents, 4 substituents, or five substituents, each substituent of which can be the same or can be different. In some embodiments, the term “aryl,” when substituted, can include a fused ring substituent. e.g., a structure such as the following:

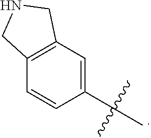 [SMILES: *c1ccc2c(c1)CNC2]

“Arylalkyl” refers to a group of -alkylene-aryl wherein the alkylene group and aryl groups are as defined herein, respectively. In some embodiments, arylalkyl is —C₁₋₃ alkyl-C₆₋₁₀ aryl. In some embodiments, arylalkyl is —C₁₋₄ alkyl-C₆₋₁₀ aryl. In some embodiments, arylalkyl is —C₁₋₄ alkyl-phenyl. Examples include, but are not limited to, benzyl, 1-phenylethyl, 4-methylbenzyl, and 1,1,-dimethyl-1-phenylmethyl. In some embodiments, arylalkyl is optionally substituted on either the alkyl group or the aryl group, with one or more substituents which are the same or are different. In some embodiments, an arylalkyl is an optionally substituted benzyl. In some embodiments an arylalkyl group has the following structure:

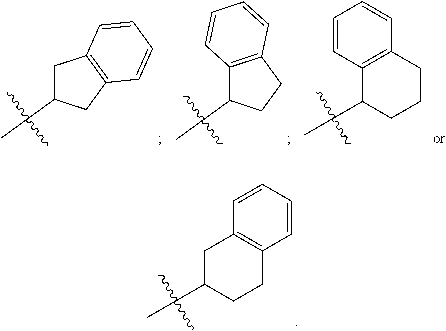 [SMILES: *C1Cc2ccccc2C1 ; *C1CCc2ccccc21 ; *C1CCCc2ccccc21 ; *C1CCc2ccccc2C1]

“Aryloxy” refers to a group with the formula —O-aryl, wherein the aryl is a group as herein defined. In some embodiments, the aryloxy group is —O—C₆₋₁₀ aryl. In some embodiments, the aryloxy is a substituted or unsubstituted phenyloxy (i.e., —O—C₆ aryl).

“Arylalkoxy” refers to a group with the formula -alkoxy-aryl, or —O-alkylene-aryl, wherein alkoxy and aryl are groups as defined herein. In some embodiments, arylalkoxy is —C₁₋₃ alkoxy-C₆₋₁₀ aryl. In some embodiments, arylalkoxy is —C₁₋₄ alkoxy-C₆₋₁₀ aryl. In some embodiments, arylalkoxy is —C₁₋₃ alkoxy-phenyl (e.g., —O-benzyl).

“Cycloalkyl” refers to a non-aromatic monocyclic or polycyclic hydrocarbon consisting solely of carbon and hydrogen atoms, which may include fused or bridged ring systems, having from three to fifteen carbon atoms, preferably having from three to ten carbon atoms, which is saturated or unsaturated, and which is attached to the rest of the molecule by a single bond. Monocyclic groups include, for example, cyclopropyl, cyclobutyl, cyclopentyl, cyclopentenyl, cyclohexyl, cyclohexenyl, cycloheptyl, and cyclooctyl. Polycyclic groups include, for example, adamantyl, norbornyl, decalinyl, and the like. In some embodiments, a cycloalkyl group may be optionally substituted by one or more substituents, wherein the one or more substituents are the same or are different.

“Oxo” refers to a ═O group. For example, an oxo connected to a carbon atom forms a carbonyl group (i.e., C—O). Alternatively, when an oxo group is attached to a heteroatom, for example, a sulfoxide, sulfone group, or N-oxide group is formed.

“Sulfido” refers to a ═S group.

“Amino” refers to a —NH₂ group.

“Acyl” refers to a —C(O)R group, wherein the R group can be an alkyl or aryl, as defined herein. “Acetyl” is an example of an acyl group wherein the R group is methyl.

“Heteroacyl” refers to a —C(O)R group, wherein the R group can be an alkyl or aryl, as defined herein, and further comprising a heteroatom such as one or more of N, S, and O.

“Alkylsulfonyl” refers to —SO₂R, wherein the R group is an alkyl group.

“Methanesulfonyl” is an example of an alkyl sulfonyl wherein the R group is methyl.

“Carboxy” refers to a —C(O)OH group.

“Carbonyl” refers to a C(═O) group, which also may be written as C(O).

“Cyano” or “nitrile” refers to a —C═N group, which also may be written as —CN.

“Hydroxy” or “hydroxyl” refers to an —OH group.

“Halo” or “halogen” refers to all of fluoro, chloro, bromo, and iodo, or to a subset of the halogen atoms (e.g., chloro, bromo, and iodo; chloro and bromo; or only chloro; etc.).

“Haloalkyl” refers to an alkyl group, as defined herein, wherein a hydrogen is substituted by one or more halo groups, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl group may be optionally substituted with one or more substituents, wherein the one or more substituents are independently the same or different.

The term “haloalkoxy,” refers to a group of formula —O-haloalkyl, wherein the haloalkyl group is as defined herein. Example haloalkoxy groups include trifluoromethoxy, difluoromethoxy, pentafluoroethoxy, and the like.

“Heterocyclyl” refers to a 3- to 18-membered non-aromatic ring which comprises two to twelve carbon atoms and one to six heteroatoms selected from the group consisting of nitrogen, oxygen, and sulfur. Unless stated otherwise, the heterocyclyl may be a monocyclic, bicyclic, tricyclic, or tetracyclic ring system, which may include fused, bridged, and spiro ring systems; the nitrogen, carbon, or sulfur atoms in the heterocyclyl group may be optionally oxidized; the nitrogen atom may be optionally quaternized; and the heterocyclyl group may be saturated or unsaturated (e.g., the heterocyclyl comprises one or more double bond, and which can alternatively be termed a “heterocycloalkenyl”). Examples of heterocyclyl groups include, but are not limited to, azetidinyl, 3-azabicyclo[3.1.0]hexan-3-yl, 1-azaspiro[3.3]heptan-1-yl, 5-azaspiro[2.3]hexan-5-yl, 2-oxa-6-azaspiro[3.3]heptan-6-yl, 1-oxa-6-azaspiro[3.4]octan-6-yl, 1-oxa-6-azaspiro[3.3]heptan-6-yl, 6-oxa-1-azaspiro-[3.3]heptan-1-yl, 6-azaspiro[3.4]octan-6-yl, 7-oxa-2-azaspiro[3.5]nonan-2-yl, 2,6-diazaspiro[3.3]heptan-2-yl, dioxolanyl, dioxinyl, thienyl[1,3]dithianyl, decahydro-isoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, 1,2,4-thiadiazol-5(4H)-ylidene, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. In certain embodiments, a heterocyclyl group may be optionally substituted with one or more substituents, wherein the one or more substituents can independently be the same or different.

“Heteroaryl” refers to a 5- to 14-membered ring system comprising hydrogen atoms, one to thirteen carbon atoms, one to six heteroatoms selected from the group consisting of nitrogen, oxygen, and sulfur, and at least one aromatic ring. For purposes of this disclosure, the heteroaryl may be a monocyclic, bicyclic, tricyclic, or tetracyclic ring system, which may include fused or bridged ring systems; the nitrogen, carbon, or sulfur atoms in the heteroaryl may be optionally oxidized; and the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). In some embodiments, the heteroaryl group can be unsubstituted or substituted. In embodiments wherein the heteroaryl is substituted, the heteroaryl is substituted with one or more substituents, each of which can be the same or can be different. In some embodiments, the term “heteroaryl” can include, e.g., structures such as the following:

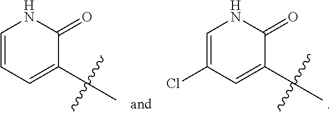 [SMILES: *c1ccc[nH]c1=O ; *c1cc(Cl)c[nH]c1=O]

“Heteroarylalkyl” refers to a group of the formula —R¹⁰⁰R¹⁰¹ where R¹⁰⁰ is an alkylene as defined herein, and R¹⁰¹ is a heteroaryl as defined herein. When specifically stated in the specification, the heteroaryl moiety of the heteroarylalkyl may be optionally substituted as defined herein. In some embodiments, the alkylene moiety of the heteroarylalkyl may be optionally substituted as defined herein.

The compounds and methods of the present disclosure are intended to encompass all pharmaceutically acceptable isotopically labelled compounds of Structure (I), having one or more atoms replaced by an atom having a different atomic mass or mass number.

Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵, ¹⁷O, ¹⁸O, ³¹P, ³²P, ³⁵S, ¹⁸F, ³⁶Cl, ¹²³I, and ¹²⁵I, respectively. These radio-labelled compounds could be useful to help determine or measure the effectiveness of the compounds, by characterizing, for example, the site or mode of action or binding affinity. Certain isotopically labelled compounds of Structure (I), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with isotopes such as deuterium, i.e., ²H, may afford certain therapeutic advantages resulting from greater metabolic stability, for example, increased in vivo half-life or reduced dosage requirements, and hence may be preferred in some circumstances. In some embodiments, the compounds of the disclosure are enriched with deuterium. Such deuterated compounds can be achieved by methods known to one skilled in the art, such as exchanging protons with deuterium, or by synthesizing the molecule with deuterium-enriched starting materials.

Substitution with positron emitting isotopes, such as ¹¹C, ¹⁸F, ¹⁵O, and ¹³N, can be useful in Positron Emission Topography (PET) studies for examining substrate receptor occupancy. Isotopically labeled compounds of Structure (I) can generally be prepared by conventional techniques known to those skilled in the art, or by processes analogous to those described in the Examples as set out below using an appropriate isotopically labeled reagent in place of the non-labeled reagent.

“Substituted” refers to a group in which one or more hydrogens are optionally replaced by a non-hydrogen group to the extent that such substitution is chemically possible. Typical substituents include, but are not limited to, halogens (F, Cl, Br, I), ═O, ═N—CN, ═N—OR, ═NR, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, and NO₂, wherein each R is independently H, C₁-C₆ alkyl, C₂-C₈ heteroalkyl, C₁-C₆ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halogens (F, Cl, Br, I), ═O, ═N—CN, ═N—OR′, ═NR′, OR′, NR′₂, SiR′₃, SR′, SO₂R′, SO₂NR′₂, NR′SO₂R′, NR′CONR′2, NR′C(O)OR′, NR′C(O)R′, CN, C(O)OR′, C(O)NR′₂, OC(O)R′, C(O)R′, and NO₂, wherein each R′ is independently H, C₁-C₆ alkyl, C₂-C₈ heteroalkyl, C₁-C₈ acyl, C₂-C₈ heteroacyl, C₆-C₁₀ aryl, or C₅-C₁₀ heteroaryl. Alkyl, alkenyl, and alkynyl groups can also be substituted by C₁-C₆ acyl, C₂-C₈ heteroacyl, C₆-C₁₀ aryl, or C₅-C₁₀ heteroaryl, each of which can be substituted by substituents appropriate for the particular group. The substituents on an aryl or heteroaryl group can be further substituted with the suitable groups described herein. Thus, for example, an arylalkyl substituent may be substituted on the aryl moiety with substituents described herein for aryl groups. An arylalkyl substituent may alternatively be substituted on the alkyl moiety with substituents described herein for alkyl groups. An arylalkyl substituent may also be substituted on both the alkyl and aryl moieties.

“Optional” or “optionally” means that the subsequently described event of circumstances may or may not occur, and that the description includes instances where said event or circumstance occurs and instances in which it said event or circumstance does not occur. For example, “optionally substituted aryl” or “substituted or unsubstituted aryl” means that the aryl may or may not be substituted, and that the description includes both substituted aryl groups and aryl groups lacking substitution (“unsubstituted”).

When a functional group is described as “optionally substituted,” and in turn, substituents on the functional group are also “optionally substituted” and so on, for the purposes of this disclosure, such iterations are limited to five. Such iterations are preferably limited to two.

“Optionally substituted,” as used herein, indicates that the particular group being described may have one, or more than one, hydrogen atoms replaced by a non-hydrogen group. In some optionally substituted groups, one hydrogen atom is replaced by a non-hydrogen group, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, and NO₂, wherein each R is independently H, C₁-C₆ alkyl, 2-6-membered heteroalkyl, C₆-C₁₀ aryl, 5-9-membered heteroaryl, or as otherwise disclosed herein. In some optionally substituted groups, more than one hydrogen atom is replaced by one or more of the same or different non-hydrogen substituent, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, and NO₂, wherein each R is independently H, C₁-C₆ alkyl, 2-6-membered heteroalkyl, C₆-C₁₀ aryl, 5-9-membered heteroaryl, or as otherwise disclosed herein. In some optionally substituted groups, all hydrogen atoms are replaced by the same or different non-hydrogen substituents, e.g., C₁-C₆ alkyl, C₂-C₆ heteroalkyl, alkynyl, halogens (F, Cl, Br, I), N₃, OR, NR₂, SiR₃, SR, SO₂R, SO₂NR₂, NRSO₂R, NRCONR₂, NRC(O)OR, NRC(O)R, CN, C(O)OR, C(O)NR₂, OC(O)R, C(O)R, oxo, and NO₂, wherein each R is independently H, C₁-C₆ alkyl, 2-6-membered heteroalkyl, C₆-C₁₀ aryl, 5-9-membered heteroaryl, or as otherwise disclosed herein. In some optionally substituted groups, one hydrogen atom, more than one hydrogen atom, or all hydrogen atoms, are replaced by a deuterium atom. Where an optional substituent is attached via a double bond, such as a carbonyl oxygen or oxo (═O), the substituent group takes up two available valence positions, so the total number of substituents that may be included is reduced according to the number of available valence positions.

“Pharmaceutically acceptable carrier,” “pharmaceutically acceptable excipient,” or “pharmaceutically acceptable carrier or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, pH adjusting agent, hydrogel, salt, inert solid, printed solid, or emulsifier, which has been approved by the United States Food and Drug Administration as being acceptable for use in humans or domestic animals.

“Pharmaceutically acceptable salt” includes both acid and base addition salts.

A pharmaceutically acceptable acid addition salt refers to salts which retain the biological effectiveness and properties of the free bases, which are not biologically, or otherwise, undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

A pharmaceutically acceptable base addition salt refers to salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts, and the like. Preferred inorganic salts are ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like. Particularly preferred organic bases are isopropylamine, diethylamine, ethanolamine, trimethylamine, dicyclohexylamine, choline, and caffeine.

A “pharmaceutical composition” refers to a formulation of a compound of the disclosure and a medium generally accepted in the art for the delivery of the compound to mammals, e.g., humans. Such a medium includes all pharmaceutically acceptable carriers, diluents, or excipients therefor.

“Treating” or “treatment” as used herein covers the treatment of the disease or condition of interest in a mammal, preferably a human, having the disease or condition of interest, and includes:
(a) preventing the disease or condition from occurring in a mammal, in particular, when such mammal is predisposed to the condition but has not yet been diagnosed as having it;
(b) inhibiting the disease or condition, i.e., arresting the disease or condition development;
(c) relieving (or ameliorating) the disease or condition, i.e., causing regression of the disease or condition; or
(d) relieving (or ameliorating) the symptoms resulting from the disease or condition, e.g., without addressing the underlying disease or condition.

A “stereoisomer” refers to a compound made up of the same atoms bonded by the same bonds, but having different three-dimensional structures, which are not interchangeable. The present disclosure contemplates various stereoisomers and mixtures thereof. The present disclosure includes enantiomers, which refers to two stereoisomers whose molecules are non-superimposable mirror images of one another. See, e.g., Smith, M. B. and J. March, March's Advanced Organic Chemistry: Reactions, Mechanisms, and Structure, 6th edition (Wiley, 2007), for a detailed description of the structure and properties of enantiomers and stereoisomers. The present disclosure includes diastereomers, which refers to two stereoisomers whose molecules are non-superimposable non-mirror images of one another.

The present disclosure includes an essentially pure enantiomer or diastereomer. The present disclosure includes mixtures of enantiomers, diastereomers, or a combination thereof. The mixture can be a racemic mixture, as known to one having skill in the art, as a 50/50 mixture of enantiomers. The mixture can also include a mixture of any other ratio or relative composition of enantiomers, diastereomers, or a combination thereof.

The compounds of the disclosure, or their pharmaceutically acceptable salts, may contain one or more stereocenter and may thus give rise to enantiomers, diastereomers, and other stereoisomeric forms that may be defined, in terms of absolute stereochemistry, as (R)- or (S)-, or as (D)- or (L)- for amino acids. The present disclosure is meant to include all such possible isomers, all combinations of such isomers, a racemic mixture, and optically pure forms. Optically active (+) and (−), (R)- and (S)-, or (D)- and (L)-isomers may be prepared using chiral synthons or chiral reagents, or resolved using conventional techniques, for example, chromatography or fractional crystallization. Conventional techniques for the preparation/isolation of individual enantiomers or diastereomers include chiral synthesis from a suitable optically pure precursor, or resolution of the mixture (e.g., racemate or the racemate of a salt or derivative) using, for example, chiral high-performance liquid chromatography (HPLC).

When the compounds described herein contain olefinic double bonds or other centers giving rise to geometric asymmetry, and unless specified otherwise, it is intended that the compounds include both E and Z geometric isomers. Likewise, all tautomeric forms are also intended to be included.

A “tautomer” refers to a constitutional isomer wherein a proton can shift from one atom of a molecule to another atom of the same molecule (e.g., keto-enol). The present disclosure includes tautomers of any said compounds.

The use of parentheses and brackets in substituent groups is used herein to conserve space. Accordingly, the use of parenthesis in a substituent group indicates that the group enclosed within the parentheses is attached directly to the atom preceding the parenthesis (e.g., —C(O)— represents a carbonyl). The use of brackets in a substituent group indicates that the group enclosed within the brackets is also attached directly to the atom preceding the brackets.

The chemical naming protocol and structure diagrams used herein are a modified form of the I.U.P.A.C. nomenclature system, using ChemDraw Professional Version 21.0.0 software program. For complex chemical names employed herein, a substituent group is named before the group to which it attaches. For example, cyclopropylethyl comprises an ethyl backbone with a cyclopropyl substituent. In chemical structure diagrams, all bonds are identified, except for some carbon atoms, which are assumed to be bonded to sufficient hydrogen atoms to complete the valency.

At certain places, the definitions or embodiments may refer to specific rings (e.g., an azetidine ring, a pyridine ring, etc.). Unless otherwise indicated, these rings can be attached to any ring member, provided that the valency of the atom is not exceeded.

When any two groups or two instances of the same substituent group are “independently” selected from a list of alternatives, the groups may be the same, or the groups may be different. For example, if R^{a} and R^{b} are independently selected from the group consisting of alkyl, fluoro, amino, and hydroxyalkyl, then a molecule with two R^{a} groups and two R^{b} groups could have all groups be an alkyl group (e.g., four different alkyl groups, or four of the same alkyl groups). Alternatively, the first R^{a} could be alkyl, the second R^{a} could be fluoro, the first R^{b} could be hydroxyalkyl, and the second R^{b} could be amino (or any other substituents taken from the group). Alternatively, both R^{a} and the first R^{b} could be fluoro, while the second R^{b} could be alkyl (i.e., some pairs of substituent groups may be the same, while other pairs may be different). Unless otherwise indicated, if two or more groups having the same definition are present, but the definition provides for alternatives, it should be understood that each occurrence of the same group is independently selected from the possible alternatives. For example, if two or more R^{a} groups are present in a compound, and the definition of R^{a} provides that R^{a} can be A, B, or C, then it should be understood that each R^{a} group present in the compound is independently chosen from A, B, and C, so that the R^{a} groups present in the compound can be the same or different.

Compounds, and salts thereof, including pharmaceutically acceptable salts, can be found together with other substances such as water and solvents (e.g., hydrates and solvates), or can be isolated.

Compounds, and salts thereof, can additionally include more than one salt form. For example, the salt of a compound having two basic groups can include e.g., two trifluoroacetic acid salts, or one trifluoroacetic acid salt and one hydrochloride salt.

### II. Compounds

In some aspects, the present disclosure provides a compound having Structure (I):

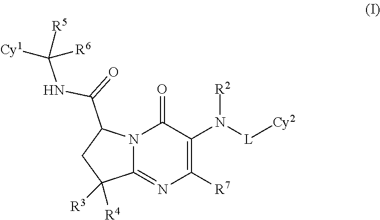

or a stereoisomer, tautomer, or pharmaceutically acceptable salt thereof, wherein:
Cy¹ is a substituted aryl, or a substituted or unsubstituted 5-10-membered heteroaryl, or
Cy¹, together with one of R⁵ or R⁶ and the carbon to which they are attached, forms a substituted or unsubstituted C₃-C₆ cycloalkyl fused to a substituted or unsubstituted 5-10-membered heteroaryl, or a substituted or unsubstituted phenyl;
Cy² is a substituted aryl, a substituted or unsubstituted C₃-C₆ cycloalkyl, a substituted or unsubstituted 5-10-membered heteroaryl, or is hydrogen;
R² is hydrogen, a substituted or unsubstituted C₁-C₃ alkyl, or a substituted or unsubstituted C₃-C₆ cycloalkyl;
R³ and R⁴ are each independently hydrogen, halogen, substituted or unsubstituted C₁-C₃ alkyl, or substituted or unsubstituted C₃-C₆ cycloalkyl, or R³ and R⁴, together with the carbon to which they are attached, form a substituted or unsubstituted C₃-C₆ cycloalkyl or a substituted or unsubstituted C₅-C₆ membered cycloalkenyl;
R⁵ and R⁶ are each independently hydrogen, C₁-C₃ alkyl, alkoxy, haloalkyl, hydroxyalkyl, haloalkoxy, or C₃-C₆ cycloalkyl;
R⁷ is selected from the group consisting of hydrogen, halogen, substituted or unsubstituted C₁-C₃ alkyl, and substituted or unsubstituted C₃-C₆ cycloalkyl;
L is hydrogen or —(CR^{8a}R^{8b})ₙ—, wherein each —(CR^{8a}R^{8b})— is independently the same or different;
R^{8a} and R^{8b} are each independently hydrogen, substituted or unsubstituted linear or branched C₁-C₃ alkyl, or R^{8a} and R^{8b}, together with the carbon to which they are attached, form a substituted or unsubstituted C₃-C₆ cycloalkyl; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms in Structure (I) is optionally replaced with a deuterium atom; and
provided that the compound of Structure (T) does not have the structure:

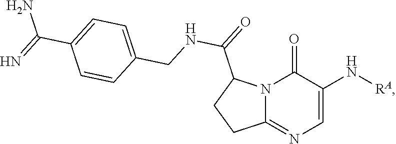

wherein R^{A} is benzyl, phenethyl, or 3-CF₃-benzyl.

In some embodiments, R² is hydrogen.

In some embodiments, R² is a substituted or unsubstituted linear or branched C₁-C₃ alkyl. In embodiments wherein R² is a substituted linear or branched C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R² is methyl. In some embodiments, R² is ethyl.

In some embodiments, R² is a substituted or unsubstituted C₃-C₆ cycloalkyl. In embodiments wherein R² is a substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, R³ and R⁴ are each independently hydrogen, halogen, substituted or unsubstituted C₁-C₃ alkyl, or substituted or unsubstituted C₃-C₆ cycloalkyl.

In some embodiments, R³ is hydrogen, halogen, substituted or unsubstituted C₁-C₃ alkyl, or substituted or unsubstituted C₃-C₆ cycloalkyl. In some embodiments, R³ is hydrogen. In some embodiments, R³ is fluoro, chloro, bromo, or iodo. In some embodiments, R³ is unsubstituted C₁-C₃ alkyl. In some embodiments, R³ is substituted C₁-C₃ alkyl. For example, the C₁-C₃ alkyl is methyl, ethyl, n-propyl, or isopropyl. In embodiments wherein R³ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R³ is unsubstituted C₃-C₆ cycloalkyl. In some embodiments, R³ is substituted C₃-C₆ cycloalkyl. For example, the C₃-C₆ cycloalkyl can be a cyclopropyl, cyclobutyl, cyclopentyl, cyclohexyl, bicyclopentyl, or bicyclohexyl. In embodiments wherein R³ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, R⁴ is hydrogen, halogen, substituted or unsubstituted C₁-C₃ alkyl, or substituted or unsubstituted C₃-C₆ cycloalkyl. In some embodiments, R⁴ is hydrogen. In some embodiments, R⁴ is fluoro, chloro, bromo, or iodo. In some embodiments, R⁴ is unsubstituted C₁-C₃ alkyl. In some embodiments, R⁴ is substituted C₁-C₃ alkyl. For example, the C₁-C₃ alkyl is methyl, ethyl, n-propyl, or isopropyl. In embodiments wherein R⁴ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R⁴ is unsubstituted C₃-C₆ cycloalkyl. In some embodiments, R⁴ is substituted C₃-C₆ cycloalkyl. For example, the C₃-C₆ cycloalkyl can be a cyclopropyl, cyclobutyl, cyclopentyl, cyclohexyl, bicyclopentyl, or bicyclohexyl. In embodiments wherein R⁴ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, R³ and R⁴ are hydrogen.

In some embodiments, R³ and R⁴, together with the carbon to which they are attached, form a substituted or unsubstituted C₃-C₆ cycloalkyl, or a substituted or unsubstituted C₅-C₆ cycloalkenyl. For example, the C₃-C₆ cycloalkyl can be a cyclopropyl, cyclobutyl, cyclopentyl, cyclohexyl, bicyclopentyl, or bicyclohexyl. In another example, the C₅-C₆ cycloalkenyl can be a cyclopentenyl or a cyclohexenyl. In embodiments wherein R³ and R⁴ form a substituted C₃-C₆ cycloalkyl or a substituted C₅-C₆ cycloalkenyl the C₃-C₆ cycloalkyl or C₅-C₆ cycloalkenyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, when a chiral center is present at the carbon atom to which R³ and R⁴ are attached, the compound has (R) stereochemistry, has (S) stereochemistry, is a racemic mixture, or comprises a mixture of (R) and (S) stereoisomers.

In some embodiments, R⁵ and R⁶ are each independently hydrogen, C₁-C₃ alkyl, alkoxy, haloalkyl, hydroxyalkyl, haloalkoxy, or C₃-C₆ cycloalkyl.

In some embodiments, R⁵ is hydrogen, C₁-C₃ alkyl, alkoxy, haloalkyl, hydroxyalkyl, haloalkoxy, or C₃-C₆ cycloalkyl. In some embodiments, R⁵ is hydrogen. In some embodiments, R⁵ is C₁-C₃ alkyl. In some embodiments, R⁵ is methyl, ethyl, n-propyl, or isopropyl. In some embodiments, R⁵ is C₁-C₃ alkoxy. In some embodiments, R⁵ is C₁-C₃ haloalkoxy. For example, the alkoxy and haloalkoxy of R⁵ is methoxy, ethoxy, propoxy, or isopropoxy. In some embodiments, R⁵ is haloalkyl. In embodiments wherein R⁵ is haloalkyl or haloalkoxy, the halo is one or more fluoro, chloro, bromo, or iodo. In some embodiments, R⁵ is C₃-C₆ cycloalkyl. In some embodiments, R⁵ is cyclopropyl, cyclobutyl, cyclopentyl, cyclohexyl, bicyclopentyl, or bicyclohexyl. In some embodiments, R⁵ is C₁-C₃ hydroxyalkyl.

In some embodiments, R⁵ is hydrogen. In some embodiments, R⁵ is methyl.

In some embodiments, R⁶ is hydrogen, C₁-C₃ alkyl, alkoxy, haloalkyl, hydroxyalkyl, haloalkoxy, or C₃-C₆ cycloalkyl. In some embodiments, R⁶ is hydrogen. In some embodiments, R⁶ is C₁-C₃ alkyl. In some embodiments, R⁶ is methyl, ethyl, n-propyl, or isopropyl. In some embodiments, R⁶ is C₁-C₃ alkoxy. In some embodiments, R⁶ is C₁-C₃ haloalkoxy. For example, the alkoxy and haloalkoxy of R⁶ is methoxy, ethoxy, propoxy, or isopropoxy. In some embodiments, R⁶ is haloalkyl. In embodiments wherein R⁶ is haloalkyl or haloalkoxy, the halo is one or more fluoro, chloro, bromo, or iodo. In some embodiments, R⁶ is C₃-C₆ cycloalkyl. In some embodiments, R⁶ is cyclopropyl, cyclobutyl, cyclopentyl, cyclohexyl, or bicyclohexyl. In some embodiments, R⁶ is C₁-C₃ hydroxyalkyl.

In some embodiments, R⁶ is hydrogen. In some embodiments, R⁶ is methyl.

In some embodiments, R⁵ and R⁶ are the same. In some embodiments, R⁵ and R⁶ are different. In some embodiments, R⁵ and R⁶ are both hydrogen. In some embodiments, one of R⁵ and R⁶ is hydrogen. In some embodiments, one of R⁵ and R⁶ is methyl, ethyl, n-propyl, or isopropyl, and the other one of R⁵ and R⁶ is hydrogen, methyl, ethyl, n-propyl, or isopropyl.

In some embodiments, when a chiral center is present at the carbon atom to which R⁵ and R⁶ are attached, the compound has (R) stereochemistry, has (S) stereochemistry, is a racemic mixture, or is a mixture of (R) and (S) stereoisomers.

In some embodiments, R⁷ is selected from the group consisting of hydrogen, halogen, substituted or unsubstituted C₁-C₃ alkyl, and substituted or unsubstituted C₃-C₆ cycloalkyl.

In some embodiments, R⁷ is hydrogen.

In some embodiments, R⁷ is fluoro, chloro, bromo, or iodo.

In some embodiments, R⁷ is unsubstituted C₁-C₃ alkyl. For example, the C₁-C₃ alkyl is methyl, ethyl, n-propyl, or isopropyl.

In some embodiments, R⁷ is substituted C₁-C₃ alkyl. In embodiments wherein R⁷ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, R⁷ is unsubstituted C₃-C₆ cycloalkyl. In some embodiments, R⁷ is substituted C₃-C₆ cycloalkyl. For example, the C₃-C₆ cycloalkyl can be a cyclopropyl, cyclobutyl, cyclopentyl, cyclohexyl, bicyclopentyl, or bicyclohexyl.

In embodiments wherein R⁷ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, R⁷ is selected from the group consisting of hydrogen, deuterium, C₁-C₃ alkyl, and C₁-C₃ haloalkyl.

In some embodiments, the compound of Structure (I) has Structure (I-A):

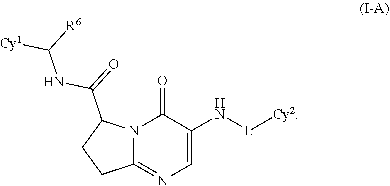

In some embodiments, the compound of Structures (I) and (I-A) has Structure (I-A-1), (I-A-2), (I-A-3), (I-A-4), or combinations thereof:

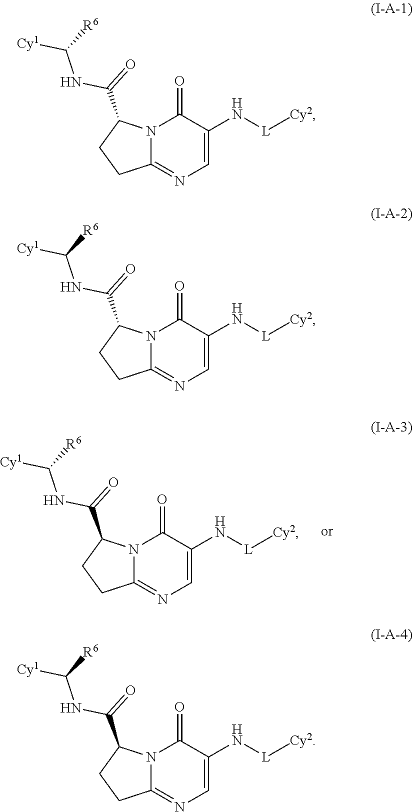

In some embodiments of Structures (I), (I-A), (I-A-1), (I-A-2), (I-A-3), and (I-A-4), R⁶ is hydrogen. In some embodiments of Structures (I-A), (I-A-1), (I-A-2), (I-A-3), and (I-A-4), R⁶ is methyl.

In some embodiments of Structure (I), Cy¹, together with one of R⁵ or R⁶ and the carbon to which they are attached, forms a substituted or unsubstituted C₃-C₆ cycloalkyl fused to a substituted or unsubstituted 5-10-membered heteroaryl, or a substituted or unsubstituted phenyl. In some embodiments, the C₃-C₆ cycloalkyl, of the C₃-C₆ cycloalkyl fused to a 5-10-membered heteroaryl or phenyl, is unsubstituted. In some embodiments, the C₃-C₆ cycloalkyl, of the C₃-C₆ cycloalkyl fused to a 5-10-membered heteroaryl or phenyl, is substituted. In embodiments wherein the C₃-C₆ cycloalkyl is substituted, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (I), Cy¹, together with one of R⁵ or R⁶ and the carbon to which they are attached, forms a substituted or unsubstituted C₅ cycloalkyl fused to a substituted or unsubstituted 5-10-membered heteroaryl, or a substituted or unsubstituted phenyl. In some embodiments, the C₅ cycloalkyl, of the C₅ cycloalkyl fused to a 5-10-membered heteroaryl or phenyl, is unsubstituted. In some embodiments, the C₅ cycloalkyl, of the C₅ cycloalkyl fused to a 5-10-membered heteroaryl or phenyl, is substituted. In embodiments wherein the C₅ cycloalkyl is substituted, the C₅ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (I), Cy¹, together with one of R⁵ or R⁶ and the carbon to which they are attached, forms a substituted or unsubstituted C₆ cycloalkyl fused to a substituted or unsubstituted 5-10-membered heteroaryl, or a substituted or unsubstituted phenyl. In some embodiments, the C₆ cycloalkyl, of the C₆ cycloalkyl fused to a 5-10-membered heteroaryl or phenyl, is unsubstituted. In some embodiments, the C₆ cycloalkyl, of the C₆ cycloalkyl fused to a 5-10-membered heteroaryl or phenyl, is substituted. In embodiments wherein the C₆ cycloalkyl is substituted, the C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (I), Cy¹, together with one of R⁵ or R⁶ and the carbon to which they are attached, forms a substituted or unsubstituted C₃-C₆ cycloalkyl fused to a substituted or unsubstituted 5-6-membered heteroaryl. In some embodiments, the C₃-C₆ cycloalkyl, of the C₃-C₆ cycloalkyl fused to a 5-6-membered heteroaryl, is unsubstituted. In some embodiments, the C₃-C₆ cycloalkyl, of the C₃-C₆ cycloalkyl fused to a 5-6-membered heteroaryl, is substituted. In embodiments wherein the C₃-C₆ cycloalkyl is substituted, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments of Structure (I), Cy¹, together with one of R⁵ or R⁶ and the carbon to which they are attached, forms a substituted or unsubstituted C₅ cycloalkyl fused to a substituted or unsubstituted 5-6-membered heteroaryl. In some embodiments, the C₅ cycloalkyl, of the C₅ cycloalkyl fused to a 5-6-membered heteroaryl, is unsubstituted. In some embodiments, the C₅ cycloalkyl, of the C₅ cycloalkyl fused to a 5-6-membered heteroaryl, is substituted. In embodiments wherein the C₅ cycloalkyl is substituted, the C₅ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (I), Cy¹, together with one of R⁵ or R⁶ and the carbon to which they are attached, forms a substituted or unsubstituted C₆ cycloalkyl fused to a substituted or unsubstituted 5-6-membered heteroaryl. In some embodiments, the C₆ cycloalkyl, of the C₆ cycloalkyl fused to a 5-6-membered heteroaryl, is unsubstituted. In some embodiments, the C₆ cycloalkyl, of the C₆ cycloalkyl fused to a 5-6-membered heteroaryl, is substituted. In embodiments wherein the C₆ cycloalkyl is substituted, the C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (I), Cy¹, together with one of R⁵ or R⁶ and the carbon to which they are attached, forms a substituted or unsubstituted C₅ cycloalkyl fused to a substituted or unsubstituted 6-membered heteroaryl. In some embodiments, the C₅ cycloalkyl, of the C₅ cycloalkyl fused to a 6-membered heteroaryl, is unsubstituted. In some embodiments, the C₅ cycloalkyl, of the C₅ cycloalkyl fused to a 6-membered heteroaryl, is substituted. In embodiments wherein the C₅ cycloalkyl is substituted, the C₅ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments, the compound of Structure (I) has Structure (I-B):

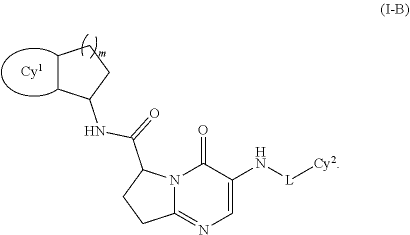

In some embodiments of Structure (I-B), m is 1. In some embodiments of Structure (I-B), m is 2.

In some embodiments, the compound of Structure (I-B) has Structure (I-B-1), (I-B-2), (I-B-3), (I-B-4), or combinations thereof:

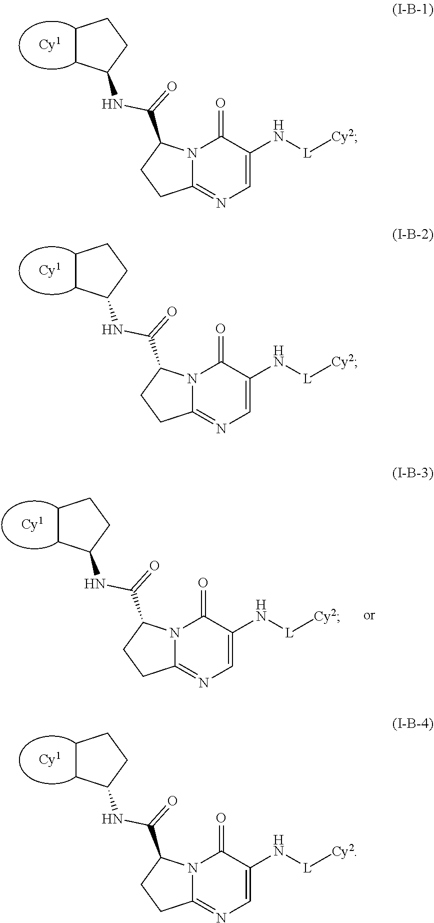

In some embodiments of Structures (I), (I-A), and (I-B), Cy¹ is an unsubstituted C₆-C₁₀ aryl. In some embodiments of Structures (I), (I-A), and (I-B), Cy¹ is an unsubstituted phenyl.

In some embodiments of Structures (I), (I-A), and (I-B), Cy¹ is a substituted C₆-C₁₀ aryl. In some embodiments of Structures (I), (I-A), and (I-B), Cy¹ is a substituted phenyl.

In some embodiments of Structures (I), (I-A), and (I-B), Cy¹ is a C₆-C₁₀ aryl substituted with one or more, 1-5, 1-4, 1-3, one or two, one, two, three, four, or five substituents independently selected from the group consisting of hydrogen, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹⁷, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the C₆-C₁₀ aryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the C₆-C₁₀ aryl,
wherein R⁸, R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (I), (I-A), and (I-B), Cy¹ is a phenyl substituted with one or more, 1-5, 1-4, 1-3, one or two, one, two, three, four, or five substituents independently selected from the group consisting of hydrogen, C(═NH)NHC(═O)OR⁸, C(═NOC(═O)OR⁸)NH₂, C(═NOH)NH₂, C(═NOC(═O)R⁸)NH₂, C(═NH)NHC(═O)NHC(═O)N(CH₃)R¹⁷, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the phenyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl,
wherein R⁸, R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (I), (I-A), and (I-B), Cy¹ is a phenyl substituted with at least one substituent selected from the group consisting of —C(═NH)NH₂, halogen, haloalkyl, aminyl, aminylalkyl, and tetrazole.

In some embodiments of Structures (I), (I-A), and (I-B), Cy¹ is a phenyl substituted with at least one substituent selected from the group consisting of —C(═NH)NH₂, chloro, fluoro, —CHF₂, —NH₂, —CF₃, —CH₂NH₂, —CH(CH₃)NH₂, and

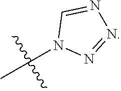 [SMILES: *n1cnnn1]

In some embodiments of Structures (I) and (I-A), Cy¹ is selected from the group consisting of:

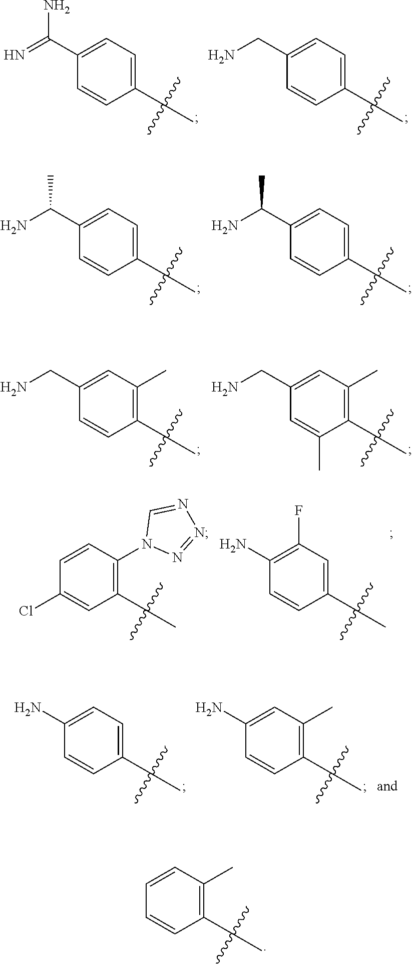

In some embodiments of Structure (I-B), Cy¹ is selected from the group consisting of:

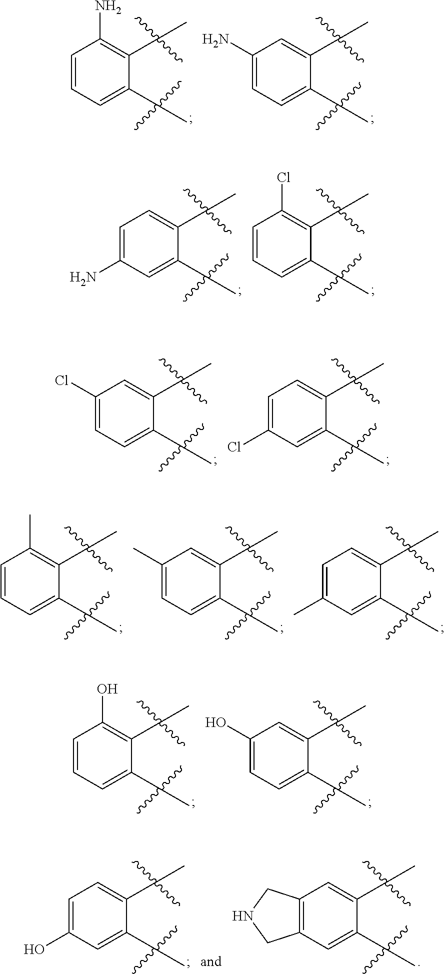

In some embodiments of Structure (I-B), Cy¹ is selected from the group consisting of:

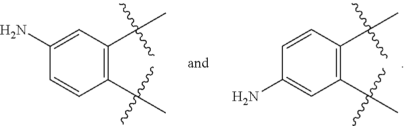 [SMILES: *c1ccc(N)cc1* ; *c1ccc(N)cc1*]

In some embodiments of Structures (I), (I-A), and (I-B), Cy¹ is a substituted or unsubstituted 5-10-membered heteroaryl.

In some embodiments of Structures (I), (I-A), and (I-B), Cy¹ is an unsubstituted 5-10-membered heteroaryl.

In some embodiments of Structures (I), (I-A), and (I-B), Cy¹ is a substituted 5-10-membered heteroaryl.

In some embodiments of Structures (I), (I-A), and (I-B), Cy¹ is a 5-10-membered heteroaryl substituted with one or more, 1-5, 1-4, 1-3, one or two, one, two, three, four, or five substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the heteroaryl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (I), (I-A), and (I-B), Cy¹ is a pyridinyl, pyrrolopyridinyl, imidazopyridinyl, thienopyridinyl, benzoimidazolyl, isoindolinyl, thiophenyl, or benzothiazolyl.

In some embodiments of Structures (I), (I-A), and (I-B), Cy¹ is a substituted or unsubstituted pyridinyl.

In some embodiments of Structures (I), (I-A), and (I-B), Cy¹ is an unsubstituted pyridinyl.

In some embodiments of Structures (I), (I-A), and (I-B), Cy¹ is a substituted pyridinyl. In some embodiments of Structures (I), (I-A), and (I-B), Cy¹ is a pyridinyl substituted with one or more, 1-4, 1-3, one or two, one, two, three, or four substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the pyridinyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the pyridinyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more, 1-4, 1-3, one or two, one, two, three, or four substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-4, 1-3, one or two, one, two, three, or four substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (I), (I-A), and (I-B), Cy¹ is a pyridinyl substituted with one or more hydrogen, deuterium, C₁₋₆ alkyl, C₁₋₆ deuterated alkyl, halogen, aminylalkyl, amino, C₁₋₃ alkoxy, C₁₋₆ haloalkyl, or combinations thereof.

In some embodiments of Structures (I), (I-A), and (I-B), Cy¹ is a pyridinyl substituted with one or more hydrogen, methyl, ethyl, CD₃, amino, aminylmethyl, F, Cl, Br, methoxy, or combinations thereof.

In some embodiments of Structure (I) and (I-A), Cy¹ has a structure selected from the group consisting of:

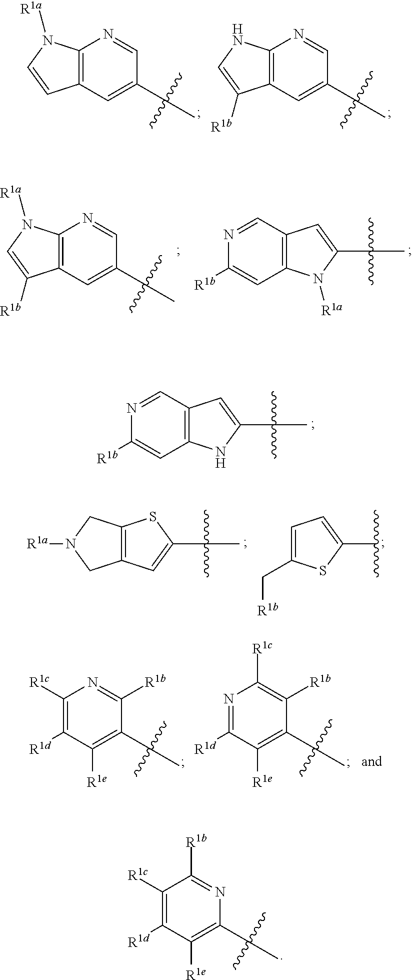

In some embodiments of Structure (I) and Structure (I-A), Cy¹ is:

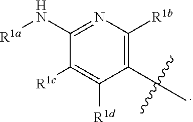

In some embodiments of Structure (I) and Structure (I-A), Cy¹ is:

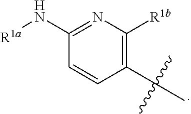

In some embodiments of Structure (I-B), Cy¹ is:

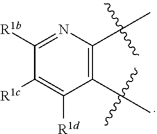

In some embodiments of Structure (I-B), Cy¹ is:

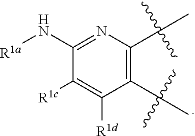

In some embodiments of Structure (I-B), Cy¹ is:

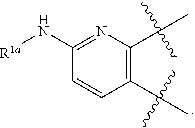

In some embodiments of Structures (I), (I-A), and (I-B), R^{1a} is selected from the group consisting of hydrogen, deuterium, C₁₋₆ alkyl, and C₁₋₆ deuterated alkyl.

In some embodiments of Structures (I), (I-A), and (I-B), R^{1a} is selected from the group consisting of hydrogen, methyl, ethyl, and CD₃.

In some embodiments of Structures (I), (I-A), and (I-B), R^{1a} is hydrogen.

In some embodiments of Structures (I), (I-A), and (I-B), R^{1b}, R^{1c}, R^{1d}, and R^{1e} are selected from the group consisting of hydrogen, deuterium, C₁₋₆ alkyl, C₁₋₆ deuterated alkyl, halogen, aminylalkyl, amino, C₁₋₃ alkoxy, and C₁₋₆ haloalkyl.

In some embodiments of Structures (I), (I-A), and (I-B), R^{1b}, R^{1c}, R^{1d}, and R^{1e} are selected from the group consisting of hydrogen, methyl, ethyl, CD₃, amino, aminylmethyl, F, Cl, Br, and methoxy.

In some embodiments of Structures (I) and (I-A), Cy¹ is selected from the group consisting of:

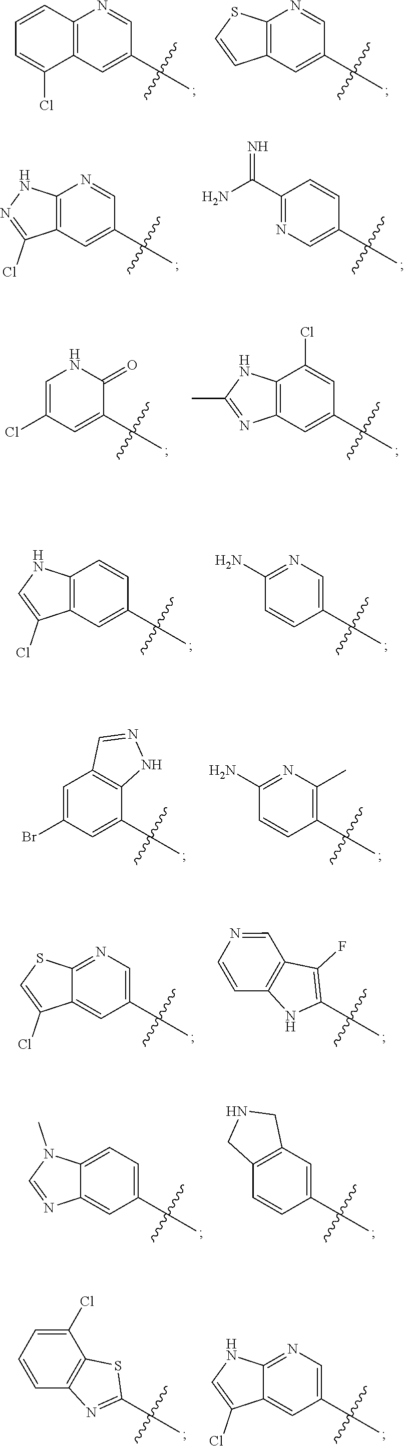

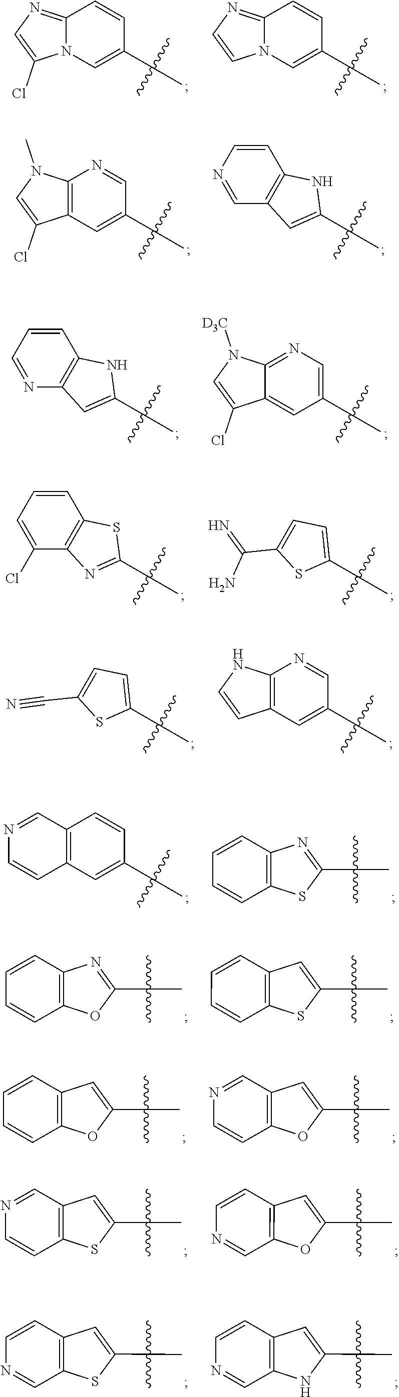

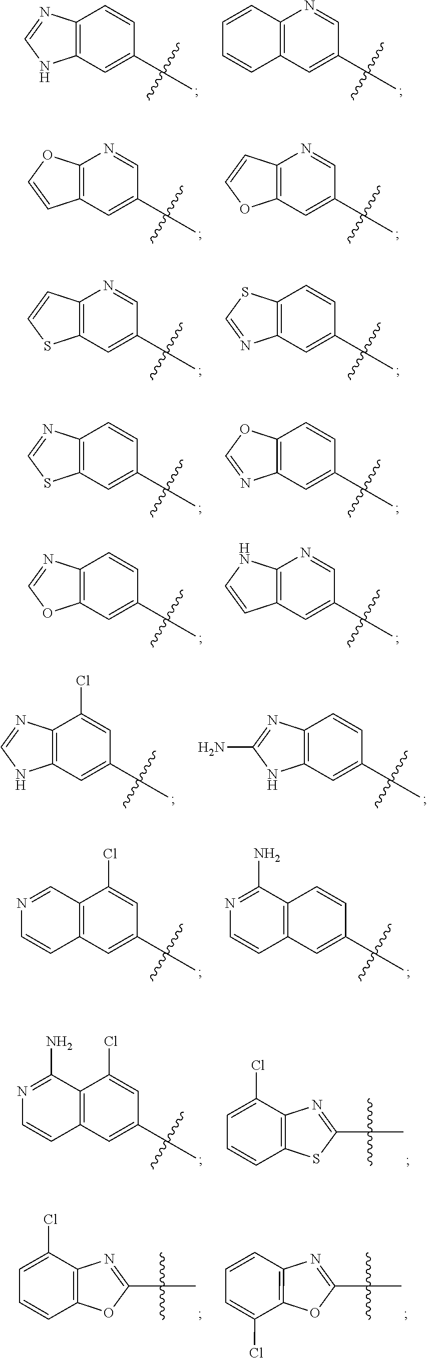

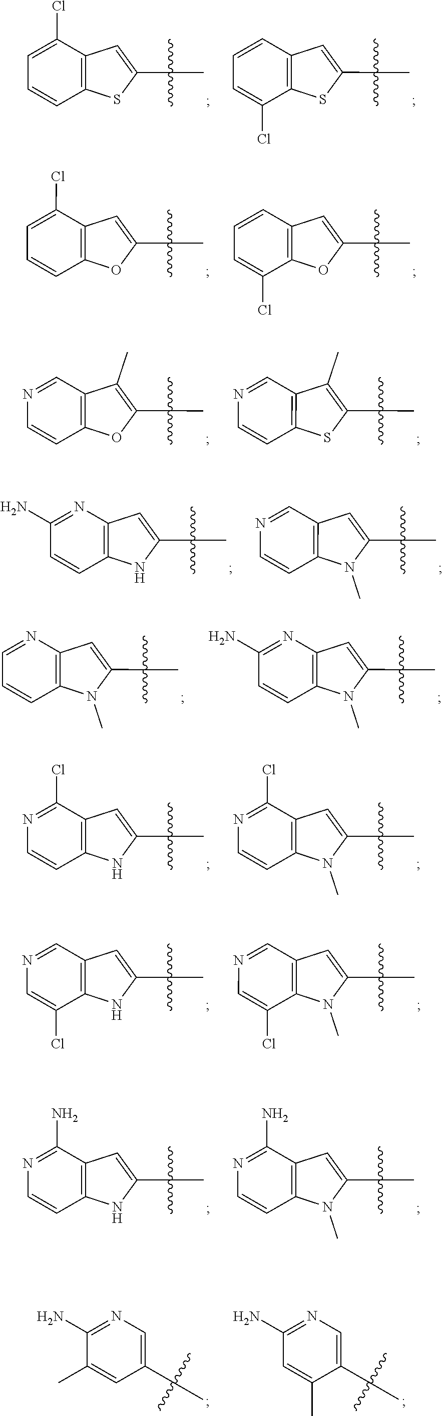

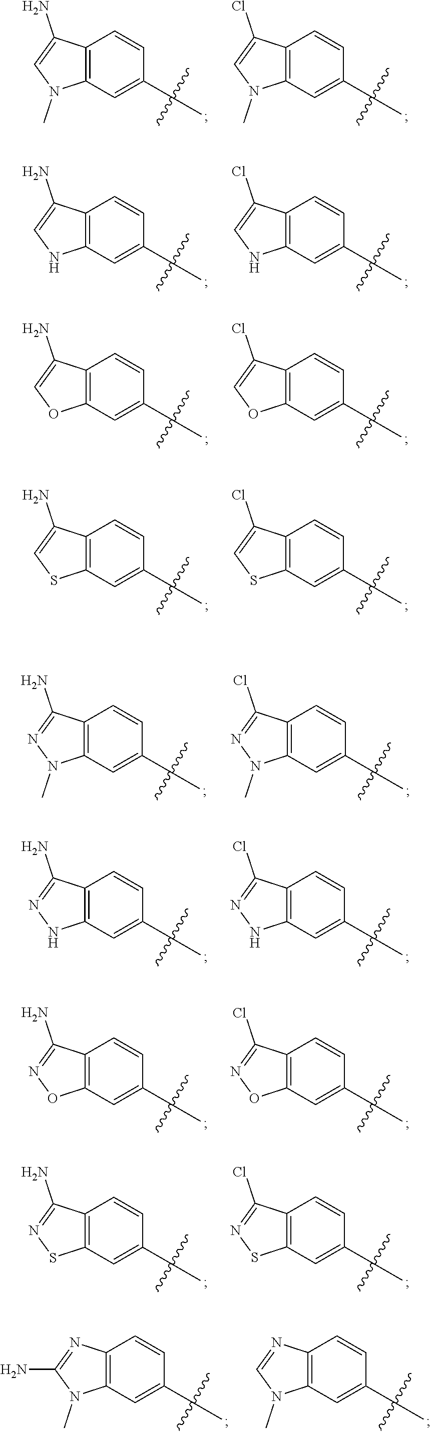

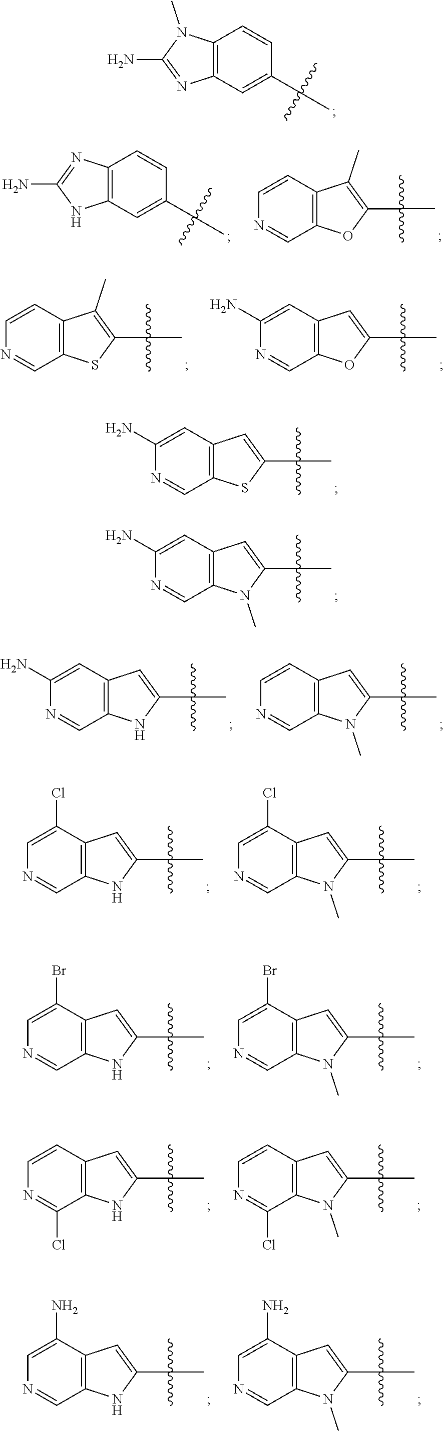

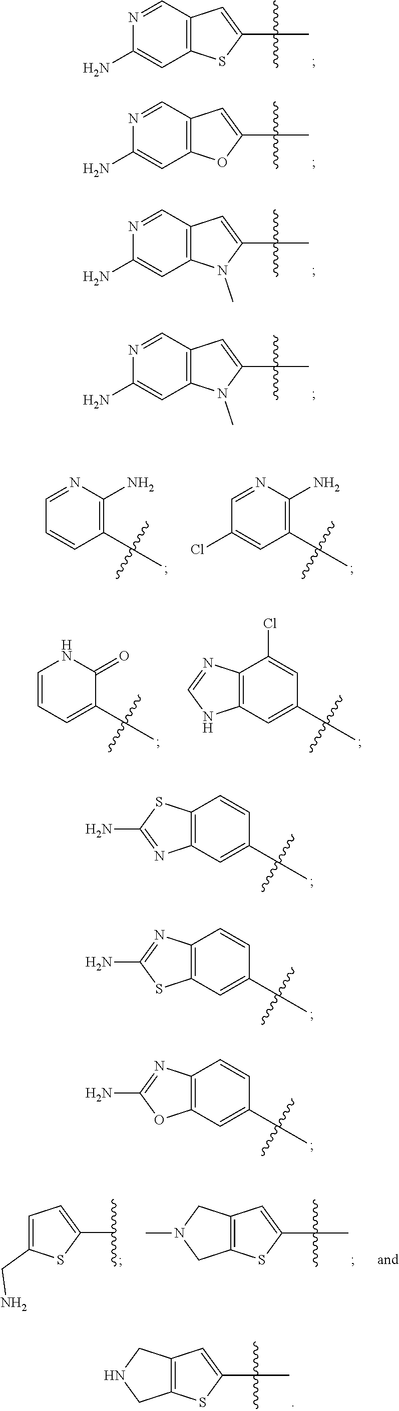

In some embodiments of Structures (I) and (I-A), Cy¹ is selected from the group consisting of:

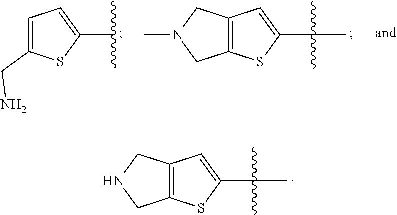

In some embodiments of Structures (I) and (I-A), Cy¹ is selected from the group consisting of:

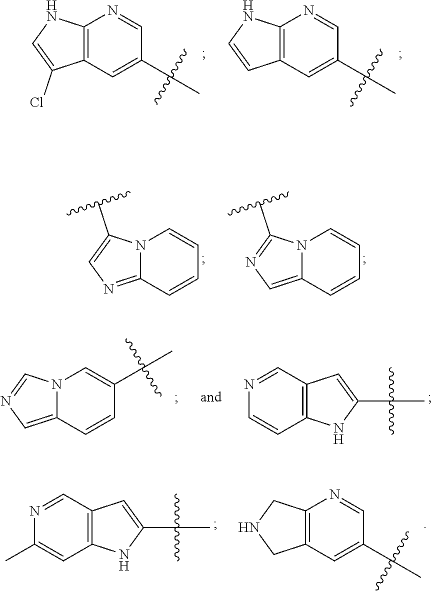

In some embodiments of Structures (I) and (I-A), Cy¹ is selected from the group consisting of:

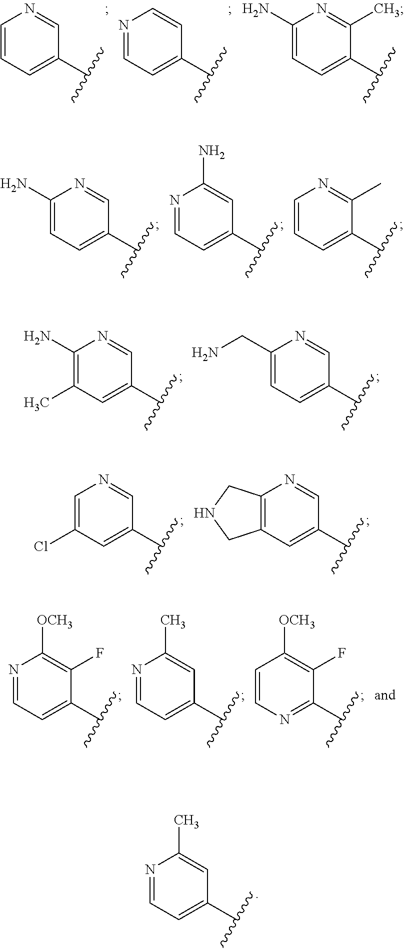

In some embodiments of Structures (I) and (I-A), Cy¹ is selected from the group consisting of:

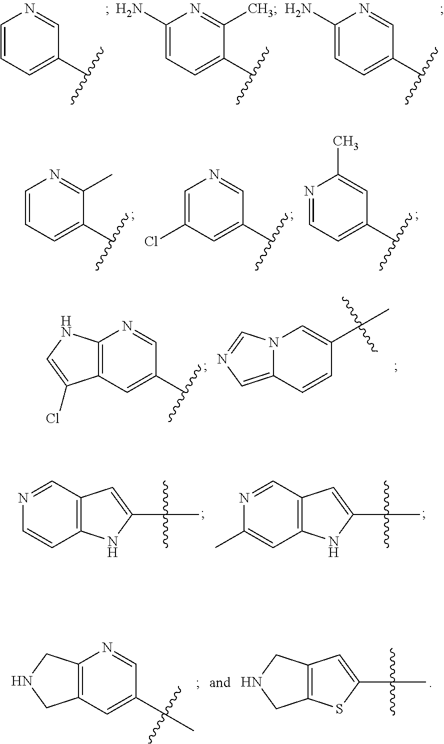

In some embodiments of Structures I and (I-B), Cy¹ is selected from the group consisting of:

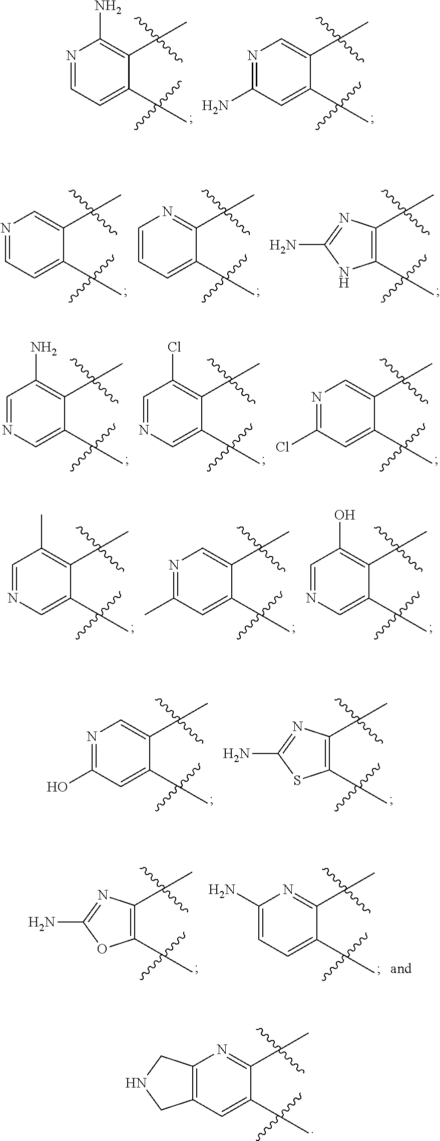

In some embodiments of Structures I and (I-B), Cy¹ is selected from the group consisting of:

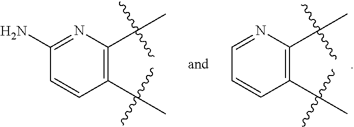 [SMILES: *c1ccc(N)nc1* ; *c1cccnc1*]

In some embodiments, the compound has Structure (I-C):

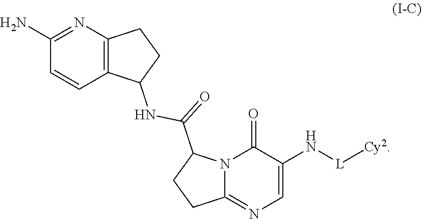

In some embodiments, the compound has Structure (I-D):

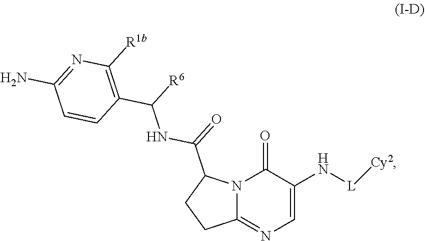

wherein R^{1b} is hydrogen or methyl, and
wherein R⁶ is hydrogen or methyl.

In some embodiments, the compound has Structure (I-E):

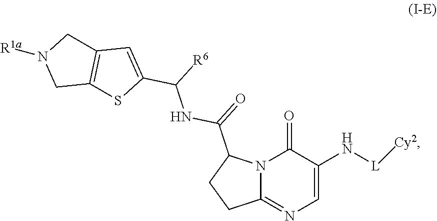

wherein R^{1a} is hydrogen or C₁-C₃ alkyl, and
wherein R⁶ is hydrogen or methyl.

In some embodiments of Structure (I-E), R^{1a} is hydrogen.

In some embodiments of Structure (I-E), R⁶ is methyl.

In some embodiments of Structures (I), (I-A), (I-B), (I-C), (I-D), and (I-E), n is 1, 2, or 3. In some embodiments of Structures (I), (I-A), (I-B), (I-C), (I-D), and (I-E), n is 1. In some embodiments of Structures (I), (I-A), (I-B), (I-C), (I-D), and (I-E), n is 2. In some embodiments of Structures (I), (I-A), (I-B), (I-C), (I-D), and (I-E), n is 3.

In some embodiments of Structures (I), (I-A), (I-B), (I-C), (I-D), and (I-E), each —(CR^{8a}R^{8b}) are independently the same or different. In some embodiments of Structures (I), (I-A), (I-B), (I-C), (I-D), and (I-E), each —(CR^{8a}R^{8b})— is the same. In some embodiments of Structures (I), (I-A), (I-B), (I-C), (I-D), and (I-E), each —(CR^{8a}R^{8b})— is different. In some embodiments of Structures (I), (I-A), (I-B), (I-C), (I-D), and (I-E), some of —(CR^{8a}R^{8b})— are the same and some of —(CR^{8a}R^{8b})— are different.

In some embodiments of Structures (I), (I-A), (I-B), (I-C), (I-D), and (I-E), R^{8a} and R &b are each independently hydrogen or substituted or unsubstituted C₁-C₃ alkyl. In such embodiments, R^{8a} and R^{8b} are the same, or R^{8a} and R^{8b} are different.

In some embodiments of Structures (I), (I-A), (I-B), (I-C), (I-D), and (I-E), R^{8a}, R^{8b}, or both R^{8a} and R^{8b}, are hydrogen.

In some embodiments of Structures (I), (I-A), (I-B), (I-C), (I-D), and (I-E), R^{8a}, R^{8b}, or both R^{8a} and R^{8b}, are unsubstituted C₁-C₃ alkyl.

In some embodiments of Structures (I), (I-A), (I-B), (I-C), (I-D), and (I-E), R^{8a}, R^{8b}, or both R^{8a} and R^{8b}, are substituted C₁-C₃ alkyl. In embodiments wherein R^{8a}, R^{8b}, or both R^{8a} and R^{8b}, are substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structures (I), (I-A), (I-B), (I-C), (I-D), and (I-E), R^{8a}, R^{8b}, or both R^{8a} and R^{8b}, are unsubstituted methyl, ethyl, n-propyl, or isopropyl.

In some embodiments of Structures (I), (I-A), (I-B), (I-C), (I-D), and (I-E), R^{8a}, R^{8b}, or both R^{8a} and R^{8b}, are substituted methyl, ethyl, n-propyl, or isopropyl. In embodiments wherein R^{8a}, R^{8b}, or both R^{8a} and R^{8b}, are substituted methyl, ethyl, n-propyl, or isopropyl, the methyl, ethyl, n-propyl, or isopropyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In embodiments wherein both of R^{8a} and R^{8b} are methyl, ethyl, n-propyl, or isopropyl, R^{8a} and R^{8b} are the same. In embodiments wherein both of R^{8a} and R^{8b} are methyl, ethyl, n-propyl, or isopropyl, R^{8a} and R^{8b} are different.

In some embodiments of Structures (I), (I-A), (I-B), (I-C), (I-D), and (I-E), one of R^{8a} and R^{8b} is hydrogen, and the other one of R^{8a} and R^{8b} is methyl.

In some embodiments of Structures (I), (I-A), (I-B), (I-C), (I-D), and (I-E), R^{8a} and R^{8b}, together with the carbon to which they are attached, form a substituted or unsubstituted C₃-C₆ cycloalkyl. In embodiments wherein R^{8a} and R^{8b} form a substituted or unsubstituted C₃-C₆ cycloalkyl, the cycloalkyl is a cyclopropyl, cyclobutyl, cyclopentyl, cyclohexyl, bicyclopentyl, or bicyclohexyl. In embodiments wherein R^{8a} and R^{8b} form a substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structures (I), (I-A), (I-B), (I-C), (I-D), and (I-E), L is —CH₂—, —CH₂CH₂—, —CH(CH₃)—, or —CH₂CH₂CH₂—. In some embodiments of Structures (I), (I-A), (I-B), (I-C), (I-D), and (I-E), L is —CH(CH₃)—. In some embodiments of Structures (I), (I-A), (I-B), (I-C), (I-D), and (I-E), L is —CH₂—.

In embodiments wherein a chiral center is present, L has (R) stereochemistry, (S) stereochemistry, is a racemic mixture, or is a mixture of (R) and (S) stereoisomers.

In some embodiments of Structures (I), (I-A), (I-B), (I-C), (I-D), and (I-E), Cy² is a substituted C₆-C₁₀ aryl.

In some embodiments of Structures (I), (I-A), (I-B), (I-C), (I-D), and (I-E), Cy² is a C₆-C₁₀ aryl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the C₆-C₁₀ aryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the C₆-C₁₀ aryl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (I), (I-A), (I-B), (I-C), (I-D), and (I-E), Cy² is a substituted phenyl.

In some embodiments of Structures (I), (I-A), (I-B), (I-C), (I-D), and (I-E), Cy² is a phenyl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the phenyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (I), (I-A), (I-B), (I-C), (I-D), and (I-E), Cy² has a structure selected from the group consisting of:

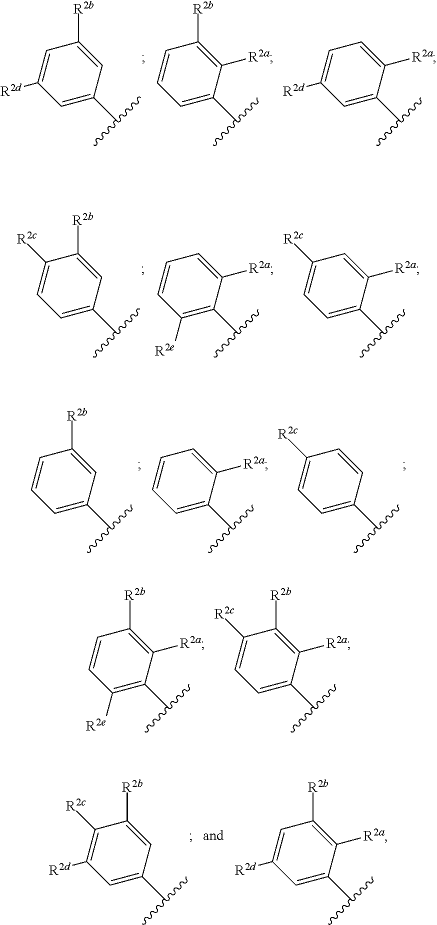

wherein R^{2a}, R^{2b}, R^{2c}, R^{2d}, and R^{2e} are each independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, and 5- or 6-membered heterocyclyl comprising 1-4 heteroatoms selected from the group consisting of N, S, and O; or
wherein any two of R^{2a}, R^{2b}, R^{2c}, R^{2d}, and R^{2e}, when attached to adjacent carbon atoms, together with the carbon atoms to which they are attached, form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl.

In some embodiments of Structures (I), (I-A), (I-B), (I-C), (I-D), and (I-E), Cy² is a phenyl independently substituted with one or more, 1-5, 1-4, 1-3, one or two, one, two, three, four, or five substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (I), (I-A), (I-B), (I-C), (I-D), and (I-E), Cy² is a phenyl substituted at two adjacent carbon atoms of the phenyl to form, together with the carbon atoms to which the substituents are attached, a dioxane fused to the phenyl, a furan fused to the phenyl, or a difluorodioxolane fused to the phenyl.

In some embodiments of Structures (I), (I-A), (I-B), (I-C), (I-D), and (I-E), Cy² is selected from the group consisting of:

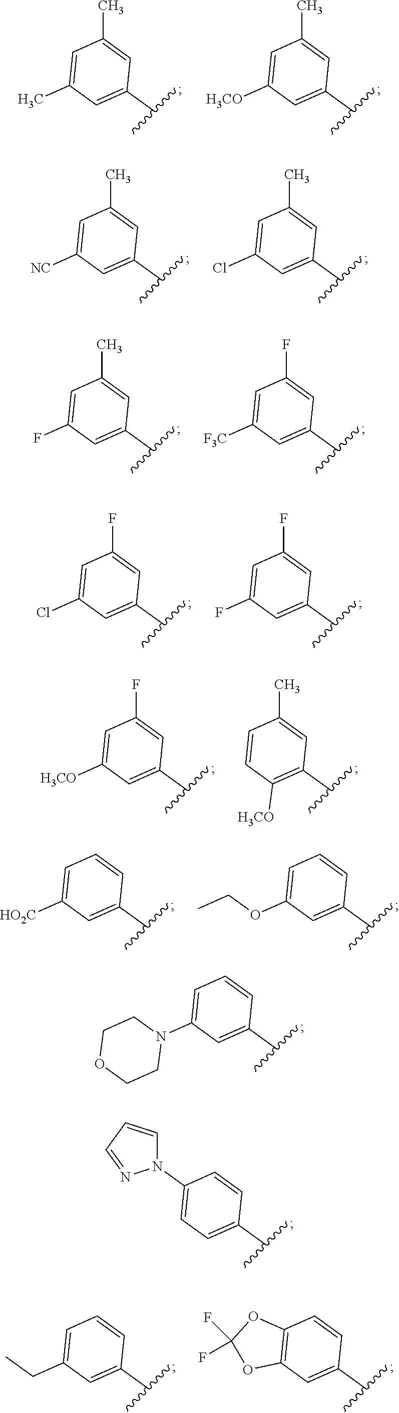

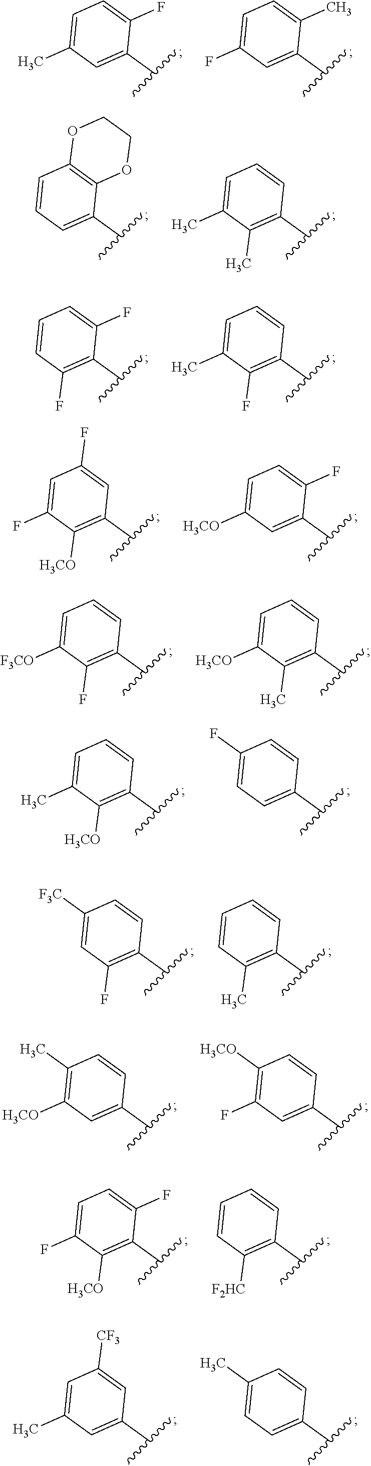

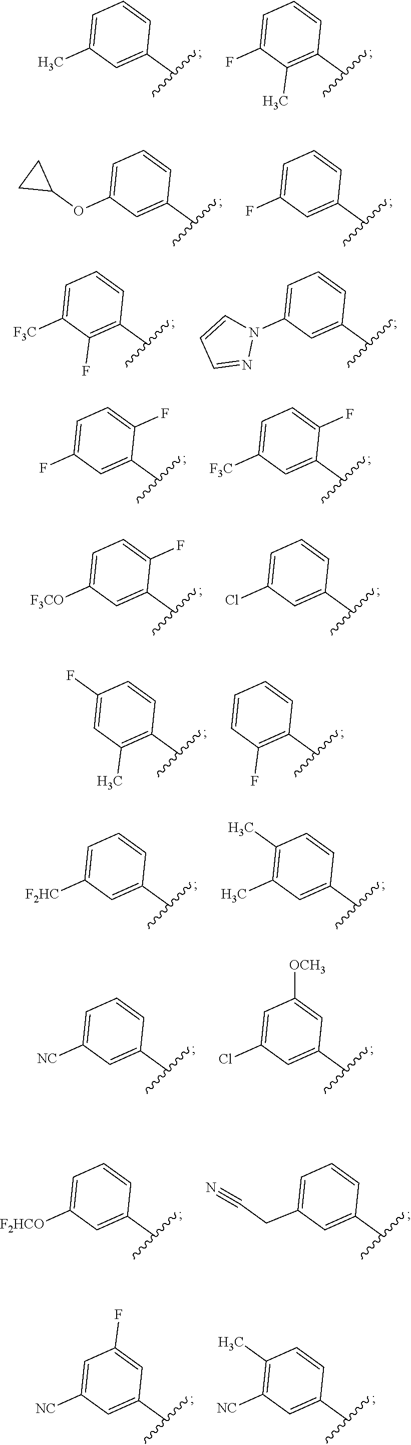

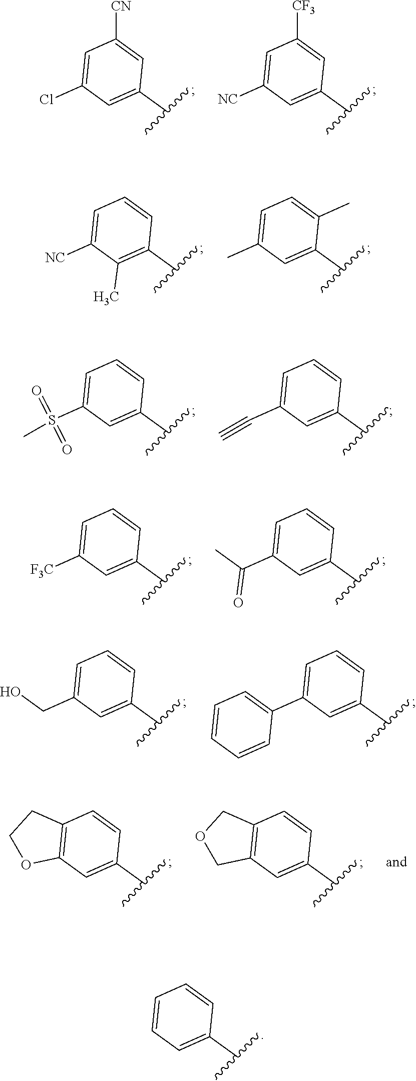

In some embodiments of Structures (I), (I-A), (I-B), (I-C), (I-D), and (I-E), Cy² is selected from the group consisting of:

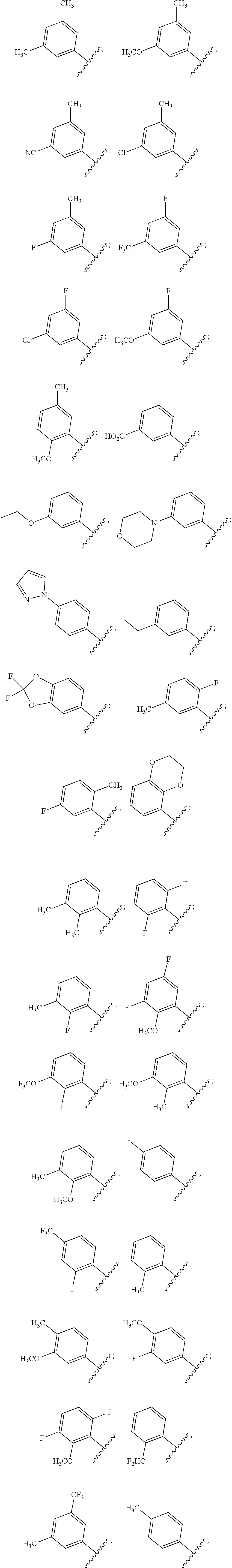

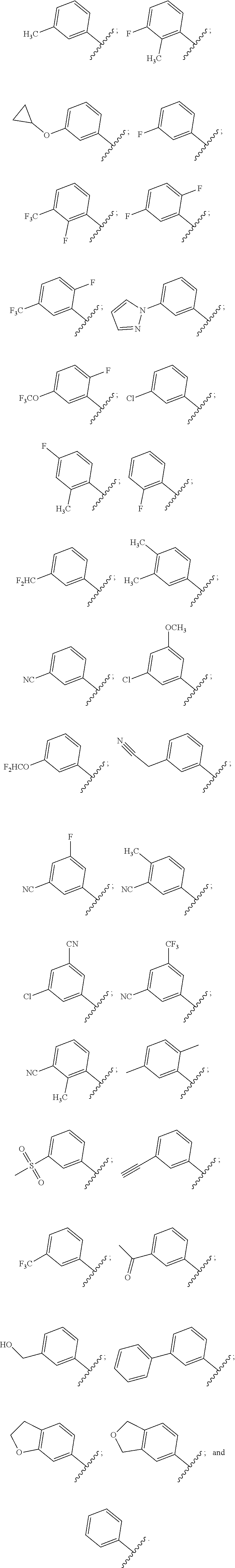

In some embodiments of Structures (I), (I-A), (I-B), (I-C), (I-D), and (I-E), Cy² is an unsubstituted C₃-C₆ cycloalkyl.

In some embodiments of Structures (I), (I-A), (I-B), (I-C), (I-D), and (I-E), Cy² is a C₃-C₆ cycloalkyl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In the foregoing embodiment, the C₃-C₆ cycloalkyl can be a cyclopropyl, cyclobutyl, cyclopentyl, cyclohexyl, bicyclopentyl, or bicyclohexyl.

In some embodiments of Structures (I), (I-A), (I-B), (I-C), (I-D), and (I-E), Cy² is a C₃-C₆ cycloalkyl substituted with one or more hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, or combinations thereof.

In the foregoing embodiment, the C₃-C₆ cycloalkyl can be a cyclopropyl, cyclobutyl, cyclopentyl, cyclohexyl, bicyclopentyl, or bicyclohexyl.

In some embodiments of Structures (I), (I-A), (I-B), (I-C), (I-D), and (I-E), Cy² is a C₅-C₆ cycloalkyl substituted with one or more methyl, ethyl, fluoro, chloro, methoxy, ethoxy, CN, COOH, hydroxymethyl, methoxycarbonyl, ethoxycarbonyl, trifluoromethyl, difluoromethyl, trifluoromethoxy, or combinations thereof.

In the foregoing embodiment, the C₅-C₆ cycloalkyl is cyclopentyl, cyclohexyl, bicyclopentyl, or bicyclohexyl.

In some embodiments of Structures (I), (I-A), (I-B), (I-C), (I-D), and (I-E), Cy² is a C₅ cycloalkyl substituted with one or more methyl, ethyl, fluoro, chloro, methoxy, ethoxy, CN, COOH, hydroxymethyl, methoxycarbonyl, ethoxycarbonyl, trifluoromethyl, difluoromethyl, trifluoromethoxy, or combinations thereof.

In the foregoing embodiment, the C₅ cycloalkyl is a cyclopentyl or bicyclopentyl.

In some embodiments of Structures (I), (I-A), (I-B), (I-C), (I-D), and (I-E), Cy² is:

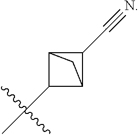 [SMILES: *C1C2CC1C2C#N]

In some embodiments of Structures (I), (I-A), (I-B), (I-C), (I-D), and (I-E), Cy² is an unsubstituted 5-10 membered heteroaryl.

In some embodiments of Structures (I), (I-A), (I-B), (I-C), (I-D), and (I-E), Cy² is a substituted 5-10 membered heteroaryl.

In some embodiments of Structures (I), (I-A), (I-B), (I-C), (I-D), and (I-E), Cy² is a 5-10 membered heteroaryl substituted with one or more, 1-4, 1-3, one or two, one, two, three, or four substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the heteroaryl, and wherein the substituted C₅-C₆ cycloalkyl fused to the heteroaryl and substituted 5- or 6-membered heterocyclic ring fused to the heteroaryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen, wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl, wherein the one or more, 1-4, 1-3, one or two, one, two, three, or four substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-4, 1-3, one or two, one, two, three, or four substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (I), (I-A), (I-B), (I-C), (I-D), and (I-E), Cy² is a 5-10 membered heteroaryl substituted with one or more substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (I), (I-A), (I-B), (I-C), (I-D), and (I-E), Cy² is an unsubstituted 5-6 membered heteroaryl.

In some embodiments of Structures (I), (I-A), (I-B), (I-C), (I-D), and (I-E), Cy² is a substituted 5-6 membered heteroaryl.

In some embodiments of Structures (I), (I-A), (I-B), (I-C), (I-D), and (I-E), Cy² is a 5-6 membered heteroaryl substituted with one or more substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (I), (I-A), (I-B), (I-C), (I-D), and (I-E), Cy² is an unsubstituted pyrazole.

In some embodiments of Structures (I), (I-A), (I-B), (I-C), (I-D), and (I-E), Cy² is a substituted pyrazole.

In some embodiments of Structures (I), (I-A), (I-B), (I-C), (I-D), and (I-E), Cy² is a pyrazole substituted with one or more substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (I), (I-A), (I-B), (I-C), (I-D), and (I-E), Cy² is a pyrazole substituted with phenyl.

In some embodiments of Structures (I), (I-A), (I-B), (I-C), (I-D), and (I-E), Cy² is:

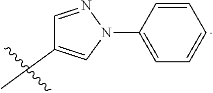 [SMILES: *c1cnn(-c2ccccc2)c1]

In some embodiments of Structures (I), (I-A), (I-C), (I-D), and (I-E), Cy² is hydrogen.

In some embodiments of Structures (I), (I-A), (I-D), and (I-E), the compound has a structure selected from the structures in Table 1 below.

In some embodiments of Structures (I), (I-B), and (I-C), the compound has a structure selected from the structures in Table 2 below.

In some aspects, the present disclosure provides a compound having Structure (II):

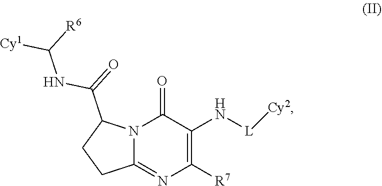

or a stereoisomer, tautomer, or pharmaceutically acceptable salt thereof, wherein:
Cy¹ is selected from the group consisting of:

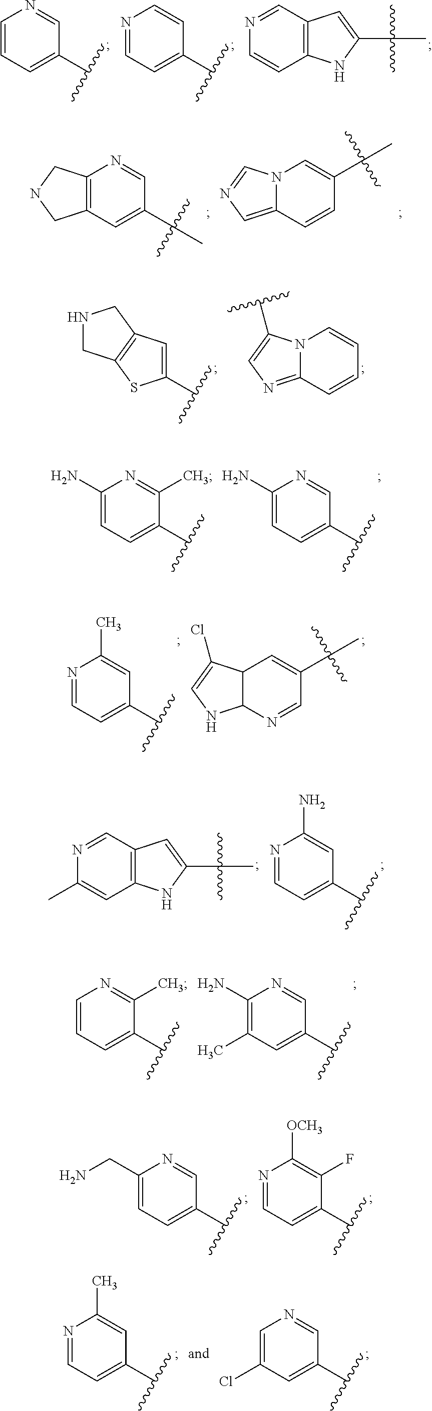

Cy² is a phenyl substituted with one or more, 1-5, 1-4, 1-3, one or two, one, two, three, four, or five substituents selected from the group consisting of hydrogen, C₂₋₆ alkenyl, C₂₋₆ alkynyl, halogen, C₁₋₆ haloalkyl, amino C₁₋₆ alkyl, C₁₋₆ alkyloxy, cyano, cyanomethyl, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or the phenyl is substituted at two adjacent carbon atoms and the substituents, together with the carbon atoms to which they are attached, form a C₅-C₆ cycloalkyl or a 5-6 membered heterocycle fused to the phenyl, each of which is optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl;
   R⁶ is hydrogen, C₁₋₃ alkyl, or C₁₋₃ haloalkyl;
   R⁷ is hydrogen, deuterium, C₁₋₆ alkyl, C₃₋₆ cycloalkyl, C₁₋₆ haloalkyl, C₃₋₆ halocycloalkyl, or halogen;
   L is —CH₂—, —CH₂CH₂—, —CH(CH₃)—, or —CH₂CH₂CH₂—, and
   wherein one or more hydrogen atoms are optionally replaced with one or more deuterium atoms.

In some embodiments of Structure (II), R⁶ is hydrogen or methyl.

In some embodiments of Structure (II), R⁷ is hydrogen.

In some embodiments of Structure (II), Cy¹ is selected from the group consisting of:

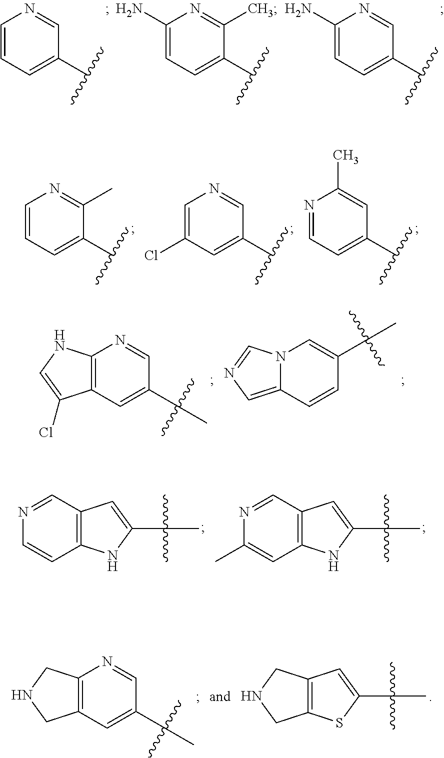

In some embodiments, the compound has Structure (II-A):

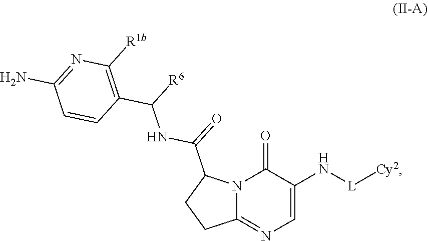

wherein R^{1b} is selected from the group consisting of hydrogen, methyl, ethyl, and CD₃, and wherein R⁶ is hydrogen or methyl.

In some embodiments of Structure (II-A), R^{1b} is hydrogen.

In some embodiments of Structure (II-A), R^{1b} is methyl.

In some embodiments of Structure (II-A), R⁶ is hydrogen.

In some embodiments of Structure (II-A), R⁶ is methyl.

In some embodiments, the compound has Structure (II-B):

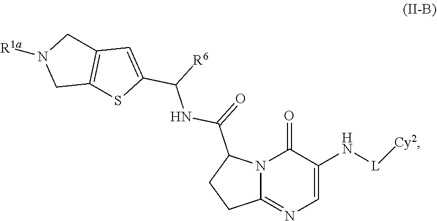

wherein R^{1a} is hydrogen or C₁-C₃ alkyl, and wherein R⁶ is hydrogen or methyl.

In some embodiments of Structure (II-B), R^{1a} is hydrogen.

In some embodiments of Structure (II-B), R⁶ is hydrogen.

In some embodiments of Structure (II-B), R⁶ is methyl.

In some embodiments, the compound has Structure (II-C):

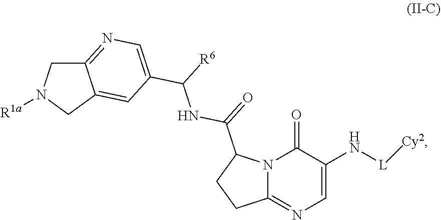

wherein R^{1a} is hydrogen or C₁-C₃ alkyl, and wherein R⁶ is hydrogen or methyl.

In some embodiments of Structure (II-C), R^{1a} is hydrogen.

In some embodiments of Structure (II-C), R⁶ is hydrogen.

In some embodiments of Structure (II-C), R⁶ is methyl.

In some embodiments, the compound has Structure (II-D):

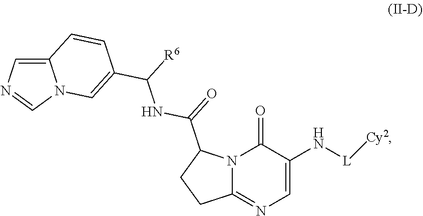

wherein R⁶ is hydrogen or methyl.

In some embodiments of Structure (II-D), R⁶ is hydrogen.

In some embodiments of Structure (II-D), R⁶ is methyl.

In some embodiments of Structures (II), (II-A), (II-B), (II-C), and (II-D), L is —CH₂— or —CH(CH₃)—.

In some embodiments of Structures (II), (II-A), (II-B), (II-C), and (II-D), L is —CH₂—.

In some embodiments of Structures (II), (II-A), (II-B), (II-C), and (II-D), L is —CH(CH₃)—.

In some embodiments of Structures (II), (II-A), (II-B), (II-C), and (II-D), Cy² has a structure selected from the group consisting of:

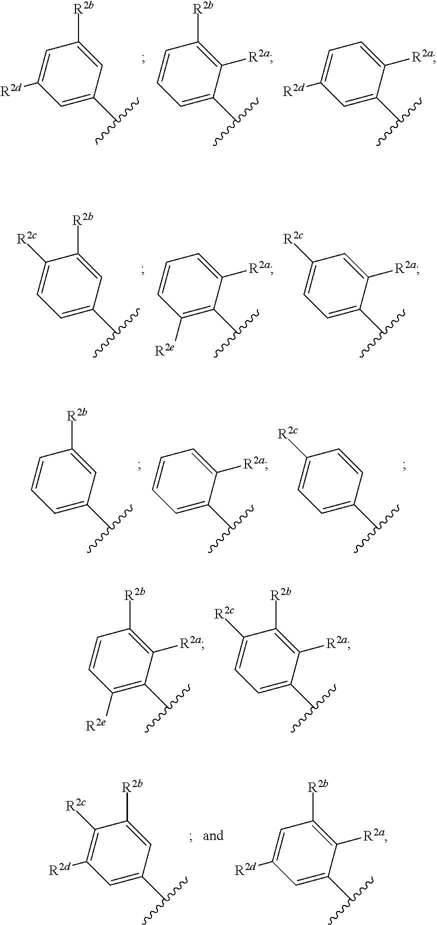

wherein R^{2a}, R^{2b}, R^{2e}, R^{2d}, and R^{2e} are each independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, and 5- or 6-membered heterocyclyl comprising 1-4 heteroatoms selected from the group consisting of N, S, and O; or
wherein when any two of R^{2a}, R^{2b}, R^{2c}, R^{2d}, and R^{2e} are attached to adjacent carbon atoms, together with the carbon atoms to which they are attached, the adjacent R^{2a}, R^{2b}, R^{2c}, R^{2d}, and R^{2e} form a substituted or unsubstituted C₅-C₆ cycloalkyl, or a substituted or unsubstituted 5-6 membered heterocyclic ring comprising 1-4 heteroatoms selected from the group consisting of N, S, and O, fused to the phenyl, and wherein the substituted C₅-C₆ cycloalkyl fused to the phenyl and substituted 5-6 membered heterocyclic ring fused to the phenyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen.

In some embodiments of Structures (II), (II-A), (II-B), (II-C), and (II-D), Cy² is selected from the group consisting of:

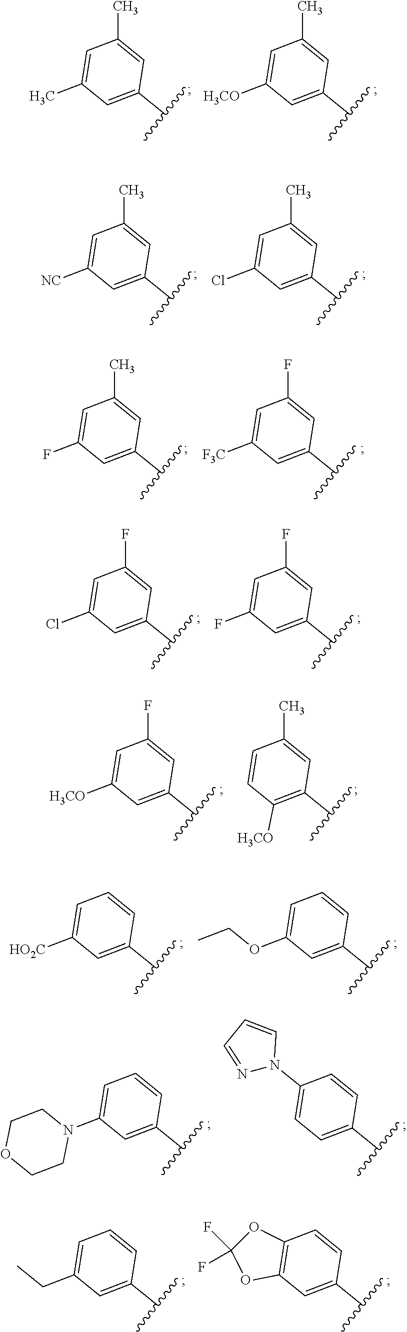

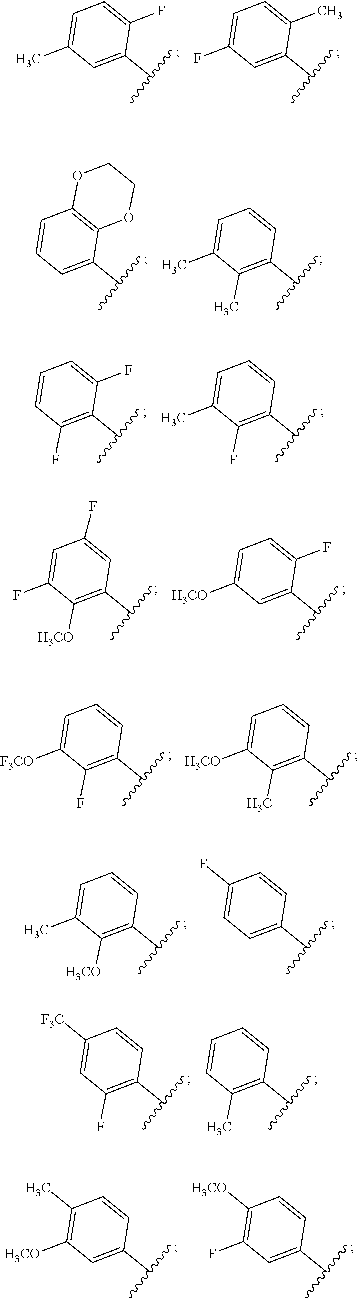

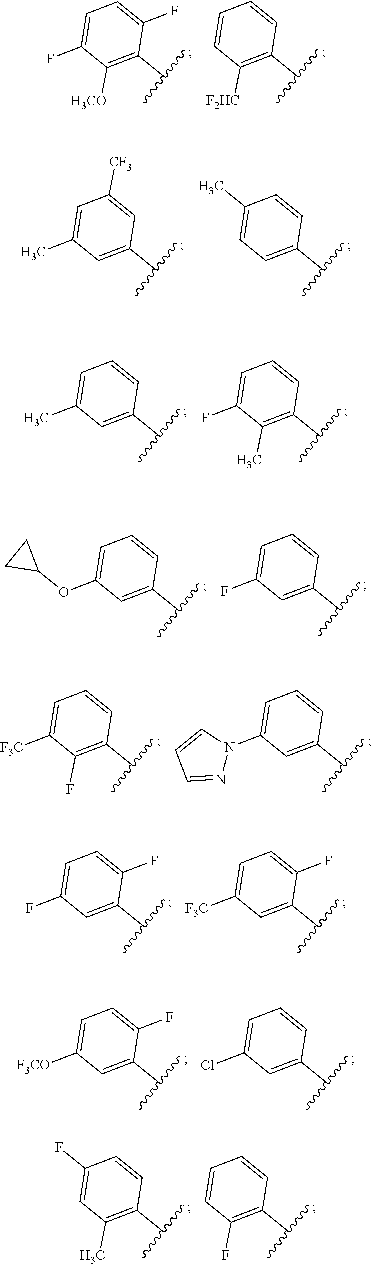

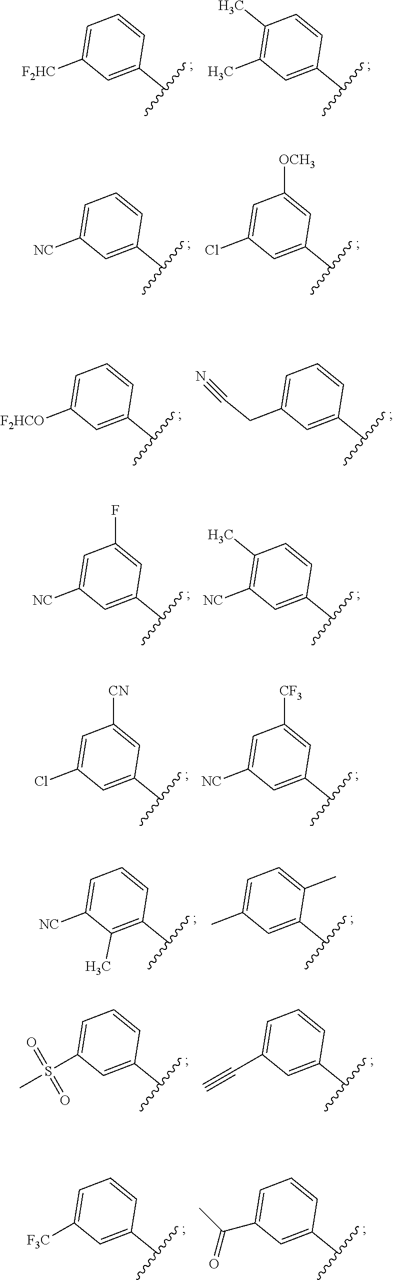

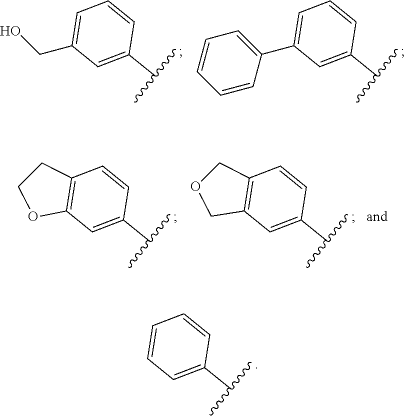

In some embodiments of Structures (II), (II-A), (II-B), (II-C), and (II-D), Cy² is selected from the group consisting of:

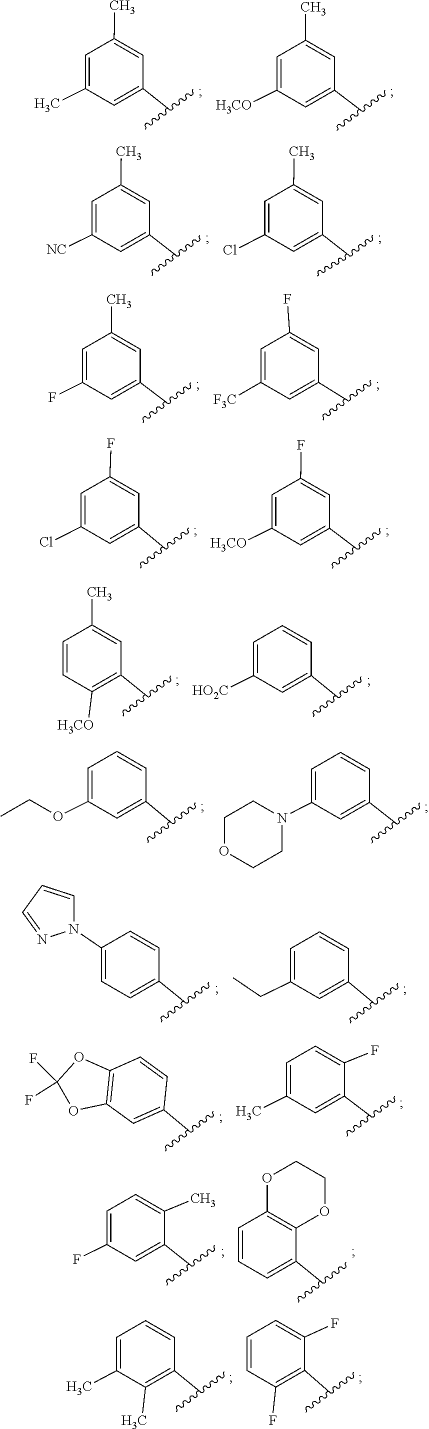

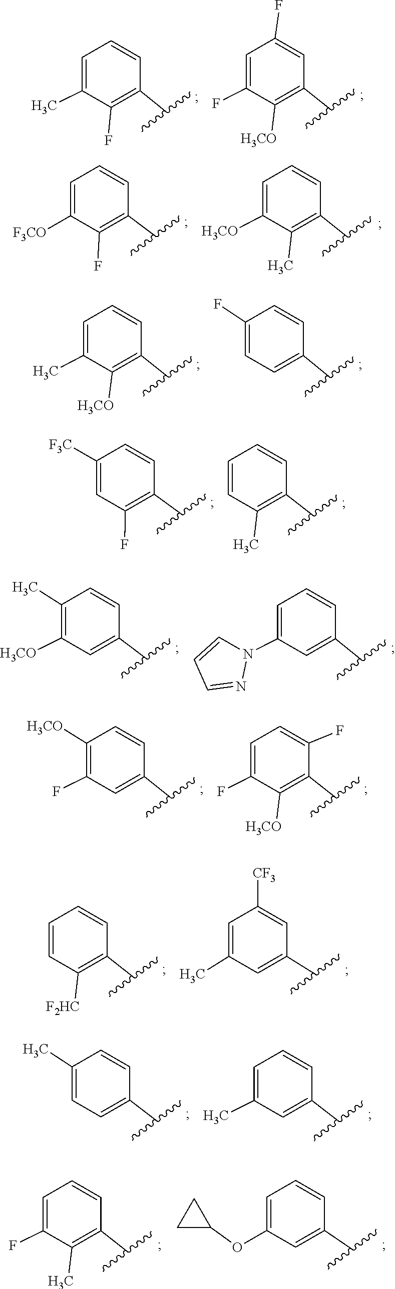

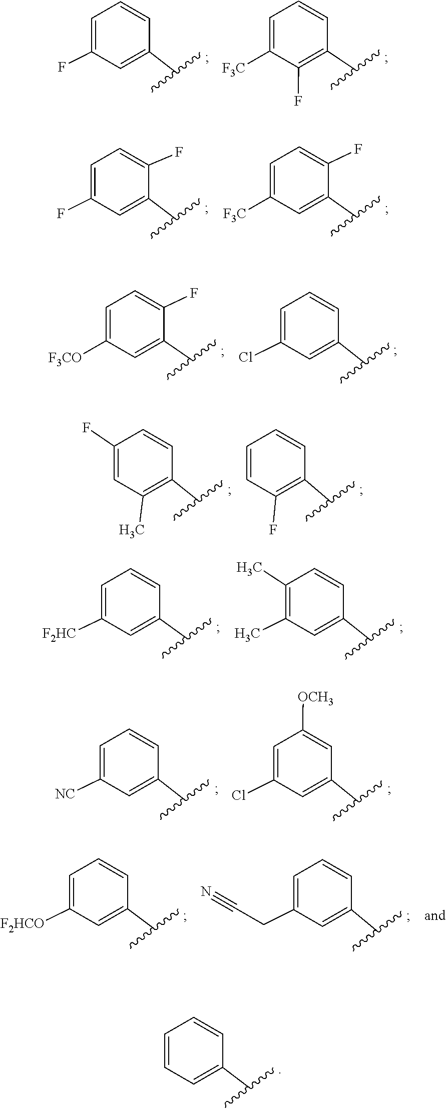

In some embodiments of Structures (II), (II-A), (II-B), (II-C), and (II-D), the compound has a structure selected from the structures in Table I below.

In some aspects, the present disclosure provides a compound having Structure (III):

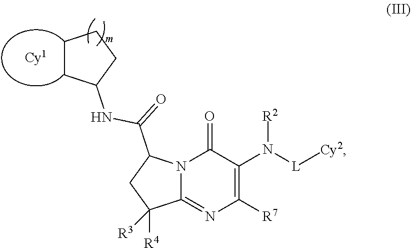

or a stereoisomer, tautomer, or pharmaceutically acceptable salt thereof, wherein:
Cy¹ is a substituted or unsubstituted aryl, or a substituted or unsubstituted 5-10-membered heteroaryl;
Cy² is a substituted aryl, a substituted or unsubstituted C₃-C₆ cycloalkyl, or a substituted or unsubstituted 5-10-membered heteroaryl;
R² is hydrogen, a substituted or unsubstituted C₁-C₃ alkyl, or a substituted or unsubstituted C₃-C₆ cycloalkyl;
R³ and R⁴ are each independently hydrogen, halogen, substituted or unsubstituted C₁-C₃ alkyl, or substituted or unsubstituted cycloalkyl, or R³ and R⁴, together with the carbon to which they are attached, form a substituted or unsubstituted C₃-C₆ cycloalkyl or a substituted or unsubstituted 5-6-membered cycloalkenyl;
R⁷ is selected from the group consisting of hydrogen, halogen, substituted or unsubstituted C₁-C₃ alkyl, and substituted or unsubstituted C₃-C₆ cycloalkyl;
L is absent or —(CR^{8a}R^{8b})ₙ—, wherein each —(CR^{8a}R^{8b})— is independently the same or different;
R^{8a} and R^{8b} are each independently hydrogen, substituted or unsubstituted C₁-C₃ alkyl, or R^{8a} and R^{8b}, together with the carbon to which they are attached, form a substituted or unsubstituted C₃-C₆ cycloalkyl;
m is 1 or 2; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms is optionally replaced with a deuterium atom.

In some embodiments of Structure (III), m is 1.

In some embodiments of Structure (III), m is 2.

In some embodiments of Structure (III), R² is a substituted or unsubstituted C₁-C₃ alkyl. In embodiments wherein R² is a substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R² is methyl. In some embodiments, R² is ethyl.

In some embodiments of Structure (III), R² is a substituted or unsubstituted C₃-C₆ cycloalkyl. In embodiments wherein R² is a substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (III), R² is hydrogen.

In some embodiments of Structure (III), R³ and R⁴ are each independently hydrogen, halogen, substituted or unsubstituted C₁-C₃ alkyl, or substituted or unsubstituted C₃-C₆ cycloalkyl.

In some embodiments of Structure (III), R³ is hydrogen, halogen, substituted or unsubstituted C₁-C₃ alkyl, or substituted or unsubstituted C₃-C₆ cycloalkyl. In some embodiments, R³ is hydrogen. In some embodiments, R³ is fluoro, chloro, bromo, or iodo. In some embodiments, R³ is unsubstituted C₁-C₃ alkyl. In some embodiments, R³ is substituted C₁-C₃ alkyl. For example, the C₁-C₃ alkyl is methyl, ethyl, n-propyl, or isopropyl. In embodiments wherein R³ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R³ is unsubstituted C₃-C₆ cycloalkyl. In some embodiments, R³ is substituted C₃-C₆ cycloalkyl. For example, the C₃-C₆ cycloalkyl can be a cyclopropyl, cyclobutyl, cyclopentyl, cyclohexyl, bicyclopentyl, or bicyclohexyl. In embodiments wherein R³ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments of Structure (III), R⁴ is hydrogen, halogen, substituted or unsubstituted C₁-C₃ alkyl, or substituted or unsubstituted C₃-C₆ cycloalkyl. In some embodiments, R⁴ is hydrogen. In some embodiments, R⁴ is fluoro, chloro, bromo, or iodo. In some embodiments, R⁴ is unsubstituted C₁-C₃ alkyl. In some embodiments, R⁴ is substituted C₁-C₃ alkyl. For example, the C₁-C₃ alkyl is methyl, ethyl, n-propyl, or isopropyl. In embodiments wherein R⁴ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy. In some embodiments, R⁴ is unsubstituted C₃-C₆ cycloalkyl. In some embodiments, R⁴ is substituted C₃-C₆ cycloalkyl. For example, the C₃-C₆ cycloalkyl can be a cyclopropyl, cyclobutyl, cyclopentyl, cyclohexyl, bicyclopentyl, or bicyclohexyl. In embodiments wherein R⁴ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (III), R³ and R⁴, together with the carbon to which they are attached, form a substituted or unsubstituted C₃-C₆ cycloalkyl, or a substituted or unsubstituted C₅-C₆ cycloalkenyl. For example, the C₃-C₆ cycloalkyl can be a cyclopropyl, cyclobutyl, cyclopentyl, cyclohexyl, bicyclopentyl, or bicyclohexyl. In another example, the C₅-C₆ cycloalkenyl can be a cyclopentenyl or a cyclohexenyl. In embodiments wherein R³ and R⁴ form a substituted C₃-C₆ cycloalkyl or a substituted C₅-C₆ cycloalkenyl the C₃-C₆ cycloalkyl or C₅-C₆ cycloalkenyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (III), when a chiral center is present at the carbon atom to which R³ and R⁴ are attached, the compound has (R) stereochemistry, has (S) stereochemistry, is a racemic mixture, or comprises a mixture of (R) and (S) stereoisomers.

In some embodiments of Structure (III), R⁷ is selected from the group consisting of hydrogen, halogen, substituted or unsubstituted C₁-C₃ alkyl, and substituted or unsubstituted C₃-C₆ cycloalkyl.

In some embodiments of Structure (III), R⁷ is hydrogen.

In some embodiments of Structure (III), R⁷ is fluoro, chloro, bromo, or iodo.

In some embodiments of Structure (III), R⁷ is unsubstituted C₁-C₃ alkyl. For example, the C₁-C₃ alkyl is methyl, ethyl, n-propyl, or isopropyl.

In some embodiments of Structure (III), R⁷ is substituted C₁-C₃ alkyl. For example, the C₁-C₃ alkyl is methyl, ethyl, n-propyl, or isopropyl. In embodiments wherein R⁷ is substituted C₁-C₃ alkyl, the C₁-C₃ alkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (III), R⁷ is unsubstituted C₃-C₆ cycloalkyl. In some embodiments, R⁷ is substituted C₃-C₆ cycloalkyl. For example, the C₃-C₆ cycloalkyl is a cyclopropyl, cyclobutyl, cyclopentyl, cyclohexyl, bicyclopentyl, or bicyclohexyl.

In embodiments wherein R⁷ is substituted C₃-C₆ cycloalkyl, the C₃-C₆ cycloalkyl is substituted with one or more of C₁-C₃ alkyl, halogen, C₁-C₃ alkoxy, or combinations thereof, including combinations of one or more same or different C₁-C₃ alkyl, one or more same or different halogen, and/or one or more same or different C₁-C₃ alkoxy.

In some embodiments of Structure (III), R⁷ is selected from the group consisting of hydrogen, deuterium, C₁-C₃ alkyl, and C₁-C₃ haloalkyl.

In some embodiments the compound has Structure (III-A):

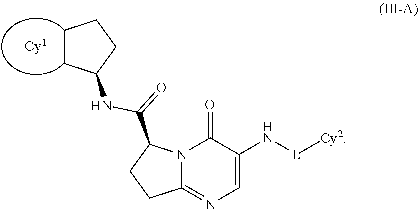

In some embodiments of Structures (III) and (III-A), the compound has the Structure:

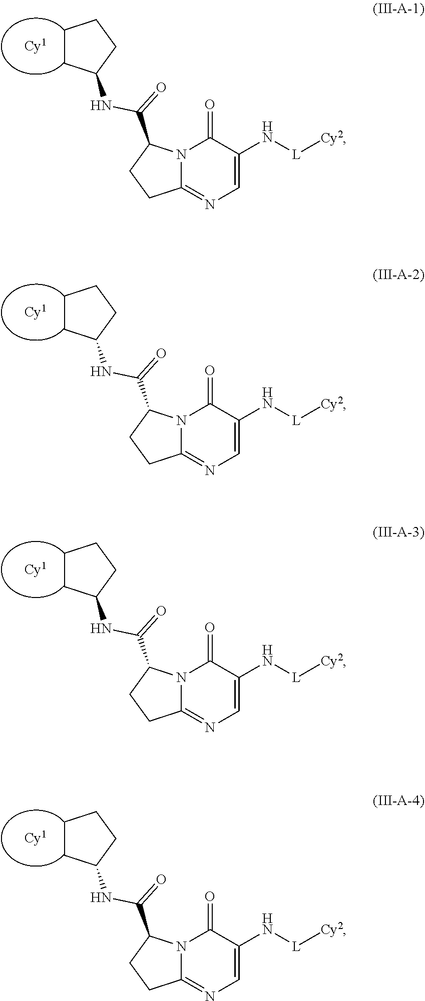

or combinations thereof.

In some embodiments of Structures (III) and (III-A), L is —CH₂—, —CH₂CH₂—, —CH(CH₃)—, or —CH₂CH₂CH₂—.

In some embodiments of Structures (III) and (III-A), L is —CH₂—.

In some embodiments of Structures (III) and (III-A), Cy¹ is a phenyl substituted with one or more, 1-5, 1-4, 1-3, one or two, one, two, three, four, or five substituents independently selected from the group consisting of hydrogen, C(NH)NH₂, C(═NH)NHC(—O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹³, C₂-C₆ alkenyl, C₂-C₆ alkynyl, halogen, C₁-C₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, N(R⁹)C(═NR¹⁰)NR¹¹R¹², S(O)R⁹, S(O)NR⁹R¹⁰, S(O)₂R⁹, C(═NR⁹)N(R¹⁰)C(O)OR¹¹, N(R⁹)S(O)₂R¹⁰, S(O)₂NR⁹R¹⁰, oxo, substituted or unsubstituted C₁-C₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10-membered heterocyclyl, or wherein when the phenyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl,
wherein R⁸, R⁹, R¹⁰, R¹¹, and R¹², are, at each occurrence, independently selected from the group consisting of hydrogen, deuterium, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, heteroarylalkyl, and heteroaryl,
wherein R¹³ is a C₆-C₁₀ aryl or 5-10-membered heteroaryl, optionally substituted with CH₂OC(═O)CH₃,
wherein when the one or more, 1-5, 1-4, 1-3, one or two, one, two, three, four, or five substituents are a substituted C₁-C₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl, the one or more, 1-5, 1-4, 1-3, one or two, one, two, three, four, or five substituents are optionally substituted with one or more substituents independently selected from the group consisting of halo, CN, OR^{e}, SR^{e}, C(O)R^{e}, C(O)NR^{e}R^{f}, C(O)OR^{e}, OC(O)R^{e}, OC(O)NR^{e}R^{f}, NR^{e}R^{f}, NR^{e}C(O)R^{f}, NR^{e}C(O)NR^{f}R^{g}, NR^{e}C(O)OR^{f}, C(═NR^{e})NR^{f}R^{g}, NR^{e}C(═NR^{f})NR^{g}R^{h}, S(O)R^{e}, S(O)NR^{e}R^{f}, S(O)₂R^{e}, NR^{e}S(O)₂R^{f}, S(O)₂NR^{e}R^{f} and oxo, wherein R^{e}, R^{f}, R^{g}, and R^{h} are, at each occurrence, independently selected from the group consisting of hydrogen, C₁-C₆ alkyl, C₂-C₆ alkenyl, C₂-C₆ alkynyl, hydroxyl, C₁-C₆ alkoxy, aryl, arylalkyl, C₁-C₆ haloalkyl, C₁-C₆ haloalkoxy, C₁-C₆ hydroxyalkyl, cycloalkyl, heterocyclyl, and heteroaryl.

In some embodiments of Structures (III) and (III-A), Cy¹ is:

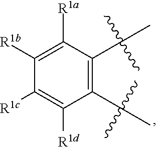

wherein R^{1a}, R^{1b}, R^{1e}, and R^{1d} are each independently selected from the group consisting of hydrogen, deuterium, C₁₋₆ alkyl, C₁₋₆ deuterated alkyl, amino, and aminylalkyl; or
wherein when any two of R^{1a}, R^{1b}, R^{1c}, and R^{1d} are connected to the phenyl at adjacent carbon atoms, the two R^{1a}, R^{1b}, R^{1c}, and R^{1d} are connected, together with the carbon atoms to which they are attached, to form a substituted or unsubstituted 5-6-membered carbocyclic ring, or a substituted or unsubstituted 5-6-membered heterocyclic ring comprising 1-4 heteroatoms selected from the group consisting of N, S, and O, fused to the phenyl.

In some embodiments of Structures (III) and (III-A), Cy¹ is selected from the group consisting of:

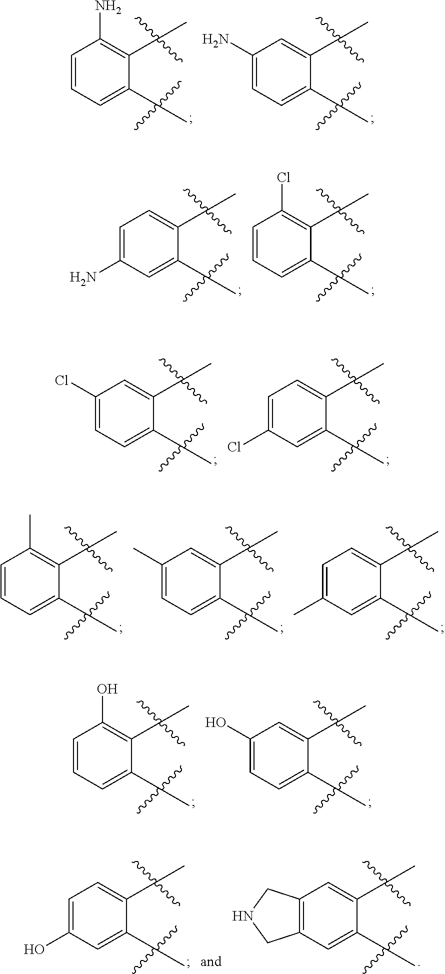

In some embodiments of Structures (III) and (III-A), Cy¹ is a phenyl substituted with amino.

In some embodiments of Structures (III) and (III-A), Cy¹ is selected from the group consisting of:

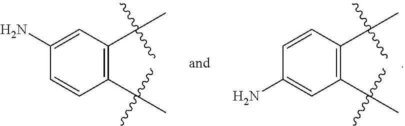 [SMILES: *c1ccc(N)cc1* ; *c1ccc(N)cc1*]

In some embodiments of Structures (III) and (III-A), Cy¹ is a substituted or unsubstituted 5-10-membered heteroaryl.

In some embodiments of Structures (III) and (III-A), Cy¹ is an unsubstituted 5-10-membered heteroaryl.

In some embodiments of Structures (III) and (III-A), Cy¹ is a 5-10-membered heteroaryl substituted with one or more, 1-5, 1-4, 1-3, one or two, one, two, three, four, or five substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the 5-10-membered heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the 5-10-membered heteroaryl, and wherein the substituted C₅-C₆ cycloalkyl fused to the 5-10-membered heteroaryl and substituted 5- or 6-membered heterocyclic ring fused to the 5-10-membered heteroaryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen, wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more, 1-5, 1-4, 1-3, one or two, one, two, three, four, or five substituents are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (III) and (III-A), Cy¹ is a substituted or unsubstituted 5-6-membered heteroaryl.

In some embodiments of Structures (III) and (III-A), Cy¹ is an unsubstituted 5-6-membered heteroaryl.

In some embodiments of Structures (III) and (III-A), Cy¹ is a 5-6-membered heteroaryl substituted with one or more, one to three, one or two, one, two, or three substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the heteroaryl, and wherein the substituted C₅-C₆ cycloalkyl fused to the 5-6-membered heteroaryl and substituted 5- or 6-membered heterocyclic ring fused to the 5-6-membered heteroaryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more, one to three, one or two, one, two, or three substituents are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, one to three, one or two, one, two, or three substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (III) and (III-A), Cy¹ is a substituted or unsubstituted pyridinyl.

In some embodiments of Structures (III) and (III-A), Cy¹ is an unsubstituted pyridinyl.

In some embodiments of Structures (III) and (III-A), Cy¹ is a pyridinyl substituted with one or more, one to three, one or two, one, two, or three substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the pyridinyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the pyridinyl, and wherein the substituted C₅-C₆ cycloalkyl fused to the pyridinyl and substituted 5- or 6-membered heterocyclic ring fused to the pyridinyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more, one to three, one or two, one, two, or three substituents re optionally substituted with one or more, one to three, one or two, one, two, or three substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, one to three, one or two, one, two, or three substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (III) and (III-A), Cy¹ has a structure selected from the group consisting of:

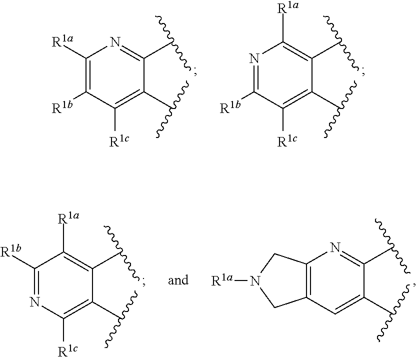

wherein R^{1a}, Rib, and R^{1c} are independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the pyridinyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the pyridinyl, and wherein the substituted C₅-C₆ cycloalkyl fused to the pyridinyl and substituted 5- or 6-membered heterocyclic ring fused to the pyridinyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the R^{1a}, R^{1b}, and R^{1c} are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the R^{1a}, R^{1b}, and R^{1c} are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

In some embodiments of Structures (III) and (III-A), Cy¹ has a structure selected from the group consisting of:

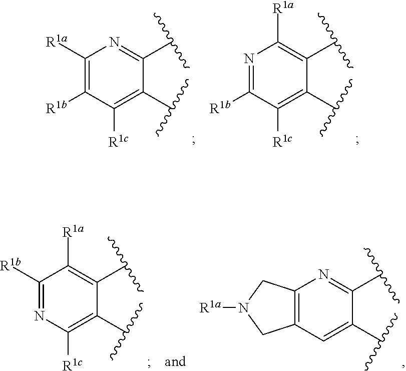

wherein R^{1a}, R^{1b}, and R^{1c} are each independently selected from the group consisting of hydrogen, C₁-C₆ alkyl, C₁-C₆ deuterated alkyl, amino, aminylalkyl, alkoxy, and halogen.

In some embodiments of Structures (III) and (III-A), Cy¹ is pyridinyl substituted with amino.

In some embodiments of Structures (III) and (III-A), Cy¹ is selected from the group consisting of:

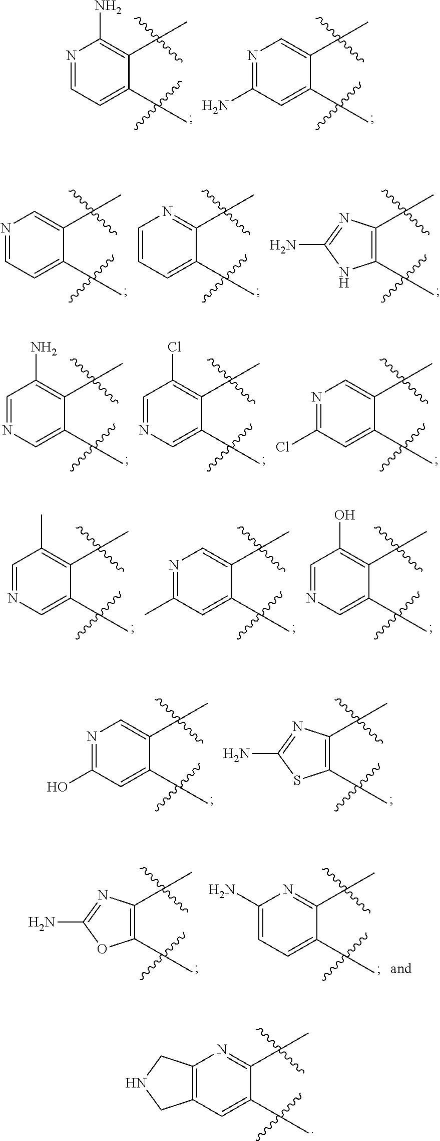

In some embodiments of Structures (TIT) and (III-A), Cy¹ is:

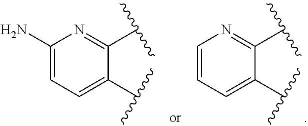 [SMILES: *c1ccc(N)nc1* ; *c1cccnc1*]

In some embodiments, the compound has Structure (III-B):

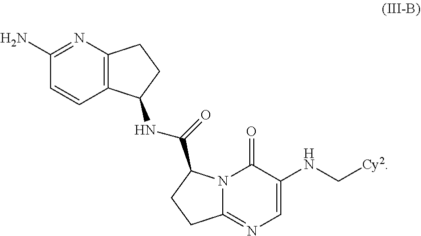

In some embodiments of Structures (III), (III-A), and (III-B), Cy² is a substituted or unsubstituted C₃-C₆ cycloalkyl.

In some embodiments of Structures (III), (III-A), and (III-B), Cy² is an unsubstituted C₃-C₆ cycloalkyl.

In some embodiments of Structures (III), (III-A), and (III-B), Cy² is a C₃-C₆ cycloalkyl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein each of the one or more, 1-5, 1-4, 1-3, one or two, one, two, three, four, or five substituents is optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo, when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In the foregoing embodiment, the C₃-C₆ cycloalkyl of Cy² is cyclopropyl, cyclobutyl, cyclopentyl, cyclohexyl, bicyclopentyl, or bicyclohexyl.

In some embodiments of Structures (III), (III-A), and (III-B), Cy² is a C₅-C₆ cycloalkyl substituted with one or more, 1-5, 1-4, 1-3, one or two, one, two, three, four, or five substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a C₅-C₆ cycloalkyl substituted with one or more, 1-5, 1-4, 1-3, one or two, one, two, three, four, or five substituents independently selected from the group consisting of hydrogen, methyl, ethyl, fluoro, chloro, methoxy, ethoxy, CN, cyanomethyl, COOH, hydroxymethyl, methoxycarbonyl, ethoxycarbonyl, trifluoromethyl, difluoromethyl, fluoromethyl, fluoromethoxy, difluoromethoxy, trifluoromethoxy, pyrazole, cyclopropoxy, morpholinyl, and combinations thereof.

In the foregoing embodiments, the C₅-C₆ cycloalkyl of Cy² is cyclopentyl, cyclohexyl, bicyclopentyl, or bicyclohexyl.

In some embodiments of Structures (III), (III-A), and (III-B), Cy² is:

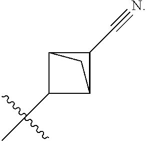 [SMILES: *C1C2CC1C2C#N]

In some embodiments of Structures (III), (III-A), and (III-B), Cy² is an unsubstituted or substituted 5-10 membered heteroaryl.

In some embodiments of Structures (III), (III-A), and (III-B), Cy² is an unsubstituted 5-10 membered heteroaryl.

In some embodiments of Structures (III), (III-A), and (III-B), Cy² is a 5-10 membered heteroaryl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the 5-10-membered heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the 5-10-membered heteroaryl, and wherein the substituted C₅-C₆ cycloalkyl fused to the 5-10-membered heteroaryl and substituted 5- or 6-membered heterocyclic ring fused to the 5-10-membered heteroaryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are each optionally substituted with one or more substituents selected from the group consisting of halogen, CN, OR¹³, SR¹³, C(O)R¹³, C(O)NR¹³R¹⁴, C(O)OR¹³, OC(O)R¹³, NR¹³C(O)R¹⁴, OC(O)NR¹³R¹⁴, NR¹³R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (III), (III-A), and (III-B), Cy² is a 5-10 membered heteroaryl substituted with one or more, 1-5, 1-4, 1-3, one or two, one, two, three, four, or five substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a 5-10 membered heteroaryl substituted with one or more, 1-5, 1-4, 1-3, one or two, one, two, three, four, or five substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (III), (III-A), and (III-B), Cy² is a 5-10 membered heteroaryl substituted at two adjacent carbon atoms of the heteroaryl to form, together with the carbon atoms to which the substituents are attached, a dioxane fused to the heteroaryl, a furan fused to the heteroaryl, or a difluorodioxolane fused to the heteroaryl.

In some embodiments of Structures (III), (III-A), and (III-B), Cy² is an unsubstituted or substituted 5-6 membered heteroaryl.

In some embodiments of Structures (III), (III-A), and (III-B), Cy² is an unsubstituted 5-6 membered heteroaryl.

In some embodiments of Structures (III), (III-A), and (III-B), Cy² is a 5-6 membered heteroaryl substituted with one or more, one to three, one or two, one, two, or three substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the 5-6-membered heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the 5-6-membered heteroaryl, and wherein the substituted C₅-C₆ cycloalkyl fused to the 5-6-membered heteroaryl and substituted 5- or 6-membered heterocyclic ring fused to the 5-6-membered heteroaryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, one to three, one or two, one, two, or three substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, one to three, one or two, one, two, or three substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (III), (III-A), and (III-B), Cy² is a 5-6 membered heteroaryl substituted with one or more, one to three, one or two, one, two, or three substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a 5-6 membered heteroaryl substituted with one or more, one to three, one or two, one, two, or three substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (III), (III-A), and (III-B), Cy² is a 5-10 membered heteroaryl substituted at two adjacent carbon atoms of the heteroaryl to form, together with the carbon atoms to which the substituents are attached, a dioxane fused with the heteroaryl, a furan fused to the heteroaryl, or a difluorodioxolane fused to the heteroaryl.

In some embodiments of Structures (III), (III-A), and (III-B), Cy² is an unsubstituted or substituted pyrazolyl.

In some embodiments of Structures (III), (III-A), and (III-B), Cy² is an unsubstituted pyrazolyl.

In some embodiments of Structures (III), (III-A), and (III-B), Cy² is a pyrazolyl substituted with one or more substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a pyrazolyl substituted with one or more substituents independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (III), (III-A), and (III-B), Cy² is:

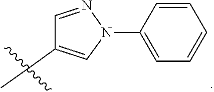 [SMILES: *c1cnn(-c2ccccc2)c1]

In some embodiments of Structures (III), (III-A), and (III-B), Cy² is a substituted C₆-C₁₀ aryl.

In some embodiments of Structures (III), (III-A), and (III-B), Cy² is a C₆-C₁₀ aryl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the C₆-C₁₀ aryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the C₆-C₁₀ aryl, and wherein the substituted C₅-C₆ cycloalkyl fused to the C₆-C₁₀ aryl and substituted 5- or 6-membered heterocyclic ring fused to the C₆-C₁₀ aryl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents is optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (III), (III-A), and (III-B), Cy² is a C₆-C₁₀ aryl substituted with one or more, 1-5, 1-4, 1-3, one or two, one, two, three, four, or five substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a C₆-C₁₀ aryl substituted with one or more, 1-5, 1-4, 1-3, one or two, one, two, three, four, or five substituents independently selected from the group consisting of methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (III), (III-A), and (III-B), Cy² is a C₆-C₁₀ aryl substituted with one or more, 1-5, 1-4, 1-3, one or two, one, two, three, four, or five substituents independently selected from the group consisting of methyl, fluoro, chloro, methoxy, CN, cyanomethyl, trifluoromethyl, difluoromethyl, phenyl, methanesulfonyl, ethynyl, hydroxymethyl, acetyl, and combinations thereof.

In some embodiments of Structures (III), (III-A), and (III-B), Cy² is a substituted phenyl.

In some embodiments of Structures (III), (III-A), and (III-B), Cy² is a phenyl substituted with one or more, 1-5, 1-4, 1-3, one or two, one, two, three, four, or five substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(—NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the phenyl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl, and wherein the substituted C₅-C₆ cycloalkyl fused to the phenyl and substituted 5- or 6-membered heterocyclic ring fused to the phenyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more, 1-5, 1-4, 1-3, one or two, one, two, three, four, or five substituents are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more, 1-5, 1-4, 1-3, one or two, one, two, three, four, or five substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structures (III), (III-A), and (III-B), Cy² is a phenyl substituted with one or more, 1-5, 1-4, 1-3, one or two, one, two, three, four, or five substituents independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof.

In some embodiments of Structures (III), (III-A), and (III-B), Cy² is a phenyl substituted with one or more, 1-5, 1-4, 1-3, one or two, one, two, three, four, or five substituents independently selected from the group consisting of methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, trifluoromethoxy, difluoromethoxy, pyrazole, cyclopropoxy, morpholinyl, phenyl, methanesulfonyl, ethynyl, hydroxymethyl, acetyl, and combinations thereof.

In some embodiments of Structures (III), (III-A), and (III-B), Cy² is a phenyl substituted with one or more, 1-5, 1-4, 1-3, one or two, one, two, three, four, or five substituents independently selected from the group consisting of methyl, fluoro, chloro, methoxy, CN, cyanomethyl, trifluoromethyl, difluoromethyl, phenyl, methanesulfonyl, ethynyl, hydroxymethyl, acetyl, and combinations thereof.

In some embodiments of Structures (III), (III-A), and (III-B), Cy² is a phenyl substituted with one or more methyl; one to five methyl groups; one to four methyl groups; one to three methyl groups; one or two methyl groups; three methyl groups; two methyl groups; or one methyl group.

In some embodiments of Structures (III), (III-A), and (III-B), Cy² has a structure selected from the group consisting of:

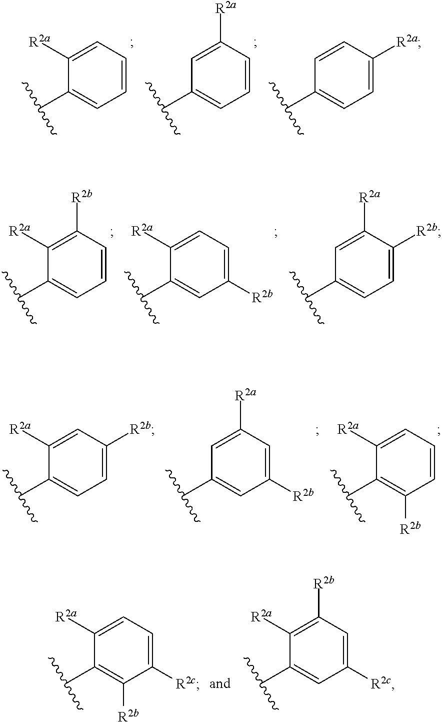

wherein R^{2a}, R^{2b}, and R^{2c} are each independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, 5- or 6-membered heterocyclyl comprising 1-4 heteroatoms selected from the group consisting of N, S, and O, and combinations thereof; or
wherein when any two of R^{2a}, R^{2b}, and R^{2e} are attached to adjacent carbon atoms, together with the carbon atoms to which they are attached, the R^{2a}, R^{2b}, and R^{2c} form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl, and wherein the substituted C₅-C₆ cycloalkyl fused to the phenyl and substituted 5- or 6-membered heterocyclic ring fused to the phenyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen.

In some embodiments of Structures (III), (III-A), and (III-B), Cy² is a phenyl substituted at two adjacent carbon atoms of the phenyl to form, together with the carbon atoms to which the substituents are attached, a dioxane fused to the phenyl, a furan fused to the phenyl, or a difluorodioxolane fused to the phenyl.

In some embodiments of Structures (III), (III-A), and (III-B), Cy² is selected from the group consisting of:

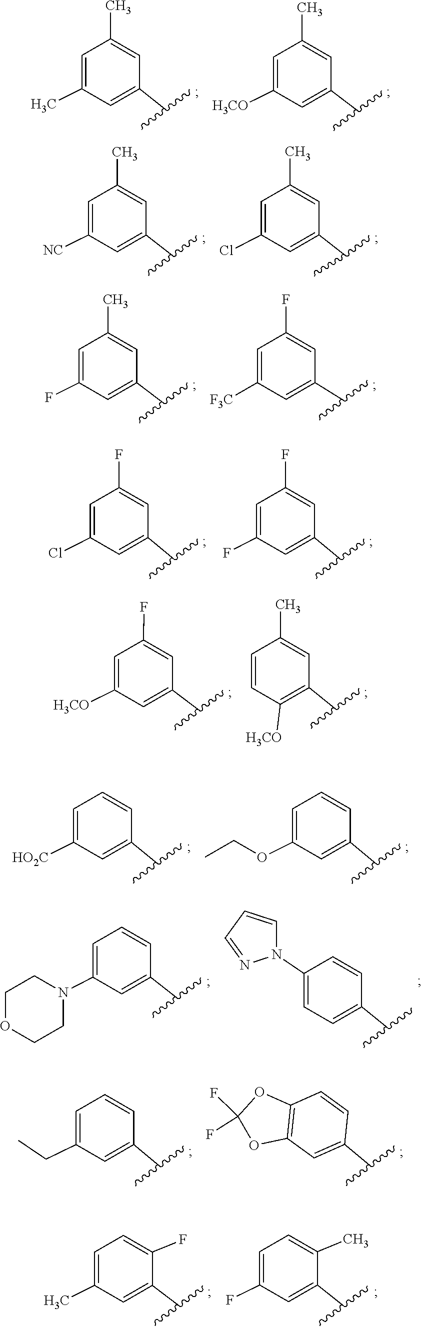

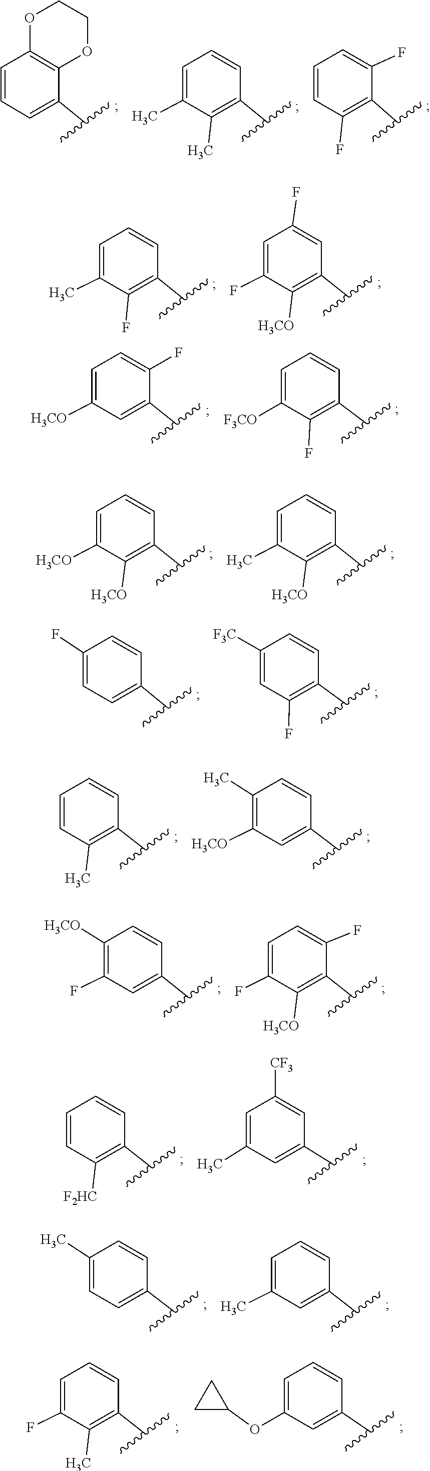

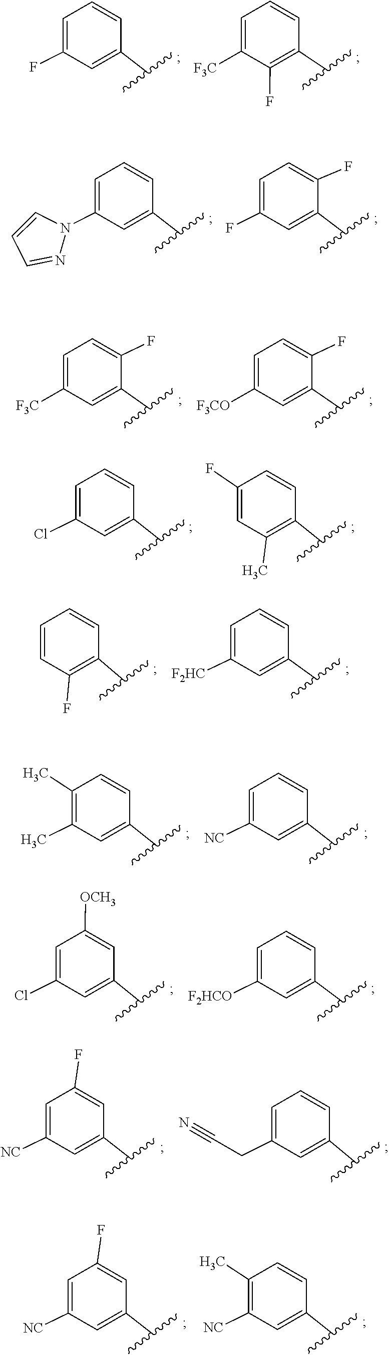

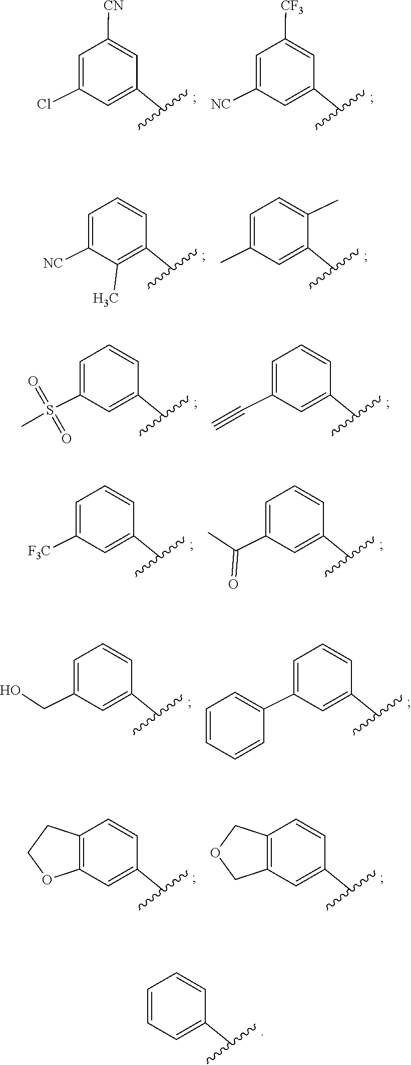

In some embodiments of Structures (III), (III-A), and (III-B), Cy² is selected from the group consisting of:

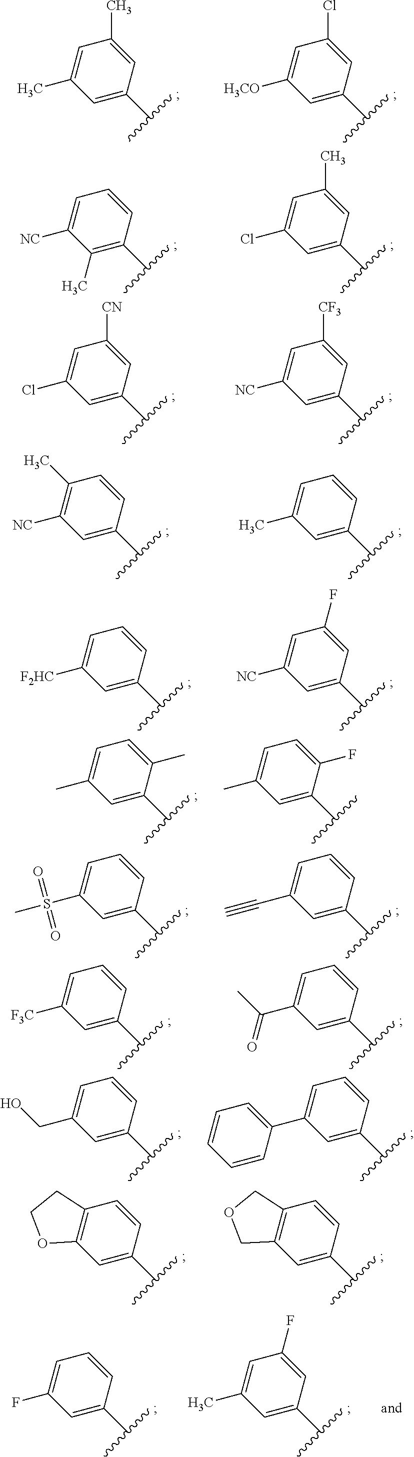

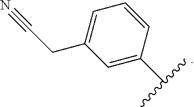 [SMILES: *c1cccc(CC#N)c1]

In some embodiments, the compound has Structure (III-C):

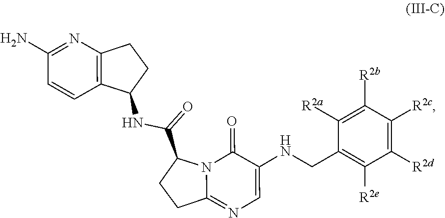

wherein R^{2a}, R²⁰, R^{2c}, R^{2d}, and R^{2e} are each independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when any two of R^{2a}, R^{2b}, R^{2e}, R^{2d}, and R^{2e} are connected to the phenyl at adjacent carbon atoms, the two R^{2a}, R^{2b}, R^{2e}, R^{2d}, and R^{2e} are connected, together with the carbon atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the phenyl, and wherein the substituted C₅-C₆ cycloalkyl fused to the phenyl and substituted 5- or 6-membered heterocyclic ring fused to the phenyl are optionally substituted with 1-4 or 1-8 substituents selected from the group consisting of C₁₋₆ alkyl, C₃₋₆ cycloalkyl, C₁₋₆ haloalkyl, C₃₋₆ halocycloalkyl, halogen, and a combination thereof, including combinations of one or more same or different C₁₋₆ alkyl, one or more same or different C₃₋₆ cycloalkyl, one or more same or different C₁₋₆ haloalkyl, one or more same or different C₃₋₆ halocycloalkyl, and/or one or more same or different halogen,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein R^{2a}, R^{2b}, R^{2c}, R^{2d}, and R^{2e} are optionally substituted with one or more substituents selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when R^{2a}, R^{2b}, R^{2e}, R^{2d}, and R^{2e} is a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

In some embodiments of Structure (III-C), R^{2a}, R^{2b}, R^{2c}, R^{2d}, and R^{2e} are each independently selected from the group consisting of hydrogen, C₁-C₃ alkyl, halogen, C₁-C₃ alkyloxy, C₃-C₆ cycloalkyloxy, CN, cyanoalkyl, COOH, CONH₂, hydroxyalkyl, C₁-C₃ alkyloxycarbonyl, haloalkyl, haloalkyloxy, aryl, C₁-C₃ alkylsulfonyl, C₂-C₆ alkynyl, C₁-C₃ acyl, 5- or 6-membered heteroaryl comprising 1-3 heteroatoms selected from the group consisting of N, S, and O, and 5- or 6-membered heterocyclyl comprising 1-4 heteroatoms selected from the group consisting of N, S, and O.

In some embodiments of Structure (III-C), R^{2a}, R^{2b}, R^{2c}, R^{2d}, and R^{2e} are each independently selected from the group consisting of hydrogen, methyl, ethyl, fluoro, chloro, methoxy, ethoxy, CN, cyanomethyl, COOH, methoxycarbonyl, ethoxycarbonyl, trifluoromethyl, difluoromethyl, fluoromethyl, fluoromethoxy, difluoromethoxy, trifluoromethoxy, pyrazole, cyclopropoxy, phenyl, methanesulfonyl, ethynyl, hydroxymethyl, acetyl, and morpholinyl.

In some embodiments of Structure (III-C), R^{2a}, R^{2b}, R^{2e}, R^{2d}, and R^{2e} are each independently selected from the group consisting of hydrogen, methyl, fluoro, chloro, methoxy, CN, cyanomethyl, phenyl, methanesulfonyl, ethynyl, hydroxymethyl, acetyl, trifluoromethyl, and difluoromethyl.

In some embodiments of Structure (III-C), R^{2a}, R^{2b}, R^{2c}, R^{2d}, and R^{2e} are each independently selected from the group consisting of hydrogen and methyl.

In some embodiments of Structure (III-C), one to five; one to four; one to three; one; two; three; four; or five of R^{2a}, R^{2b}, R^{2e}, R^{2d}, and R^{2e} are methyl.

In some embodiments of Structure (III-C), one of R^{2a}, R^{2b}, R^{2c}, R^{2d}, and R^{2e} is methyl. In some embodiments of Structure (III-C), two of R^{2a}, R^{2b}, R^{2e}, R^{2d}, and R^{2e} are methyl. In some embodiments of Structure (III-C), three of R^{2a}, R^{2b}, R^{2c}, R^{2d}, and R^{2e} are methyl.

In some embodiments of Structure (III-C), R^{2a} and R^{2b}, R^{2b} and R^{2c}, R^{2c} and R^{2d}, R^{2d} and R^{2e}, or a combination thereof, together with the carbon atoms to which R^{2a}, R^{2b}, R^{2c}, R^{2d} and R^{2e} are attached, form a dioxane fused with the phenyl, a furan fused to the phenyl, or a difluorodioxolane fused to the phenyl.

In some embodiments of Structure (III-C), the phenyl with R^{2a}, R^{2b}, R^{2c}, R^{2d}, and R^{2e} has a structure selected from the group consisting of:

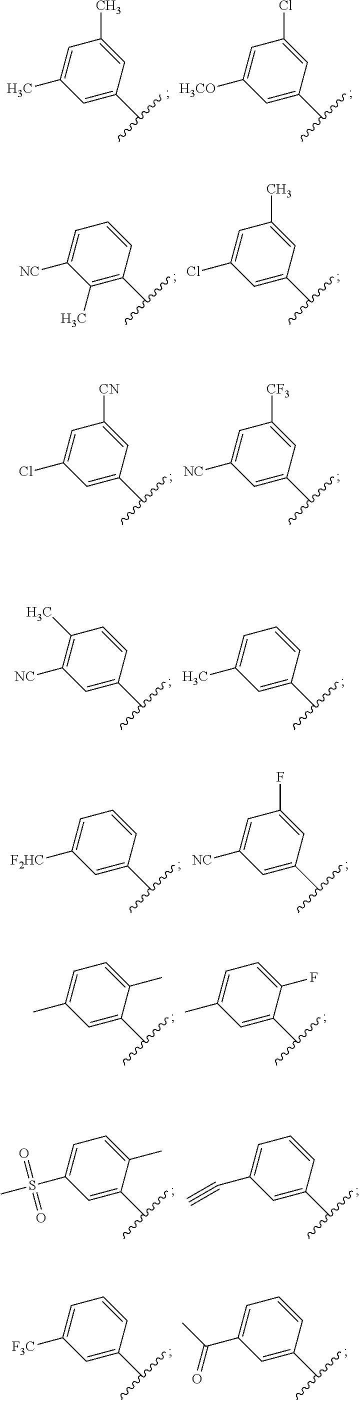

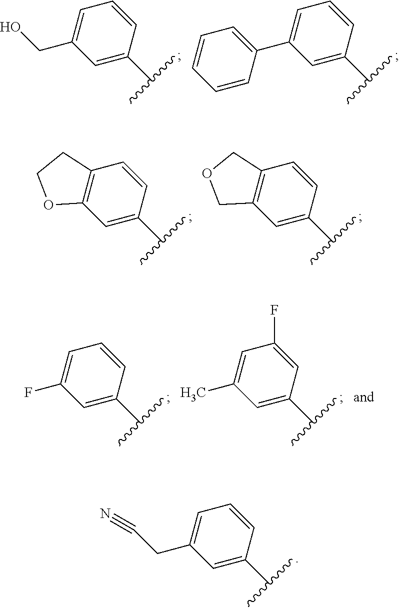

In some embodiments of Structures (III), (III-A), (III-B), and (III-C), the compound has a structure selected from the structures in Table 2 below.

In some embodiments of Structures (I), (I-A), (I-B), (I-C), (I-D), (I-E), (II), (II-A), (II-B), (II-C), (II-D), (III), (III-A), (III-B), and (III-C), one or more hydrogen atoms are replaced with one or more deuterium atoms.

The compounds of Structures (I), (I-A), (I-B), (I-C), (I-D), (I-E), (II), (II-A), (II-B), (II-C), (II-D), (III), (III-A), (III-B), and (III-C), and embodiments thereof, are useful in the methods and uses of the disclosure, optionally in the form of a salt, such as a pharmaceutically acceptable salt, or as a stereoisomer, or tautomer.

Certain embodiments provide a pharmaceutically acceptable salt of the indicated chemical compound (e.g., a hydrogen halide, such as a hydrogen chloride). Examples of pharmaceutically acceptable salts are set forth in, e.g., Burge, S. M. et al., J. Pharm. Sci 1977, 66, 1-19. Pharmaceutically acceptable salts include chlorides, bromides, iodides, formates, acetates, propionates, oxalates, malonates, succinates, fumarates, maleates, tartrates, citrates, benzoates, phthalates, sulfonates, arylsulfonates, alkylsulfonates, salts of fatty acids, and the like. Salts can be prepared by a variety of methods known to the skilled artisan, including a precipitation with, or an in-solution exposure to, an acid (e.g., treatment with gaseous HCl or an HCl solution), or a base.

In some embodiments of Structures (I), (I-A), (I-B), (I-C), (I-D), (I-E), (II), (II-A), (II-B), (II-C), (II-D), (III), (III-A), (III-B), and (III-C), the salt of the pharmaceutically acceptable salt is selected from the group consisting of trifluoroacetic acid, hydrogen chloride, acetic acid, hydrogen bromide, sulfuric acid, phosphoric acid, maleic acid, fumaric acid, lactic acid, tartric acid, citric acid, and gluconic acid.

In some embodiments of Structures (I), (I-A), (I-B), (I-C), (I-D), (I-E), (II), (II-A), (II-B), (II-C), (II-D), (III), (III-A), (III-B), and (III-C), the salt of the pharmaceutically acceptable salt is selected from the group consisting of trifluoroacetic acid, hydrogen chloride, and acetic acid.

In some embodiments of Structures (I), (I-A), (I-B), (I-C), (I-D), (I-E), (II), (II-A), (II-B), (II-C), (II-D), (III), (III-A), (III-B), and (III-C), the salt is a trifluoroacetic acid salt. In some embodiments of Structures (I), (I-A), (I-B), (I-C), (I-D), (I-E), (II), (II-A), (II-B), (II-C), (II-D), (III), (III-A), (III-B), and (III-C), the salt of the pharmaceutically acceptable salt is hydrogen chloride.

In some aspects, the compound of the disclosure is a compound of Table 1 and/or Table 2 below.

In some embodiments, the compound of Structures (I), (I-A), (I-B), (I-C), (I-D), (I-E), (II), (II-A), (II-B), (II-C), (II-D), (III), (III-A), (III-B), and (III-C), and embodiments thereof, are as set forth in the Examples, including the compounds listed in Table 1 and Table 2.

In some embodiments, the compounds of Structures (I), (I-A), (I-B), (I-C), (I-D), (I-E), (II), (II-A), (II-B), (II-C), (II-D), (III), (III-A), (III-B), and (III-C), and embodiments thereof, are provided in the form of a pharmaceutical composition comprising the compound, a stereoisomer, tautomer, or a salt thereof, such as a pharmaceutically acceptable salt, and at least one pharmaceutically acceptable carrier or excipient.

In some embodiments, the compounds of Structures (I), (I-A), (I-B), (I-C), (I-D), (I-E), (II), (II-A), (II-B), (II-C), (II-D), (III), (III-A), (III-B), and (III-C), and embodiments thereof, are set forth as a stereochemically pure enantiomer or diastereomer (e.g., an optically active compound with one or more stereocenter(s)). Unless specifically indicated otherwise, any compound with one or more stereocenters is intended to include and to describe each of the pure (+) and (−) enantiomers, any other diastereomers, mixtures that are enriched in an enantiomer or diastereomer (e.g., 5%, 10%, 20%, 30%, 40%, 50%, 60%, 70%, 75%, 80%, 85%, 90%, or 95% enantiomeric or diastereomeric excess), and a racemic mixture of enantiomers or diastereomers.

In some embodiments, the compound is a prodrug. A prodrug is a compound that is converted to a biologically active form under physiological conditions, often by hydrolysis, oxidation, or reduction (e.g., ester to acid form; carbamate to amino or hydroxy group; hydroxyamidine to amidine) Exemplary prodrugs are set forth in, e.g., Tilley, J. W., “Prodrugs of Benzamide,” Prodrugs 2007, 191-222; Peterlin-Masic et al. Curr. Pharma. Design 2006, 12, 73-91. Prodrugs for the amidine group include amidoximes, O-alkylamidoximes, acylamidines, carbamates, 1,2,4-oxadiazolin-4-ones, and the like.

### III. Methods of Inhibiting MASP-2

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful as inhibitors of MASP-2 and for therapeutic use.

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful in methods of treating a disease or disorder treatable by inhibiting MASP-2.

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful in methods for inhibiting MASP-2.

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful in methods for inhibiting MASP-2 complement activation in a subject.

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful in methods for inhibiting MASP-2 or MASP-2 complement activation in a subject by administering to the subject a therapeutically effective amount of the compound effective to inhibit MASP-2 or MASP-2 complement activation.

The compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are useful in the manufacture of medicaments for treating a disease or disorder treatable by inhibiting MASP-2.

The present disclosure also provides methods of treating a disease or disorder treatable by inhibiting MASP-2, comprising administering to a subject in need thereof a therapeutically effective amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof.

In some embodiments, the compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, is used after a subject has been diagnosed as being in need of treatment for a lectin complement-associated disease or disorder.

In some embodiments, the compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, are selected as compounds which exhibit selectivity for MASP-2 over thrombin, the method comprising administering the compound as described herein. In some embodiments, the selectivity ratio of MASP-2:thrombin is at least 1.1:1, 1.25:1, 1.5:1, 1.75:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, or 30:1.

In some aspects, the present disclosure provides a method for inhibiting MASP-2 in a subject, comprising administering to the subject a compound of Structure (I):

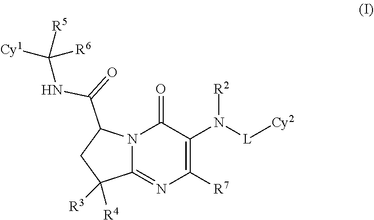

or a stereoisomer, tautomer, or pharmaceutically acceptable salt thereof, wherein:
Cy¹ is a substituted aryl or a substituted or unsubstituted 5-10-membered heteroaryl, or
Cy¹ together with one of R⁵ or R⁶ and the carbon to which they are attached form a substituted or unsubstituted C₃-C₆ cycloalkyl fused to a substituted or unsubstituted 5-10-membered heteroaryl, or a substituted or unsubstituted phenyl;
Cy² is a substituted aryl, a substituted or unsubstituted C₃-C₆ cycloalkyl, a substituted or unsubstituted 5-10-membered heteroaryl, or is hydrogen;
R² is hydrogen, a substituted or unsubstituted C₁-C₃ alkyl, or a substituted or unsubstituted cycloalkyl;
R³ and R⁴ are each independently hydrogen, halogen, substituted or unsubstituted C₁-C₃ alkyl, or substituted or unsubstituted cycloalkyl, or R³ and R⁴, together with the carbon to which they are attached, form a substituted or unsubstituted C₃-C₆ cycloalkyl or a substituted or unsubstituted C₅-C₆ cycloalkenyl;
R⁵ and R⁶ are each independently hydrogen, C₁-C₃ alkyl, alkoxy, haloalkyl, hydroxyalkyl, haloalkoxy, or C₃-C₆ cycloalkyl;
R⁷ is selected from the group consisting of hydrogen, halogen, substituted or unsubstituted alkyl, and substituted or unsubstituted cycloalkyl;
L is hydrogen or —(CR^{8a}R^{8b})ₙ—, wherein each —(CR^{8a}R^{8b})— is independently the same or different;
R^{8a} and R^{8b} are each independently hydrogen, substituted or unsubstituted linear or branched C₁-C₃ alkyl, or R^{8a} and R^{8b}, together with the carbon to which they are attached, form a substituted or unsubstituted C₃-C₆ cycloalkyl; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms in Structure (I) is optionally replaced with a deuterium atom; and
provided that the compound of Structure (I) does not have the structure

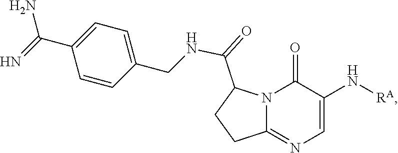

wherein R^{A} is benzyl, phenethyl, or 3-CF₃-benzyl.

In some embodiments, the present disclosure provides a method for inhibiting MASP-2 in a subject, comprising administering to the subject a compound of any one of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof.

In some embodiments, the present disclosure provides a method for inhibiting MASP-2 in a subject, comprising administering to the subject a compound having a structure in Table 1.

In some embodiments, the present disclosure provides a method for inhibiting MASP-2 in a subject, comprising administering to the subject a compound having a structure in Table 2.

In some embodiments, the present disclosure provides a method for inhibiting MASP-2 in a subject, comprising administering as a pharmaceutical composition a compound as disclosed herein, its stereoisomer, tautomer, or pharmaceutically acceptable salt, and a pharmaceutically acceptable carrier or excipient.

### IV. Methods of Treatment

In some aspects, the present disclosure provides a method of treating or preventing a subject suffering from, or at risk for developing, a MASP-2-associated disease or disorder, such as a MASP-2-dependent complement-associated disease or disorder, comprising administering an inhibitor of MASP-2.

The compound can be a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, as disclosed herein.

As described in U.S. Pat. Nos. 7,919,094; 8,840,893; 8,652,477; 8,951,522, 9,011,860, 9,475,885, 9,644,035, 9,644,035, 10,736,960, 10,059,776, 10,870,708; U.S. Patent Application Publication Nos. U.S. 2013/0344073, U.S. 2015/0166675, U.S. 2017/0137537, U.S. 2017/0166660, U.S. 2017/0253667, U.S. 2018/0105604, and U.S. 2020/0140570; and PCT Publication Nos. WO 2018/045054, WO 2019/036460, and WO 2021/178902 (each of which Omeros Corporation was the Applicant, the Applicant of the instant application; each of which, except U.S. Pat. No. 10,870,708 Omeros Corporation was the Assignee, the Assignee of the instant application; and each of which is hereby incorporated by reference in its entirety), MASP-2-dependent complement activation has been implicated as contributing to the pathogenesis of numerous acute and chronic disease states. For example, as described in U.S. Pat. No. 8,951,522, the primary function of the complement system, a part of the innate immune system, is to protect the host against infectious agents.

However, inappropriate or over-activation of the complement system can lead to serious disease, such as thrombotic microangiopathies (TMAs, including aHUS, TTP and HUS) in which endothelial damage, as well as fibrin and platelet-rich thrombi in the microvasculature, leads to organ damage.

The lectin pathway plays a dominant role in activating complement under conditions of endothelial cell stress or injury, and prevents the activation of MASP-2. The lectin pathway halts the sequence of enzymatic reactions that leads to the formation of the membrane attack complex, platelet activation, and leukocyte recruitment. As described in U.S. Pat. No. 8,652,477, in addition to initiation of the lectin pathway, MASP-2 can also activate the coagulation system, wherein prothrombin is cleaved to thrombin.

Accordingly, in some embodiments, the method comprises administering to a subject suffering from, or at risk for developing, a MASP-2-dependent complement-associated disease or disorder, an amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, in an amount sufficient to inhibit MASP-2 dependent complement activation in a subject, to thereby treat or prevent the disease or disorder. In some embodiments, the method can further comprise, prior to administering a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof to a subject, determining that the subject is afflicted with, or at risk for developing, the lectin complement-associated disease or disorder.

In some embodiments, the MASP-2-dependent complement-associated disease or disorder is selected from the group consisting of a thrombotic microangiopathy (TMA), a renal condition, an inflammatory reaction resulting from tissue or organ transplantation, an ischemia reperfusion injury, a complication associated with diabetes, a cardiovascular disease or disorder, an inflammatory gastrointestinal disorder, a pulmonary disorder, an ophthalmic disease or disorder, disseminated intravascular coagulation, graft-versus-host disease, veno-occlusive disease, diffuse alveolar hemorrhage, idiopathic pneumonia syndrome, capillary leak syndrome, engraftment syndrome, fluid overload, and a combination thereof.

In some embodiments, the MASP-2-dependent complement-associated disease or disorder is selected from the group consisting of a thrombotic microangiopathy (TMA) thrombotic thrombocytopenia purpura (TTP), refractory TTP, Upshaw-Schulman Syndrome (USS), hemolytic uremic syndrome (HUS), atypical hemolytic syndrome (aHUS), non-Factor H-dependent atypical hemolytic syndrome, aHUS secondary to an infection, plasma therapy-resistant aHUS, a TMA secondary to cancer, a TMA secondary to chemotherapy, a TMA secondary to transplantation, a TMA associated with hematopoietic stem cell transplant, and a combination thereof.

In some embodiments, the MASP-2-dependent complement-associated disease or disorder is graft-versus-host disease. In some embodiments, the method comprises administering to a subject suffering from or at risk for developing graft-versus-host disease (GVHD), including acute GVHD, chronic GVHD or steroid-resistant GVHD an amount of a compound of the disclosure in an amount sufficient to inhibit MASP-2 dependent complement activation in the subject to thereby treat or prevent the disease or disorder. In some embodiments, the subject suffering from, or at risk for developing, GVHD has previously undergone, is undergoing, or will undergo a hematopoietic stem cell transplant.

In some embodiments, the MASP-2-dependent complement-associated disease or disorder is diffuse alveolar hemorrhage (DAH). In some embodiments, the method comprises administering to a subject suffering from, or at risk for developing diffuse alveolar hemorrhage (DAH) an amount of a compound of the disclosure in an amount sufficient to inhibit MASP-2 dependent complement activation in the subject to thereby treat or prevent the disease or disorder. In some embodiments, the subject suffering from, or at risk for developing DAH has previously undergone, is undergoing, or will undergo a hematopoietic stem cell transplant.

In some embodiments, the MASP-2-dependent complement-associated disease or disorder is veno-occlusive disease (VOD). In some embodiments, the method comprises administering to a subject suffering from, or at risk for developing veno-occlusive disease (VOD) an amount of a compound of the disclosure in an amount sufficient to inhibit MASP-2 dependent complement activation in the subject to thereby treat or prevent the disease or disorder. In some embodiments, the subject suffering from, or at risk for developing, VOD has previously undergone, is undergoing, or will undergo a hematopoietic stem cell transplant.

In some embodiments, the method comprises administering to a subject suffering from, or at risk for developing idiopathic pneumonia syndrome (IPS) an amount of a compound of the disclosure in an amount sufficient to inhibit MASP-2 dependent complement activation in the subject to thereby treat or prevent the disease or disorder. In some embodiments, the subject suffering from, or at risk for developing, IPS has previously undergone, is undergoing, or will undergo a hematopoietic stem cell transplant.

In some embodiments, the method comprises administering to a subject suffering from, or at risk for developing capillary leak syndrome (CLS) an amount of a compound of the disclosure in an amount sufficient to inhibit MASP-2 dependent complement activation in the subject to thereby treat or prevent the disease or disorder. In some embodiments, the subject suffering from, or at risk for developing CLS has previously undergone, is undergoing, or will undergo a hematopoietic stem cell transplant.

In some embodiments, the method comprises administering to a subject suffering from, or at risk for developing engraftment syndrome (ES) an amount of a compound of the disclosure in an amount sufficient to inhibit MASP-2 dependent complement activation in the subject to thereby treat or prevent the disease or disorder. In some embodiments, the subject suffering from, or at risk for developing ES has previously undergone, is undergoing, or will undergo a hematopoietic stem cell transplant.

In some embodiments, the method comprises administering to a subject suffering from, or at risk for developing fluid overload (FO) an amount of a compound of the disclosure in an amount sufficient to inhibit MASP-2 dependent complement activation in the subject to thereby treat or prevent the disease or disorder. In some embodiments, the subject suffering from, or at risk for developing FO has previously undergone, is undergoing, or will undergo a hematopoietic stem cell transplant.

In some embodiments, the method comprises administering to a subject suffering from, or at risk for developing, any of the above-referenced diseases or conditions an amount of a compound as disclosed in PCT Application No. PCT/US19/34225, which is hereby incorporated in its entirety.

In some embodiments, the MASP-2-dependent complement-associated disease or disorder is a renal condition. In some embodiments, the renal condition is selected from the group consisting of mesangioproliferative glomerulonephritis, membranous glomerulonephritis, membranoproliferative glomerulonephritis (mesangiocapillary glomerulonephritis), acute post infectious glomerulonephritis (poststreptococcal glomerulonephritis), C3 glomerulopathy, cryoglobulinemic glomerulonephritis, pauci-immune necrotizing crescentic glomerulonephritis, lupus nephritis, Henoch-Schonlein purpura nephritis, IgA nephropathy, tubule-interstitial disease, or combinations thereof.

In some embodiments, the MASP-2-dependent complement-associated disease or disorder is renal fibrosis (e.g., tubulointerstitial fibrosis) and/or proteinuria in a subject suffering from or at risk for developing chronic kidney disease, chronic renal failure, glomerular disease (e.g., focal segmental glomerulosclerosis), an immune complex disorder (e.g., IgA nephropathy, membranous nephropathy), lupus nephritis, nephrotic syndrome, diabetic nephropathy, tubulointerstitial damage and glomerulonepthritis (e.g., C3 glomerulopathy), or a disease or condition associated with proteinuria, including, but not limited to, nephrotic syndrome, pre-eclampsia, eclampsia, toxic lesions of kidneys, amyloidosis, collagen vascular diseases (e.g., systemic lupus erythematosus), dehydration, glomerular diseases (e.g., membranous glomerulonephritis, focal segmental glomerulonephritis, C3 glomerulopathy, minimal change disease, lipoid nephrosis), strenuous exercise, stress, benign orthostatis (postural) proteinuria, focal segmental glomerulosclerosis, IgA nephropathy (i.e., Berger's disease), IgM nephropathy, membranoproliferative glomerulonephritis, membranous nephropathy, minimal change disease, sarcoidosis, Alport's syndrome, diabetes mellitus (diabetic nephropathy), drug-induced toxicity (e.g., NSAIDS, nicotine, penicillamine, lithium carbonate, gold and other heavy metals, ACE inhibitors, antibiotics (e.g., adriamycin), opiates (e.g., heroin), or other nephrotoxins), Fabry's disease, infections (e.g., HIV, syphilis, hepatitis A, B or C, poststreptococcal infection, urinary schistosomiasis), aminoaciduria, Fanconi syndrome, hypertensive nephrosclerosis, interstitial nephritis, sickle cell disease, hemoglobinuria, multiple myeloma, myoglobinuria, organ rejection (e.g., kidney transplant rejection), ebola hemorrhagic fever, Nail patella syndrome, familial Mediterranean fever, HELLP syndrome, systemic lupus erythematosus, Wegener's granulomatosis, Rheumatoid arthritis, Glycogen storage disease type 1, Goodpasture's syndrome, Henoch-Schonlein purpura, urinary tract infection which has spread to the kidneys, Sjogren's syndrome or post-infections glomerulonepthritis.

In some embodiments, the MASP-2-dependent complement-associated disease or disorder is selected from the group consisting of renal fibrosis, proteinuria, or combinations thereof.

In some embodiments, the MASP-2-dependent complement-associated disease or disorder is an inflammatory reaction resulting from tissue or solid organ transplantation, including allotransplantation or xenotransplantation of whole organs (e.g., kidney, heart, liver, pancreas, lung, cornea, and the like) or tissue grafts (e.g., valves, tendons, bone marrow, and the like).

In some embodiments, the MASP-2-dependent complement-associated disease or disorder is an ischemia reperfusion injury (I/R), including myocardial I/R, gastrointestinal I/R, renal I/R, and I/R following an aortic aneurism repair, I/R associated with cardiopulmonary bypass, cerebral I/R, stroke, organ transplant or reattachment of severed or traumatized limbs or digits, spinal cord injury, revascularization to transplants and/or replants, complex regional pain syndrome, and hemodynamic resuscitation following shock, surgical procedures, or similar, or combinations thereof.

In some embodiments, the MASP-2-dependent complement-associated disease or disorder is a complication associated with non-obese diabetes (Type-1 diabetes or Insulin-dependent diabetes mellitus) and/or complications associated with Type-1 or Type-2 (adult onset) diabetes including diabetic angiopathy, diabetic neuropathy, diabetic retinopathy, diabetic macular edema, and the like, or combinations thereof.

In some embodiments, the MASP-2-dependent complement-associated disease or disorder is a cardiovascular disease or disorder, including Henoch-Schonlein purpura nephritis, systemic lupus erythematosus-associated vasculitis, vasculitis associated with rheumatoid arthritis (also called malignant rheumatoid arthritis), immune complex vasculitis, and Takayasu's disease; dilated cardiomyopathy; diabetic angiopathy; Kawasaki's disease (arteritis); venous gas embolus (VGE); and inhibition of restenosis following stent placement, rotational atherectomy, percutaneous transluminal coronary angioplasty (PTCA), and the like; or combinations thereof.

In some embodiments, the MASP-2-dependent complement-associated disease or disorder is an inflammatory gastrointestinal disorder, including pancreatitis, diverticulitis and bowel disorders including Crohn's disease, ulcerative colitis, irritable bowel syndrome, inflammatory bowel disease (IBD), or similar, or combinations thereof.

In some embodiments, the MASP-2-dependent complement-associated disease or disorder is a pulmonary disorder, including acute respiratory distress syndrome, transfusion-related acute lung injury, ischemia/reperfusion acute lung injury, chronic obstructive pulmonary disease, asthma, Wegener's granulomatosis, antiglomerular basement membrane disease (Goodpasture's disease), meconium aspiration syndrome, aspiration pneumonia, bronchiolitis obliterans syndrome, idiopathic pulmonary fibrosis, acute lung injury secondary to burn, non-cardiogenic pulmonary edema, transfusion-related respiratory depression, emphysema, and the like, or combinations thereof.

In some embodiments, the MASP-2-dependent complement-associated disease or disorder is an extracorporeal exposure-triggered inflammatory reaction and the method comprises treating a subject undergoing an extracorporeal circulation procedure or pretreating a subject prior to undergoing an extracorporeal circulation procedure. In some embodiments, the extracorporeal circulation procedure includes hemodialysis, plasmapheresis, leukopheresis, extracorporeal membrane oxygenation (ECMO), heparin-induced extracorporeal membrane oxygenation LDL precipitation (HELP), cardiopulmonary bypass (CPB), and the like, or combinations thereof. In some embodiments, the method further comprises treating a subject undergoing an extracorporeal circulation procedure.

In some embodiments, the MASP-2-dependent complement-associated disease or disorder is selected from inflammatory or non-inflammatory arthritides and other musculoskeletal disorders, e.g., osteoarthritis, rheumatoid arthritis, juvenile rheumatoid arthritis, gout, neuropathic arthropathy, psoriatic arthritis, ankylosing spondylitis or other spondyloarthropathies and crystalline arthropathies, muscular dystrophy, systemic lupus erythematosus (SLE), or similar, or combinations thereof.

In some embodiments, the MASP-2-dependent complement-associated disease or disorder is a skin disorder, for example, psoriasis, autoimmune bullous dermatoses, eosinophilic spongiosis, bullous pemphigoid, epidermolysis bullosa acquisita, atopic dermatitis, herpes gestationis, and other skin disorders. In some embodiments, the MASP-2-dependent complement-associated disease or disorder is a thermal burn, chemical burn, or combinations thereof, including capillary leakage caused thereby.

In some embodiments, the MASP-2-dependent complement-associated disease or disorder is a peripheral nervous system (PNS) and/or central nervous system (CNS) disorder or injury including multiple sclerosis (MS), myasthenia gravis (MG), Huntington's disease (HD), amyotrophic lateral sclerosis (ALS), Guillain Barre syndrome, reperfusion following stroke, complex regional pain syndrome, degenerative discs, spinal cord injury, cerebral trauma, Parkinson's disease (PD), Alzheimer's disease (AD), Miller-Fisher syndrome, cerebral trauma and/or hemorrhage, traumatic brain injury, demyelination, meningitis, or similar, or combinations thereof.

In some embodiments, the MASP-2-dependent complement-associated disease or disorder is sepsis or a condition resulting from sepsis including severe sepsis, septic shock, acute respiratory distress syndrome resulting from sepsis, hemolytic anemia, systemic inflammatory response syndrome, hemorrhagic shock, or the like, or combinations thereof.

In some embodiments, the MASP-2-dependent complement-associated disease or disorder is a urogenital disorder including painful bladder disease, sensory bladder disease, chronic abacterial cystitis and interstitial cystitis, male and female infertility, placental dysfunction and miscarriage, pre-eclampsia, or similar, as well as combinations thereof. In some embodiments, the MASP-2-dependent complement-associated disease or disorder is an inflammatory reaction in a subject being treated with chemotherapeutics, radiation therapy, or combinations thereof.

In some embodiments, the MASP-2-dependent complement-associated disease or disorder is an inflammatory reaction in a subject being treated with chemotherapeutics and/or radiation therapy, including for the treatment of cancerous conditions.

In some embodiments, the MASP-2-dependent complement-associated disease or disorder is an angiogenesis-dependent cancer, including a solid tumor(s), blood borne tumor(s), high-risk carcinoid tumors, tumor metastases, and the like, or combinations thereof.

In some embodiments, the MASP-2-dependent complement-associated disease or disorder is an angiogenesis-dependent benign tumor, including hemangiomas, acoustic neuromas, neurofibromas, trachomas, carcinoid tumors, pyogenic granulomas, or similar, or combinations thereof.

In some embodiments, the MASP-2-dependent complement-associated disease or disorder is an endocrine disorder including Hashimoto's thyroiditis, stress, anxiety, other potential hormonal disorders involving regulated release of prolactin, growth or insulin-like growth factor, adrenocorticotropin from the pituitary, or similar, or combinations thereof.

In some embodiments, the MASP-2-dependent complement-associated disease or disorder is an ophthalmic disease or disorder including age-related macular degeneration, glaucoma, endophthalmitis, and the like, or combinations thereof.

In some embodiments, the MASP-2-dependent complement-associated disease or disorder is an ocular angiogenic disease or disorder including age-related macular degeneration, uveitis, ocular melanoma, corneal neovascularization, primary pterygium, HSV stromal keratitis, HSV-1-induced corneal lymphangiogenesis, proliferative diabetic retinopathy, diabetic macular edema, retinopathy of prematurity, retinal vein occlusion, corneal graft rejection, neovascular glaucoma, vitreous hemorrhage secondary to proliferative diabetic retinopathy, neuromyelitis optica, rubeosis, or similar, or combinations thereof.

In some embodiments, the MASP-2-dependent complement-associated disease or disorder is disseminated intravascular coagulation (DIC) or other complement mediated coagulation disorder, including DIC secondary to sepsis, severe trauma, including neurological trauma (e.g., acute head injury; see Kumura et al, Acta Neurochirurgica 55:23-28 (1987), infection (e.g., bacterial, viral, fungal, parasitic), cancer, obstetrical complications, liver disease, severe toxic reaction (e.g., snake bite, insect bite, transfusion reaction), shock, heat stroke, transplant rejection, vascular aneurysm, hepatic failure, cancer treatment by chemotherapy or radiation therapy, burn, or accidental radiation exposure.

In some embodiments, the MASP-2-dependent complement-associated disease or disorder is selected from the group consisting of acute radiation syndrome, dense deposit disease, Degos Disease, Catastrophic Antiphospholipid Syndrome (CAPS), Behcet's disease, cryoglobulinemia, paroxysmal nocturnal hemoglobinuria (“PNH”), cold agglutinin disease, or combinations thereof.

In some embodiments, the MASP-2-dependent complement-associated disease or disorder is atypical hemolytic uremic syndrome (aHUS).

In some embodiments, the MASP-2-dependent complement-associated disease or disorder is hematopoietic stem cell transplant-associated TMA.

In some embodiments, the MASP-2-dependent complement-associated disease or disorder is immunoglobulin A nephropathy (IgAN).

In some embodiments, the MASP-2-dependent complement-associated disease or disorder is lupus nephritis (LN).

In some embodiments, the MASP-2-dependent complement-associated disease or disorder is COVID-19 induced acute respiratory distress syndrome (ARDS), COVID-19 induced pneumonia, COVID-19 or similar systemic infectious disease, or Long COVID.

In some embodiments, the method comprises administering to a subject suffering from, or at risk for developing a disease, disorder or condition associated with fibrin-induced activation of the complement system and the associated activation of the coagulation and/or contact systems an amount of a compound according to any one of the foregoing embodiments (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) in an amount sufficient to inhibit MASP-2 dependent complement activation in the subject to thereby treat or prevent the disease or disorder.

In some embodiments, the subject is suffering from, or at risk of developing, a disease, disorder or condition associated with complement-related inflammation, excessive coagulation or contact system activation initiated by fibrin or activated platelets. In some embodiments, the subject is suffering from, or at risk of developing, a disease or disorder selected from the group consisting of arterial thrombosis, venous thrombosis, deep vein thrombosis, post-surgical thrombosis, restenosis following coronary artery bypass graft and/or an interventional cardiovascular procedure (e.g., angioplasty or stent placement), atherosclerosis, plaque rupture, plaque instability, restenosis, hypotension, acute respiratory distress syndrome (ARDS), systemic inflammatory response syndrome (SIRS), disseminated intravascular coagulation (DIC), veno-occlusive disease (VOD), thrombotic microangiopathy, lupus nephritis, superficial thrombophlebitis, Factor V Leiden mutation, ischemic/reperfusion injury, human immunodeficiency virus (HIV) infection, undergoing hormone-replacement therapy (HRT), Alzheimer's disease and/or suffering from a hypercoagulable state.

In some embodiments, the subject is suffering from, or at risk for developing an acquired hypercoagulable state due to at least one or more of the following: undergoing therapy with a drug selected from the group consisting of 5-FU, GM-CSF, cisplatin, heparin, COX-2 inhibitor, contrast media, corticosteroids and antipsychotics; venous stasis (immobilization, surgery, etc.), antiphospholipid syndrome, cancer (promyelocytic leukemia, lung, breast, prostate, pancreas, stomach and colon tumors), tissue injury due to trauma or surgery, presence of a catheter in a central vein, acquired deficiency of a protein involved in clot formation (e.g., protein C), paroxysmal nocturnal hemoglobinuria (PNH), elevated levels of homocysteine, heart failure, presence of a mechanical valve, pulmonary hypertension with in situ thrombosis, atrial fibrillation, heparin-induced thrombocytopenia (HIT), heparin-induced thrombocytopenia and thrombosis (HITT), Kawasaki disease with in situ thrombus, Takayasu arteritis with in situ thrombus, thrombophilia of metastatic cancer, elevated Factor VIII levels, pregnancy, inflammatory bowel disease (IBD), or due to a genetic defect that causes or increases the risk of developing, a hypercoagulable state, such as a genetic defect selected from the group consisting of a Prothrombin 20210 gene mutation, an MTHFR mutation, a deficiency of protein C, a deficiency of protein S, a deficiency of protein A, a deficiency of protein Z, an antithrombin deficiency, and a genetic disorder producing thrombophilia.

In some embodiments, the subject is suffering from, or at risk for developing, a disease or disorder that is amenable to treatment with a kallikrein inhibitor. In some embodiments, the subject is suffering from, or at risk for developing a disease or disorder amenable to treatment with a kallikrein inhibitor is selected from the group consisting of hereditary angioedema, diabetic macular edema and bleeding during cardiopulmonary bypass. In some embodiments, the subject is suffering from, or at risk for developing, a disease or disorder that is amenable to treatment with a thrombin inhibitor, such as arterial thrombosis, venous thrombosis, pulmonary embolism, atrial fibrillation, heparin-induced thrombocytopenia, conversion from one anticoagulant to another, or off-label use for extracorporeal circuit patency of continuous renal replacement therapy (CRRT) in critically ill subjects with HIT (maintenance).

In some embodiments, the subject has previously experienced, is currently suffering from, or is at risk for developing atrial fibrillation and the MASP-2 inhibitory compound (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) is administered in an amount sufficient to reduce the risk of stroke in said subject. In some embodiments, the subject is suffering from, or at risk for developing, a disease or disorder that is amenable to treatment with a factor XII inhibitor, such as deep vein thrombosis (both primary prophylaxis and extended therapy), pulmonary embolism, nonvalvular atrial fibrillation, prevention of recurrent ischemia after acute coronary syndrome in subjects with or without atrial fibrillation, end-stage renal disease, cerebral ischemia, angina, or to reduce or prevent clotting associated with medical devices (e.g., valves, small caliber grafts, etc.) and/or extracorporeal circuits.

In some embodiments, the subject has previously experienced, is currently suffering from, or is at risk for developing nonvalvular atrial fibrillation and the MASP-2 inhibitory compound (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) is administered in an amount sufficient to reduce the risk of stroke and/or embolism in said subject. In some embodiments, the subject has an acquired disease or disorder that increases the propensity for thromboembolism, such as a disease or disorder selected from the group consisting of atherosclerosis, antiphospholipid antibodies, cancer (e.g., promyelocytic leukemia, lung, breast, prostate, pancreatic, stomach and colon), hyperhomocysteinemia, infection, tissue injury, venous stasis (such as due to surgery, orthopedic or paralytic immobilization, heart failure, pregnancy, or obesity) and a subject taking oral contraceptives that contain estrogen.

In some embodiments, the subject is in need of anticoagulant therapy and the MASP-2 inhibitory compound (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) is used as a replacement for standard anticoagulant therapy (e.g., Warfarin). In some embodiments, the subject has a condition that normally prohibits standard anticoagulant therapy, such as CNS amyloid angiopathy. In some embodiments of the method, the MASP-2 inhibitory compound is administered as a bridging agent perioperatively in a subject otherwise on standard anticoagulation therapy. In some embodiments, the subject has sickle cell disease which is a vaso-occlusive disorder involving activation of platelets.

Atypical hemolytic uremic syndrome (aHUS) is part of a group of conditions termed “Thrombotic microangiopathies.” In the atypical form of HUS (aHUS), the disease is associated with defective complement regulation and can be either sporadic or familial. Familial cases of aHUS are associated with mutations in genes coding for complement activation or complement regulatory proteins, including complement factor H, factor I, factor B, membrane cofactor CD46 as well as complement factor H-related protein 1 (CFHR1) and complement factor H-related protein 3 (CFHR3). (Zipfel, P. F., et al., PloS Genetics 3(3):e41 (2007)). The unifying feature of this diverse array of genetic mutations associated with aHUS is a predisposition to enhanced complement activation on cellular or tissue surfaces. A subject is a risk for developing aHUS upon the onset of at least one or more symptoms indicative of aHUS (e.g., the presence of anemia, thrombocytopenia and/or renal insufficiency) and/or the presence of thrombotic microangiopathy in a biopsy obtained from the subject. The determination of whether a subject is at risk for developing aHUS comprises determining whether the subject has a genetic predisposition to developing aHUS, which may be carried out by assessing genetic information (e.g. from a database containing the genotype of the subject), or performing at least one genetic screening test on the subject to determine the presence or absence of a genetic marker associated with aHUS (i.e., determining the presence or absence of a genetic mutation associated with aHUS in the genes encoding complement factor H (CFH), factor I (CFI), factor B (CFB), membrane cofactor CD46, C3, complement factor H-related protein 1 (CFHR1), or THBD (encoding the anticoagulant protein thrombodulin) or complement factor H-related protein 3 (CFHR3), or complement factor H-related protein 4 (CFHR4)) either via genome sequencing or gene-specific analysis (e.g., PCR analysis), and/or determining whether the subject has a family history of aHUS. Methods of genetic screening for the presence or absence of a genetic mutation associated with aHUS are well established, for example, see Noris M et al. “Atypical Hemolytic-Uremic Syndrome,” 2007 Nov. 16 [Updated 2011 Mar. 10]. In: Pagon R A, Bird T D, Dolan C R, et al., editors. GeneReviews™, Seattle (WA): University of Washington, Seattle.

Hematopoietic stem cell transplant-associated TMA (HSCT-TMA) is a life-threatening complication that is triggered by endothelial injury. The kidney is the most commonly affected organ, though HSCT-TMA can be a multi-system disease that also involves the lung, bowel, heart, and brain. The occurrence of even mild TMA is associated with long-term renal impairment. Development of post-allogeneic HSCT-associated TMA differs in frequency based on varying diagnostic criteria and conditioning and graft-versus-host disease prophylaxis regimens, with calcineurin inhibitors being the most frequent drugs implicated (Ho V T et al., Biol Blood Marrow Transplant, 11(8):571-5, 2005). Treatment with the MASP-2 inhibitory antibody narsoplimab is being investigated in clinical trials of lectin pathway-mediated conditions where MASP-2 is believed to play a key role in pathophysiology. In hematopoietic stem cell transplantation-associated thrombotic microangiopathy (HSCT-TMA), plasma MASP-2 levels are elevated versus healthy controls, and narsoplimab has been shown to reduce HSCT-TMA plasma-mediated endothelial damage in a tissue culture model (Elhadad S, et al., MASP2 levels are elevated in thrombotic microangiopathies: association with microvascular endothelial cell injury and suppression by anti-MASP2 antibody narsoplimab. Clin Exp Immunol (2021) 203(1):96-104). In a pivotal clinical trial for HSCT-TMA, once-weekly narsoplimab treatment resulted in 61% response rate based on improvement in laboratory TMA markers and clinical benefit, indicating clinically relevant resolution of HSCT-TMA pathophysiology (Khaled S K, et al.. Narsoplimab, a mannan-binding lectin-associated serine protease-2 inhibitor, for the treatment of adult hematopoietic stem-cell transplantation-associated thrombotic microangiopathy. J Clin Oncol (2022) 40(22):2447-57).

Immunoglobulin A nephropathy (IgAN) is an autoimmune kidney disease resulting in intrarenal inflammation and kidney injury. IgAN is the most common primary glomerular disease globally. With an annual incidence of approximately 2.5 per 100,000, it is estimated that 1 in 1400 persons in the U.S. will develop IgAN. As many as 40% of subjects with IgAN will develop end-stage renal disease (ESRD). Subjects typically present with microscopic hematuria with mild to moderate proteinuria and variable levels of renal insufficiency (Wyatt R. J., et al., N Engl J Med 36S(25):2402-4, 2013). Clinical markers such as impaired kidney function, sustained hypertension, and heavy proteinuria (over 1 g per day) are associated with poor prognosis (Goto M et al., Nephrol Dial Transplant 24(10):3068-74, 2009; Berthoux F. et al., J Am Soc Nephrol 22(4):752-61, 2011). Proteinuria is the strongest prognostic factor independent of other risk factors in multiple large observational studies and prospective trials (Coppo R. et al., J Nephrol 18(5):503-12, 2005; Reich H. N., et al., J Am Soc Nephrol 18(12):3177-83, 2007). It is estimated that 15-20% of subjects reach ESRD within 10 years of disease onset if left untreated (D'Amico G., Am J Kidney Dis 36(2):227-37, 2000). The diagnostic hallmark of IgAN is the predominance of IgA deposits, alone or with IgG, IgM, or both, in the glomerular mesangium. In a Phase 2 clinical trial for severe IgA nephropathy, once-weekly narsoplimab treatment was well tolerated; after 3 years of follow-up, the annualized rate of kidney function decline was slowed in narsoplimab-treated patients versus a matched external comparator group (5.2 vs 8.6 mL/min/yr, respectively) and urine protein excretion decreased by 38% from baseline in narsoplimab-treated patients (Lafayette R A, et al, Safety, tolerability and efficacy of narsoplimab, a novel MASP-2 inhibitor for the treatment of IgA nephropathy. Kidney Int Rep (2020) 5(11):2032-41; Lafayette R A, et al., Long-term phase 2 efficacy of the MASP-2 inhibitor narsoplimab for treatment of severe IgA nephropathy [abstract]. J Am Soc Nephrol (2021) 32(Suppl):B10). Based on this evidence, narsoplimab appears to be beneficial in the treatment of lectin pathway-mediated diseases.

A main complication of systemic lupus erythematosus (SLE) is nephritis, also known as lupus nephritis, which is classified as a secondary form of glomerulonephritis. Up to 60% of adults with SLE have some form of kidney involvement later in the course of the disease (Koda-Kimble et al., Koda-Kimble and Young's Applied Therapeutics: the clinical use of drugs, 10th Ed, Lippincott Williams & Wilkins: pages 792-9, 2012) with a prevalence of 20-70 per 100,000 people in the U.S. Lupus nephritis often presents in subjects with other symptoms of active SLE, including fatigue, fever, rash, arthritis, serositis, or central nervous system disease (Pisetsky D. S. et al., Med Clin North Am 81(1): 113-28, 1997). Some subjects have asymptomatic lupus nephritis; however, during regular follow-up, laboratory abnormalities such as elevated serum creatinine levels, low albumin levels, or urinary protein or sediment suggest active lupus nephritis.

Deficiency or blockade of MASP-2 in experimental models has also been shown to have beneficial effects in ischemic reperfusion injury (see e.g., Schwaeble W J, et al. Targeting of mannan binding lectin-associated serine protease-2 confers protection from myocardial and gastrointestinal ischemia/reperfusion injury. Proc Natl Acad Sci USA (2011) 108(18):7523-8; Clark J E, et al., Cardioprotection by an anti-MASP-2 antibody in a murine model of myocardial infarction. Open Heart (2018) 5(1):e000652; Orsini F, et al., Mannan binding lectin-associated serine protease-2 (MASP-2) critically contributes to post-ischemic brain injury independent of MASP-1. J Neuroinflammation (2016) 13(1):213; and Asgari E, et al., Mannan-binding lectin-associated serine protease 2 is critical for the development of renal ischemia reperfusion injury and mediates tissue injury in the absence of complement C4. FASEB J (2014) 28(9):3996-4003); myocardial infarction (see e.g., Clark J E, et al., Cardioprotection by an anti-MASP-2 antibody in a murine model of myocardial infarction. Open Heart (2018) 5(1):e000652); stroke (see e.g., Orsini F, et al., Mannan binding lectin-associated serine protease-2 (MASP-2) critically contributes to post-ischemic brain injury independent of MASP-1. J Neuroinflammation (2016) 13(1):213); transplantation (see e.g., Asgari E, et al., Mannan-binding lectin-associated serine protease 2 is critical for the development of renal ischemia reperfusion injury and mediates tissue injury in the absence of complement C4. FASEB J (2014) 28(9):3996-4003); renal diseases (see e.g., Alghadban S, et al., Absence of the Lectin Activation Pathway of Complement Ameliorates Proteinuria-Induced Renal Injury. Front Immunol (2019) 10(2238):2238); rheumatoid arthritis (see e.g., Banda N K, et al., Deconstructing the lectin pathway in the pathogenesis of experimental inflammatory arthritis: Essential role of the lectin ficolin B and mannose-binding protein-associated serine protease 2. J Immunol (2017) 199(5): 1835-45); TMA (see e.g., Elhadad S, et al., MASP2 levels are elevated in thrombotic microangiopathies: association with microvascular endothelial cell injury and suppression by anti-MASP2 antibody narsoplimab. Clin Exp Immunol (2021) 203(1):96-104); and sickle cell disease (see e.g., Belcher J D et al., MASP-2 and MASP-3 inhibitors block complement activation, inflammation, and microvascular stasis in a murine model of vaso-occlusion in sickle cell disease. Transl Res (2022) 249:1-12).

Methods of Inhibiting the Activation of Microglial Cells for the Prevention or Treatment of Neurodegenerative Diseases

Microglial cells are brain-resident macrophages that play a vital role in the differentiation and maturation of neuronal cells by controlling the process of axonal pruning and by maintaining the homeostasis of central nervous system (CNS) tissue through the removal of apoptotic and injured cells and cellular debris (Parkhurst C N, et al., Microglia promote learning-dependent synapse formation through brain-derived neurotrophic factor. Cell 2013; 155:1596-609), clearance of pathogens, tissue repair, and the release of cytokines and chemokines to attract peripheral immune cells to the infected or injured brain tissue (Fetler L, Neuroscience AS. Brain under surveillance: the microglia patrol. Science 2005; 309:392-3).

During homeostasis, microglia use their ramified processes to survey the microenvironment in real time for potential signals that warrant further action. Mature microglia in the postnatal brain use a wide range of surface molecules to respond quickly to their extracellular environment, including cytokines, chemokines, purines, hormones, and neurotransmitters (Tay, T. L., et al., Microglia across the lifespan: from origin to function in brain development, plasticity and cognition. J. Physiol. 595, 1929-1945 (2017). Microglial activation is tightly regulated through receptor-ligand interactions (Hanisch, U. K. & Kettenmann, H. Microglia: active sensor and versatile effector cells in the normal and pathologic brain Nat. Neurosci. 10, 1387-1394 (2007). In the adult brain, microglia display remarkable efficiency in clearing dead cells and excess cellular material, and microglial phagocytosis shapes adult hippocampal neurogenesis (Sierra, A. et al. Microglia shape adult hippocampal neurogenesis through apoptosis-coupled phagocytosis. Cell Stem Cell 7, 483-495 (2010). A rising number of studies have shown microglial roles in synapse formation, pruning and elimination, and regulation of synaptic function. Synapse elimination occurs during normal brain development, which involves the removal of unnecessary excitatory and inhibitory synaptic connections (Liu, Y. J. et al. Microglia elimination increases neural circuit connectivity and activity in adult mouse cortex. J. Neurosci 41, 1274-1287 (2021). This elimination process is vital for the formation of mature and efficient neuronal circuits during normal brain development (Paolicelli, R. C. et al. Synaptic pruning by microglia is necessary for normal brain development. Science 333, 1456-1458 (2011). Complement cascade proteins, broadly expressed in the developing brain, localize to specific subsets of immature synapses and mediate their elimination (Stevens, B. et al. The classical complement cascade mediates CNS synapse elimination. Cell 131, 1164-1178 (2007). Microglia phagocytose complement-tagged synapses through the complement receptor (C3R) pathway, which is crucial for accurate synaptic connection. Importantly, interruption of this pruning mechanism causes long-lasting damage to brain circuitry and synaptic connections (Stevens, B. et al. The classical complement cascade mediates CNS synapse elimination. Cell 131, 1164-1178 (2007).

Microglia respond to CNS injuries and diseases with complex reactions, commonly called activation. Importantly, microglial activation occurs during brain injuries and is associated with neuroinflammation and lesion expansion. It is also a hallmark of neurodegenerative diseases, including Alzheimer's disease, Parkinson's disease, multiple system atrophy, amyotrophic lateral sclerosis, fronto-temporal dementia, progressive supranuclear palsy, cortico-basal degeneration, vascular dementia, dementia with Lewy bodies (Lewy body dementia) and Huntington's disease. Dysregulation of synaptic elimination is involved in the pathogenesis of neurodegenerative diseases (Koffie, R. M., Hyman, B. T. & Spires-Jones, T. L. Alzheimer's disease: synapses gone cold. Mol. Neurodegener. 6, 63 (2011). Synaptic loss precedes neuronal loss and is considered a more accurate indicator of cognitive decline in AD (DeTure, M. A & Dickson, D. W. The neuropathological diagnosis of Alzheimer's disease. Mol. Neurodegener. 14, 32 (2019). In neurodegenerative diseases, reactive microglia found near protein aggregates such as Aβ plaques are involved in synapse loss and neuronal damage. Eliminating microglia or attenuating microglial activation in neurodegenerative diseases restored spine number and synaptic integrity and improved functional outcomes (Wilton, D. K., Dissing-Olesen, L. & Stevens, B. Neuron-glia signaling in synapse elimination. Annu Rev. Neurosci. 42, 107-127 (2019). Further, it is generally recognized that microglia activation plays a contributory role and/or initiates the neurodegenerative process (Harry G J Microglia in Neurodegenerative Events—An Initiator or a Significant Other? International Journal of Molecular Sciences. 2021; 22(11): 5818).

Microglia constitutively express complement receptors. It has been demonstrated that in the case of SARS-COV-2-induced brain inflammation, inhibition of MASP-2, the effector enzyme of the lectin pathway of complement, alleviates microglial activation and brain inflammation markers (Youssif M Ali, et al., Inhibition of the Lectin Pathway of Complement Activation Reduces Acute Respiratory Distress Syndrome Severity in a Mouse Model of SARS-COV-2 Infection, The Journal of Infectious Diseases, 2023). Therefore, lectin pathway hyperactivation is thought to be involved in initiation of brain inflammation, including in various cases of neurodegenerative diseases, and MASP-2 inhibitors may prove useful in the treatment or prevention of such diseases.

The lectin pathway also has been shown to contribute critically to the post-traumatic inflammatory pathology following traumatic brain injury (TBI) with the highest degree of protection achieved through the absence of MASP-2, the effector enzyme of the lectin pathway, underscoring the potential therapeutic utility of therapeutics targeting MASP-2 in TBI (Mercurio D, et al., Targeted deletions of complement lectin pathway genes improve outcome in traumatic brain injury, with MASP-2 playing a major role. Acta Neuropathol Commm. 2020 Oct. 28; 8(1): 174).

Accordingly, in some embodiments, the method comprises inhibiting microglial activation in a subject suffering from, or at risk for developing a neurodegenerative disease or disorder, comprising administering to the subject an amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, in an amount sufficient to inhibit microglial activation in said subject. In some embodiments, the method can further comprise, prior to administering a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof to a subject, determining that the subject is afflicted with, or at risk for developing a neurodegenerative disease or disorder. In some embodiments, the method comprises treating a subject suffering from, or at risk of developing, a neurodegenerative disease or disorder selected from the group consisting of Alzheimer's disease, Parkinson's disease, multiple system atrophy, amyotrophic lateral sclerosis, fronto-temporal dementia, progressive supranuclear palsy, cortico-basal degeneration, vascular dementia, dementia with Lewy bodies (Lewy body dementia), Huntington's disease and Long COVID with an amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt thereof sufficient to treat or prevent the neurodegenerative disease in said subject.

### VI. Compositions, Dosage, and Administration

The compounds as described herein (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) can be administered in a manner compatible with the dosage formulation, and in such amount as will be effective or suitable for prevention or treatment. The quantity to be administered depends on a variety of factors, including, e.g., the age, body weight, physical activity, and diet of the individual, and the desired effect. In certain embodiments, the size of the dose may also be determined by the existence, nature, and extent of any adverse side effects that accompany the administration of the compound in a particular individual.

It will be understood, however, that the specific dose level and frequency of dosage for any particular subject may be varied by a physician and will depend upon a variety of factors, including the activity of the specific compound employed, the metabolic stability and length of action of that compound, the age, body weight, hereditary characteristics, general health, sex, diet, mode, and time of administration, rate of excretion, drug combination, the severity of the particular condition, and the host undergoing therapy.

In certain embodiments, the dose may take the form of solid, semi-solid, liquid, or gas forms. In certain embodiments, the unit dosage form is suitable for ease of administration and uniformity of dosage.

As used herein, the term “unit dosage form” refers to physically discrete units suitable as unitary dosages for humans and other mammals, suited as unitary dosages for the subject to be treated, with each unit containing a predetermined quantity of an active agent calculated to produce the desired onset, tolerability, efficacious and/or therapeutic effects, in association with a suitable pharmaceutical excipient (e.g., an ampoule). In addition, more concentrated dosage forms may be prepared, from which the more dilute unit dosage forms may then be produced.

The compounds described herein (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) can be administered to a subject in need of prevention or treatment using methods known in the art, such as by oral administration or by injection. The injection can be, e.g., subcutaneous, intravenous, intraperitoneal, or intramuscular. As described herein, parenteral formulations can be prepared in a unit dosage form.

The pharmaceutical compositions of the present application comprise a therapeutically effective amount of a compound of the present disclosure (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) formulated together with one or more pharmaceutically acceptable carriers or excipient. As used herein, the term “pharmaceutically acceptable carrier” means a non-toxic, inert solid, semi-solid or liquid filler, diluent, encapsulating material or formulation auxiliary of any type. The pharmaceutical compositions of this application can be administered to humans and other animals orally, rectally, parenterally, intracistemally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations include, for example, sterile injectable aqueous or oleaginous suspensions formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

To prolong the effect of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, it can be desirable to slow the absorption of the compound from subcutaneous or intramuscular injection. This may be accomplished using a liquid suspension of crystalline or amorphous material with poor water solubility. The rate of absorption of the drug then depends upon its rate of dissolution which, in turn, may depend upon, for example, crystal size and crystalline form. Alternatively, delayed absorption of a parenterally administered compound form is accomplished by dissolving or suspending the compound in an oil vehicle.

Solid compositions of a similar type may also be employed as fillers in soft and hard filled gelatin capsules using such excipients as lactose or milk sugar as well as high molecular weight polyethylene glycols, and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the active compound may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound as disclosed herein (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) include ointments, pastes, creams, lotions, gels, powders, solutions, sprays, inhalants, or patches. For example, the active component may be ad-mixed under sterile conditions with a pharmaceutically acceptable carrier or excipient, and any needed preservatives or buffers as may be required.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, according to any one of the foregoing embodiments, in such amounts and for such time as is necessary to achieve the desired result. As is well understood in the medical arts, a therapeutically effective amount of a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, will be at a reasonable benefit/risk ratio applicable to any medical treatment.

In general, compounds (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) will be administered in therapeutically effective amounts via any of the usual and acceptable modes known in the art, either singly or in combination with one or more other therapeutic agents. A therapeutically effective amount may vary widely depending on the severity of the disease, the age and relative health of the subject, the potency of the compound used, and other factors.

In general, satisfactory results are indicated to be obtained systemically at daily dosages of from about 0.03 to 2.5 ^{mg}/_{kg} of body weight. An indicated daily dosage in the larger mammal, e.g., humans, is in the range from about 0.5 mg to about 250 mg, about 5 mg to about 150 mg, about 5 mg to about 100 mg, about 10 mg to about 75 mg, about 10 mg to about 50 mg, about 10, about 20, about 30, about 40, or about 50 mg, conveniently administered, e.g., in divided doses up to four times a day or in retard form. Suitable unit dosage forms for oral administration comprise from ca. 1 to 60 mg active ingredient.

In certain embodiments, a therapeutic amount or dose of the compound (e.g., compounds of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof) may range from about 0.1 mg/kg to about 500 ^{mg}/_{kg}, alternatively from about 1 ^{mg}/_{kg} to about 50 ^{mg}/_{kg}. In general, treatment regimens according to the present application comprise administration to a subject in need of such treatment from about 10 mg to about 1000 mg of the compound(s) per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained when the symptoms have been alleviated to the desired level, or treatment may cease. The subject may, however, require intermittent treatment on a long-term basis upon any recurrence of disease symptoms.

It will be understood, however, that the total daily usage of the compounds (e.g., a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof), will be decided by an attending physician within the scope of sound medical judgment. The specific inhibitory dose for any particular subject will depend upon a variety of factors, including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

This disclosure also provides for a pharmaceutical combination, e.g., a kit, comprising:
a) a first agent which is a compound of Structures (I), (I-A), (I-B), (I-C), (I-D), (I-E), (II), (II-A), (II-B), (II-C), (II-D), (III), (III-A), (III-B), and (III-C), or a stereoisomer, tautomer, or pharmaceutically acceptable salt, and embodiments thereof, as disclosed herein, in free form or in a pharmaceutically acceptable salt form, and
b) at least one co-agent.

The kit can comprise instructions for its administration.

Methods for preparing such dosage forms are known to those skilled in the art (see, e.g., REMINGTON'S PHARMACEUTICAL SCIENCES, 18th ED., Mack Publishing Co., Easton, PA (1990)). The dosage forms typically include a conventional pharmaceutical carrier or excipient, and may additionally include other medicinal agents, carriers, adjuvants, diluents, tissue permeation enhancers, solubilizers, and the like. Appropriate excipients can be tailored to the particular dosage form and route of administration, by methods well known in the art (see, e.g., REMINGTON'S PHARMACEUTICAL SCIENCES, 18th ED., Mack Publishing Co., Easton, PA (1990)).

### EXAMPLES

The following examples are provided by way of illustration only and not by way of limitation. Those of skill in the art will readily recognize a variety of noncritical parameters which could be changed or modified to yield essentially similar results.

### General Methods

Compounds described herein, including salts thereof, can be prepared using known organic synthesis techniques and can be synthesized according to any of numerous possible synthetic routes, such as those illustrated in the Examples.

The reactions for preparing compounds described herein can be carried out in suitable solvents which can be readily selected by one of skill in the art of organic synthesis. Suitable solvents can be substantially non-reactive with the starting materials or reactants, the intermediates, or the products, at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups is described in, e.g., Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6th Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin layer chromatography (TLC).

The particular synthetic methods used in the Examples provide general guidance in connection with preparing the compounds of the disclosure. One skilled in the art would understand that the preparations can be modified or optimized using general knowledge of organic chemistry to prepare various compounds within the scope of the present disclosure.

Starting materials, reagents, and intermediates whose synthesis is not described herein are either commercially available, known in the literature, or may be prepared by methods known to one skilled in the art.

It will be appreciated by one skilled in the art that the processes described herein are not the exclusive means by which compounds of the disclosure may be synthesized, and that a broad repertoire of synthetic organic reactions is available to be potentially employed in synthesizing compounds of the disclosure. The person skilled in the art knows how to select and implement appropriate synthetic routes. Suitable synthetic methods of starting materials, intermediates, and products may be identified by reference to the literature, including reference sources such as: Advances in Heterocyclic Chemistry, Vols. 1-107 (Elsevier, 1963-2012); Journal of Heterocyclic Chemistry, Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2nd Edition, 2004); Katritzky et al. (Ed.), Comprehensive Heterocyclic Chemistry (Pergamon Press, 1984); Katritzky et al., Comprehensive Heterocyclic Chemistry II (Pergamon Press, 1996); Smith et al., March's Advanced Organic Chemistry: Reactions, Mechanisms, and Structure, 6th Ed. (Wiley, 2007); Trost et al. (Ed.), Comprehensive Organic Synthesis (Pergamon Press, 1991).

If not otherwise stated, chromatography refers to flash chromatography conducted on silica gel. “Amine column” refers to flash chromatography conducted on Redisep Rf Gold® high performance amine column. HPLC purification refers to high performance liquid chromatography performed by one of two methods.

HPLC Method 1: A Gilson preparative reverse phase HPLC system with the combination of UV/ELS detectors (254 nm and 280 nm) and ThermoFisher Hypersil GOLD™ Agilent (21.2× 250 mm) 5 μm C18 column; eluents consisted of a mixture of water and acetonitrile (with 0.05% trifluoroacetic acid); flow rate was typically 20 mL/min with a linear gradient of acetonitrile in water from 2-90% acetonitrile over 45 min; and the injection volume ranged from 1 to 3 mL, with a maximum 20 mg sample per injection.

HPLC Method 2: A Waters™ preparative reverse phase HPLC system with the combination of UV/MS detectors (254 nm and 280 nm) and XBridge Prep (19×50 mm) C₁₈ 10 μM OBD column; eluents consisted of a mixture of water and acetonitrile (with 0.05% trifluoroacetic acid); flow rate was typically 50 mL/min with a linear gradient of acetonitrile in water from 5-95% acetonitrile over 8 min; and the injection volume ranged from 0.2 to 1 mL with a maximum 20 mg sample per injection.

### Abbreviations

μ micro
° ° C. degrees Celsius
ACN acetonitrile
anhyd anhydrous
aq aqueous
Ar argon
atm atmosphere(s)
ave average
Boc tert-butoxycarbonyl
calc calculated
concd concentrated
cmp no compound number
DCC N, N′-dicyclohexylcarbodiimide
DCE 1,2-dichloroethane
DCM dichloromethane
DIEA N,N-diisopropylethylamine
DMAP 4-(N,N-dimethylamino)pyridine
DMF dimethylformamide
DMSO dimethylsulfoxide
ES electrospray
Et ethyl
Et₂O diethyl ether
EtOAc ethyl acetate
Ex Example
g gram(s)
h hour(s)
HATU N-[(Dimethylamino)-1H-1,2,3-triazolo-[4,5-b]pyridin-1-ylmethylene]-N-methylmethanaminium hexafluorophosphate N-oxide
HPLC high-performance liquid chromatography
HOAc acetic acid
L liter(s)
m milli
M molar
MeCN acetonitrile
MeOH methanol
min minutes(s)
mL milliliter
mmol millimole
mol mole; molecular (as in mol wt)
MS mass spectrometry
N normal
NHS N-hydroxysuccinimide
NMR nuclear magnetic resonance
o ortho
p para
Ph phenyl
prep preparatory
psi pounds per square inch
RT room temperature (e.g., ˜20-23° C.)
sat saturated
temp temperature
tert tertiary
TFA trifluoroacetic acid
THF tetrahydrofuran
vac vacuo (or vacuum)

### Example 1

### Preparation of (S)—N-((6-amino-2-methylpyridin-3-yl)methyl)-3-((3,5-dimethylbenzyl)amino)-4-oxo-4,6,7,8-tetrahydropyrrolo[1,2-a]pyrimidine-6-carboxamide (Compound 1)

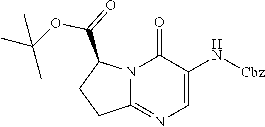 [SMILES: CC(C)(C)OC(=O)[C@@H]1CCc2ncc(NC(=O)OCc3ccccc3)c(=O)n21]

The above compound was synthesized according to the procedural details found on pages 19-21 of patent US 2004/0006065 A.

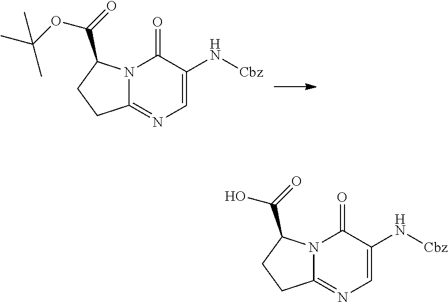 [SMILES: CC(C)(C)OC(=O)[C@@H]1CCc2ncc(NC(=O)OCc3ccccc3)c(=O)n21 ; O=C(Nc1cnc2n(c1=O)[C@H](C(=O)O)CC2)OCc1ccccc1]

Step 1: (S)-3-(((benzyloxy)carbonyl)amino)-4-oxo-4,6,7,8-tetrahydro-pyrrolo[1,2-a]pyrimidine-6-carboxylate (2.00 g, 5.19 mmol) was dissolved in DCM (10 mL) and treated with TFA (4.80 mL, 62.3 mmol). After stirring for 18 hours at 20° C., 0.5 mL of TFA was added to the above mixture and the reaction mixture was stirred for an additional hours. 0.2 mL of TFA was added and the reaction stirred for 16 h. The mixture was concentrated, and the crude material was used without further purification.

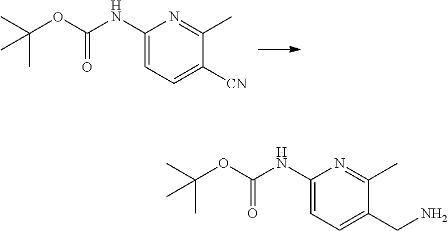 [SMILES: Cc1nc(NC(=O)OC(C)(C)C)ccc1C#N ; Cc1nc(NC(=O)OC(C)(C)C)ccc1CN]

Step 2: tert-Butyl (5-cyano-6-methylpyridin-2-yl)carbamate (10 g, 43 mmol) and Pd/C (0.23 g, 10% wt, 0.21 mmol) in glacial acetic acid was stirred in a Parr hydrogenation apparatus at 60 psi for 48 hours. The reaction was filtered through Celite® and the eluent evaporated in vacuo. The residue was dissolved in chloroform, basified to pH 13 with 10% aq NaOH, then extracted 3× with chloroform. The solution was concentrated and purified by column 10 chromatography (6-10% DCM/7M NH₃—MeOH) to afford the product tert-butyl (5-(aminomethyl)-6-methylpyridin-2-yl)carbamate (2.89 g. 14% yield).

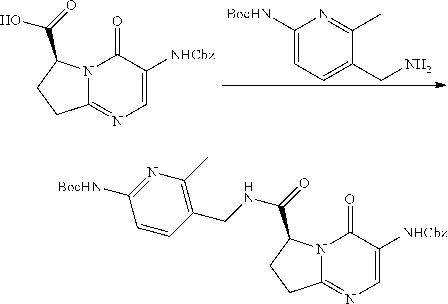 [SMILES: O=C(Nc1cnc2n(c1=O)[C@H](C(=O)O)CC2)OCc1ccccc1 ; Cc1nc(NC(=O)OC(C)(C)C)ccc1CN ; Cc1nc(NC(=O)OC(C)(C)C)ccc1CNC(=O)[C@@H]1CCc2ncc(NC(=O)OCc3ccccc3)c(=O)n21]

Step 3: (S)-3-(((Benzyloxy)carbonyl)amino)-4-oxo-4,6,7,8-tetrahydro-pyrrolo-[1,2-a]pyrimidine-6-carboxylic acid (370 mg, 1.12 mmol) was dissolved in DCM (17 mL) and DMF (1.5 mL), and treated with N-hydroxysuccinimide (155 mg, 1.35 mmol) and DCC (278 mg, 1.35 mmol). After stirring for 1 hour, tert-butyl (5-(aminomethyl)-6-methylpyridin-2-yl)carbamate (400 mg, 1.69 mmol) was added. After stirring for an additional 15 hours, the reaction was filtered through a medium fritted funnel and concentrated. The residue was purified by column chromatography (60-100% EtOAc-heptane then 10-30% MeOH-DCM) to provide benzyl (S)-(6-(((6-((tert-butoxycarbonyl)amino)-2-methylpyridin-3-yl)methyl)carbamoyl)-4-oxo-4,6,7,8-tetrahydropyrrolo[1,2-a]pyrimidin-3-yl)carbamate (520 mg, 84% yield).

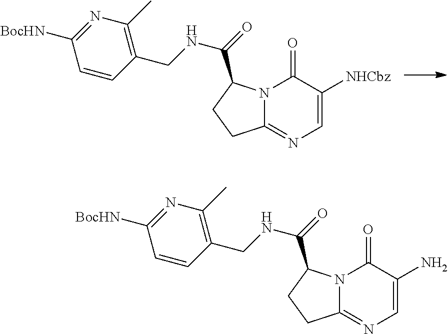 [SMILES: Cc1nc(NC(=O)OC(C)(C)C)ccc1CNC(=O)[C@@H]1CCc2ncc(NC(=O)OCc3ccccc3)c(=O)n21 ; Cc1nc(NC(=O)OC(C)(C)C)ccc1CNC(=O)[C@@H]1CCc2ncc(N)c(=O)n21]

Step 4: Benzyl (S)-(6-(((6-((tert-butoxycarbonyl)amino)-2-methylpyridin-3-yl)methyl)carbamoyl)-4-oxo-4,6,7,8-tetrahydropyrrolo[1,2-a]pyrimidin-3-yl)carbamate (2 g, 3.646 mmol) was dissolved in MeOH (20 mL) and EtOAc (20 mL). The mixture was purged with Ar, and Pd/C (194.0 mg, 10% wt, 182.3 μmol) was added. The reaction vessel was placed under vacuum and backfilled with H₂ 3× and left to stir under 1 atm H₂ for 2.5 days. The reaction mixture was filtered through a syringe filter and concentrated to afford tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (1.30 g, 3.14 mmol, 86% yield).

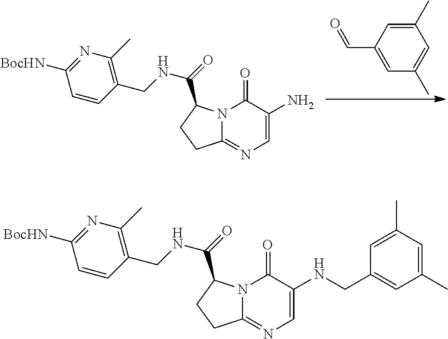

Step 5: To a solution of tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (200 mg, 483 μmol) and 3,5-dimethylbenzaldehyde (129 mg, 130 μL, 965 μmol) in DCE (8 mL) was added HOAc (166 μL, 2.90 mmol). After stirring for 30 minutes, sodium triacetoxyborohydride (307 mg, 1.45 mmol) was added. After stirring for 2 hours at 20° C., the reaction mixture was diluted in DCM and washed with sat. NaHCO₃, brine and dried over Na₂SO₄, filtered, and concentrated. The residue was purified by column chromatography (0-100% EtOAc-heptane and 0-30% MeOH-DCM) to provide tert-butyl (S)-(5-((3-((3,5-dimethylbenzyl)amino)-4-oxo-4,6,7,8-tetrahydro-pyrrolo[1,2-a]pyrimidine-6-carboxamido)methyl)-6-methylpyridin-2-yl)carbamate (220 mg. 85% yield).

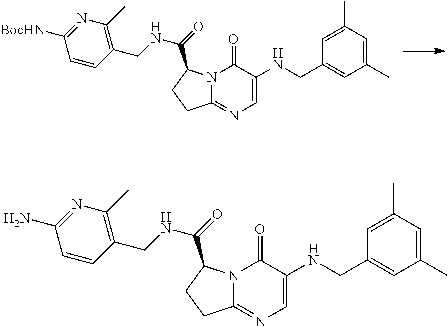 [SMILES: Cc1cc(C)cc(CNc2cnc3n(c2=O)[C@H](C(=O)NCc2ccc(NC(=O)OC(C)(C)C)nc2C)CC3)c1 ; Cc1cc(C)cc(CNc2cnc3n(c2=O)[C@H](C(=O)NCc2ccc(N)nc2C)CC3)c1]

Step 6: To a solution of tert-butyl (S)-(5-((3-((3,5-dimethylbenzyl)amino)-4-oxo-4,6,7,8-tetrahydropyrrolo[1,2-a]pyrimidine-6-carboxamido)methyl)-6-methylpyridin-2-yl)carbamate (220 mg, 413 μmol) in DCM (13 mL) was added TFA (1.88 g, 1.27 mL, 16.5 mmol). After stirring for 18 hours at 20° C., the reaction mixture was concentrated. The residue was purified by column chromatography (amine column, 0-50% MeOH-DCM) and the final purification by column chromatography (0-50% MeOH-DCM) provided (S)—N-((6-amino-2-methylpyridin-3-yl)methyl)-3-((3,5-dimethyl-benzyl)amino)-4-oxo-4,6,7,8-tetrahydropyrrolo[1,2-a]pyrimidine-6-carboxamide (114 mg, 64% yield).

### Example 2

### Preparation of (S)—N-((6-amino-2-methylpyridin-3-yl)methyl)-3-((3-methoxy-5-methylbenzyl)amino)-4-oxo-4,6,7,8-tetrahydropyrrolo[1,2-a]pyrimidine-6-carboxamide (Compound 2)

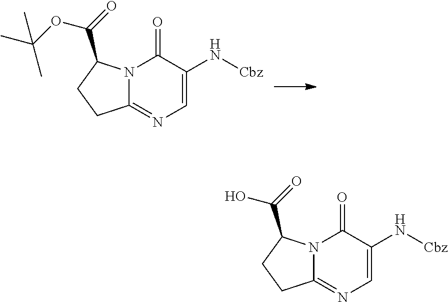 [SMILES: CC(C)(C)OC(=O)[C@@H]1CCc2ncc(NC(=O)OCc3ccccc3)c(=O)n21 ; O=C(Nc1cnc2n(c1=O)[C@H](C(=O)O)CC2)OCc1ccccc1]

Step 1: tert-Butyl (S)-3-(((benzyloxy)carbonyl)amino)-4-oxo-4,6,7,8-tetrahydro-pyrrolo[1,2-a]pyrimidine-6-carboxylate (2.00 g, 5.19 mmol) was dissolved in DCM (10 mL) and treated with TFA (5.92 g, 4.00 mL, 51.9 mmol). After stirring for 18 hours at 20° C., the reaction mixture was concentrated. The crude material was used without further purification

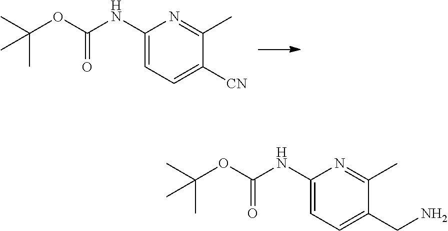 [SMILES: Cc1nc(NC(=O)OC(C)(C)C)ccc1C#N ; Cc1nc(NC(=O)OC(C)(C)C)ccc1CN]

Step 2: A mixture of tert-butyl (5-cyano-6-methylpyridin-2-yl)carbamate (5.14 g, 22.0 mmol) and Pd/C (234 mg, 10% wt, 220 μmol) in glacial acetic acid was stirred in a Parr hydrogenation apparatus at 60 psi for 48 hours. The reaction was filtered through Celite® and the was evaporated in vacuo. The residue was dissolved in DCM, then was basified with sat. NaHCO₃. The organic material was extracted twice with DCM and the combined organics were concentrated to afford a brown oil. The material was purified by column chromatography (amine column, 10% DCM/MeOH) to afford tert-butyl (5-(aminomethyl)-6-methylpyridin-2-yl)carbamate (2.81 g, 36% yield) as a white oily solid.

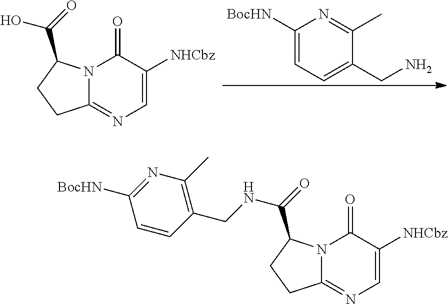 [SMILES: O=C(Nc1cnc2n(c1=O)[C@H](C(=O)O)CC2)OCc1ccccc1 ; Cc1nc(NC(=O)OC(C)(C)C)ccc1CN ; Cc1nc(NC(=O)OC(C)(C)C)ccc1CNC(=O)[C@@H]1CCc2ncc(NC(=O)OCc3ccccc3)c(=O)n21]

Step 3: (S)-3-(((Benzyloxy)carbonyl)amino)-4-oxo-4,6,7,8-tetrahydro-pyrrolo-[1,2-a]pyrimidine-6-carboxylic acid (1.8 g, 5.5 mmol) was dissolved in 80 mL of DCM and 5 mL DMF and treated with N-hydroxysuccinimide (0.69 g, 6.0 mmol) and DCC (1.2 g, 6.0 mmol). After stirring for 1 hour, tert-butyl (5-(aminomethyl)-6-methylpyridin-2-yl)carbamate (1.53 g, 6.45 mmol) was added. After stirring for 15 hours, the reaction was filtered through a medium fritted funnel and concentrated. The residue was purified by column chromatography (60-100% EtOAc-heptane and 10-30% MeOH-DCM) to provide benzyl (S)-(6-(((6-((tert-butoxycarbonyl)amino)-2-methylpyridin-3-yl)methyl)carbamoyl)-4-oxo-4,6,7,8-tetrahydropyrrolo[1,2-a]pyrimidin-3-yl)carbamate (1.0 g, 33% yield).

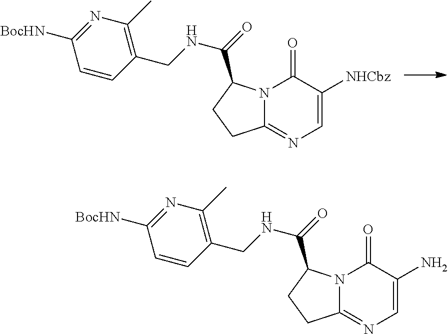 [SMILES: Cc1nc(NC(=O)OC(C)(C)C)ccc1CNC(=O)[C@@H]1CCc2ncc(NC(=O)OCc3ccccc3)c(=O)n21 ; Cc1nc(NC(=O)OC(C)(C)C)ccc1CNC(=O)[C@@H]1CCc2ncc(N)c(=O)n21]

Step 4: Benzyl (S)-(6-(((6-((tert-butoxycarbonyl)amino)-2-methyl-pyridin-3-yl)methyl)carbamoyl)-4-oxo-4,6,7,8-tetrahydropyrrolo[1,2-a]pyrimidin-3-yl)carbamate (1000 mg, 1.823 mmol) was dissolved in MeOH (11 mL) and EtOAc (11 mL). The mixture was purged with Ar, and Pd/C (96 mg, 10% wt, 91.14 μmol) was added. The reaction vessel was placed under vacuum and backfilled with H₂ 3× then left to stir under 1 atm of H₂ overnight. The reaction mixture was filtered through a syringe filter and concentrated to afford tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (697 mg, 92% yield).

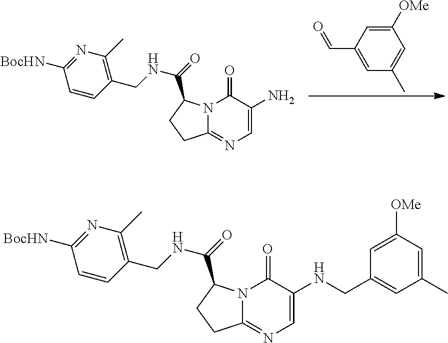 [SMILES: Cc1nc(NC(=O)OC(C)(C)C)ccc1CNC(=O)[C@@H]1CCc2ncc(N)c(=O)n21 ; COc1cc(C)cc(C=O)c1 ; COc1cc(C)cc(CNc2cnc3n(c2=O)[C@H](C(=O)NCc2ccc(NC(=O)OC(C)(C)C)nc2C)CC3)c1]

Step 5: To a solution of tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (198 mg, 478 μmol) and 3-methoxy-5-methylbenzaldehyde (143 mg, 135 μL, 955 μmol) in DCE (8 mL) was added HOAc (164 μL, 2.87 mmol). After stirring for 30 min, sodium triacetoxyborohydride (304 mg, 1.43 mmol) was added. After stirring for 2 hours at 20° C., the reaction mixture was diluted in DCM and washed with sat. NaHCO₃, brine, and dried over Na₂SO₄, filtered, and concentrated. The residue was purified by column chromatography (0-100% EtOAc-heptane and 0-50% MeOH-DCM) to provide tert-butyl (S)-(5-((3-((3-methoxy-5-methylbenzyl)amino)-4-oxo-4,6,7,8-tetrahydropyrrolo[1,2-a]pyrimidine-6-carboxamido)methyl)-6-methylpyridin-2-yl)carbamate (210 mg, 80% yield).

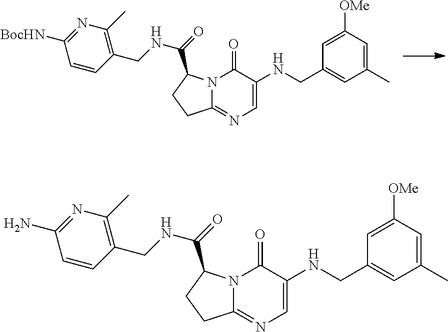 [SMILES: COc1cc(C)cc(CNc2cnc3n(c2=O)[C@H](C(=O)NCc2ccc(NC(=O)OC(C)(C)C)nc2C)CC3)c1 ; COc1cc(C)cc(CNc2cnc3n(c2=O)[C@H](C(=O)NCc2ccc(N)nc2C)CC3)c1]

Step 6: To a solution of tert-butyl (S)-(5-((3-((3-methoxy-5-methyl-benzyl)amino)-4-oxo-4,6,7,8-tetrahydropyrrolo[1,2-a]pyrimidine-6-carboxamido)-methyl)-6-methylpyridin-2-yl)carbamate (210 mg, 383 μmol) in DCM (10 mL) was added 0.3 mL of TFA. After stirring for 18 hours at 20° C., the reaction mixture was concentrated. The residue was purified by preparatory HPLC method 2 followed by column chromatography (amine column, 0-50% MeOH-DCM) to provide (S)—N-((6-amino-2-methylpyridin-3-yl)methyl)-3-((3-methoxy-5-methylbenzyl)amino)-4-oxo-4,6,7,8-tetrahydropyrrolo[1,2-a]pyrimidine-6-carboxamide (95 mg, 55% yield).

### Example 3

### Preparation of (S)—N-((6-amino-2-methylpyridin-3-yl)methyl)-3-((2-fluoro-5-methylbenzyl)amino)-4-oxo-4,6,7,8-tetrahydropyrrolo[1,2-a]pyrimidine-6-carboxamide (Compound 3)

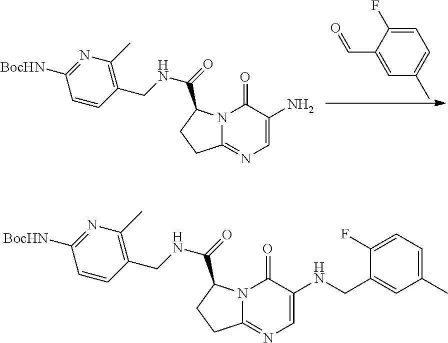

Step 1: To a solution of tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (208 mg, 502 μmol, prepared according to Ex. 1) and 2-fluoro-5-methylbenzaldehyde (139 mg, 0.13 mL, 1.00 mmol) in DCE (8 mL) was added HOAc (172 μL, 3.01 mmol). After stirring for 30 min, sodium triacetoxyborohydride (319 mg, 1.51 mmol) was added. After stirring for 2 hours at 20° C., the reaction mixture was diluted with DCM and washed with sat. NaHCO₃, brine and dried over Na₂SO₄, filtered, and concentrated. The residue was purified by column chromatography (0-100% EtOAc-heptane then 0-30% MeOH-DCM) to provide tert-butyl (S)-(5-((3-((2-fluoro-5-methylbenzyl)amino)-4-oxo-4,6,7,8-tetrahydropyrrolo[1,2-a]pyrimidine-6-carboxamido)methyl)-6-methylpyridin-2-yl)carbamate (204 mg. 76% yield).

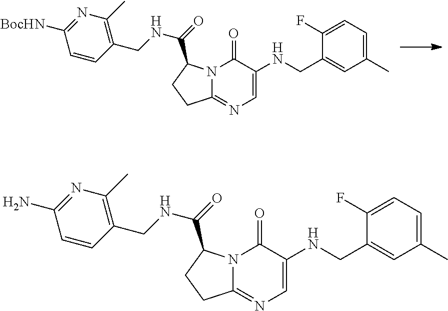 [SMILES: Cc1ccc(F)c(CNc2cnc3n(c2=O)[C@H](C(=O)NCc2ccc(NC(=O)OC(C)(C)C)nc2C)CC3)c1 ; Cc1ccc(F)c(CNc2cnc3n(c2=O)[C@H](C(=O)NCc2ccc(N)nc2C)CC3)c1]

Step 2: To a solution of tert-butyl (S)-(5-((3-((2-fluoro-5-methylbenzyl)amino)-4-oxo-4,6,7,8-tetrahydropyrrolo[1,2-a]pyrimidine-6-carboxamido)methyl)-6-methyl-pyridin-2-yl)carbamate (204 mg, 380 μmol) in DCM (10 mL) was added 0.3 mL of TFA. After stirring for 18 hours at 20° C., the reaction mixture was concentrated. The residue was purified by preparatory HPLC method 2 with final purification by column chromatography (amine column, 0-50% MeOH-DCM) to provide (S)—N-((6-amino-2-methylpyridin-3-yl)methyl)-3-((2-fluoro-5-methylbenzyl)amino)-4-oxo-4,6,7,8-tetrahydropyrrolo[1,2-a]pyrimidine-6-carboxamide (65 mg, 39% yield).

### Example 4

### Preparation of (S)—N-((6-aminopyridin-3-yl)methyl)-3-((3,5-carboxamide (Compound 4)

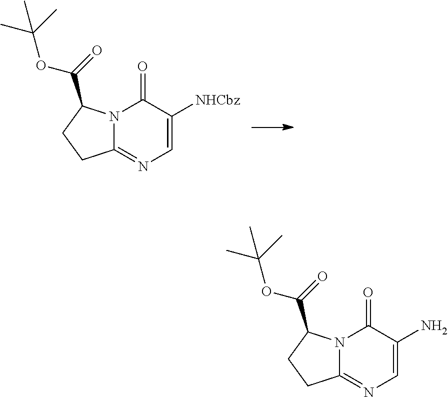 [SMILES: CC(C)(C)OC(=O)[C@@H]1CCc2ncc(NC(=O)OCc3ccccc3)c(=O)n21 ; CC(C)(C)OC(=O)[C@@H]1CCc2ncc(N)c(=O)n21]

Step 1: tert-Butyl (S)-3-(((benzyloxy)carbonyl)amino)-4-oxo-4,6,7,8-tetrahydro-pyrrolo[1,2-a]pyrimidine-6-carboxylate (500 mg, 1.30 mmol) was dissolved in MeOH (30 mL) and purged with Ar. Pd/C (13.8 mg, 10% wt, 13.0 μmol) was added, then the flask evacuated and backfilled with H₂ 3 times. After stirring for 2 hours at 20° C., the mixture was filtered and concentrated to provide tert-butyl (S)-3-amino-4-oxo-4,6,7,8-tetrahydropyrrolo[1,2-a]pyrimidine-6-carboxylate (326 mg, 100% yield).

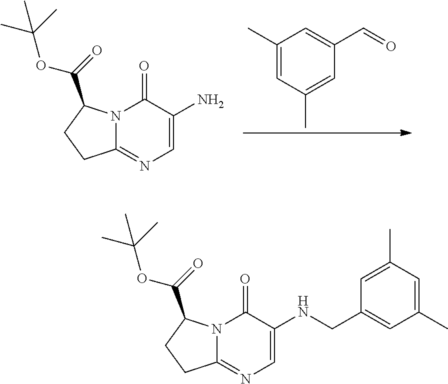

Step 2: To a solution of tert-butyl (S)-3-amino-4-oxo-4,6,7,8-tetrahydro-pyrrolo[1,2-a]pyrimidine-6-carboxylate (326 mg, 1.30 mmol) and 3,5-dimethylbenzaldehyde (348 mg, 349 μL, 2.59 mmol) in DCE (15 mL) was added HOAc (446 μL, 7.78 mmol). After stirring for 30 min, sodium triacetoxyborohydride (825 mg, 3.89 mmol) was added. After stirring for 1 hour at 20° C., the mixture was diluted in DCM and washed with sat. NaHCO₃, brine, dried over Na₂SO₄, filtered, and concentrated. The residue was purified by column chromatography (0-100% EtOAc-heptane) to provide tert-butyl (S)-3-((3,5-dimethylbenzyl)amino)-4-oxo-4,6,7,8-tetrahydropyrrolo-[1,2-a]pyrimidine-6-carboxylate (447 mg. 93% yield).

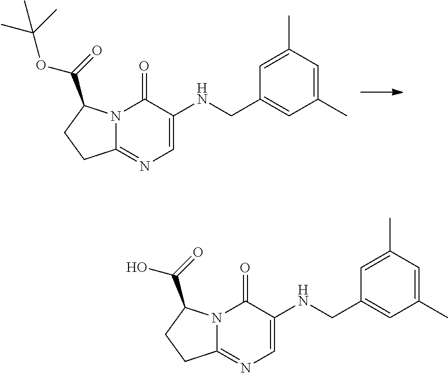 [SMILES: Cc1cc(C)cc(CNc2cnc3n(c2=O)[C@H](C(=O)OC(C)(C)C)CC3)c1 ; Cc1cc(C)cc(CNc2cnc3n(c2=O)[C@H](C(=O)O)CC3)c1]

Step 3: tert-Butyl (S)-3-((3,5-dimethylbenzyl)amino)-4-oxo-4,6,7,8-tetrahydro-pyrrolo[1,2-a]pyrimidine-6-carboxylate (447 mg, 1.21 mmol) was dissolved in DCM (6 mL) and treated with TFA (3.03 g, 2.05 mL, 26.6 mmol). After 8 hours, 0.2 mL of TFA was added and the reaction was stirred overnight. The mixture was concentrated to provide crude (S)-3-((3,5-dimethylbenzyl)amino)-4-oxo-4,6,7,8-tetrahydropyrrolo[1,2-a]pyrimidine-6-carboxylic acid (370 mg, 98%) which was applied to the next step without further purification.

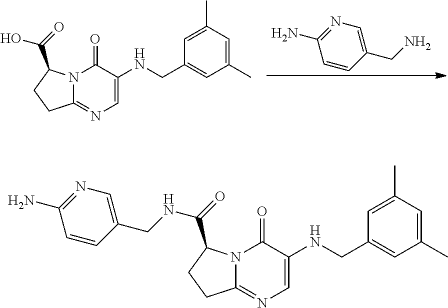 [SMILES: Cc1cc(C)cc(CNc2cnc3n(c2=O)[C@H](C(=O)O)CC3)c1 ; NCc1ccc(N)nc1 ; Cc1cc(C)cc(CNc2cnc3n(c2=O)[C@H](C(=O)NCc2ccc(N)nc2)CC3)c1]

Step 4: To a solution of (S)-3-((3,5-dimethylbenzyl)amino)-4-oxo-4,6,7,8-tetrahydropyrrolo[1,2-a]pyrimidine-6-carboxylic acid (61 mg, 0.19 mmol) in DMF (3 mL) was added HATU (0.15 g, 0.39 mmol) and DIEA (75 mg, 0.10 mL, 0.58 mmol). After stirring for 10 min, 5-(aminomethyl)pyridin-2-amine (29 mg, 0.23 mmol) was added. After stirring for 18 hours at 20° C., the reaction mixture was concentrated and the residue was purified by column chromatography (amine column, 0-100% MeOH-DCM). Additional purification by column chromatography (0-50% MeOH-DCM), preparatory HPLC method 2 with final purification by column chromatography (amine column, 0-50% MeOH-DCM) provided (S)—N-((6-aminopyridin-3-yl)methyl)-3-((3,5-dimethylbenzyl)amino)-4-oxo-4,6,7,8-tetrahydropyrrolo[1,2-a]pyrimidine-6-carboxamide (82 mg, 100% yield).

### Example 5

### Preparation of (S)—N-(5-chloro-2-(1H-tetrazol-1-yl)benzyl)-3-((3,5-carboxamide (Compound 5)

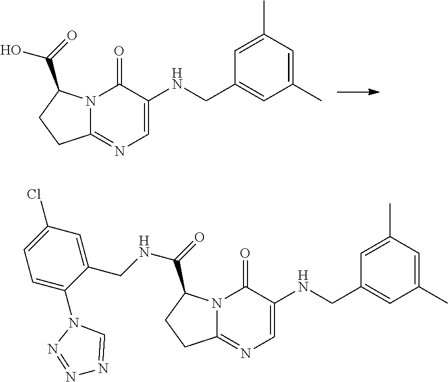 [SMILES: Cc1cc(C)cc(CNc2cnc3n(c2=O)[C@H](C(=O)O)CC3)c1 ; Cc1cc(C)cc(CNc2cnc3n(c2=O)[C@H](C(=O)NCc2cc(Cl)ccc2-n2cnnn2)CC3)c1]

Step 1: To a solution of (S)-3-((3,5-dimethylbenzyl)amino)-4-oxo-4,6,7,8-tetrahydropyrrolo[1,2-a]pyrimidine-6-carboxylic acid (33 mg, 0.11 mmol, prepared according to Ex. 4) in DMF (1.5 mL) was added HATU (80 mg, 0.21 mmol) and DIEA (41 mg, 55 μL, 0.32 mmol). After stirring for 10 min, (5-chloro-2-(1H-tetrazol-1-yl)phenyl)methanamine (26 mg, 0.13 mmol) was added. After stirring for 18 hours at 20° C., the reaction was concentrated, and the residue was purified by column chromatography (0-100% EtOAc-heptane then 15% MeOH-DCM). The residue was repurified by preparatory HPLC method 2 to provide (S)—N-(5-chloro-2-(1H-tetrazol-1-yl)benzyl)-3-((3,5-dimethylbenzyl)amino)-4-oxo-4,6,7,8-tetrahydropyrrolo[1,2-a]pyrimidine-6-carboxamide trifluoroacetic acid (23.3 mg, 44% yield).

### Example 6

### Preparation of (S)—N-((1H-pyrrolo[3,2-c]pyridin-2-yl)methyl)-3-((3,5-dimethylbenzyl)amino)-4-oxo-4,6,7,8-tetrahydropyrrolo[1,2-a]pyrimidine-6-carboxamide (Compound 6)

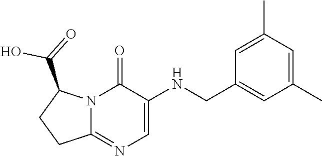 [SMILES: Cc1cc(C)cc(CNc2cnc3n(c2=O)[C@H](C(=O)O)CC3)c1]

Steps 1-3: The title compound was prepared according to steps 1-3 of Example 4, except that the acid was purified by column chromatography (MeOH-DCM) to give (S)-3-((3,5 dimethylbenzyl)amino)-4-oxo-4,6,7,8-tetrahydropyrrolo[1,2-a]pyrimidine-6-carboxylic acid (92% yield).

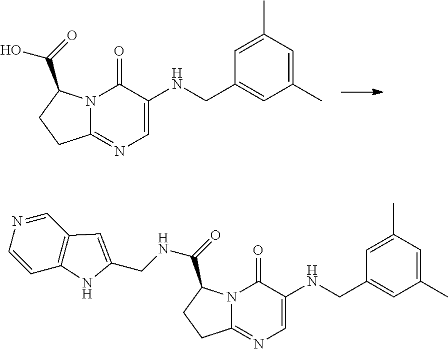 [SMILES: Cc1cc(C)cc(CNc2cnc3n(c2=O)[C@H](C(=O)O)CC3)c1 ; Cc1cc(C)cc(CNc2cnc3n(c2=O)[C@H](C(=O)NCc2cc4cnccc4[nH]2)CC3)c1]

Step 4: A 20 mL scintillation vial was charged with (S)-3-((3,5 dimethylbenzyl)amino)-4-oxo-4,6,7,8-tetrahydropyrrolo[1,2-a]pyrimidine-6-carboxylic acid (47 mg, 0.15 mmol), (1H-pyrrolo[3,2-c]pyridin-2-yl)methanamine (26 mg, 0.18 mmol) and DMF, followed by HATU (63 mg, 0.17 mmol) and DIEA (39 mg, 52 μL, 0.30 mmol). The mixture was stirred at room temperature overnight and concentrated in vacuo. The crude material was purified by column chromatography (0-50% MeOH-DCM) 2 times to provide (S)—N-((1H-pyrrolo[3,2-c]pyridin-2-yl)methyl)-3-((3,5-dimethylbenzyl)amino)-4-oxo-4,6,7,8-tetrahydropyrrolo[1,2-a]pyrimidine-6-carboxamide (23.2 mg, 26% yield).

### Example 7

### Preparation of (S)-3-((3,5-dimethylbenzyl)amino)-N-((6-methyl-1H-pyrrolo[3,2-c]pyridin-2-yl)methyl)-4-oxo-4,6,7,8-tetrahydropyrrolo[1,2-a]pyrimidine-6-carboxamide, trifluoroacetate (Compound 7)

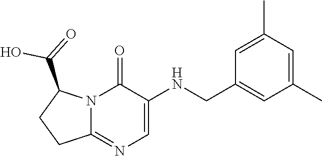 [SMILES: Cc1cc(C)cc(CNc2cnc3n(c2=O)[C@H](C(=O)O)CC3)c1]

Steps 1-3: The title compound was prepared according to steps 1-3 of Example 6.

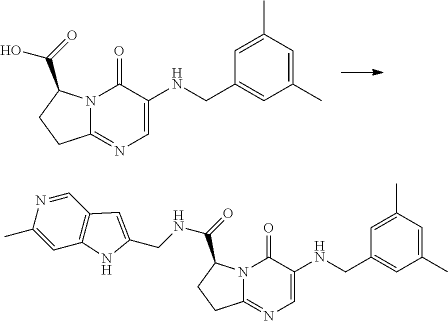

Step 4: A 20 mL scintillation vial was charged with (S)-3-((3,5-dimethylbenzyl)amino)-4-oxo-4,6,7,8-tetrahydropyrrolo[1,2-a]pyrimidine-6-carboxylic acid (47 mg, 0.15 mmol), (6-methyl-1H-pyrrolo[3,2-c]pyridin-2-yl)methanamine (29 mg, 0.18 mmol), prepared according to the procedure disclosed in PCT Publication No. WO/2019/231935, and DMF, followed by DIEA (39 mg, 52 μL, 0.30 mmol) and HATU (63 mg, 0.17 mmol). The mixture was stirred at room temperature overnight and concentrated in vacuo. The crude material was purified by column chromatography (amine column, 0-100% MeOH-DCM) and re-purified by column chromatography (0-50% MeOH-DCM). Then the final purification by preparatory HPLC method 2 provided (S)-3-((3,5-dimethylbenzyl)amino)-N-((6-methyl-1H-pyrrolo[3,2-c]pyridin-2-yl)methyl)-4-oxo-4,6,7,8-tetrahydropyrrolo[1,2-a]pyrimidine-6-carboxamide, trifluoroacetate (34.3 mg, 40% yield)

### Example 8

### Preparation of (S)—N-((6-amino-2-methylpyridin-3-yl)methyl)-3-((3-cyano-5-methylbenzyl)amino)-4-oxo-4,6,7,8-tetrahydropyrrolo[1,2-a]pyrimidine-6-carboxamide (Compound 8)

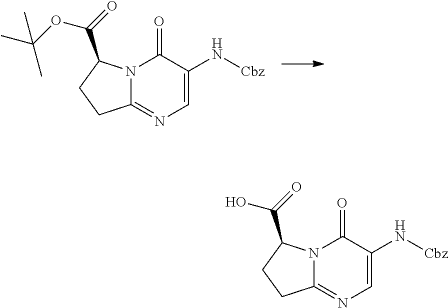 [SMILES: CC(C)(C)OC(=O)[C@@H]1CCc2ncc(NC(=O)OCc3ccccc3)c(=O)n21 ; O=C(Nc1cnc2n(c1=O)[C@H](C(=O)O)CC2)OCc1ccccc1]

Step 2: tert-Butyl (S)-3-(((benzyloxy)carbonyl)amino)-4-oxo-4,6,7,8-tetra-hydropyrrolo[1,2-a]pyrimidine-6-carboxylate (2.00 g, 5.19 mmol) was dissolved in DCM and treated with TFA (5.92 g, 4.00 mL, 51.9 mmol) and triisopropylsilane (822 mg, 5.19 mmol). The mixture was allowed to stir at room temperature overnight. The crude material was concentrated twice to afford a brown oil. The material was then triturated with Et₂O and filtered to afford the product as an off-white solid (1.71 g, 100% yield).

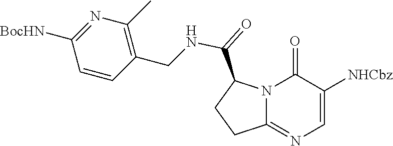 [SMILES: Cc1nc(NC(=O)OC(C)(C)C)ccc1CNC(=O)[C@@H]1CCc2ncc(NC(=O)OCc3ccccc3)c(=O)n21]

Steps 3-4: The title compound was prepared according to steps 3-4 of Example 1.

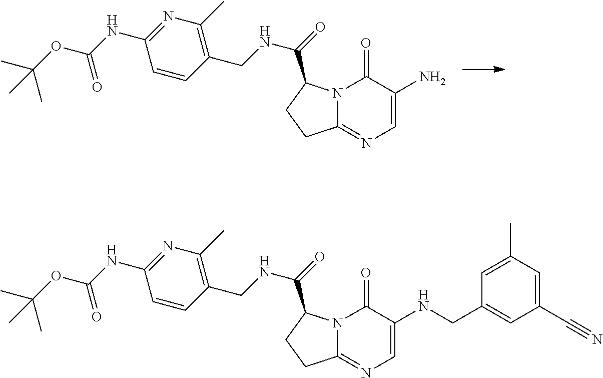 [SMILES: Cc1nc(NC(=O)OC(C)(C)C)ccc1CNC(=O)[C@@H]1CCc2ncc(N)c(=O)n21 ; Cc1cc(C#N)cc(CNc2cnc3n(c2=O)[C@H](C(=O)NCc2ccc(NC(=O)OC(C)(C)C)nc2C)CC3)c1]

Step 3: To a solution of tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (70 mg, 0.17 mmol, prepared according to Ex. 1) and 3-formyl-5-methylbenzonitrile (49 mg, 0.34 mmol) in 2 mL of 1,2-dichloroethane was added HOAc (58 μL, 1.0 mmol). After 30 minutes, sodium triacetoxyborohydride (0.11 g, 0.51 mmol) was added. The solution was stirred at RT for 2 h. The mixture was diluted with a saturated NaHCO₃ solution and extracted with DCM three times. The combined organics were washed with brine, dried over Na₂SO₄, filtered, and concentrated to afford the pure product (92 mg, 100% yield).

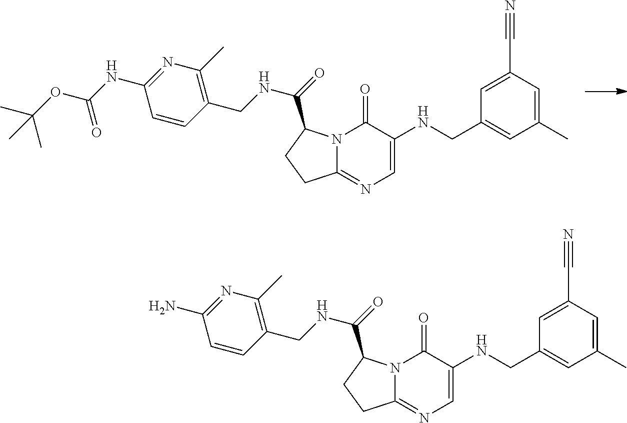 [SMILES: Cc1cc(C#N)cc(CNc2cnc3n(c2=O)[C@H](C(=O)NCc2ccc(NC(=O)OC(C)(C)C)nc2C)CC3)c1 ; Cc1cc(C#N)cc(CNc2cnc3n(c2=O)[C@H](C(=O)NCc2ccc(N)nc2C)CC3)c1]

Step 4: tert-Butyl (S)-(5-((3-((3-cyano-5-methylbenzyl)amino)-4-oxo-4,6,7,8-tetrahydropyrrolo[1,2-a]pyrimidine-6-carboxamido)methyl)-6-methylpyridin-2-yl)carbamate (92 mg, 0.17 mmol) was dissolved in DCM and treated with TFA (0.19 g, 0.13 mL, 1.7 mmol). The reaction was stirred at room temperature for 72 hours. The crude material was concentrated, then basified with Et₃N. The product was purified by chromatography (amine column, 0-100% EtOAc-heptane, then 0-100% MeOH-DCM), then chromatographed again (0-100% MeOH-DCM) and lyophilized to afford (S)—N-((6-amino-2-methylpyridin-3-yl)methyl)-3-((3-cyano-5-methylbenzyl)amino)-4-oxo-4,6,7,8-tetrahydropyrrolo[1,2-a]pyrimidine-6-carboxamide as a white fluffy powder (43.8 mg, 58% yield).

The following compounds were prepared according to the foregoing procedure using the appropriate aldehyde, except that the final product was treated with a small amount of TFA to afford the TFA salt:

| | Cmp |
|---|---|
| Compound Name | No. |
| (S)-N-((6-amino-2-methylpyridin-3-yl)methyl)-3-((2-methoxy-5- | 9 |
| methylbenzyl)amino)-4-oxo-4,6,7,8-tetrahydropyrrolo[1,2- | |
| a]pyrimidine-6-carboxamide, trifluoroacetate | |

### Example 9

### Preparation of (S)—N-((6-amino-2-methylpyridin-3-yl)methyl)-3-((2-methoxy-3-methylbenzyl)amino)-4-oxo-4,6,7,8-tetrahydropyrrolo[1,2-a]pyrimidine-6-carboxamide, trifluoroacetate (Compound 10)

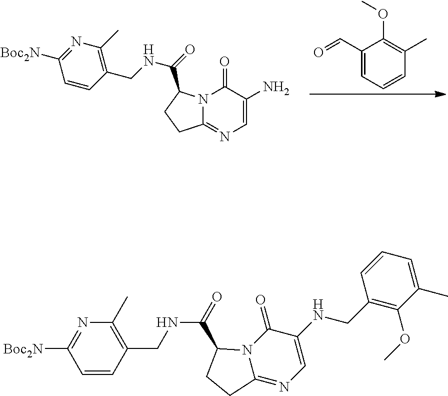

Step 1: To a solution of tert-butyl (S)-(5-((3-amino-4-oxo-4,6,7,8-tetrahydro-pyrrolo[1,2-a]pyrimidine-6-carboxamido)methyl)-6-methylpyridin-2-yl)(tert-butoxy-carbonyl)carbamate (80 mg, 0.16 mmol, prepared according to Example 8) and 2-methoxy-3-methylbenzaldehyde (47 mg, 44 μL, 0.31 mmol) in 2 mL of 1,2-dichloroethane was added HOAc (53 μL, 0.93 mmol). After 1 hour, sodium triacetoxyborohydride (99 mg, 0.47 mmol) was added. The solution was stirred overnight at room temperature. The mixture was diluted with saturated NaHCO₃ solution and extracted with DCM three times. The combined organics were washed with brine, dried over Na₂SO₄, filtered, and concentrated. The semi-pure material was purified by column chromatography (0-100% MeOH-DCM) to give tert-butyl (S)-(tert-butoxycarbonyl)(5-((3-((2-methoxy-3-methylbenzyl)amino)-4-oxo-4,6,7,8-tetrahydropyrrolo[1,2-a]pyrimidine-6-carboxamido)methyl)-6-methylpyridin-2-yl)carbamate as a colorless oil (69 mg. 68% yield).

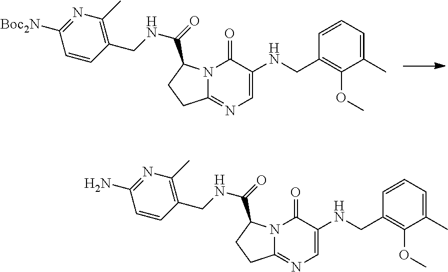

Step 2: tert-Butyl (S)-(tert-butoxycarbonyl)(5-((3-((2-methoxy-3-methylbenzyl)-amino)-4-oxo-4,6,7,8-tetrahydropyrrolo[1,2-a]pyrimidine-6-carboxamido)methyl)-6-methylpyridin-2-yl)carbamate (0.10 g, 0.16 mmol) was dissolved in HCl in MeOH (0.29 g, 2.7 mL, 3 molar, 8.0 mmol), allowed to stir at room temperature for 2 days. The material was concentrated in vacuo and purified by preparatory HPLC method 2 to afford (S)—N-((6-amino-2-methylpyridin-3-yl)methyl)-3-((2-methoxy-3-methylbenzyl)amino)-4-oxo-4,6,7,8-tetrahydropyrrolo[1,2-a]pyrimidine-6-carboxamide, trifluoroacetate (35.7 mg, 40% yield).

The following compounds were prepared according to the foregoing procedure using the appropriate aldehyde and amine starting materials, except that the final step was concentrated down after reaction:

| | Cmp |
|---|---|
| Compound Name | No. |
| (S)-N-((6-amino-2-methylpyridin-3-yl)methyl)-3-((3- | 11 |
| morpholinobenzyl)amino)-4-oxo-4,6,7,8-tetrahydropyrrolo[1,2- | |
| a]pyrimidine-6-carboxamide, hydrochloride | |

### Example 10

### Preparation of (S)—N-((6-amino-2-methylpyridin-3-yl)methyl)-3-(((2,2-difluorobenzo[D][1,3]dioxol-5-yl)methyl)amino)-4-oxo-4,6,7,8-tetrahydropyrrolo[1,2-a]pyrimidine-6-carboxamide, trifluoroacetate (Compound 12)

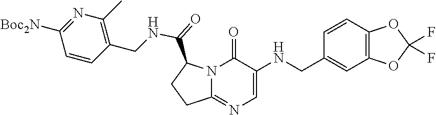

Steps 1-5: The title compound was prepared according to steps 1-5 of Example 9 with the appropriate amine starting material.

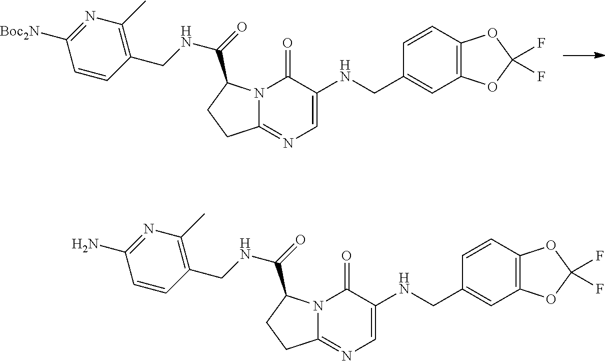

Step 6: tert-Butyl (S)-(tert-butoxycarbonyl)(5-((3-(((2,2-difluorobenzo-[d][1,3]dioxol-5-yl)methyl)amino)-4-oxo-4,6,7,8-tetrahydropyrrolo[1,2-a]pyrimidine-6-carboxamido)methyl)-6-methylpyridin-2-yl)carbamate (0.11 g, 0.16 mmol) was dissolved in DCM and cooled to 0° C. The mixture was treated with TFA (0.12 mL, 1.6 mmol), allowed to warm to room temperature and stirred overnight. The crude material was concentrated twice to dryness and lyophilized for 72 hours to afford of (S)—N-((6-amino-2-methylpyridin-3-yl)methyl)-3-(((2,2-difluorobenzo[d][1,3]dioxol-5-yl)methyl)amino)-4-oxo-4,6,7,8-tetrahydropyrrolo[1,2-a]pyrimidine-6-carboxamide, trifluoroacetate (54 mg, 56% yield).

The following compounds were prepared according to the foregoing procedure using the appropriate aldehyde and amine starting materials:

| | Cmp |
|---|---|
| Compound Name | No. |
| (S)-3-(((6-(((6-amino-2-methylpyridin-3-yl)methyl)carbamoyl)-4- | 13 |
| oxo-4,6,7,8-tetrahydropyrrolo[1,2-a]pyrimidin-3- | |
| yl)amino)methyl)benzoic acid | |
| (S)-N-((6-amino-2-methylpyridin-3-yl)methyl)-3-(((2,3- | 14 |
| dihydrobenzo[b][1,4]dioxin-5-yl)methyl)amino)-4-oxo-4,6,7,8- | |
| tetrahydropyrrolo[1,2-a]pyrimidine-6-carboxamide | |

### Example 11

### Preparation of (S)—N—((S)-1-(6-aminopyridin-3-yl)ethyl)-3-((3,5-dimethylbenzyl)amino)-4-oxo-4,6,7,8-tetrahydropyrrolo[1,2-a]pyrimidine-6-carboxamide (Compound 15)

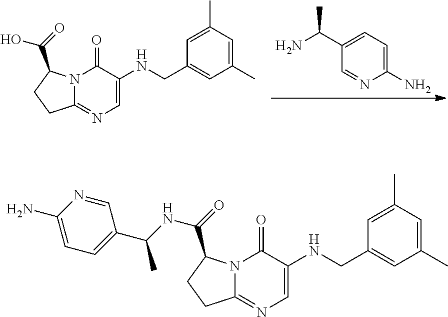

Step 4: A 20 mL scintillation vial was charged with (S)-3-((3,5-dimethylbenzyl)amino)-4-oxo-4,6,7,8-tetrahydropyrrolo[1,2-a]pyrimidine-6-carboxylic acid (47 mg, 0.15 mmol, prepared according to Ex. 4), (S)-5-(1-aminoethyl)pyridin-2-amine (25 mg, 0.18 mmol) and DMF, followed by HATU (63 mg, 0.17 mmol) and DIEA (39 mg, 52 μL, 0.30 mmol). The mixture was stirred at room temperature overnight. The crude material was concentrated and purified by column chromatography (amine column, 0-100% MeOH-DCM) to afford of (S)—N—((S)-1-(6-aminopyridin-3-yl)ethyl)-3-((3,5-dimethylbenzyl)amino)-4-oxo-4,6,7,8-tetrahydropyrrolo[1,2-a]pyrimidine-6-carboxamide (27 mg, 42% yield).

The following compounds were prepared according to the foregoing procedure using the appropriate amine starting materials:

| | Cmp |
|---|---|
| Compound Name | No. |
| (S)-N-((R)-1-(6-aminopyridin-3-yl)ethyl)-3-((3,5-dimethyl- | 16 |
| benzyl)amino)-4-oxo-4,6,7,8-tetrahydropyrrolo[1,2-a]pyrimidine-6- | |
| carboxamide | |

### Example 12

### Preparation of (S)—N-((5-chloropyridin-3-yl)methyl)-3-((3,5-dimethylbenzyl)amino)-4-oxo-4,6,7,8-tetrahydropyrrolo[1,2-a]pyrimidine-6-carboxamide, trifluoroacetate (Compound 17)

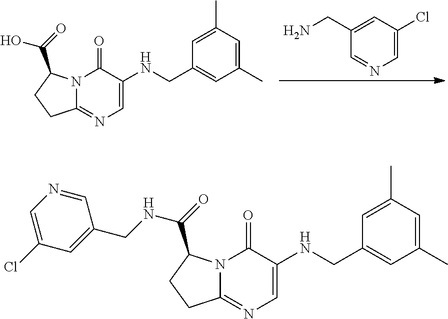 [SMILES: Cc1cc(C)cc(CNc2cnc3n(c2=O)[C@H](C(=O)O)CC3)c1 ; NCc1cncc(Cl)c1 ; Cc1cc(C)cc(CNc2cnc3n(c2=O)[C@H](C(=O)NCc2cncc(Cl)c2)CC3)c1]

Step 1: A 20 mL scintillation vial was charged with (S)-3-((3,5-dimethyl-benzyl)amino)-4-oxo-4,6,7,8-tetrahydropyrrolo[1,2-a]pyrimidine-6-carboxylic acid (47 mg, 0.15 mmol, prepared according to Ex. 4), (5-chloropyridin-3-yl)methanamine (26 mg, 0.18 mmol) and DMF, followed by HATU (63 mg, 0.17 mmol) and DIEA (39 mg, 52 μL, 0.30 mmol). The reaction mixture was stirred at room temperature overnight. (5-chloropyridin-3-yl)methanamine (26 mg, 0.18 mmol) and HATU (63 mg, 0.17 mmol) were added to the mixture and continued to stir overnight. The crude material was concentrated and purified by column chromatography (amine column, 100% EtOAc). Semi-pure fractions were collected and re-purified by column chromatography, and then purified by preparatory HPLC method 2 to afford (S)—N-((5-chloropyridin-3-yl)methyl)-3-((3,5-dimethylbenzyl)amino)-4-oxo-4,6,7,8-tetrahydropyrrolo[1,2-a]pyrimidine-6-carboxamide, trifluoroacetate as a white solid (14 mg, 17% yield).

### Example 13

### Preparation of (S)—N-((3-chloro-1H-pyrrolo[2,3-b]pyridin-5-yl)methyl)-3-((3,5-dimethylbenzyl)amino)-4-oxo-4,6,7,8-tetrahydropyrrolo[1,2-a]pyrimidine-6-carboxamide, trifluoracetate (Compound 18)

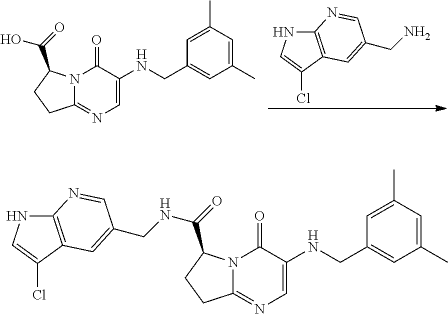

Step 4: A 20 mL scintillation vial was charged with (S)-3-((3,5-dimethylbenzyl)amino)-4-oxo-4,6,7,8-tetrahydropyrrolo[1,2-a]pyrimidine-6-carboxylic acid (47 mg, 0.15 mmol, prepared according to Ex. 4), (3-chloro-1H-pyrrolo[2,3-b]pyridin-5-yl)methanamine, di-trifluoroacetate (74 mg, 0.18 mmol) and DMF, followed by DIEA (78 mg, 0.10 mL, 0.60 mmol) and HATU (63 mg, 0.17 mmol). The resulting mixture was stirred at room temperature overnight. The crude material was purified by column chromatography (amine column, 0-100% MeOH-DCM), which was re-purified by column chromatography (0-50% MeOH-DCM). The corresponding mixture was purified by preparatory HPLC method 2 to provide (S)—N-((3-chloro-1H-pyrrolo[2,3-b]pyridin-5-yl)methyl)-3-((3,5-dimethylbenzyl)amino)-4-oxo-4,6,7,8-tetrahydropyrrolo[1,2-a]pyrimidine-6-carboxamide, trifluoroacetate (34.8 mg, 39% yield).

### Example 14

### Preparation of (S)-3-((3,5-dimethylbenzyl)amino)-N-((2-methylpyridin-4-yl)methyl)-4-oxo-4,6,7,8-tetrahydropyrrolo[1,2-a]pyrimidine-6-carboxamide (Compound 19)

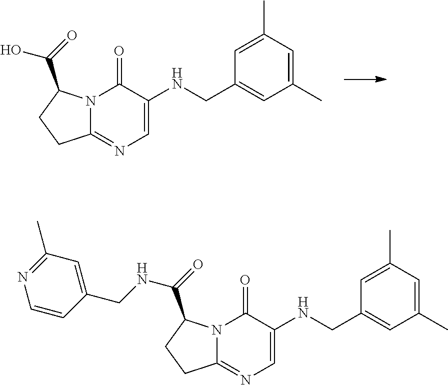 [SMILES: Cc1cc(C)cc(CNc2cnc3n(c2=O)[C@H](C(=O)O)CC3)c1 ; Cc1cc(C)cc(CNc2cnc3n(c2=O)[C@H](C(=O)NCc2ccnc(C)c2)CC3)c1]

Step 4: To a solution of (S)-3-((3,5-dimethylbenzyl)amino)-4-oxo-4,6,7,8-tetra-hydropyrrolo[1,2-a]pyrimidine-6-carboxylic acid (46 mg, 0.15 mmol, prepared according to Ex. 4) in DMF (2.5 mL) was added HATU (0.11 g, 0.29 mmol) and DIEA (57 mg, 77 μL, 0.44 mmol). After stirring for 10 min, 4-(aminomethyl)-2-methylpyridine (22 mg, 0.18 mmol) was added. After stirring for 18 hour at 20° C., the reaction mixture was concentrated and the residue was purified by column chromatography (amine column, 0-100% MeOH-DCM) and additional column chromatography (0-50% MeOH-DCM) to provide (S)-3-((3,5-dimethylbenzyl)amino)-N-((2-methylpyridin-4-yl)methyl)-4-oxo-4,6,7,8-tetrahydropyrrolo[1,2-a]pyrimidine-6-carboxamide (34.6 mg, 56% yield).

### Example 15

### Preparation of (S)-3-((3,5-dimethylbenzyl)amino)-N-(imidazo[1,5-a]pyridin-6-ylmethyl)-4-oxo-4,6,7,8-tetrahydropyrrolo[1,2-a]pyrimidine-6-carboxamide (Compound 20)

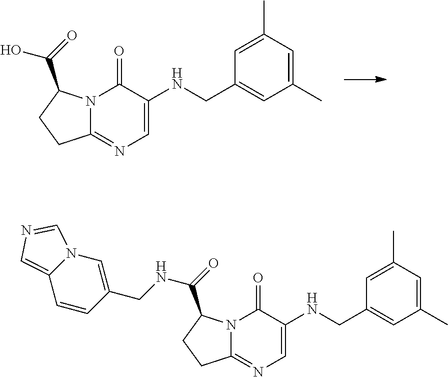 [SMILES: Cc1cc(C)cc(CNc2cnc3n(c2=O)[C@H](C(=O)O)CC3)c1 ; Cc1cc(C)cc(CNc2cnc3n(c2=O)[C@H](C(=O)NCc2ccc4cncn4c2)CC3)c1]

Step 1: To a solution of (S)-3-((3,5-dimethylbenzyl)amino)-4-oxo-4,6,7,8-tetra-hydropyrrolo[1,2-a]pyrimidine-6-carboxylic acid (41 mg, 0.13 mmol, prepared according to Ex. 4) in DMF (2.0 mL) was added HATU (0.10 g, 0.26 mmol) and DIEA (85 mg, 0.11 mL, 0.65 mmol). After stirring for 10 min, {imidazo[1,5-a]pyridin-6-yl}methanamine dihydrochloride (35 mg, 0.16 mmol) was added. After stirring for 18 hour at 20° C., the reaction mixture was concentrated and the residue was purified by column chromatography (amine column, 0-100% MeOH-DCM) and then additional column chromatography (0-50% MeOH-DCM) provided (S)-3-((3,5-dimethylbenzyl)amino)-N-(imidazo[1,5-a]pyridin-6-ylmethyl)-4-oxo-4,6,7,8-tetrahydropyrrolo[1,2-a]pyrimidine-6-carboxamide (23.1 mg, 40% yield).

### Example 16

### Preparation of (S)—N-((5,6-dihydro-4H-thieno[2,3-c]pyrrol-2-yl)methyl)-3-((3,5-dimethylbenzyl)amino)-4-oxo-4,6,7,8-tetrahydropyrrolo[1,2-a]pyrimidine-6-carboxamide, trifluoroacetate (Compound 21)

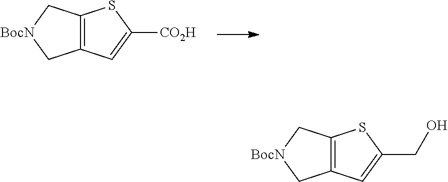 [SMILES: CC(C)(C)OC(=O)N1Cc2cc(C(=O)O)sc2C1 ; CC(C)(C)OC(=O)N1Cc2cc(CO)sc2C1]

Step 1: A brown mixture of 5-(tert-butoxycarbonyl)-5,6-dihydro-4//-thieno[2,3-c]pyrrole-2-carboxylic acid (5.0 g, 18.6 mmol) in THF (42 mL) was set under nitrogen and cooled to 0° C. A solution of borane tetrahydrofuran complex (6.9 g, 80 mL, 1 molar, 80 mmol) was added dropwise. The reaction was stirred at 0° C. for 5 min and warmed to room temp while stirring for 16 hours. The reaction was quenched with anhydrous methanol at 0° C. then stirred at room temp for 1 hour. Volatiles were evaporated under reduced pressure. The crude product was dissolved in CH₂Cl₂ and adsorbed onto silica gel. Purification by chromatography (0-100% EtOAc-hexane) afforded tert-butyl 2-(hydroxymethyl)-4,6-dihydro-5H-thieno[2,3-c]pyrrole-5-carboxylate (3.58 g, 75% yield) as a light yellow solid.

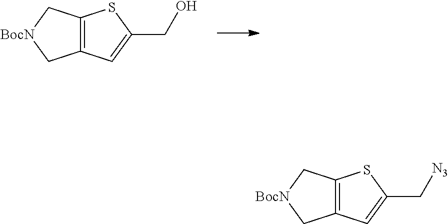 [SMILES: CC(C)(C)OC(=O)N1Cc2cc(CO)sc2C1 ; CC(C)(C)OC(=O)N1Cc2cc(CN=[N+]=[N-])sc2C1]

Step 2: A suspension of tert-butyl 2-(hydroxymethyl)-4,6-dihydro-5H-thieno[2,3-c]pyrrole-5-carboxylate (3.58 g, 14.00 mmol) in THF (35 mL) was put under nitrogen and treated with diphenylphosphoryl azide (5.8 g, 4.5 mL, 21 mmol) at room temp. After cooling to 0° C., 1,8-diazabicyclo[5.4.0]undec-7-ene (3.3 g, 3.2 mL, 21 mmol) was added. The reaction flask was sealed and warmed to room temp for about 5 min. The resulting solution was heated at 65° C. for 16 hours. The reaction was quenched with water and extracted with EtOAc 3 times. The organic layers were combined, washed with brine, dried over anhydrous Na₂SO₄, filtered, and evaporated under reduced pressure. The crude product was dissolved in CH₂Cl₂ and adsorbed onto silica gel. Purification by column chromatography (0-40% EtOAc-heptane) afforded tert-butyl 2-(azidomethyl)-4,6-dihydro-5H-thieno[2,3-c]pyrrole-5-carboxylate (3.82 g, 97% yield) as a white solid.

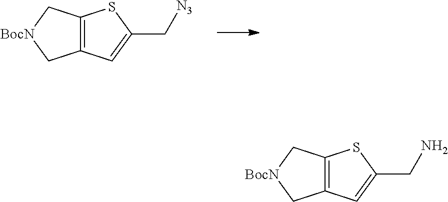 [SMILES: CC(C)(C)OC(=O)N1Cc2cc(CN=[N+]=[N-])sc2C1 ; CC(C)(C)OC(=O)N1Cc2cc(CN)sc2C1]

Step 3: A solution of tert-butyl 2-(azidomethyl)-4,6-dihydro-5H-thieno[2,3-c]pyrrole-5-carboxylate (3.82 g, 13.6 mmol) in THF (48 mL) was set under nitrogen, treated with water (5.4 g, 5.4 mL, 0.30 mol), and followed by addition of triphenylphosphine (5.37 g, 20.46 mmol) at room temp. The reaction mixture was stirred at room temp for 3 days. The reaction was quenched with 1 M KHSO₄ solution and washed with ether 3 times. The aqueous layer was treated with 5 N NaOH solution to pH about 11 and were extracted with EtOAc 3 times. The organic layers were combined, washed with brine, dried over anhyd Na₂SO₄, filtered, and evaporated under reduced pressure. The crude product was dissolved in CH₂Cl₂ and adsorbed onto silica gel. Purification by chromatography (0-10% 7 N NH₃ MeOH—CH₂Cl₂) afforded tert-butyl 2-(aminomethyl)-4,6-dihydro-5H-thieno[2,3-c]pyrrole-5-carboxylate (2.54 g, 73%) as a white solid.

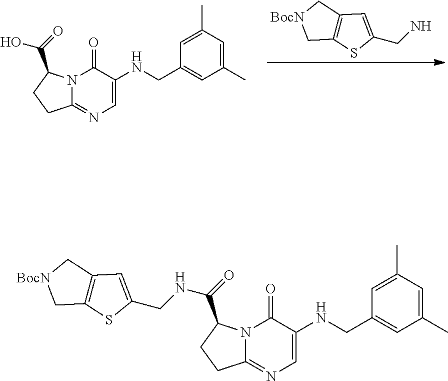 [SMILES: Cc1cc(C)cc(CNc2cnc3n(c2=O)[C@H](C(=O)O)CC3)c1 ; CC(C)(C)OC(=O)N1Cc2cc(CN)sc2C1 ; Cc1cc(C)cc(CNc2cnc3n(c2=O)[C@H](C(=O)NCc2cc4c(s2)CN(C(=O)OC(C)(C)C)C4)CC3)c1]

Step 4: To a solution of (S)-3-((3,5-dimethylbenzyl)amino)-4-oxo-4,6,7,8-tetrahydropyrrolo[1,2-a]pyrimidine-6-carboxylic acid (41 mg, 0.13 mmol, prepared according to Ex. 4) in DMF (2.5 mL) was added HATU (0.10 g, 0.26 mmol) and DIEA (51 mg, 68 μL, 0.39 mmol). After stirring for 10 min, tert-butyl 2-(aminomethyl)-4,6-dihydro-5H-thieno[2,3-c]pyrrole-5-carboxylate (40 mg, 0.16 mmol) was added. After stirring for 18 hour at 20° C., the reaction mixture was concentrated and the residue was purified by column chromatography (0-100% EtOAc-heptane) to provide tert-butyl (S)-2-((3-((3,5-dimethylbenzyl)amino)-4-oxo-4,6,7,8-tetrahydropyrrolo[1,2-a]pyrimidine-6-carboxamido)methyl)-4,6-dihydro-5H-thieno[2,3-c]pyrrole-5-carboxylate (61 mg, 85% yield).

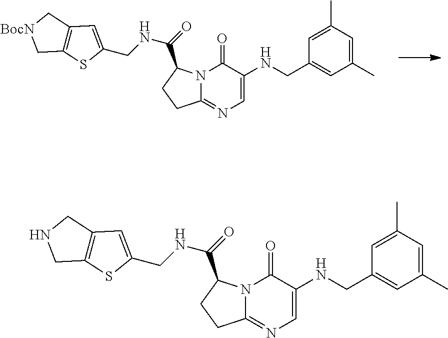 [SMILES: Cc1cc(C)cc(CNc2cnc3n(c2=O)[C@H](C(=O)NCc2cc4c(s2)CN(C(=O)OC(C)(C)C)C4)CC3)c1 ; Cc1cc(C)cc(CNc2cnc3n(c2=O)[C@H](C(=O)NCc2cc4c(s2)CNC4)CC3)c1]

Step 5: To a 0° C. solution of tert-butyl (S)-2-((3-((3,5-dimethyl-benzyl)amino)-4-oxo-4,6,7,8-tetrahydropyrrolo[1,2-a]pyrimidine-6-carboxamido)-methyl)-4,6-dihydro-5H-thieno[2,3-c]pyrrole-5-carboxylate (61 mg, 0.11 mmol) in DCM (3 mL) was added TFA (0.51 g, 0.34 mL, 4.4 mmol). After stirring for 3 hours at 20° C., the reaction mixture was concentrated. The residue was purified by preparatory HPLC method 2 to provide (S)—N-((5,6-dihydro-4H-thieno[2,3-c]pyrrol-2-yl)methyl)-3-((3,5-dimethylbenzyl)amino)-4-oxo-4,6,7,8-tetrahydropyrrolo[1,2-a]pyrimidine-6-carboxamide, trifluoracetate (31 mg, 50% yield).

### Example 17

### Preparation of (S)—N-((6,7-dihydro-5H-pyrrolo[3,4-b]pyridin-3-yl)methyl)-3-((3,5-dimethylbenzyl)amino)-4-oxo-4,6,7,8-tetrahydropyrrolo[1,2-a]pyrimidine-6-carboxamide, trifluoroacetate (Compound 22)

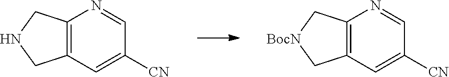 [SMILES: N#Cc1cnc2c(c1)CNC2 ; CC(C)(C)OC(=O)N1Cc2cc(C#N)cnc2C1]

Step 1: To a solution of 6,7-dihydro-5H-pyrrolo[3,4-b]pyridine-3-carbonitrile, HCl (100 mg, 551 μmol) in DCM (4 mL) was added Boc₂O (120 mg, 126 μL, 551 μmol) and DMAP (67.3 mg, 551 μmol). After stirring for 1 hour at 20° C., the reaction mixture was concentrated. The residue was purified by flash column chromatography (0-100% EtOAc-heptane) to provide tert-butyl 3-cyano-5,7-dihydro-6H-pyrrolo[3,4-b]pyridine-6-carboxylate (120 mg, 89% yield).

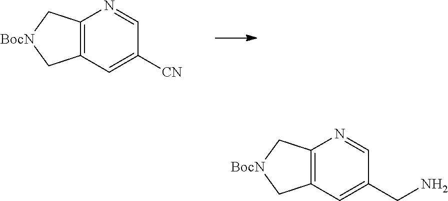 [SMILES: CC(C)(C)OC(=O)N1Cc2cc(C#N)cnc2C1 ; CC(C)(C)OC(=O)N1Cc2cc(CN)cnc2C1]

Step 2: To a solution of tert-butyl 3-cyano-5,7-dihydro-6H-pyrrolo[3,4-b]pyridine-6-carboxylate (120 mg, 426 μmol) in methanol (1.5 mL) was added 7 N NH₃—MeOH (7 mL). The mixture was degassed with vac/Ar 2 times. Raney-Ni (˜100 mg) was added. The mixture was degassed with vac/H₂. After stirring for 18 hours at 20° C., the reaction mixture was degassed with vac/Ar and filtered through a Celite® plug with MeOH. The combined filtrate was concentrated. The residue was taken up in 5% H₂O—MeOH and filtered through a 0.2 um filter to provide tert-butyl 3-(aminomethyl)-5,7-dihydro-6H-pyrrolo[3,4-b]pyridine-6-carboxylate (106 mg, 100% yield), which was used for the next step without further purification.

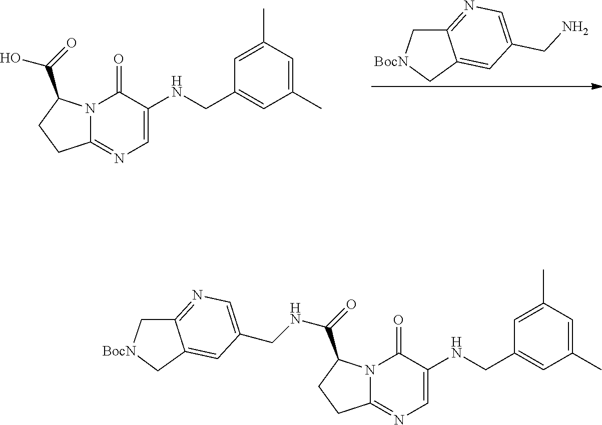 [SMILES: Cc1cc(C)cc(CNc2cnc3n(c2=O)[C@H](C(=O)O)CC3)c1 ; CC(C)(C)OC(=O)N1Cc2cc(CN)cnc2C1 ; Cc1cc(C)cc(CNc2cnc3n(c2=O)[C@H](C(=O)NCc2cnc4c(c2)CN(C(=O)OC(C)(C)C)C4)CC3)c1]

Step 3: To a solution of (S)-3-((3,5-dimethylbenzyl)amino)-4-oxo-4,6,7,8-tetrahydropyrrolo[1,2-a]pyrimidine-6-carboxylic acid (52 mg, 0.17 mmol, prepared according to Ex. 4) in DMF (2.0 mL) was added HATU (0.13 g, 0.33 mmol) and DIEA (64 mg, 87 μL, 0.50 mmol). After stirring for 10 min, tert-butyl 3-(aminomethyl)-5,7-dihydro-6H-pyrrolo[3,4-b]pyridine-6-carboxylate (50 mg, 0.20 mmol) was added. After stirring for 18 hours at 20° C., the reaction mixture was concentrated and the residue was purified by column chromatography to provide tert-butyl (S)-3-((3-((3,5-dimethylbenzyl)amino)-4-oxo-4,6,7,8-tetrahydropyrrolo[1,2-a]pyrimidine-6-carboxamido)methyl)-5,7-dihydro-6H-pyrrolo[3,4-b]pyridine-6-carboxylate (90 mg, 100% yield).

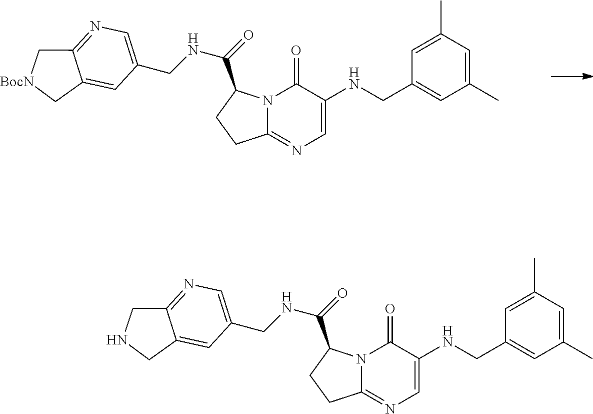 [SMILES: Cc1cc(C)cc(CNc2cnc3n(c2=O)[C@H](C(=O)NCc2cnc4c(c2)CN(C(=O)OC(C)(C)C)C4)CC3)c1 ; Cc1cc(C)cc(CNc2cnc3n(c2=O)[C@H](C(=O)NCc2cnc4c(c2)CNC4)CC3)c1]

Step 4: To a 0° ° C. solution of tert-butyl (S)-3-((3-((3,5-dimethylbenzyl)amino)-4-oxo-4,6,7,8-tetrahydropyrrolo[1,2-a]pyrimidine-6-carboxamido)methyl)-5,7-dihydro-6H-pyrrolo[3,4-b]pyridine-6-carboxylate (90 mg, 0.17 mmol) in DCM (4 mL) was added TFA (0.57 g, 0.38 mL, 5.0 mmol). After stirring for 3 hours at 20° ° C., the reaction mixture was concentrated. The residue was purified by preparatory HPLC method 2 to provide (S)—N-((6,7-dihydro-5H-pyrrolo[3,4-b]pyridin-3-yl)methyl)-3-((3,5-dimethyl-benzyl)amino)-4-oxo-4,6,7,8-tetrahydropyrrolo[1,2-a]pyrimidine-6-carboxamide trifluoroacetate (27.5 mg, 30% yield).

### Example 18

### Preparation of (S)-3-((3,5-dimethylbenzyl)amino)-N-((2-methylpyridin-3-yl)methyl)-4-oxo-4,6,7,8-tetrahydropyrrolo[1,2-a]pyrimidine-6-carboxamide, trifluoroacetate (Compound 23)

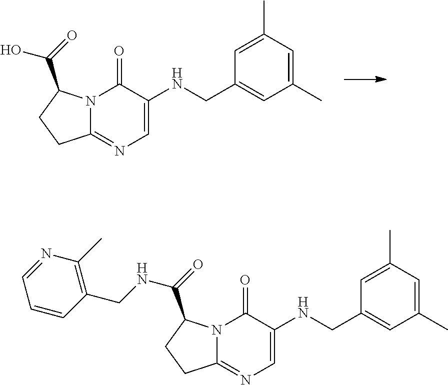 [SMILES: Cc1cc(C)cc(CNc2cnc3n(c2=O)[C@H](C(=O)O)CC3)c1 ; Cc1cc(C)cc(CNc2cnc3n(c2=O)[C@H](C(=O)NCc2cccnc2C)CC3)c1]

Step 1: To a solution of (S)-3-((3,5-dimethylbenzyl)amino)-4-oxo-4,6,7,8-tetra-hydropyrrolo[1,2-a]pyrimidine-6-carboxylic acid (46 mg, 0.15 mmol, prepared according to Ex. 4) in DMF (2.5 mL) was added HATU (0.11 g, 0.29 mmol) and DIEA (57 mg, 77 μL, 0.44 mmol). After stirring for 10 min, (2-methyl-3-pyridyl)methanamine (18 mg, 0.15 mmol) was added. After stirring for an additional 18 hours at 20° C., the reaction was concentrated and the residue was purified by column chromatography (amine column, 0-100% MeOH-DCM). Semi-pure fractions were collected and additional column chromatography (0-50% MeOH-DCM) was performed. Final purification was performed by preparatory HPLC method 2 to provide (S)-3-((3,5-dimethylbenzyl)amino)-N-((2-methylpyridin-3-yl)-methyl)-4-oxo-4,6,7,8-tetrahydropyrrolo[1,2-a]pyrimidine-6-carboxamide, trifluoroacetate (34.6 mg, 56% yield).

### Example 19

### Preparation of (S)—N-((6-amino-2-methylpyridin-3-yl)methyl)-4-oxo-3-(((1-phenyl-1H-pyrazol-4-yl)methyl)amino)-4,6,7,8-tetrahydropyrrolo[1,2-a]pyrimidine-6-carboxamide, trifluoroacetate (Compound 24)

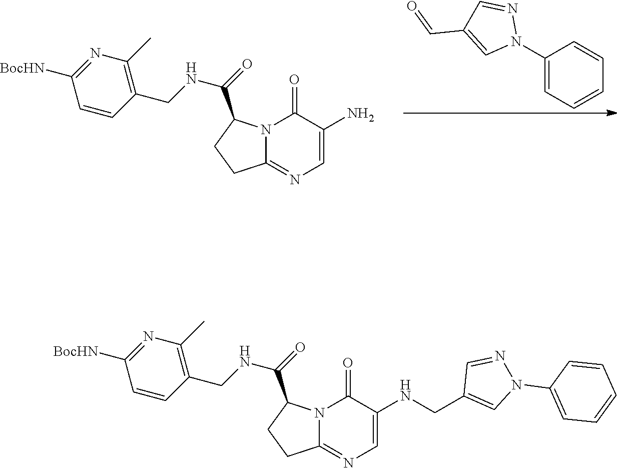 [SMILES: Cc1nc(NC(=O)OC(C)(C)C)ccc1CNC(=O)[C@@H]1CCc2ncc(N)c(=O)n21 ; O=Cc1cnn(-c2ccccc2)c1 ; Cc1nc(NC(=O)OC(C)(C)C)ccc1CNC(=O)[C@@H]1CCc2ncc(NCc3cnn(-c4ccccc4)c3)c(=O)n21]

Step 1: To a solution of tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (60 mg, 0.14 mmol, prepared according to Ex. 2) and 1-phenyl-1H-pyrazole-4-carbaldehyde (50 mg, 0.29 mmol) in DCE (2 mL) was added HOAc (50 μL, 0.87 mmol). After stirring for 30 min, sodium triacetoxyborohydride (92 mg, 0.43 mmol) was added. After stirring for an additional 1 hour at 20° C., the reaction mixture was diluted in DCM and washed with sat. NaHCO₃, brine, dried over Na₂SO₄, filtered, and concentrated to dryness. The residue was purified by column chromatography (0-100% EtOAc-heptane and 0-30% MeOH-DCM) to provide tert-butyl (S)-(6-methyl-5-((4-oxo-3-(((1-phenyl-1H-pyrazol-4-yl)methyl)amino)-4,6,7,8-tetrahydropyrrolo[1,2-a]pyrimidine-6-carboxamido)methyl)-pyridin-2-yl)carbamate (74 mg, 90% yield).

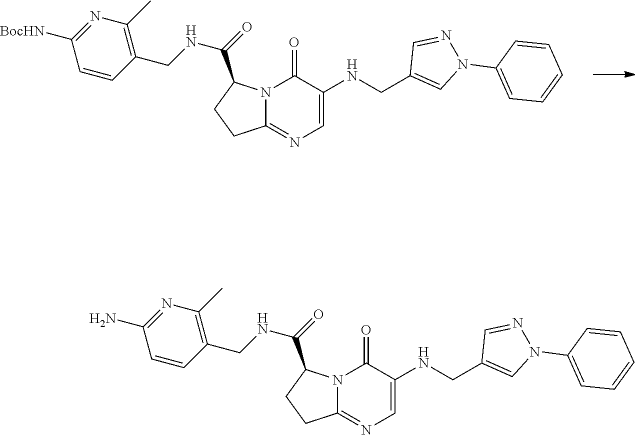 [SMILES: Cc1nc(NC(=O)OC(C)(C)C)ccc1CNC(=O)[C@@H]1CCc2ncc(NCc3cnn(-c4ccccc4)c3)c(=O)n21 ; Cc1nc(N)ccc1CNC(=O)[C@@H]1CCc2ncc(NCc3cnn(-c4ccccc4)c3)c(=O)n21]

Step 2: To a 0° C. solution of tert-butyl (S)-(6-methyl-5-((4-oxo-3-(((1-phenyl-1H-pyrazol-4-yl)methyl)amino)-4,6,7,8-tetrahydropyrrolo[1,2-a]pyrimidine-6-carboxamido)methyl)pyridin-2-yl)carbamate (74 mg, 0.13 mmol) in DCM (4 mL) was added TFA (0.59 g, 0.40 mL, 5.2 mmol). After stirring for 3 hours at 20° C., the reaction mixture was concentrated. The residue was purified by preparatory HPLC method 2 to provide (S)—N-((6-amino-2-methylpyridin-3-yl)methyl)-4-oxo-3-(((1-phenyl-1H-pyrazol-4-yl)methyl)amino)-4,6,7,8-tetrahydropyrrolo[1,2-a]pyrimidine-6-carboxamide, trifluoroacetate (64.4 mg, 85% yield).

The following compounds were prepared according to the foregoing procedure using the appropriate aldehyde starting materials:

| | Cmp |
|---|---|
| Compound Name | No. |
| (S)-N-((6-amino-2-methylpyridin-3-yl)methyl)-3-((3-ethyl- | 26 |
| benzyl)amino)-4-oxo-4,6,7,8-tetrahydropyrrolo[1,2-a]pyrimidine-6- | |
| carboxamide, trifluoroacetate | |

### Example 20

### Preparation of (S)—N—((R)-1-(6-aminopyridin-3-yl)ethyl)-3-((3-fluoro-5-methylbenzyl)amino)-4-oxo-4,6,7,8-tetrahydropyrrolo[1,2-a]pyrimidine-6-carboxamide, DI-trifluoroacetate(Compound 25)

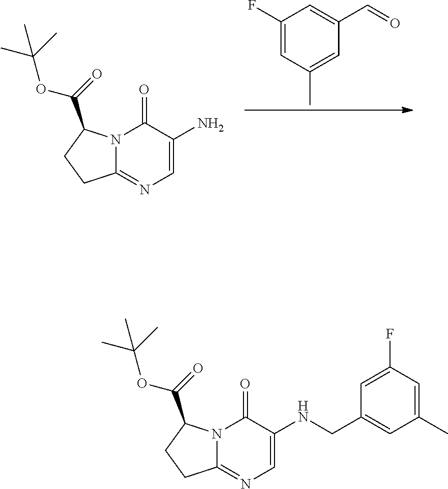

Step 1: To a solution of tert-butyl (S)-3-amino-4-oxo-4,6,7,8-tetrahydro-pyrrolo[1,2-a]pyrimidine-6-carboxylate (60 mg, 0.24 mmol, prepared according to Ex. 4) and 3-fluoro-5-methylbenzaldehyde (66 mg, 58 μL, 0.48 mmol) in DCE (3 mL) was added HOAc (82 μL, 1.4 mmol). After stirring for 30 min, sodium triacetoxyborohydride (0.15 g, 0.72 mmol) was added. After stirring for 1 hour at 20° C., the reaction mixture was diluted in DCM and washed with sat. NaHCO₃, brine, dried over Na₂SO₄, filtered, and concentrated to dryness. The residue was purified by column chromatography (0-100% EtOAc-heptane) to provide tert-butyl (S)-3-((3-fluoro-5-methylbenzyl)amino)-4-oxo-4,6,7,8-tetrahydropyrrolo[1,2-a]pyrimidine-6-carboxylate (48 mg, 54% yield).

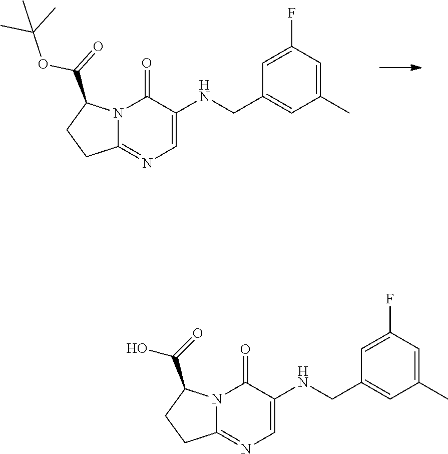 [SMILES: Cc1cc(F)cc(CNc2cnc3n(c2=O)[C@H](C(=O)OC(C)(C)C)CC3)c1 ; Cc1cc(F)cc(CNc2cnc3n(c2=O)[C@H](C(=O)O)CC3)c1]

Step 2: tert-Butyl (S)-3-((3-fluoro-5-methylbenzyl)amino)-4-oxo-4,6,7,8-tetra-hydropyrrolo[1,2-a]pyrimidine-6-carboxylate (48 mg, 0.13 mmol) was dissolved in DCM (2 mL) and treated with TFA (0.44 g, 0.30 mL, 3.9 mmol). After stirring for 3 hours, 0.3 mL of TFA was added and stirred overnight. The mixture was concentrated to provide the crude (S)-3-((3-fluoro-5-methylbenzyl)amino)-4-oxo-4,6,7,8-tetrahydropyrrolo[1,2-a]pyrimidine-6-carboxylic acid (41 mg, 100% yield) which was applied to the next step without further purification.

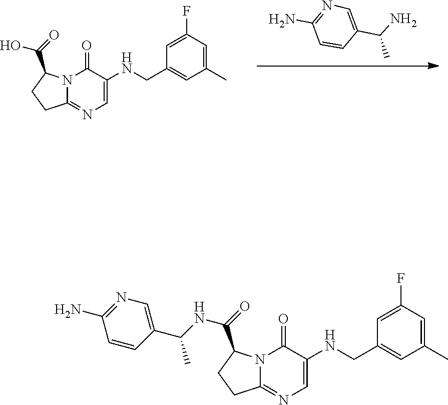

Step 3: To a solution of (S)-3-((3-fluoro-5-methylbenzyl)amino)-4-oxo-4,6,7,8-tetrahydropyrrolo[1,2-a]pyrimidine-6-carboxylic acid (41 mg, 0.13 mmol) in DMF (2.0 mL) was added HATU (98 mg, 0.26 mmol) and DIEA (84 mg, 0.11 mL, 0.65 mmol). After stirring for 10 min, (R)-5-(1-aminoethyl)pyridin-2-amine, hydrochloride (27 mg, 0.16 mmol) was added. After stirring for an additional 18 hours at 20° C., the reaction mixture was concentrated and the residue was purified by column chromatography (amine column, 0-30% MeOH-DCM) and preparatory HPLC method 2 to provide (S)—N—((R)-1-(6-aminopyridin-3-yl)ethyl)-3-((3-fluoro-5-methylbenzyl)amino)-4-oxo-4,6,7,8-tetrahydropyrrolo[1,2-a]pyrimidine-6-carboxamide, di-trifluoroacetate (30.2 mg, 35% yield).

### Example 21

### Preparation of (S)—N-((6-amino-2-methylpyridin-3-yl)methyl)-3-((2-(difluoromethyl)benzyl)amino)-4-oxo-4,6,7,8-tetrahydropyrrolo[1,2-a]pyrimidine-6-carboxamide, di-trifluoroacetate (Compound 27)

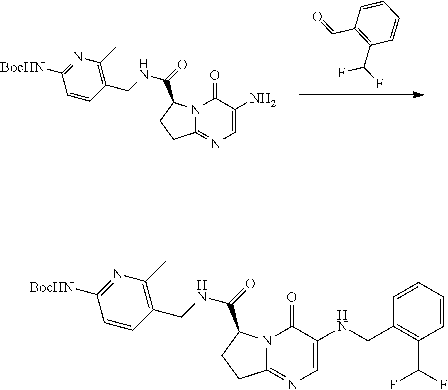 [SMILES: Cc1nc(NC(=O)OC(C)(C)C)ccc1CNC(=O)[C@@H]1CCc2ncc(N)c(=O)n21 ; O=Cc1ccccc1C(F)F ; Cc1nc(NC(=O)OC(C)(C)C)ccc1CNC(=O)[C@@H]1CCc2ncc(NCc3ccccc3C(F)F)c(=O)n21]

Step 1: To a solution of tert-butyl (S)-(5-((3-amino-4-oxo-4,6,7,8-tetrahydro-pyrrolo[1,2-a]pyrimidine-6-carboxamido)methyl)-6-methylpyridin-2-yl)carbamate (50 mg, 0.12 mmol, prepared according to Ex. 1) and 2-(difluoromethyl)benzaldehyde (38 mg, 29 μL 0.24 mmol) in DCE (2 mL) was added HOAc (41 μL, 0.72 mmol). After stirring for 30 min, sodium triacetoxyborohydride (77 mg, 0.36 mmol) was added. After stirring for 2 hours at 20° C., the reaction mixture was diluted in DCM and washed with sat. NaHCO₃, brine, dried over Na₂SO₄, filtered, and concentrated to dryness. The residue was purified by column chromatography (0-100% EtOAc-heptane and 0-30% MeOH-DCM) to provide tert-butyl (S)-(5-((3-((2-(difluoromethyl)benzyl)amino)-4-oxo-4,6,7,8-tetrahydropyrrolo[1,2-a]pyrimidine-6-carboxamido)methyl)-6-methylpyridin-2-yl)carbamate (62 mg, 93% yield).

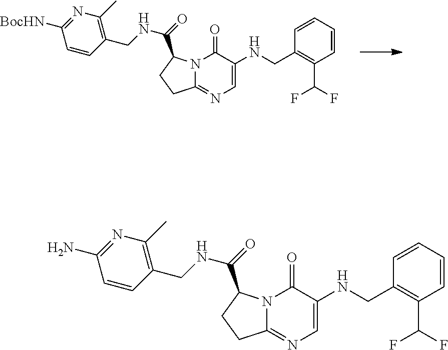 [SMILES: Cc1nc(NC(=O)OC(C)(C)C)ccc1CNC(=O)[C@@H]1CCc2ncc(NCc3ccccc3C(F)F)c(=O)n21 ; Cc1nc(N)ccc1CNC(=O)[C@@H]1CCc2ncc(NCc3ccccc3C(F)F)c(=O)n21]

Step 2: To a solution of tert-butyl (S)-(5-((3-((2-(difluoromethyl)benzyl)amino)-4-oxo-4,6,7,8-tetrahydropyrrolo[1,2-a]pyrimidine-6-carboxamido)methyl)-6-methylpyridin-2-yl)carbamate (62 mg, 0.11 mmol) in DCM (3.5 mL) was added TFA (0.51 g, 0.34 mL, 4.5 mmol). After stirring for 18 hours at 20° C., the reaction mixture was concentrated. The residue was purified by column chromatography (amine column, 0-50% MeOH-DCM) and reverse phase column chromatography. The final purification by preparatory HPLC method 2 provided (S)—N-((6-amino-2-methylpyridin-3-yl)methyl)-3-((2-(difluoromethyl)benzyl)amino)-4-oxo-4,6,7,8-tetrahydropyrrolo[1,2-a]pyrimidine-6-carboxamide, di-trifluoroacetate (6.8 mg, 9% yield).

The following compounds were prepared according to the foregoing procedure using the appropriate aldehyde starting materials except that purification in step 6 consisted of preparatory HPLC method 2 purification:

| | Cmp |
|---|---|
| Compound Name | No. |
| (S)-N-((6-amino-2-methylpyridin-3-yl)methyl)-3-((2,5- | 28 |
| difluorobenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((3-methyl-5- | 29 |
| (trifluoromethyl)benzyl)amino)-4-oxo-4,6,7,8-tetrahydro- | |
| pyrrolo[1,2-a]pyrimidine-6-carboxamide, di-trifluoroacetate | |
| (S)-N-((6-amino-2-methylpyridin-3-yl)methyl)-3-((2,3- | 30 |
| dimethylbenzyl)amino)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((5-fluoro-2- | 31 |
| methylbenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((2,6- | 32 |
| difluorobenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((2-fluoro-3- | 33 |
| methylbenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((3-fluoro-5- | 34 |
| methylbenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((3,5-difluoro-2- | 35 |
| methoxybenzyl)amino)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((3,6-difluoro-2- | 36 |
| methoxybenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((3- | 37 |
| cyclopropoxybenzyl)amino)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((2-fluoro-5- | 38 |
| (trifluoromethyl)benzyl)amino)-4-oxo-4,6,7,8-tetrahydro- | |
| pyrrolo[1,2-a]pyrimidine-6-carboxamide, di-trifluoroacetate | |
| (S)-N-((6-amino-2-methylpyridin-3-yl)methyl)-3-((2-fluoro-3- | 39 |
| (trifluoromethoxy)benzyl)amino)-4-oxo-4,6,7,8-tetrahydro- | |
| pyrrolo[1,2-a]pyrimidine-6-carboxamide, di-trifluoroacetate | |
| (S)-3-((3-(1H-pyrazol-1-yl)benzyl)amino)-N-((6-amino-2- | 40 |
| methylpyridin-3-yl)methyl)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((2-fluoro-5- | 41 |
| (trifluoromethoxy)benzyl)amino)-4-oxo-4,6,7,8-tetrahydro- | |
| pyrrolo[1,2-a]pyrimidine-6-carboxamide, di-trifluoroacetate | |
| (S)-N-((6-amino-2-methylpyridin-3-yl)methyl)-3-((2-fluoro-3- | 42 |
| (trifluoromethyl)benzyl)amino)-4-oxo-4,6,7,8-tetrahydro- | |
| pyrrolo[1,2-a]pyrimidine-6-carboxamide, di-trifluoroacetate | |
| (S)-N-((6-amino-2-methylpyridin-3-yl)methyl)-3-((2-fluoro-4- | 43 |
| (trifluoromethyl)benzyl)amino)-4-oxo-4,6,7,8-tetrahydro- | |
| pyrrolo[1,2-a]pyrimidine-6-carboxamide, di-trifluoroacetate | |
| (S)-3-((4-(1H-pyrazol-1-yl)benzyl)amino)-N-((6-amino-2- | 44 |
| methylpyridin-3-yl)methyl)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((3-methoxy-4- | 45 |
| methylbenzyl)amino)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((3-fluoro-4- | 46 |
| methoxybenzyl)amino)-4-oxo-4,6,7,8-tetrahydropyrrolo[1,2- | |
| a]pyrimidine-6-carboxamide, di-trifluoroacetate | |
| (S)-N-((6-amino-2-methylpyridin-3-yl)methyl)-3-((3-fluoro-5- | 47 |
| (trifluoromethyl)benzyl)amino)-4-oxo-4,6,7,8-tetrahydro- | |
| pyrrolo[1,2-a]pyrimidine-6-carboxamide, di-trifluoroacetate | |
| (S)-N-((6-amino-2-methylpyridin-3-yl)methyl)-3-((3-methoxy-2- | 48 |
| methylbenzyl)amino)-4-oxo-4,6,7,8-tetrahydropyrrolo[1,2- | |
| a]pyrimidine-6-carboxamide, trifluoroacetate | |
| (S)-N-((6-amino-2-methylpyridin-3-yl)methyl)-3-((3-fluoro-5- | 49 |
| methoxybenzyl)amino)-4-oxo-4,6,7,8-tetrahydropyrrolo[1,2- | |
| a]pyrimidine-6-carboxamide, di-trifluoroacetate | |

### Example 22

### Preparation of (S)—N-((5,6-dihydro-4H-thieno[2,3-C]pyrrol-2-yl)methyl)-3-((2-fluoro-5-methylbenzyl)amino)-4-oxo-4,6,7,8-tetrahydropyrrolo[1,2-a]pyrimidine-6-carboxamide, di-trifluoroacetate (Compound 50)

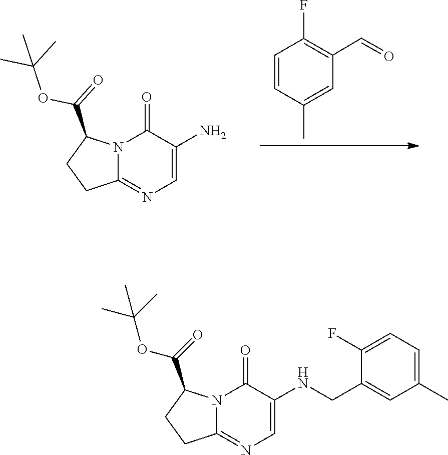

Step 1: To a solution of tert-butyl (S)-3-amino-4-oxo-4,6,7,8-tetrahydro-pyrrolo[1,2-a]pyrimidine-6-carboxylate (68 mg, 0.27 mmol, prepared according to Ex. 4) and 2-fluoro-5-methylbenzaldehyde (75 mg, 68 μL, 0.54 mmol) in DCE (3 mL) was added HOAc (93 μL, 1.6 mmol). After stirring for 30 min, sodium triacetoxyborohydride (0.17 g, 0.81 mmol) was added. After stirring for 2 hours at 20° C., the reaction mixture was diluted in DCM and washed with sat. NaHCO₃, brine, dried over Na₂SO₄, filtered, and concentrated to dryness. The residue was purified by column chromatography (0-100% EtOAc-heptane) to provide tert-butyl (S)-3-((2-fluoro-5-methylbenzyl)amino)-4-oxo-4,6,7,8-tetrahydropyrrolo[1,2-a]pyrimidine-6-carboxylate (60 mg 59% yield)

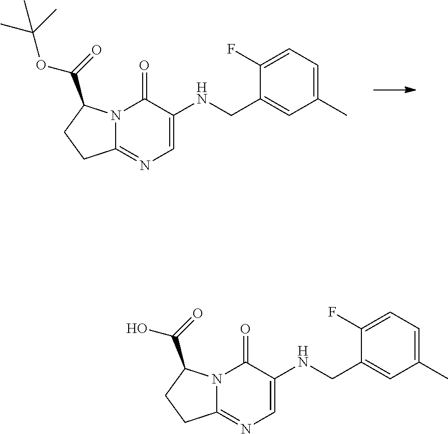 [SMILES: Cc1ccc(F)c(CNc2cnc3n(c2=O)[C@H](C(=O)OC(C)(C)C)CC3)c1 ; Cc1ccc(F)c(CNc2cnc3n(c2=O)[C@H](C(=O)O)CC3)c1]

Step 2: tert-Butyl (S)-3-((2-fluoro-5-methylbenzyl)amino)-4-oxo-4,6,7,8-tetra-hydropyrrolo[1,2-a]pyrimidine-6-carboxylate (60 mg, 0.16 mmol) was dissolved in DCM (3 mL) and treated with TFA (0.37 mL, 4.8 mmol). After stirring for 18 hours at 20° C. the mixture was concentrated to provide the crude (S)-3-((2-fluoro-5-methylbenzyl)amino)-4-oxo-4,6,7,8-tetrahydropyrrolo[1,2-a]pyrimidine-6-carboxylic acid (51 mg, 100% yield) which was applied to the next step without further purification.

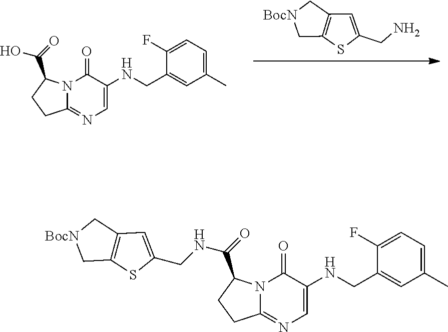 [SMILES: Cc1ccc(F)c(CNc2cnc3n(c2=O)[C@H](C(=O)O)CC3)c1 ; CC(C)(C)OC(=O)N1Cc2cc(CN)sc2C1 ; Cc1ccc(F)c(CNc2cnc3n(c2=O)[C@H](C(=O)NCc2cc4c(s2)CN(C(=O)OC(C)(C)C)C4)CC3)c1]

Step 3: To a solution of (S)-3-((2-fluoro-5-methylbenzyl)amino)-4-oxo-4,6,7,8-tetrahydropyrrolo[1,2-a]pyrimidine-6-carboxylic acid (51 mg, 0.16 mmol) in DMF (3 mL) was added HATU (0.12 g, 0.32 mmol) and DIEA (62 mg, 84 μL, 0.48 mmol). After stirring for 10 minutes, tert-butyl 2-(aminomethyl)-4,6-dihydro-5H-thieno[2,3-c]pyrrole-5-carboxylate (49 mg, 0.19 mmol, prepared according to Ex. 16) was added. After stirring for 18 hour at 20° C., the reaction was concentrated and the residue was purified by column chromatography (0-100% EtOAc-heptane) to provide tert-butyl (S)-2-((3-((2-fluoro-5-methylbenzyl)amino)-4-oxo-4,6,7,8-tetrahydropyrrolo[1,2-a]pyrimidine-6-carboxamido)methyl)-4,6-dihydro-5H-thieno[2,3-c]pyrrole-5-carboxylate (88 mg, 99% yield).

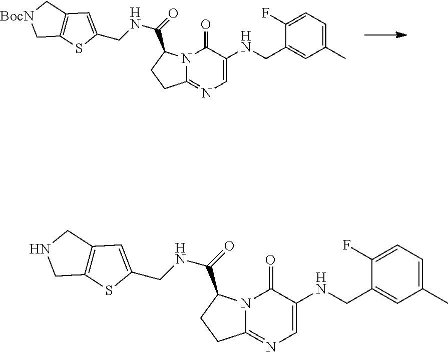 [SMILES: Cc1ccc(F)c(CNc2cnc3n(c2=O)[C@H](C(=O)NCc2cc4c(s2)CN(C(=O)OC(C)(C)C)C4)CC3)c1 ; Cc1ccc(F)c(CNc2cnc3n(c2=O)[C@H](C(=O)NCc2cc4c(s2)CNC4)CC3)c1]

Step 4: To a solution of tert-butyl (S)-2-((3-((2-fluoro-5-methylbenzyl)amino)-4-oxo-4,6,7,8-tetrahydropyrrolo[1,2-a]pyrimidine-6-carboxamido)methyl)-4,6-dihydro-5H-thieno[2,3-c]pyrrole-5-carboxylate (88 mg, 0.16 mmol) in DCM (4 mL) was added TFA (0.49 mL, 6.4 mmol). After stirring for 3 hours at 20° C., the reaction mixture was concentrated. The residue was purified by preparatory HPLC method 2 to provide (S)—N-((5,6-dihydro-4H-thieno[2,3-c]pyrrol-2-yl)methyl)-3-((2-fluoro-5-methylbenzyl)amino)-4-oxo-4,6,7,8-tetrahydropyrrolo[1,2-a]pyrimidine-6-carboxamide, di-trifluoroacetate (49.2 mg, 45% yield).

### Example 23

### Preparation of (S)—N—((R)-1-(6-aminopyridin-3-yl)ethyl)-3-((3-methyl-5-(trifluoromethyl)benzyl)amino)-4-oxo-4,6,7,8-tetrahydropyrrolo[1,2-a]pyrimidine-6-carboxamide. DI-trifluoroacetate (Compound) 51)

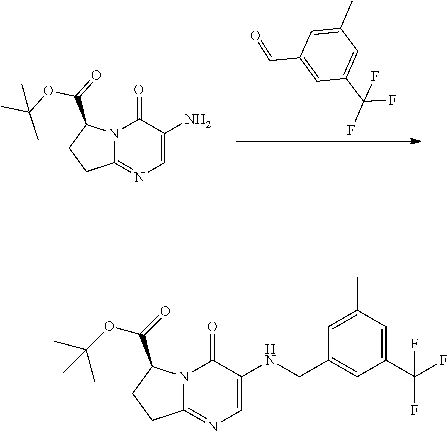 [SMILES: CC(C)(C)OC(=O)[C@@H]1CCc2ncc(N)c(=O)n21 ; Cc1cc(C=O)cc(C(F)(F)F)c1 ; Cc1cc(CNc2cnc3n(c2=O)[C@H](C(=O)OC(C)(C)C)CC3)cc(C(F)(F)F)c1]

Step 1: To a solution of tert-butyl (S)-3-amino-4-oxo-4,6,7,8-tetrahydro-pyrrolo[1,2-a]pyrimidine-6-carboxylate (68 mg, 0.27 mmol, prepared according to Ex. 4) and 3-methyl-5-(trifluoromethyl)benzaldehyde (0.15 g, 0.81 mmol) in DCE (3 mL) was added HOAc (93 μL, 1.6 mmol). After stirring for 30 min, sodium triacetoxyborohydride (0.34 g, 1.6 mmol) was added. After stirring for 2 hours at 20° C., the reaction mixture was diluted in DCM and washed with sat. NaHCO₃, brine, dried over Na₂SO₄, filtered, and concentrated to dryness. The residue was purified by column chromatography (0-100% EtOAc-heptane and 0-30% MeOH-DCM) to provide tert-butyl (S)-3-((3-methyl-5-(trifluoromethyl)benzyl)amino)-4-oxo-4,6,7,8-tetrahydropyrrolo[1,2-a]pyrimidine-6-carboxylate (93 mg, 81% yield).

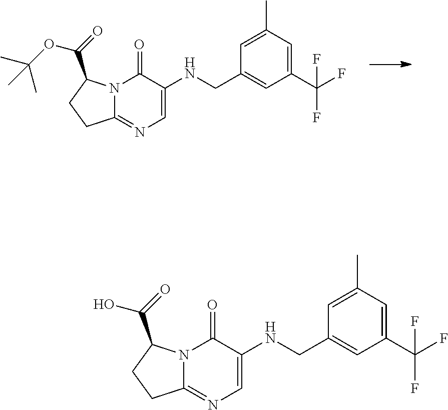 [SMILES: Cc1cc(CNc2cnc3n(c2=O)[C@H](C(=O)OC(C)(C)C)CC3)cc(C(F)(F)F)c1 ; Cc1cc(CNc2cnc3n(c2=O)[C@H](C(=O)O)CC3)cc(C(F)(F)F)c1]

Step 2: To a solution of tert-butyl (S)-3-((3-methyl-5-(trifluoromethyl)-benzyl)amino)-4-oxo-4,6,7,8-tetrahydropyrrolo[1,2-a]pyrimidine-6-carboxylate (93 mg, 0.22 mmol) in DCM (5 mL) was added TFA (0.51 mL, 6.6 mmol). After stirring for 18 hour at 20 ºC, the reaction mixture was concentrated to provide (S)-3-((3-methyl-5-(trifluoromethyl)benzyl)amino)-4-oxo-4,6,7,8-tetrahydropyrrolo[1,2-a]pyrimidine-6-carboxylic acid (107 mg, quantitative).

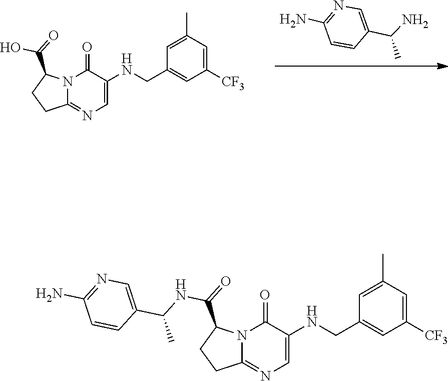

Step 3: To a solution of (S)-3-((3-methyl-5-(trifluoromethyl)benzyl)amino)-4-oxo-4,6,7,8-tetrahydropyrrolo[1,2-a]pyrimidine-6-carboxylic acid (107 mg, 291 μmol) in DMF (3 mL) was added DIEA (188 mg, 254 μL, 1.46 mmol). After stirring for 30 min, HATU (222 mg, 583 μmol) was added and stirred for 10 min. (R)-5-(1-aminoethyl)pyridin-2-amine, hydrochloride (60.7 mg, 350 μmol) was added to the above mixture. After stirring for 18 hours at 20° C., the reaction mixture was concentrated and the residue was purified by column chromatography (amine column, 0-30% MeOH-DCM) and preparatory HPLC method 2 to provide (S)—N—((R)-1-(6-aminopyridin-3-yl)ethyl)-3-((3-methyl-5-(trifluoromethyl)benzyl)amino)-4-oxo-4,6,7,8-tetrahydro-pyrrolo[1,2-a]pyrimidine-6-carboxamide, di-trifluoroacetate (53.3 mg, 26% yield).

### Example 24

### Preparation of (S)—N-((6-amino-2-methylpyridin-3-yl)methyl)-3-((4-fluorobenzyl)amino)-4-oxo-4,6,7,8-tetrahydropyrrolo[1,2-a]pyrimidine-6-carboxamide (Compound 52)

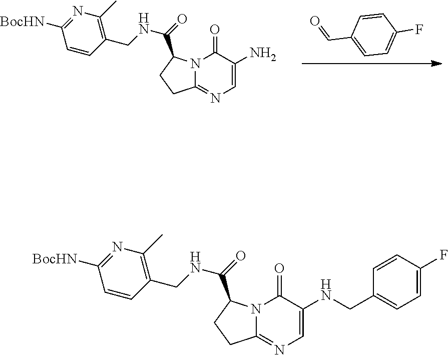 [SMILES: Cc1nc(NC(=O)OC(C)(C)C)ccc1CNC(=O)[C@@H]1CCc2ncc(N)c(=O)n21 ; O=Cc1ccc(F)cc1 ; Cc1nc(NC(=O)OC(C)(C)C)ccc1CNC(=O)[C@@H]1CCc2ncc(NCc3ccc(F)cc3)c(=O)n21]

Step 1: To a flask containing tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (44 mg, 0.11 mmol, prepared according to Ex. 1) was added DCE (3 mL), 4-fluorobenzaldehyde (0.22 mL, 2.1 mmol) and acetic acid (36 μL, 0.63 mmol) under argon. The reaction was stirred at room temp for 40 minutes then sodium triacetoxyborohydride (67 mg, 0.32 mmol) was added. Additional sodium triacetoxyborohydride (156 mg, 736 μmol) was added and the reaction was stirred for 1 hour. The mixture was diluted with DCM and washed with sat NaHCO₃ twice, brine, and dried over Na₂SO₄. The organic layer was collected, filtered, and concentrated in vacuo. The material was chromatographed (5-100% EtOAc-heptane then 0-20% MeOH-DCM). Additional column chromatography (0-50% MeOH-DCM) gave tert-butyl (S)-(5-((3-((4-fluorobenzyl)amino)-4-oxo-4,6,7,8-tetrahydropyrrolo[1,2-a]pyrimidine-6-carboxamido)methyl)-6-methylpyridin-2-yl)carbamate (38.1 mg. 69% yield) as a white solid.

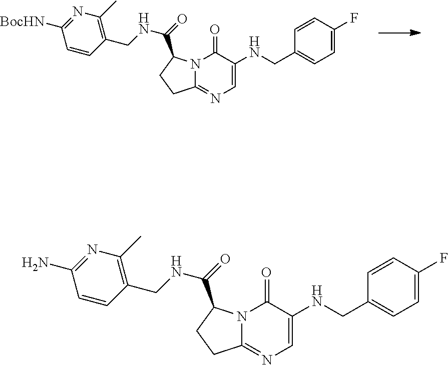 [SMILES: Cc1nc(NC(=O)OC(C)(C)C)ccc1CNC(=O)[C@@H]1CCc2ncc(NCc3ccc(F)cc3)c(=O)n21 ; Cc1nc(N)ccc1CNC(=O)[C@@H]1CCc2ncc(NCc3ccc(F)cc3)c(=O)n21]

Step 2: To a solution of tert-butyl (S)-(5-((3-((4-fluorobenzyl)amino)-4-oxo-4,6,7,8-tetrahydropyrrolo[1,2-a]pyrimidine-6-carboxamido)methyl)-6-methylpyridin-2-yl)carbamate (38.1 mg, 72.9 μmol) in DCM (3 mL) at room temp was added TFA (0.3 mL, 4 mmol). The reaction was stirred at room temp for 22 hours. The material was concd then re-suspended in a 2:1 ratio of MeCN—H₂O (3.5 mL), filtered through a 0.2 μM syringe filter, and purified by preparatory HPLC method 2. The product was dissolved in MeOH and 7 N NH₃—MeOH was added. The material was concd then chromatographed (0-10% 1:1 MeOH: 7 N NH₃—MeOH in DCM) to give (S)—N-((6-amino-2-methylpyridin-3-yl)methyl)-3-((4-fluorobenzyl)amino)-4-oxo-4,6,7,8-tetrahydropyrrolo[1,2-a]pyrimidine-6-carboxamide (3.0 mg, 10% yield) as a white powder.

### Example 25

### Preparation of (S)—N-((6-amino-2-methylpyridin-3-yl)methyl)-3-((3-fluorobenzyl)amino)-4-oxo-4,6,7,8-tetrahydropyrrolo[1,2-a]pyrimidine-6-carboxamide (Compound 53)

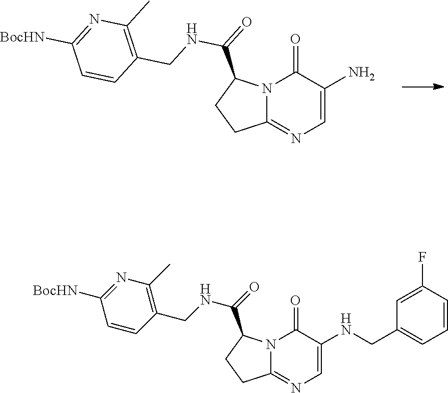 [SMILES: Cc1nc(NC(=O)OC(C)(C)C)ccc1CNC(=O)[C@@H]1CCc2ncc(N)c(=O)n21 ; Cc1nc(NC(=O)OC(C)(C)C)ccc1CNC(=O)[C@@H]1CCc2ncc(NCc3cccc(F)c3)c(=O)n21]

Step 1: To a flask containing tert-butyl (S)-(5-((3-amino-4-oxo-4,6,7,8-tetrahydropyrrolo[1,2-a]pyrimidine-6-carboxamido)methyl)-6-methylpyridin-2-yl)carbamate (66.3 mg, 160 μmol, prepared according to Ex. 1) was added DCE (3 mL), 3-fluorobenzaldehyde (34 μL, 0.32 mmol) and acetic acid (55 μL, 0.96 mmol). The reaction was stirred at room temperature for 1 h, then sodium triacetoxyborohydride (117 mg, 552 μmol) was added. After 3 hours, the mixture was diluted with DCM and washed twice with sat NaHCO₃, brine, and then dried over Na₂SO₄. The material was dissolved in MeOH/DCM, absorbed onto samplet, and dried under vacuum. The material was purified by column chromatography (0-40% isopropanol-DCM). Pure fractions were collected and concentrated to afford tert-butyl (S)-(5-((3-((3-fluorobenzyl)amino)-4-oxo-4,6,7,8-tetrahydropyrrolo[1,2-a]pyrimidine-6-carboxamido)methyl)-6-methylpyridin-2-yl)carbamate (41.4 mg, 50% yield) isolated as a partially crystalline solid/glass.

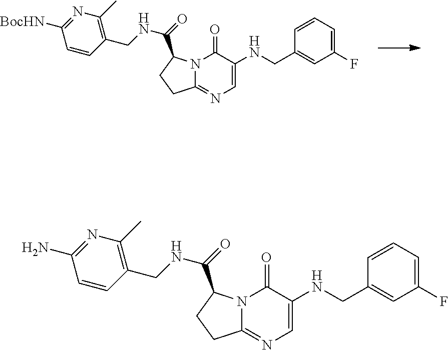 [SMILES: Cc1nc(NC(=O)OC(C)(C)C)ccc1CNC(=O)[C@@H]1CCc2ncc(NCc3cccc(F)c3)c(=O)n21 ; Cc1nc(N)ccc1CNC(=O)[C@@H]1CCc2ncc(NCc3cccc(F)c3)c(=O)n21]

Step 2: To a solution of tert-butyl (S)-(5-((3-((3-fluorobenzyl)amino)-4-oxo-4,6,7,8-tetrahydropyrrolo[1,2-a]pyrimidine-6-carboxamido)methyl)-6-methylpyridin-2-yl)carbamate (41.4 mg, 79.2 μmol) in DCM (3 mL) at room temp was added TFA (0.3 mL, 4 mmol). After stirring for 20 h at room temp, the solution was concentrated in vacuo. The crude material was dissolved in 2:1 MeCN—H₂O and one drop of TFA then purified by preparatory HPLC method 2. Solution was concentrated in vacuo and purified by chromatography (10 g Biotage column with 0-5% MeOH-DCM then 0-10% 1:1 7 N NH₃—MeOH:MeOH in DCM). Pure fractions were lyophilized to afford (S)—N-((6-amino-2-methylpyridin-3-yl)methyl)-3-((3-fluorobenzyl)amino)-4-oxo-4,6,7,8-tetrahydropyrrolo[1,2-a]pyrimidine-6-carboxamide (14.5 mg, 43% yield) isolated as a white solid.

### Example 26

### Preparation of (S)—N-((6-amino-2-methylpyridin-3-yl)methyl)-3-((2-methylbenzyl)amino)-4-oxo-4,6,7,8-tetrahydropyrrolo[1,2-a]pyrimidine-6-carboxamide (Compound 54)

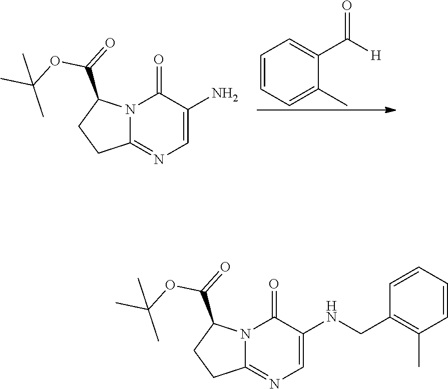

Step 1: To a solution of tert-butyl (S)-3-amino-4-oxo-4,6,7,8-tetrahydro-pyrrolo[1,2-a]pyrimidine-6-carboxylate (100 mg, 0.4 mmol, prepared according to Ex. 4) in DCM (4 ml) was added 2-methylbenzaldehyde (96 mg, 0.8 mmol) and acetic acid (143 mg, 2.4 mmol). After stirring for 30 minutes, sodium triacetoxyborohydride (253 mg, 1.19 mmol) was added and stirring was continued for 2 hr. The reaction was diluted with DCM (15 mL) followed by addition of sat. NaHCO₃ (15 mL) and stirring was continued for 30 min. The aqueous layer was extracted with additional DCM (15 mL) and the combined organic layers were dried over anhydr Na₂SO₄, filtered, and evaporated. The material was purified by column chromatography (70% EtOAc-hexane) to afford tert-butyl (S)-3-((2-methylbenzyl)amino)-4-oxo-4,6,7,8-tetrahydropyrrolo[1,2-a]pyrimidine-6-carboxylate (125 mg, 88% yield) as a white solid.

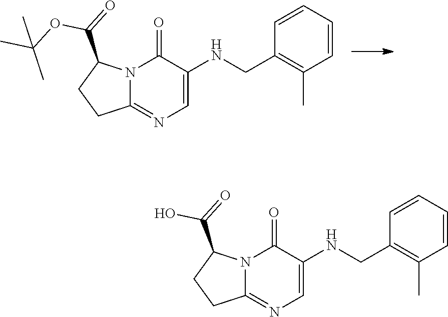 [SMILES: Cc1ccccc1CNc1cnc2n(c1=O)[C@H](C(=O)OC(C)(C)C)CC2 ; Cc1ccccc1CNc1cnc2n(c1=O)[C@H](C(=O)O)CC2]

Step 2: To a solution of tert-butyl (S)-3-((2-methylbenzyl)amino)-4-oxo-4,6,7,8-tetrahydropyrrolo[1,2-a]pyrimidine-6-carboxylate (119 mg, 0.34 mmol) in DCM (2 ml) was added TFA (2 ml) with stirring. After 5 hours the solution was evaporated to dryness giving (S)-3-((2-methylbenzyl)amino)-4-oxo-4,6,7,8-tetrahydropyrrolo[1,2-a]pyrimidine-6-carboxylic acid, trifluoroacetate (138 mg, 334 μmol, assumed 100% yield) as an oil.

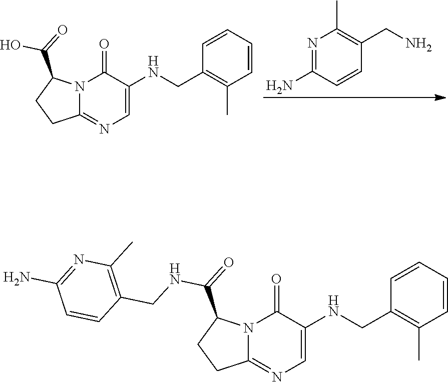

Step 4: To a solution of (S)-3-((2-methylbenzyl)amino)-4-oxo-4,6,7,8-tetra-hydropyrrolo[1,2-a]pyrimidine-6-carboxylic acid, trifluoracetate (138 mg, 0.334 mmol) in DMF (6 ml) was added NHS (169 mg, 1.47 mmol), DCC (303 mg, 1.47 mmol), and 5-(aminomethyl)-6-methylpyridin-2-amine (220 mg, 1.6 mmol). The solution was stirred for 16 hours at room temp and then evaporated to dryness. The residue was dissolved in 5% MeOH/CH₂Cl₂ (2 ml) and filtered. The solution was evaporated to dryness followed by purification using flash chromatography (4% 7 N NH₃—MeOH/CH₂Cl₂) to afford (S)—N-((6-amino-2-methylpyridin-3-yl)methyl)-3-((2-methylbenzyl)amino)-4-oxo-4,6,7,8-tetrahydropyrrolo[1,2-a]pyrimidine-6-carboxamide (54 mg, 39% yield) as an off-white solid.

The following compounds were prepared according to the foregoing procedure using the appropriate aldehyde starting materials:

| | Cmp |
|---|---|
| Compound Name | No. |
| (S)-N-((6-amino-2-methylpyridin-3-yl)methyl)-3-((3-chloro-5- | 55 |
| methylbenzyl)amino)-4-oxo-4,6,7,8-tetrahydropyrrolo[1,2- | |
| a]pyrimidine-6-carboxamide | |

### Example 27

### Preparation of (S)—N-((6-amino-2-methylpyridin-3-yl)methyl)-3-((3-chloro-5-fluorobenzyl)amino)-4-oxo-4,6,7,8-tetrahydropyrrolo[1,2-a]pyrimidine-6-carboxamide (Compound 56)

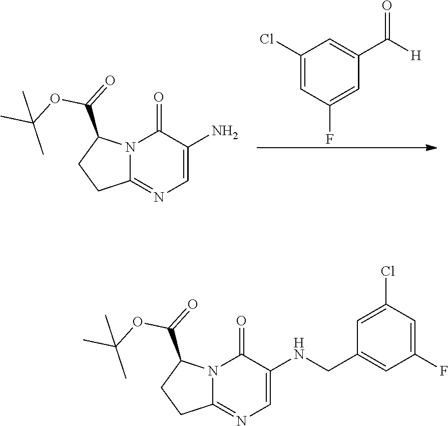 [SMILES: CC(C)(C)OC(=O)[C@@H]1CCc2ncc(N)c(=O)n21 ; O=Cc1cc(F)cc(Cl)c1 ; CC(C)(C)OC(=O)[C@@H]1CCc2ncc(NCc3cc(F)cc(Cl)c3)c(=O)n21]

Step 1: To a solution of tert-butyl (S)-3-amino-4-oxo-4,6,7,8-tetrahydro-pyrrolo[1,2-a]pyrimidine-6-carboxylate (100 mg, 0.4 mmol, prepared according to Ex. 4) in DCE (4 ml) was added 3-chloro-5-fluorobenzaldehyde (126 mg, 0.8 mmol) and acetic acid (143 mg, 2.4 mmol). After stirring for 30 minutes, sodium triacetoxyborohydride (422 mg, 1.99 mmol) was added and stirring was continued for 16 hours. The reaction was diluted with DCM (15 mL) followed by addition of sat. NaHCO₃ (15 mL) and stirring was continued for 30 minutes. The aqueous layer was extracted with additional DCM (15 mL) and the combined organic layers were dried over anhydr Na₂SO₄, filtered, and evaporated. The material was purified by column chromatography (50% EtOAc-hexanes) to afford tert-butyl (S)-3-((3-chloro-5-fluorobenzyl)amino)-4-oxo-4,6,7,8-tetrahydropyrrolo[1,2-a]pyrimidine-6-carboxylate (131 mg, 84% yield) as a white solid.

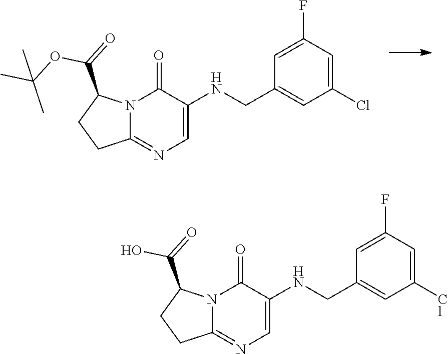 [SMILES: CC(C)(C)OC(=O)[C@@H]1CCc2ncc(NCc3cc(F)cc(Cl)c3)c(=O)n21 ; O=C(O)[C@@H]1CCc2ncc(NCc3cc(F)cc(Cl)c3)c(=O)n21]

Step 2: To a solution of tert-butyl (S)-3-((3-chloro-5-fluorobenzyl)amino)-4-oxo-4,6,7,8-tetrahydropyrrolo[1,2-a]pyrimidine-6-carboxylate (105 mg, 0.27 mmole) in DCM (2 ml) was added TFA (2 ml) with stirring. After 5 hours, the solution was evaporated to dryness giving (S)-3-((3-chloro-5-fluorobenzyl)amino)-4-oxo-4,6,7,8-tetrahydropyrrolo[1,2-a]pyrimidine-6-carboxylic acid, trifluoroacetate (120 mg, assumed 100% yield) as an oil.

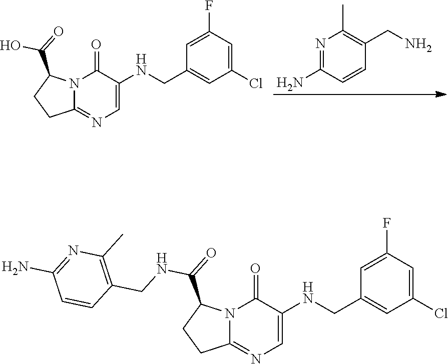

Step 3: To a solution of (S)-3-((3-chloro-5-fluorobenzyl)amino)-4-oxo-4,6,7,8-tetrahydropyrrolo[1,2-a]pyrimidine-6-carboxylic acid, trifluoroacetate (120 mg, 0.27 mmol) in DMF (6 ml) was added HATU (247 mg, 0.65 mmol), Et₃N (110 mg, 1.08 mmol), and 5-(aminomethyl)-6-methylpyridin-2-amine (97 mg, 0.70 mmole.) The solution was stirred for 16 hours at room temp and then evaporated to dryness followed by purification using flash chromatography (4% 7 N NH₃—MeOH/CH₂Cl₂) giving (S)—N-((6-amino-2-methylpyridin-3-yl)methyl)-3-((3-chloro-5-fluorobenzyl)amino)-4-oxo-4,6,7,8-tetrahydropyrrolo[1,2-a]pyrimidine-6-carboxamide (54 mg, 44% yield) as a white solid.

### Example 28

### Preparation of (S)—N-((6-amino-2-methylpyridin-3-yl)methyl)-3-((3-ethoxybenzyl)amino)-4-oxo-4,6,7,8-tetrahydropyrrolo[1,2-a]pyrimidine-6-carboxamide (Compound 57)

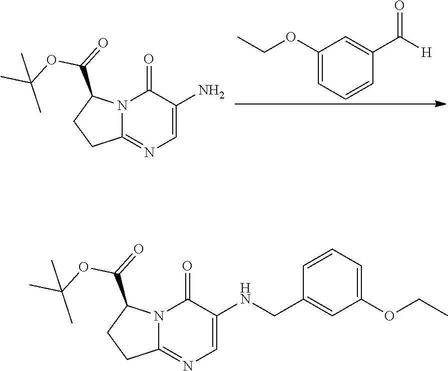 [SMILES: CC(C)(C)OC(=O)[C@@H]1CCc2ncc(N)c(=O)n21 ; CCOc1cccc(C=O)c1 ; CCOc1cccc(CNc2cnc3n(c2=O)[C@H](C(=O)OC(C)(C)C)CC3)c1]

Step 1: To a solution of tert-butyl (S)-3-amino-4-oxo-4,6,7,8-tetrahydro-pyrrolo[1,2-a]pyrimidine-6-carboxylate (100 mg, 0.4 mmol, prepared according to Ex. 4) in DCM (4 ml) was added 3-ethoxybenzaldehyde (120 mg, 0.8 mmol) and acetic acid (143 mg, 2.4 mmol). The reaction mixture was stirred for 30 minutes. Sodium triacetoxyborohydride (253 mg, 1.19 mmol) was added and stirring was continued for 2 hours. The reaction was diluted with DCM (15 ml) followed by addition of sat. NaHCO₃ (15 ml) and stirring was continued for 30 min. The aqueous layer was extracted with additional DCM (15 ml) and the combined organic layers were dried over anhydr Na₂SO₄, filtered, and evaporated. The material was purified by column chromatography (50% EtOAc-hexanes) to afford tert-butyl (S)-3-((3-ethoxybenzyl)amino)-4-oxo-4,6,7,8-tetrahydropyrrolo[1,2-a]pyrimidine-6-carboxylate (122 mg, 86% yield) as a white solid.

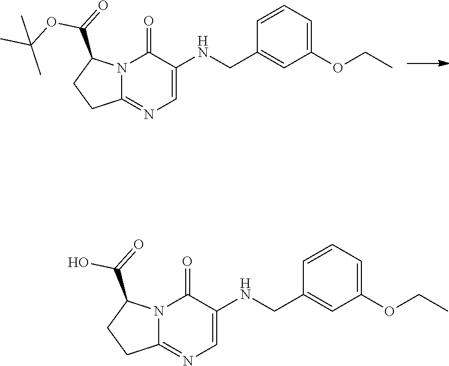 [SMILES: CCOc1cccc(CNc2cnc3n(c2=O)[C@H](C(=O)OC(C)(C)C)CC3)c1 ; CCOc1cccc(CNc2cnc3n(c2=O)[C@H](C(=O)O)CC3)c1]

Step 2: To a solution of tert-butyl (S)-3-((3-ethoxybenzyl)amino)-4-oxo-4,6,7,8-tetrahydropyrrolo[1,2-a]pyrimidine-6-carboxylate (105 mg, 0.27 mmol) in DCM (2 ml) was added TFA (2 ml) with stirring. After 5 hours, the solution was evaporated to dryness giving (S)-3-((3-ethoxybenzyl)amino)-4-oxo-4,6,7,8-tetrahydropyrrolo[1,2-a]pyrimidine-6-carboxylic acid, trifluoroacetetate (121 mg, assumed 100% yield) as an oil.

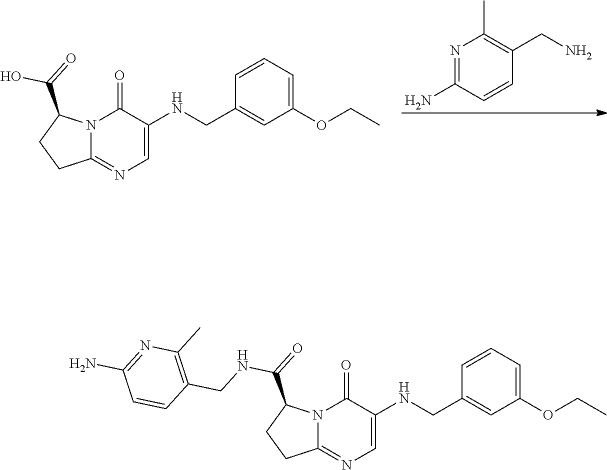

Step 3: To a solution of (S)-3-((3-ethoxybenzyl)amino)-4-oxo-4,6,7,8-tetrahydro-pyrrolo[1,2-a]pyrimidine-6-carboxylic acid-trifluoroacetic acid (120 mg, 0.27 mmol) in DMF (6 ml) was added HATU (247 mg, 0.65 mmol), Et₃N (110 mg, 1.08 mmol), and 5-(aminomethyl)-6-methylpyridin-2-amine (97 mg, 0.70 mmol). The solution was stirred for 16 hours at room temp and then evaporated to dryness followed by purification using flash chromatography (4% 7 N NH₃—MeOH/CH₂Cl₂) giving (S)—N-((6-amino-2-methylpyridin-3-yl)methyl)-3-((3-ethoxybenzyl)amino)-4-oxo-4,6,7,8-tetrahydropyrrolo[1,2-a]pyrimidine-6-carboxamide (54 mg, 44% yield) as a white solid.

### Example 29

### Preparation of (S)—N-((6-amino-2-methylpyridin-3-yl)methyl)-3-((3-methylbenzyl)amino)-4-oxo-4,6,7,8-tetrahydropyrrolo[1,2-a]pyrimidine-6-carboxamide (Compound 60)

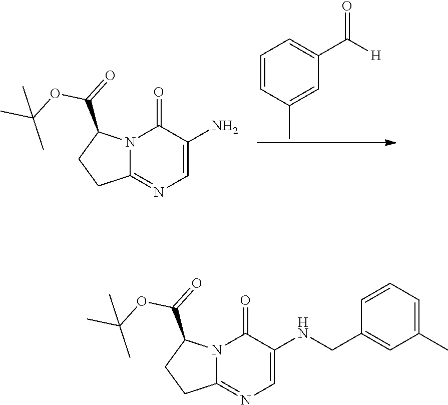

Step 1: To a solution of tert-butyl (S)-3-amino-4-oxo-4,6,7,8-tetrahydro-pyrrolo[1,2-a]pyrimidine-6-carboxylate (100 mg, 0.4 mmol, prepared according to Ex. 4) in DCM (4 ml) was added 3-methylbenzaldehyde (96 mg, 0.8 mmol) and acetic acid (143 mg, 2.4 mmol). After stirring for 30 minutes, sodium triacetoxyborohydride (253 mg, 1.19 mmol) was added. The reaction continued to stir for 2 hours. The reaction was diluted with DCM (15 ml) followed by addition of sat. NaHCO₃ (15 ml) and stirring was continued for 30 min. The aqueous layer was extracted with additional DCM (15 ml) and the combined organic layers were dried and evaporated to dryness. Column chromatography (70% EtOAc/hexanes) gave tert-butyl (S)-3-((3-methylbenzyl)amino)-4-oxo-4,6,7,8-tetrahydropyrrolo[1,2-a]pyrimidine-6-carboxylate (119 mg, 84% yield) as a white solid.

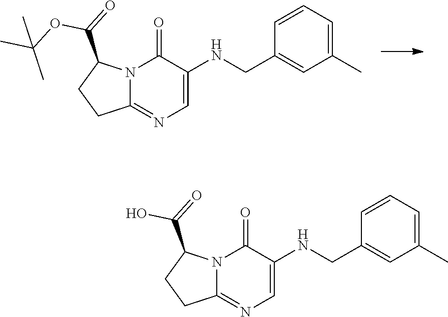 [SMILES: Cc1cccc(CNc2cnc3n(c2=O)[C@H](C(=O)OC(C)(C)C)CC3)c1 ; Cc1cccc(CNc2cnc3n(c2=O)[C@H](C(=O)O)CC3)c1]

Step 2: To a solution of tert-butyl (S)-3-((3-methylbenzyl)amino)-4-oxo-4,6,7,8-tetrahydropyrrolo[1,2-a]pyrimidine-6-carboxylate (119 mg, 0.34 mmol) in DCM (2 ml) was added TFA (2 ml). The reaction mixture was stirred for 5 hours. The solution was evaporated to dryness giving (S)-3-((3-methylbenzyl)amino)-4-oxo-4,6,7,8-tetrahydropyrrolo[1,2-a]pyrimidine-6-carboxylic acid (100 mg. 100% yield) as a white solid.

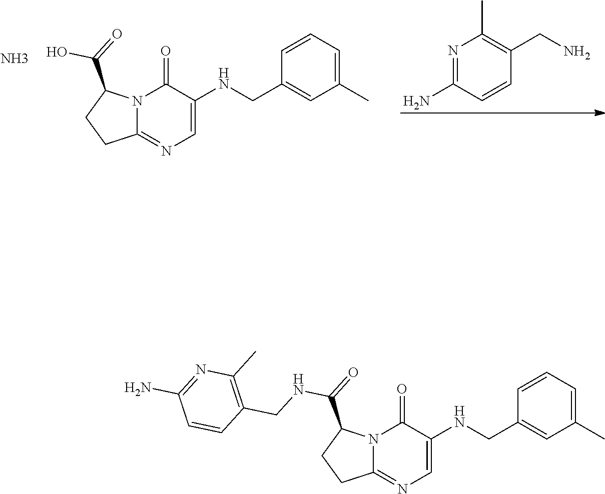

Step 3: To a solution of (S)-3-((3-methylbenzyl)amino)-4-oxo-4,6,7,8-tetra-hydropyrrolo[1,2-a]pyrimidine-6-carboxylic acid, ammonium salt (80 mg, 0.25 mmol) in DMF (3 ml) was added NHS (32 mg, 0.28 mmol) and 5-(aminomethyl)-6-methylpyridin-2-amine (52 mg, 0.38 mmol) with stirring until dissolved. DCC (57 mg, 0.28 mmol) was added and the solution was stirred for 16 hours at room temp and then evaporated to dryness. The residue was dissolved in 5% MeOH/CH₂Cl₂ (2 ml) and filtered to remove DCU. Evaporation to dryness followed by purification using flash chromatography (6% 7 N NH₃—MeOH/CH₂Cl₂) gave (S)—N-((6-amino-2-methylpyridin-3-yl)methyl)-3-((3-methylbenzyl)amino)-4-oxo-4,6,7,8-tetrahydropyrrolo[1,2-a]pyrimidine-6-carboxamide (32 mg, 76 μmol, 30% yield) as an off-white solid.

The following compounds were prepared according to the foregoing procedure using the appropriate aldehyde starting materials:

| | Cmp |
|---|---|
| Compound Name | No. |
| (S)-N-((6-amino-2-methylpyridin-3-yl)methyl)-3-((4-methyl- | 61 |
| benzyl)amino)-4-oxo-4,6,7,8-tetrahydropyrrolo[1,2-a]pyrimidine-6- | |
| carboxamide | |

### Example 30

### Preparation of (S)—N—((R)-2-amino-6,7-dihydro-5H-cyclopenta[b]pyridin-5-yl)-3-((3,5-dimethylbenzyl)amino)-4-oxo-4,6,7,8-tetrahydropyrrolo[1,2-a]pyrimidine-6-carboxamide (Compound 81)

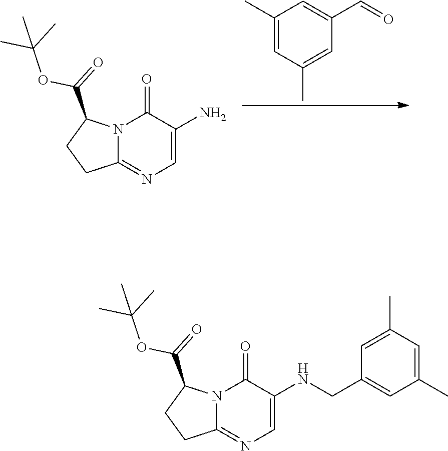

Step 1: To a solution of tert-butyl (S)-3-amino-4-oxo-4,6,7,8-tetrahydro-pyrrolo[1,2-a]pyrimidine-6-carboxylate (3.3 g, 13 mmol) and 3,5-dimethylbenzaldehyde (2.6 mL, 20 mmol) in DCM (60 mL) was added HOAc (2.3 mL, 39 mmol). After stirring for 30 min, sodium triacetoxyborohydride (8.3 g, 39 mmol) was added and the reaction continued for 2 h at room temp. The mixture was diluted with DCM followed by addition of sat. aq NaHCO₃ and stirring was continued for 30 min. The aqueous layer was extracted with additional DCM and washed with brine. The combined organic layers were dried over anhydr Na₂SO₄, filtered, and concentrated. The residue was purified by column chromatography (0-100% EtOAc-heptane) to provide tert-butyl (S)-3-((3,5-dimethylbenzyl)amino)-4-oxo-4,6,7,8-tetrahydropyrrolo-[1,2-a]pyrimidine-6-carboxylate (4.14 g. 85% yield).

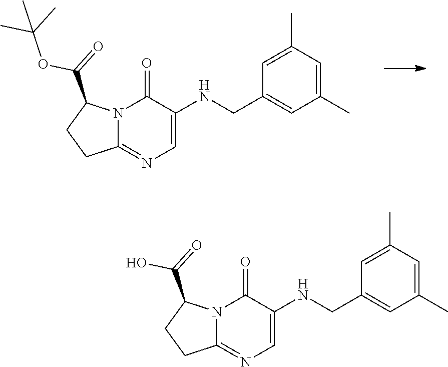 [SMILES: Cc1cc(C)cc(CNc2cnc3n(c2=O)[C@H](C(=O)OC(C)(C)C)CC3)c1 ; Cc1cc(C)cc(CNc2cnc3n(c2=O)[C@H](C(=O)O)CC3)c1]

Step 2: tert-Butyl (S)-3-((3,5-dimethylbenzyl)amino)-4-oxo-4,6,7,8-tetrahydro-pyrrolo[1,2-a]pyrimidine-6-carboxylate (4.1 g, 11 mmol) was dissolved in DCM (20 mL) and treated with TFA (20 mL, 263 mmol). After stirring for 5 h at room temp, the reaction mixture was concentrated and the residue was purified by column chromatography (20% MeOH-DCM) to provide a thick oil. Diethyl ether (30 mL) was added then sonicated and the solution was allowed to sit overnight. The precipitate was collected by filtration to provide (S)-3-((3,5-dimethylbenzyl)amino)-4-oxo-4,6,7,8-tetrahydropyrrolo[1,2-a]pyrimidine-6-carboxylic acid, 1.5 trifluoroacetate (4.7 g, 87% yield) as an off-white solid.

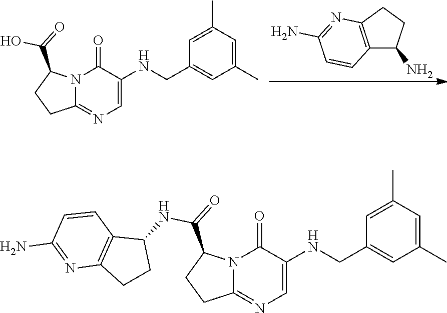 [SMILES: Cc1cc(C)cc(CNc2cnc3n(c2=O)[C@H](C(=O)O)CC3)c1 ; Nc1ccc2c(n1)CC[C@H]2N ; Cc1cc(C)cc(CNc2cnc3n(c2=O)[C@H](C(=O)N[C@@H]2CCc4nc(N)ccc42)CC3)c1]

Step 3: To an ice-cold solution of (S)-3-((3,5-dimethylbenzyl)amino)-4-oxo-4,6,7,8-tetrahydropyrrolo[1,2-a]pyrimidine-6-carboxylic acid, 1.5 trifluoroacetate (700 mg, 1.45 mmol) in DMF (18 mL) was added DIEA (1.01 mL, 5.78 mmol). After stirring for 30 min at the same temperature, HATU (1.10 g, 2.89 mmol) was added. After an additional 10 min, (R)-6,7-dihydro-5H-cyclopenta[b]pyridine-2,5-diamine, di-hydrochloride (385 mg, 1.73 mmol) was added. After allowing to warm to room temp and stirring for 18 h, the reaction mixture was concentrated. The residue was purified by column chromatography (amine column, 0-50% MeOH-DCM). Additional purification by column chromatography (0-70% MeOH-DCM) provided (S)—N—((R)-2-amino-6,7-dihydro-5H-cyclopenta[b]pyridin-5-yl)-3-((3,5-dimethylbenzyl)amino)-4-oxo-4,6,7,8-tetrahydropyrrolo[1,2-a]pyrimidine-6-carboxamide (578 mg, 90% yield).

The following compounds were prepared according to the foregoing procedure using the appropriate amine starting materials:

| | Cmp. |
|---|---|
| Compound Name | No. |
| (S)-N-((R)-1-(6-amino-2-methylpyridin-3-yl)ethyl)-3-((3,5- | 64 |
| dimethylbenzyl)amino)-4-oxo-4,6,7,8-tetrahydropyrrolo[1,2- | |
| a]pyrimidine-6-carboxamide | |

### Example 31

### Preparation of (S)—N—((R)-2-amino-6,7-dihydro-5H-cyclopenta[b]pyridin-5-yl)-3-((3-methylbenzyl)amino)-4-oxo-4,6,7,8-tetrahydropyrrolo[1,2-a]pyrimidine-6-carboxamide (Compound 82)

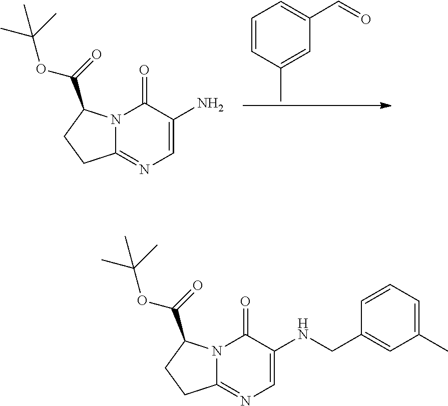

Step 1: To a solution of tert-butyl (S)-3-amino-4-oxo-4,6,7,8-tetrahydro-pyrrolo[1,2-a]pyrimidine-6-carboxylate (1.66 g, 6.606 mmol) and 3-methylbenzaldehyde (1.17 mL, 9.91 mmol) in DCM (50 mL) was added HOAc (1.134 mL, 19.8 mmol). After stirring for 30 min, sodium triacetoxyborohydride (4.2 g, 19.8 mmol) was added. After stirring for 2 h at room temp, the mixture was diluted with DCM followed by addition of sat. aq NaHCO₃. Stirring was continued for 30 min, then the aqueous layer was extracted with additional DCM and washed with brine. The combined organic layers were dried over anhydr Na₂SO₄, filtered, and concentrated. The residue was purified by column chromatography (0-100% EtOAc-heptane) to provide tert-butyl (S)-3-((3-methylbenzyl)amino)-4-oxo-4,6,7,8-tetrahydropyrrolo[1,2-a]pyrimidine-6-carboxylate (2.27 g, 97% yield).

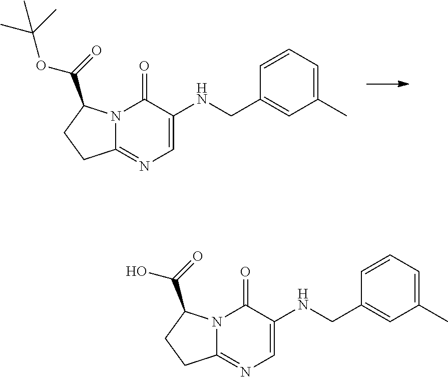 [SMILES: Cc1cccc(CNc2cnc3n(c2=O)[C@H](C(=O)OC(C)(C)C)CC3)c1 ; Cc1cccc(CNc2cnc3n(c2=O)[C@H](C(=O)O)CC3)c1]

Step 2: tert-Butyl (S)-3-((3-methylbenzyl)amino)-4-oxo-4,6,7,8-tetrahydropyrrolo[1,2-a]pyrimidine-6-carboxylate (2.27 g, 6.39 mmol) was dissolved in DCM (120 mL) and treated with TFA (14.8 mL, 192 mmol). After stirring for 18 h at room temp, the reaction mixture was concentrated, the residue was purified by column chromatography (0-50% MeOH-DCM), and the product was lyophilized to provide (S)-3-((3-methylbenzyl)amino)-4-oxo-4,6,7,8-tetrahydropyrrolo[1,2-a]pyrimidine-6-carboxylic acid, 1.5 trifluoroacetate (1.65 g, 55% yield) as an off-white solid.

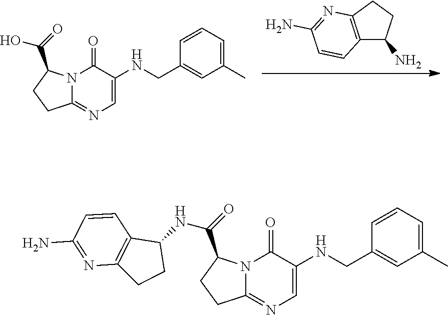 [SMILES: Cc1cccc(CNc2cnc3n(c2=O)[C@H](C(=O)O)CC3)c1 ; Nc1ccc2c(n1)CC[C@H]2N ; Cc1cccc(CNc2cnc3n(c2=O)[C@H](C(=O)N[C@@H]2CCc4nc(N)ccc42)CC3)c1]

Step 3: To an ice-cold solution of (S)-3-((3-methylbenzyl)amino)-4-oxo-4,6,7,8-tetrahydropyrrolo[1,2-a]pyrimidine-6-carboxylic acid, 1.5 trifluoroacetate (1.0 g, 2.13 mmol) in DMF (18 mL) was added DIEA (1.48 mL, 8.5 mmol). After stirring for 30 min at the same temperature, HATU (1.62 g, 4.25 mmol) was added. After stirring for an additional 10 min, (R)-6,7-dihydro-5H-cyclopenta[b]pyridine-2,5-diamine, di-hydrochloride (385 mg, 1.73 mmol) was added. After allowing the mixture to warm to room temp and stir for 18 h, it was concentrated and the residue was purified by column chromatography (amine column, 0-50% MeOH-DCM). Additional purification by column chromatography (10-25% MeOH-DCM) provided (S)—N—((R)-2-amino-6,7-dihydro-5H-cyclopenta[b]pyridin-5-yl)-3-((3-methylbenzyl)amino)-4-oxo-4,6,7,8-tetrahydropyrrolo[1,2-a]pyrimidine-6-carboxamide (741 mg, 81% yield).

The following compounds were prepared according to the foregoing procedure using the appropriate aldehyde starting materials:

| | Cmp |
|---|---|
| Compound Name | No. |
| (S)-N-((R)-2-amino-6,7-dihydro-5H-cyclopenta[b]pyridin-5-yl)-3- | 84 |
| ((3-(difluoromethyl)benzyl)amino)-4-oxo-4,6,7,8-tetrahydro- | |
| pyrrolo[1,2-a]pyrimidine-6-carboxamide | |

### Example 32

### Preparation of (S)-3-((3-chloro-5-methylbenzyl)amino)-N-((6,7-dihydro-5H-pyrrolo[3,4-b]pyridin-3-yl)methyl)-4-oxo-4,6,7,8-tetrahydropyrrolo[1,2-a]pyrimidine-6-carboxamide, di-trifluoroacetate (Compound 65)

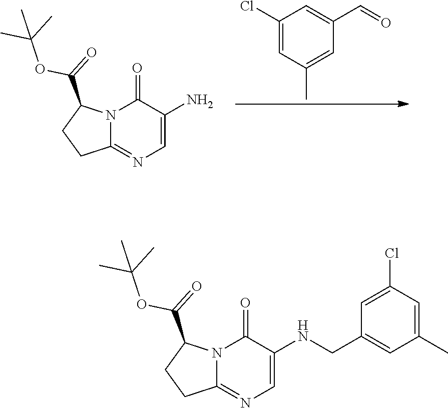

Step 1: To a solution of tert-butyl (S)-3-amino-4-oxo-4,6,7,8-tetrahydro-pyrrolo[1,2-a]pyrimidine-6-carboxylate (200 mg, 796 μmol) and 3-chloro-5-methylbenzaldehyde (0.15 mL, 1.19 mmol) in DCM (6 mL) was added HOAc (137 μL, 2.39 mmol). After stirring for 30 min, sodium triacetoxyborohydride (506 mg, 2.39 mmol) was added. After stirring for 2 h at room temp, the mixture was diluted in DCM followed by addition of sat. aq NaHCO₃. After stirring for 30 min, the aqueous layer was extracted with additional DCM and washed with brine. The combined organic layers were dried over anhydr Na₂SO₄, filtered, and concentrated. The residue was purified by column chromatography (0-100% EtOAc-heptane) to provide tert-butyl (S)-3-((3-chloro-5-methylbenzyl)amino)-4-oxo-4,6,7,8-tetrahydropyrrolo[1,2-a]pyrimidine-6-carboxylate (218 mg, 70% yield).

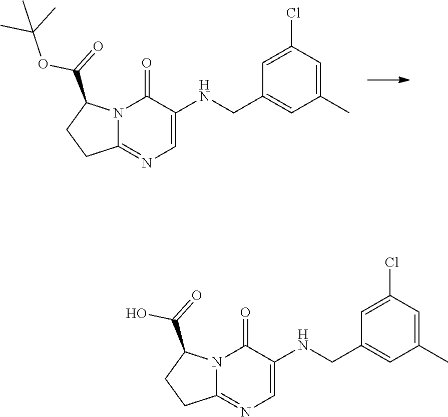 [SMILES: Cc1cc(Cl)cc(CNc2cnc3n(c2=O)[C@H](C(=O)OC(C)(C)C)CC3)c1 ; Cc1cc(Cl)cc(CNc2cnc3n(c2=O)[C@H](C(=O)O)CC3)c1]

Step 2: tert-Butyl (S)-3-((3-chloro-5-methylbenzyl)amino)-4-oxo-4,6,7,8-tetrahydropyrrolo[1,2-a]pyrimidine-6-carboxylate (218 mg, 0.56 mmol) was dissolved in DCM (10 mL) and treated with TFA (1.91 g, 1.29 mL, 16.8 mmol). After stirring for 18 h at room temp, the reaction mixture was concentrated, the residue was purified by column chromatography (0-50% MeOH-DCM), and the product was lyophilized to provide (S)-3-((3-chloro-5-methylbenzyl)amino)-4-oxo-4,6,7,8-tetrahydropyrrolo[1,2-a]pyrimidine-6-carboxylic acid, 1.5 trifluoroacetate (195 mg, 69% yield) as an off-white solid.

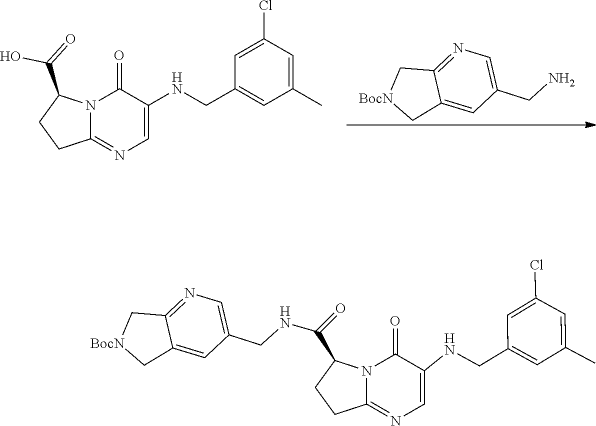 [SMILES: Cc1cc(Cl)cc(CNc2cnc3n(c2=O)[C@H](C(=O)O)CC3)c1 ; CC(C)(C)OC(=O)N1Cc2cc(CN)cnc2C1 ; Cc1cc(Cl)cc(CNc2cnc3n(c2=O)[C@H](C(=O)NCc2cnc4c(c2)CN(C(=O)OC(C)(C)C)C4)CC3)c1]

Step 3: To an ice-cold solution of (S)-3-((3-chloro-5-methylbenzyl)amino)-4-oxo-4,6,7,8-tetrahydropyrrolo[1,2-a]pyrimidine-6-carboxylic acid, 1.5 trifluoroacetate (40 mg, 79 μmol) in DMF (2.0 mL) was added DIEA (51 mg, 69 μL, 0.4 mmol). After stirring for 30 min at the same temperature, HATU (60 mg, 0.16 mmol) was added. After an additional 10 min reaction time, tert-butyl 3-(aminomethyl)-5,7-dihydro-6H-pyrrolo[3,4-b]pyridine-6-carboxylate (24 mg, 95 μmol, prepared according to steps 1-2 of Example 17) was added. After allowing to warm to room temp and stirring for 18 hours, the reaction mixture was concentrated and the residue was purified by column chromatography (0-100% EtOAc-heptane) to provide tert-butyl (S)-3-((3-((3-chloro-5-methylbenzyl)amino)-4-oxo-4,6,7,8-tetrahydropyrrolo[1,2-a]pyrimidine-6-carboxamido)methyl)-5,7-dihydro-6H-pyrrolo[3,4-b]pyridine-6-carboxylate (45 mg, 100% yield).

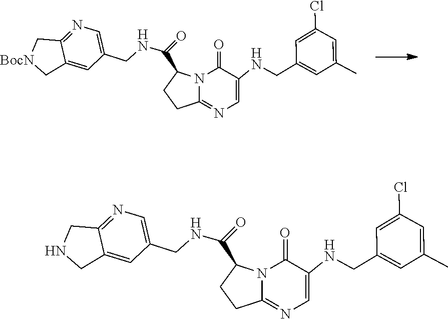 [SMILES: Cc1cc(Cl)cc(CNc2cnc3n(c2=O)[C@H](C(=O)NCc2cnc4c(c2)CN(C(=O)OC(C)(C)C)C4)CC3)c1 ; Cc1cc(Cl)cc(CNc2cnc3n(c2=O)[C@H](C(=O)NCc2cnc4c(c2)CNC4)CC3)c1]

Step 4: To an ice-cold solution of tert-butyl (S)-3-((3-((3-chloro-5-methylbenzyl)amino)-4-oxo-4,6,7,8-tetrahydropyrrolo[1,2-a]pyrimidine-6-carboxamido)methyl)-5,7-dihydro-6H-pyrrolo[3,4-b]pyridine-6-carboxylate (45 mg, 80 μmol) in DCM (2 mL) was added TFA (0.18 mL, 2.4 mmol). After allowing to warm to room temp and stirring for 3 h, the reaction mixture was concentrated. The residue was purified by preparatory HPLC method 2 to provide (S)-3-((3-chloro-5-methylbenzyl)amino)-N-((6,7-dihydro-5H-pyrrolo[3,4-b]pyridin-3-yl)methyl)-4-oxo-4,6,7,8-tetrahydropyrrolo[1,2-a]pyrimidine-6-carboxamide, di-trifluoroacetate (55 mg, 64% yield).

### Example 33

### Preparation of (S)—N—((R)-2-amino-6,7-dihydro-5H-cyclopenta[b]pyridin-5-yl)-3-((3-chloro-5-methylbenzyl)amino)-4-oxo-4,6,7,8-tetrahydropyrrolo[1,2-a]pyrimidine-6-carboxamide (Compound 83)

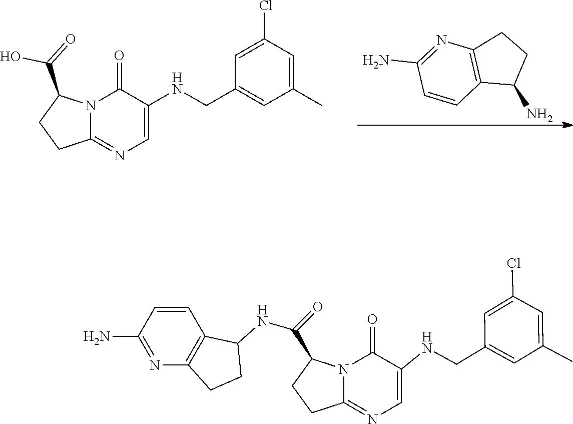 [SMILES: Cc1cc(Cl)cc(CNc2cnc3n(c2=O)[C@H](C(=O)O)CC3)c1 ; Nc1ccc2c(n1)CC[C@H]2N ; Cc1cc(Cl)cc(CNc2cnc3n(c2=O)[C@H](C(=O)NC2CCc4nc(N)ccc42)CC3)c1]

Step 1: To an ice-cold solution of (S)-3-((3-chloro-5-methylbenzyl)amino)-4-oxo-4,6,7,8-tetrahydropyrrolo[1,2-a]pyrimidine-6-carboxylic acid, 1.5 trifluoroacetate (60 mg, 0.12 mmol, prepared according to steps 1-2 of Example 32) in DMF (2.0 mL) was added DIEA (77 mg, 0.1 mL, 0.59 mmol). After stirring for 30 min at the same temperature, HATU (90 mg, 0.24 mmol) was added. After stirring for an additional 10 min, (R)-6,7-dihydro-5H-cyclopenta[b]pyridine-2,5-diamine, di-hydrochloride (32 mg, 0.14 mmol) was added. After allowing to warm to room temp and stirring for 18 h, the reaction mixture was concentrated and the residue was purified by column chromatography (amine column, 0-50% MeOH-DCM). Additional purification by column chromatography (0-50% MeOH-DCM) provided (S)—N—((R)-2-amino-6,7-dihydro-5H-cyclopenta[b]pyridin-5-yl)-3-((3-chloro-5-methylbenzyl)amino)-4-oxo-4,6,7,8-tetrahydropyrrolo[1,2-a]pyrimidine-6-carboxamide (44.5 mg, 81% yield).

The following compounds were prepared according to the foregoing procedure using the appropriate amine starting materials:

| | Cmp |
|---|---|
| Compound Name | No. |
| (S)-3-((3-chloro-5-methylbenzyl)amino)-N-((R)-6,7-dihydro-5H- | 85 |
| cyclopenta[b]pyridin-5-yl)-4-oxo-4,6,7,8-tetrahydropyrrolo[1,2- | |
| a]pyrimidine-6-carboxamide | |

The following compounds were prepared according to the foregoing procedure using the appropriate amine starting materials, except that the final product was purified by preparatory HPLC method 2.

| | Cmp |
|---|---|
| Compound Name | No. |
| (S)-3-((3-chloro-5-methylbenzyl)amino)-4-oxo-N-(pyridin-3- | 66 |
| ylmethyl)-4,6,7,8-tetrahydropyrrolo[1,2-a]pyrimidine-6- | |
| carboxamide, trifluoroacetate | |

### Example 34

### Preparation of (S)—N-((6-amino-2-methylpyridin-3-yl)methyl)-3-(ethyl(3-methoxy-5-methylbenzyl)amino)-4-oxo-4,6,7,8-tetrahydropyrrolo[1,2-a]pyrimidine-6-carboxamide (Compound 67)

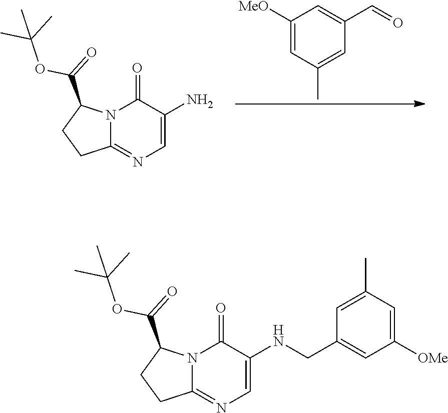 [SMILES: CC(C)(C)OC(=O)[C@@H]1CCc2ncc(N)c(=O)n21 ; COc1cc(C)cc(C=O)c1 ; COc1cc(C)cc(CNc2cnc3n(c2=O)[C@H](C(=O)OC(C)(C)C)CC3)c1]

Step 1: To a solution of tert-butyl (S)-3-amino-4-oxo-4,6,7,8-tetrahydro-pyrrolo[1,2-a]pyrimidine-6-carboxylate (200 mg, 796 μmol) and 3-methoxy-5-methylbenzaldehyde (0.15 mL, 1.19 mmol) in DCM (6 mL) was added HOAc (137 μL, 2.39 mmol). After stirring for 30 min, sodium triacetoxyborohydride (506 mg, 2.39 mmol) was added. After an additional 2 h reaction time, the mixture was diluted with DCM followed by addition of sat. aq NaHCO₃ and stirring was continued for 30 min. The aqueous layer was extracted with DCM and washed with brine. The combined organic layers were dried over anhydr Na₂SO₄, filtered, and concentrated. The residue was purified by column chromatography (0-100% EtOAc-heptane) to provide tert-butyl (S)-3-((3-methoxy-5-methylbenzyl)amino)-4-oxo-4,6,7,8-tetrahydropyrrolo[1,2-a]pyrimidine-6-carboxylate (290 mg, 94% yield).

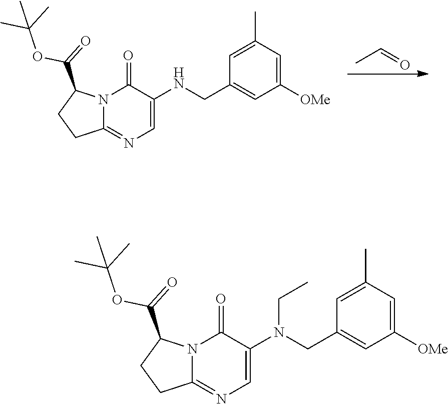

Step 2: To a solution of tert-butyl (S)-3-((3-methoxy-5-methylbenzyl)amino)-4-oxo-4,6,7,8-tetrahydropyrrolo[1,2-a]pyrimidine-6-carboxylate (145 mg, 376 μmol) and acetaldehyde (ca. 2% in DMF, 3.17 mL, 1.13 mmol) in DCM (3 mL) was added HOAc (64.6 μL, 1.13 mmol). After stirring for 30 min, sodium triacetoxyborohydride (506 mg, 2.39 mmol) was added. After stirring for 18 h at room temp, the mixture was diluted with DCM followed by addition of sat. aq NaHCO₃; stirring was continued for 30 min. The aqueous layer was extracted with DCM and washed with brine. The combined organic layers were dried over anhydr Na₂SO₄, filtered, and concentrated. The residue was purified by column chromatography (0-100% EtOAc-heptane) to provide tert-butyl (S)-3-(ethyl(3-methoxy-5-methylbenzyl)amino)-4-oxo-4,6,7,8-tetrahydropyrrolo[1,2-a]pyrimidine-6-carboxylate (150 mg, 96% yield).

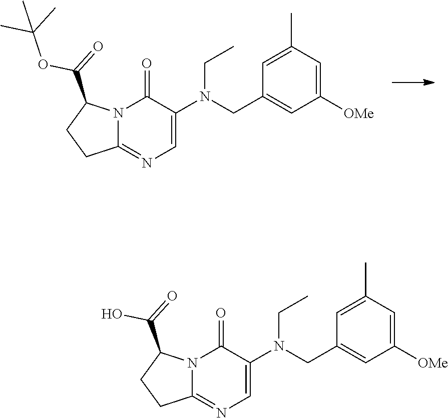 [SMILES: CCN(Cc1cc(C)cc(OC)c1)c1cnc2n(c1=O)[C@H](C(=O)OC(C)(C)C)CC2 ; CCN(Cc1cc(C)cc(OC)c1)c1cnc2n(c1=O)[C@H](C(=O)O)CC2]

Step 3: tert-Butyl (S)-3-(ethyl(3-methoxy-5-methylbenzyl)amino)-4-oxo-4,6,7,8-tetrahydropyrrolo[1,2-a]pyrimidine-6-carboxylate (245 mg, 592 μmol) was dissolved in DCM (12 mL) and treated with TFA (1.37 mL, 17.8 mmol). After stirring for 18 h at room temp, the reaction mixture was concentrated and the residue was purified by column chromatography (0-100% EtOAc-heptane, then 0-50% MeOH-DCM). The product was lyophilized to provide (S)-3-(ethyl(3-methoxy-5-methylbenzyl)amino)-4-oxo-4,6,7,8-tetrahydropyrrolo[1,2-a]pyrimidine-6-carboxylic acid, 1.5 trifluoroacetate (90.9 mg, 29% yield).

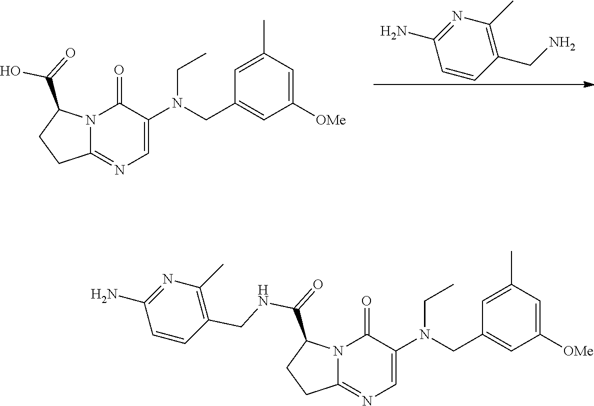

Step 4: To an ice-cold solution of (S)-3-(ethyl(3-methoxy-5-methylbenzyl)amino)-4-oxo-4,6,7,8-tetrahydropyrrolo[1,2-a]pyrimidine-6-carboxylic acid, 1.5 trifluoroacetate (70 mg, 0.13 mmol) in DMF (2.0 mL) was added DIEA (0.12 mL, 0.66 mmol). After stirring for 30 min, HATU (100 mg, 0.26 mmol) was added. After stirring for 10 min at the same temperature, 5-(aminomethyl)-6-methylpyridin-2-amine (22 mg, 0.16 mmol) was added. The reaction was allowed to warm to room temp and stirred for 18 h. Then it was concentrated and the residue was purified by column chromatography (amine, 0-30% MeOH-DCM). Additional purification by column chromatography (0-70% MeOH-DCM) provided (S)—N-((6-amino-2-methylpyridin-3-yl)methyl)-3-(ethyl(3-methoxy-5-methylbenzyl)amino)-4-oxo-4,6,7,8-tetrahydropyrrolo[1,2-a]pyrimidine-6-carboxamide (11 mg, 12% yield).

The following compounds were prepared according to the foregoing procedure using the appropriate aldehyde starting material, except that the final product was purified by preparatory HPLC method 2.

| | Cmp |
|---|---|
| Compound Name | No. |
| (S)-N-((6-amino-2-methylpyridin-3-yl)methyl)-3-((2-fluoro- | 68 |
| benzyl)amino)-4-oxo-4,6,7,8-tetrahydropyrrolo[1,2-a]pyrimidine-6- | |
| carboxamide, di-trifluoroacetate | |

### Example 35

### Preparation of (6S)—N—((R)-2-amino-6,7-dihydro-5H-cyclopenta[b]pyridin-5-yl)-3-(((4-cyanobicyclo[1.1.1]pentan-2-yl)methyl)amino)-4-oxo-4,6,7,8-tetrahydropyrrolo[1,2-a]pyrimidine-6-carboxamide (Compound 86)

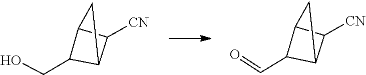 [SMILES: N#CC1C2CC1C2CO ; N#CC1C2CC1C2C=O]

Step 1: To an ice-cold solution of 4-(hydroxymethyl)bicyclo[1.1.1]pentane-2-carbonitrile (120 mg, 96.8 μL, 974 μmol) in DCM (5 mL), was added Dess-Martin periodinane (1.03 g, 2.44 mmol) and 2 drops of H₂O. After allowing to warm to room temp and stirring for 18 h, the reaction was quenched with sat. aq NaHCO₃/sat. aq Na₂S203/Et₂O (v/v/v=1:1:1), then stirred until the cloudy mixture turned clear. The organic phase was separated, and the aqueous layer was extracted with Et₂O. The combined organic layers were washed with brine, dried over anhydr Na₂SO₄, filtered, and concentrated to provide crude 4-formylbicyclo[1.1.1]pentane-2-carbonitrile (118 mg, 100% yield) which was used for the next step without further purification

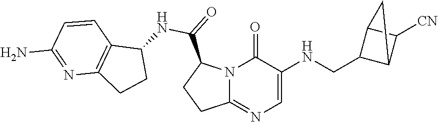 [SMILES: N#CC1C2CC1C2CNc1cnc2n(c1=O)[C@H](C(=O)N[C@@H]1CCc3nc(N)ccc31)CC2]

Steps 2-4: The title compound was prepared according to steps 1-3 of Example 30 with the appropriate amine starting material.

### Example 36

### Preparation of (6S)—N-((5,6-dihydro-4H-thieno[2,3-c]pyrrol-2-yl)methyl)-4-oxo-3-((1-(m-tolyl)ethyl)amino)-4,6,7,8-tetrahydropyrrolo[1,2-a]pyrimidine-6-carboxamide, di-trifluoroacetate, Isomer 1 AND Isomer 2 (Compound 69, Compound 70)

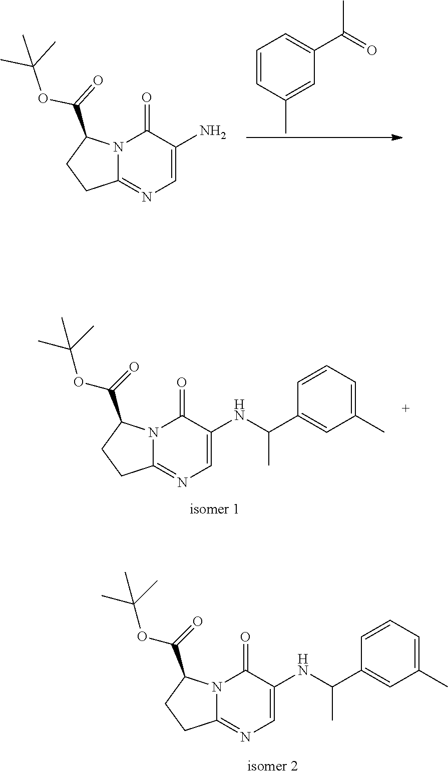

Step 1: To a solution of tert-butyl (S)-3-amino-4-oxo-4,6,7,8-tetrahydro-pyrrolo[1,2-a]pyrimidine-6-carboxylate (337 mg, 1.34 mmol) and 1-(m-tolyl)ethan-1-one (152 μL, 1.12 mmol) in DCM (5 mL) was added DIEA (0.19 mL, 1.12 mmol). After stirring for 30 min at room temp, trichlorosilane (233 μL, 2.24 mmol) was added. The mixture was stirred for 40 h, then quenched with sat. aq NaHCO₃ and extracted with DCM. The combined extracts were washed with H₂O, dried over anhydr Na₂SO₄, filtered, and concentrated. The residue was purified by column chromatography (0-100% EtOAc-heptane) to provide tert-butyl (6S)-4-oxo-3-((1-(m-tolyl)ethyl)amino)-4,6,7,8-tetrahydropyrrolo[1,2-a]pyrimidine-6-carboxylate (isomer 1 [less polar], 62 mg, 30% yield) and tert-butyl (6S)-4-oxo-3-((1-(m-tolyl)ethyl)amino)-4,6,7,8-tetrahydropyrrolo[1,2-a]pyrimidine-6-carboxylate (isomer 2 [more polar], 61 mg, 30% yield).

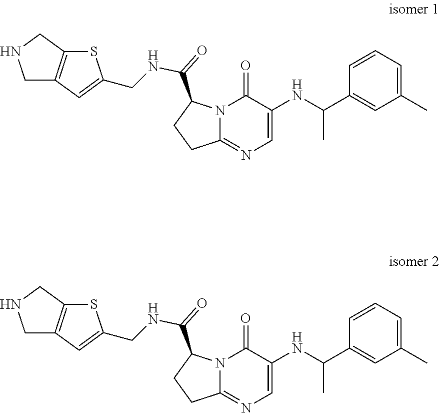 [SMILES: Cc1cccc(C(C)Nc2cnc3n(c2=O)[C@H](C(=O)NCc2cc4c(s2)CNC4)CC3)c1 ; Cc1cccc(C(C)Nc2cnc3n(c2=O)[C@H](C(=O)NCc2cc4c(s2)CNC4)CC3)c1]

Steps 2-4: Each of the title compounds, isomer 1 and 2, were prepared separately according to steps 2-4 of Example 32 with the appropriate amine starting material (prepared according to steps 1-3 of Example 16).

### Example 37

### Preparation of (S)—N-((6-amino-2-methylpyridin-3-yl)methyl)-3-((3-(difluoromethyl)benzyl)amino)-4-oxo-4,6,7,8-tetrahydropyrrolo[1,2-a]pyrimidine-6-carboxamide (Compound 71)

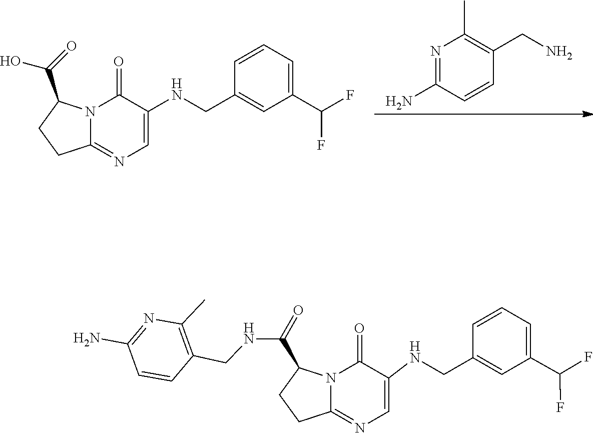

Step 1: To a solution of (S)-3-((3-(difluoromethyl)benzyl)amino)-4-oxo-4,6,7,8-tetrahydropyrrolo[1,2-a]pyrimidine-6-carboxylic acid, 1.5 trifluoroacetic acid (prepared according to steps 1-2 of Example 30, 106 mg, 0.236 mmol) in DMF (2 ml) was added HATU (215 mg, 0.56 mmol), Et₃N (96 mg, 0.94 mmol), and 5-(aminomethyl)-6-methylpyridin-2-amine (84 mg, 0.61 mmol.) The solution was stirred for 16 h at room temp then evaporated to dryness. Purification using chromatography (6% MeOH—NH₃/CH₂Cl₂) gave (S)—N-((6-amino-2-methylpyridin-3-yl)methyl)-3-((3-(difluoromethyl)benzyl)amino)-4-oxo-4,6,7,8-tetrahydropyrrolo[1,2-a]pyrimidine-6-carboxamide (54 mg, 50% yield) as a pale-yellow solid.

The following compounds were prepared according to the foregoing procedures using the appropriate starting materials:

| | Cmp |
|---|---|
| Compound Name | No. |
| (S)-N-((6-amino-2-methylpyridin-3-yl)methyl)-3-((3,4- | 72 |
| dimethylbenzyl)amino)-4-oxo-4,6,7,8-tetrahydropyrrolo[1,2- | |
| a]pyrimidine-6-carboxamide | |
| (S)-N-((6-amino-2-methylpyridin-3-yl)methyl)-3-((3- | 73 |
| cyanobenzyl)amino)-4-oxo-4,6,7,8-tetrahydropyrrolo[1,2- | |
| a]pyrimidine-6-carboxamide | |
| (S)-N-((6-amino-2-methylpyridin-3-yl)methyl)-3-((3-chloro-5- | 74 |
| methoxybenzyl)amino)-4-oxo-4,6,7,8-tetrahydropyrrolo[1,2- | |
| a]pyrimidine-6-carboxamide | |
| (S)-N-((6-amino-2-methylpyridin-3-yl)methyl)-3-((3- | 75 |
| (difluoromethoxy)benzyl)amino)-4-oxo-4,6,7,8-tetrahydro- | |
| pyrrolo[1,2-a]pyrimidine-6-carboxamide | |

The following compounds were prepared according to the foregoing procedures using the appropriate starting materials:

| | Cmp |
|---|---|
| Compound Name | No. |
| (S)-N-((R)-1-(6-aminopyridin-3-yl)ethyl)-3-((3-chloro-5- | 76 |
| methoxybenzyl)amino)-4-oxo-4,6,7,8-tetrahydropyrrolo[1,2- | |
| a]pyrimidine-6-carboxamide | |
| (R)-N-((R)-1-(6-aminopyridin-3-yl)ethyl)-3-((3-chloro-5- | 77 |
| methoxybenzyl)amino)-4-oxo-4,6,7,8-tetrahydropyrrolo[1,2- | |
| a]pyrimidine-6-carboxamide | |

The following compounds were prepared according to Example 30 using the appropriate starting materials and purified using chromatography (6% 7 N NH₃-MeOH/CH₂Cl₂). Further purification using chromatography (8% MeOH/EtOAc) was conducted as needed.

| | Cmp |
|---|---|
| Compound Name | No. |
| (S)-N-((R)-2-amino-6,7-dihydro-5H-cyclopenta[b]pyridin-5-yl)-3- | 87 |
| ((3-chloro-5-methoxybenzyl)amino)-4-oxo-4,6,7,8-tetrahydro- | |
| pyrrolo[1,2-a]pyrimidine-6-carboxamide | |
| (S)-N-((R)-2-amino-6,7-dihydro-5H-cyclopenta[b]pyridin-5-yl)-3- | 88 |
| ((3-cyano-4-methylbenzyl)amino)-4-oxo-4,6,7,8-tetrahydro- | |
| pyrrolo[1,2-a]pyrimidine-6-carboxamide | |
| (S)-N-((R)-2-amino-6,7-dihydro-5H-cyclopenta[b]pyridin-5-yl)-3- | 89 |
| ((3-chloro-5-cyanobenzyl)amino)-4-oxo-4,6,7,8-tetrahydro- | |
| pyrrolo[1,2-a]pyrimidine-6-carboxamide | |
| (S)-N-((R)-2-amino-6,7-dihydro-5H-cyclopenta[b]pyridin-5-yl)-3- | 90 |
| ((3-cyano-5-(trifluoromethyl)benzyl)amino)-4-oxo-4,6,7,8- | |
| tetrahydropyrrolo[1,2-a]pyrimidine-6-carboxamide | |
| (S)-N-((R)-2-amino-6,7-dihydro-5H-cyclopenta[b]pyridin-5-yl)-3- | 91 |
| ((3-(cyanomethyl)benzyl)amino)-4-oxo-4,6,7,8-tetrahydro- | |
| pyrrolo[1,2-a]pyrimidine-6-carboxamide | |
| (S)-N-((6-amino-2-methylpyridin-3-yl)methyl)-3-((3- | 80 |
| (cyanomethyl)benzyl)amino)-4-oxo-4,6,7,8-tetrahydropyrrolo[1,2- | |
| a]pyrimidine-6-carboxamide | |
| (S)-N-((R)-2-amino-6,7-dihydro-5H-cyclopenta[b]pyridin-5-yl)-3- | 92 |
| ((3-cyano-2-methylbenzyl)amino)-4-oxo-4,6,7,8-tetrahydro- | |
| pyrrolo[1,2-a]pyrimidine-6-carboxamide | |
| (S)-N-((R)-2-amino-6,7-dihydro-5H-cyclopenta[b]pyridin-5-yl)-3- | 93 |
| ((3-cyano-5-fluorobenzyl)amino)-4-oxo-4,6,7,8-tetrahydro- | |
| pyrrolo[1,2-a]pyrimidine-6-carboxamide | |

### Example 38

### Additional Compounds

Compound numbers 94-107 were synthesized by procedures analogous to the procedures described in the foregoing examples, and were evaluated by the procedures described in the examples which follow.

### Example 39

### Exemplary Compounds

The exemplary compounds of the disclosure, as disclosed herein and produced by the Examples above, are shown in Table 1 and Table 2, below.

TABLE 1
Exemplary compounds of the disclosure

| | | | Exact | |
|---|---|---|---|---|
| | | | Observed | |
| | | | Mass | Exact |
| Cmp | | | (ES+; | Calc. |
| No. | Structure | Salt | M + H) | Mass |
| 1 | 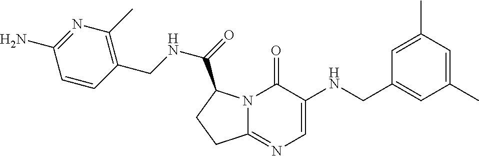 [SMILES: Cc1cc(C)cc(CNc2cnc3n(c2=O)[C@H](C(=O)NCc2ccc(N)nc2C)CC3)c1] | | 433.27 | 432.23 |
| 2 | 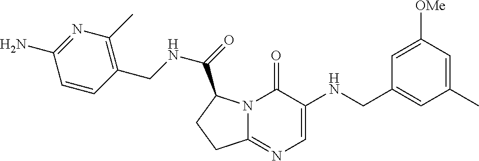 [SMILES: COc1cc(C)cc(CNc2cnc3n(c2=O)[C@H](C(=O)NCc2ccc(N)nc2C)CC3)c1] | | 449.23 | 448.22 |
| 3 | 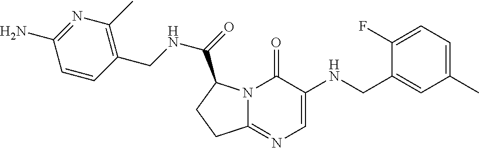 [SMILES: Cc1ccc(F)c(CNc2cnc3n(c2=O)[C@H](C(=O)NCc2ccc(N)nc2C)CC3)c1] | | 437.23 | 436.20 |
| 4 | 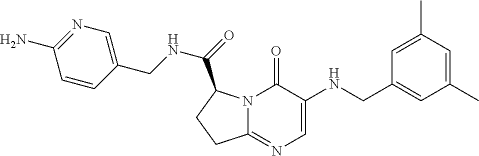 [SMILES: Cc1cc(C)cc(CNc2cnc3n(c2=O)[C@H](C(=O)NCc2ccc(N)nc2)CC3)c1] | | 419.22 | 418.21 |
| 5 | 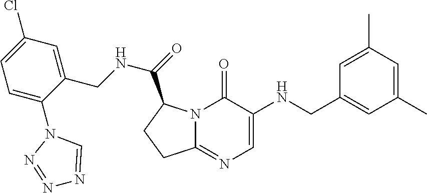 [SMILES: Cc1cc(C)cc(CNc2cnc3n(c2=O)[C@H](C(=O)NCc2cc(Cl)ccc2-n2cnnn2)CC3)c1] | | 505.16 | 504.18 |
| 6 | 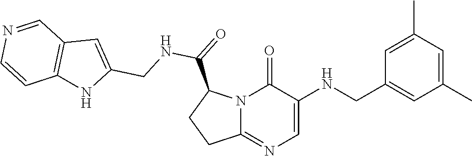 [SMILES: Cc1cc(C)cc(CNc2cnc3n(c2=O)[C@H](C(=O)NCc2cc4cnccc4[nH]2)CC3)c1] | | 443.22 | 442.21 |
| 7 | 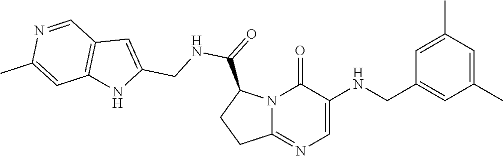 | TFA | 456.23 | 455.26 |
| 8 | 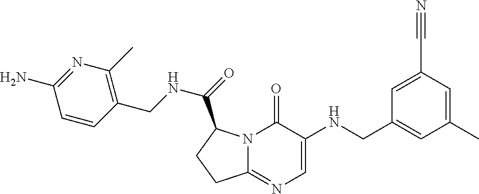 [SMILES: Cc1cc(C#N)cc(CNc2cnc3n(c2=O)[C@H](C(=O)NCc2ccc(N)nc2C)CC3)c1] | | 444.21 | 443.21 |
| 9 | 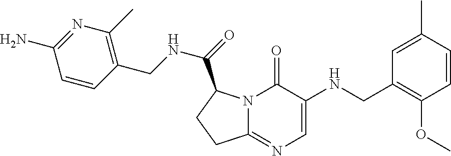 [SMILES: COc1ccc(C)cc1CNc1cnc2n(c1=O)[C@H](C(=O)NCc1ccc(N)nc1C)CC2] | TFA | 449.22 | 448.22 |
| 10 | 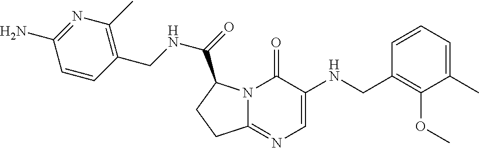 [SMILES: COc1c(C)cccc1CNc1cnc2n(c1=O)[C@H](C(=O)NCc1ccc(N)nc1C)CC2] | TFA | 449.66 | 448.22 |
| 11 | 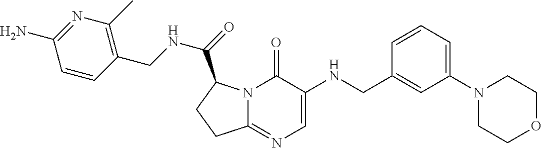 [SMILES: Cc1nc(N)ccc1CNC(=O)[C@@H]1CCc2ncc(NCc3cccc(N4CCOCC4)c3)c(=O)n21] | HCl | 490.64 | 489.25 |
| 12 | 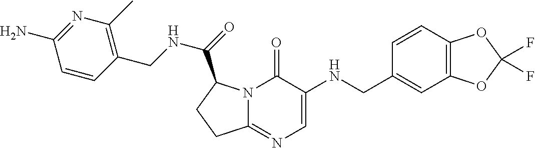 [SMILES: Cc1nc(N)ccc1CNC(=O)[C@@H]1CCc2ncc(NCc3ccc4c(c3)OC(F)(F)O4)c(=O)n21] | TFA | 485.54 | 484.17 |
| 13 | 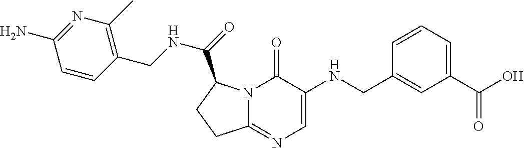 [SMILES: Cc1nc(N)ccc1CNC(=O)[C@@H]1CCc2ncc(NCc3cccc(C(=O)O)c3)c(=O)n21] | TFA | 449.66 | 448.19 |
| 14 | 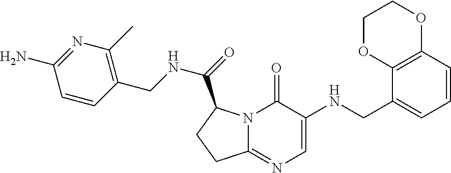 [SMILES: Cc1nc(N)ccc1CNC(=O)[C@@H]1CCc2ncc(NCc3cccc4c3OCCO4)c(=O)n21] | | 463.55 | 462.20 |
| 15 | 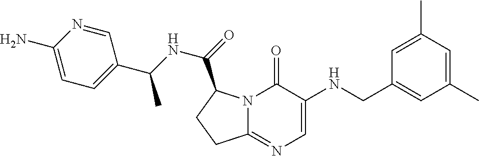 [SMILES: Cc1cc(C)cc(CNc2cnc3n(c2=O)[C@H](C(=O)N[C@@H](C)c2ccc(N)nc2)CC3)c1] | | 433.20 | 432.23 |
| 16 | 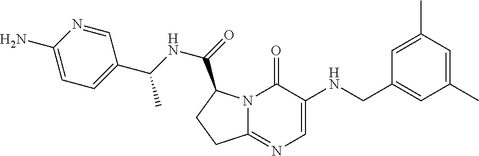 [SMILES: Cc1cc(C)cc(CNc2cnc3n(c2=O)[C@H](C(=O)N[C@H](C)c2ccc(N)nc2)CC3)c1] | TFA | 433.20 | 432.23 |
| 17 | 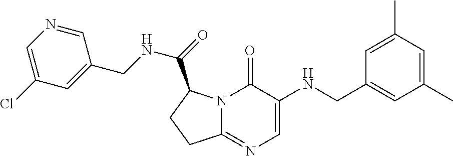 [SMILES: Cc1cc(C)cc(CNc2cnc3n(c2=O)[C@H](C(=O)NCc2cncc(Cl)c2)CC3)c1] | TFA | 438.15 | 437.16 |
| 18 | 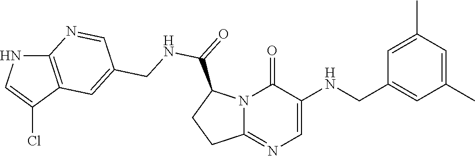 [SMILES: Cc1cc(C)cc(CNc2cnc3n(c2=O)[C@H](C(=O)NCc2cnc4[nH]cc(Cl)c4c2)CC3)c1] | TFA | 478.54 | 476.17 |
| 19 | 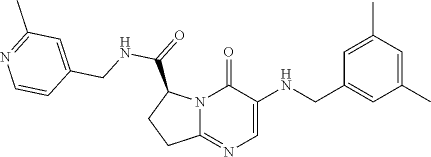 [SMILES: Cc1cc(C)cc(CNc2cnc3n(c2=O)[C@H](C(=O)NCc2ccnc(C)c2)CC3)c1] | | 418.67 | 417.22 |
| 20 | 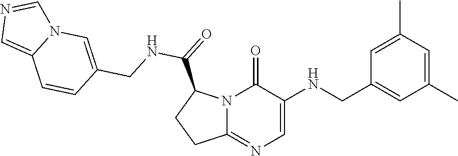 [SMILES: Cc1cc(C)cc(CNc2cnc3n(c2=O)[C@H](C(=O)NCc2ccc4cncn4c2)CC3)c1] | | 443.56 | 442.21 |
| 21 | 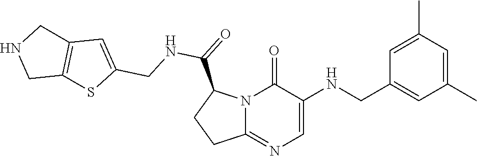 [SMILES: Cc1cc(C)cc(CNc2cnc3n(c2=O)[C@H](C(=O)NCc2cc4c(s2)CNC4)CC3)c1] | TFA | 450.22 | 449.19 |
| 22 | 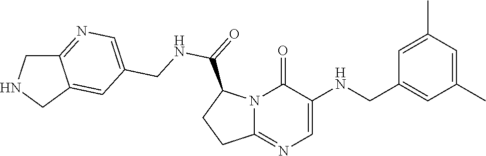 [SMILES: Cc1cc(C)cc(CNc2cnc3n(c2=O)[C@H](C(=O)NCc2cnc4c(c2)CNC4)CC3)c1] | TFA | 445.21 | 444.23 |
| 23 | 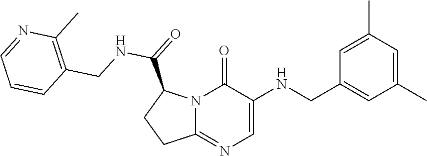 [SMILES: Cc1cc(C)cc(CNc2cnc3n(c2=O)[C@H](C(=O)NCc2cccnc2C)CC3)c1] | | 418.20 | 417.22 |
| 24 | 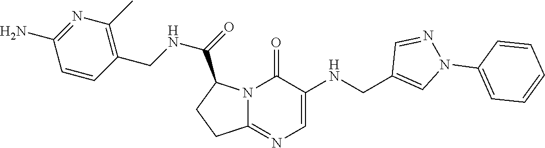 | TFA | 471.23 | 470.22 |
| 25 | 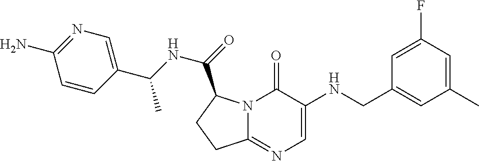 [SMILES: Cc1cc(F)cc(CNc2cnc3n(c2=O)[C@H](C(=O)N[C@H](C)c2ccc(N)nc2)CC3)c1] | 2TFA | 437.23 | 436.20 |
| 26 | 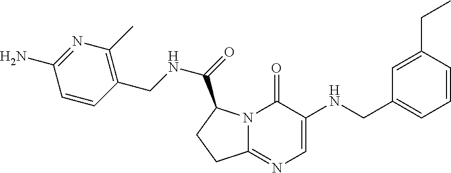 [SMILES: CCc1cccc(CNc2cnc3n(c2=O)[C@H](C(=O)NCc2ccc(N)nc2C)CC3)c1] | TFA | 433.23 | 432.23 |
| 27 | 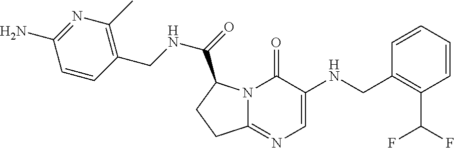 [SMILES: Cc1nc(N)ccc1CNC(=O)[C@@H]1CCc2ncc(NCc3ccccc3C(F)F)c(=O)n21] | 2TFA | 455.19 | 454.19 |
| 28 | 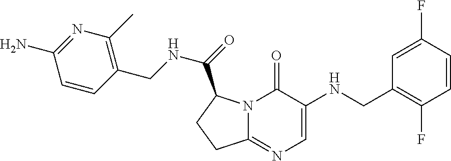 [SMILES: Cc1nc(N)ccc1CNC(=O)[C@@H]1CCc2ncc(NCc3cc(F)ccc3F)c(=O)n21] | 2TFA | 441.19 | 440.18 |
| 29 | 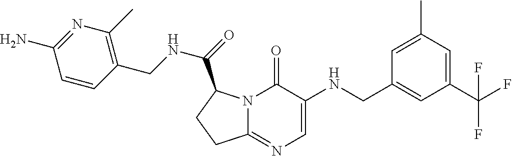 [SMILES: Cc1cc(CNc2cnc3n(c2=O)[C@H](C(=O)NCc2ccc(N)nc2C)CC3)cc(C(F)(F)F)c1] | 2TFA | 487.24 | 486.20 |
| 30 | 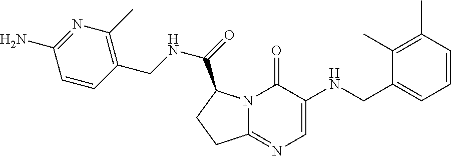 [SMILES: Cc1cccc(CNc2cnc3n(c2=O)[C@H](C(=O)NCc2ccc(N)nc2C)CC3)c1C] | TFA | 433.25 | 432.23 |
| 31 | 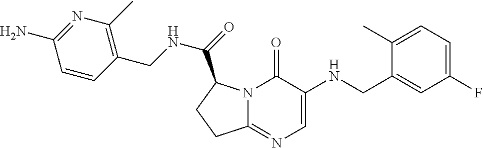 [SMILES: Cc1ccc(F)cc1CNc1cnc2n(c1=O)[C@H](C(=O)NCc1ccc(N)nc1C)CC2] | 2TFA | 437.20 | 436.20 |
| 32 | 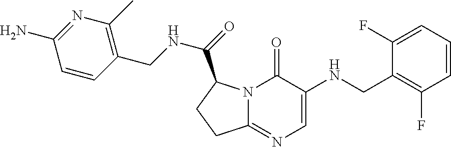 [SMILES: Cc1nc(N)ccc1CNC(=O)[C@@H]1CCc2ncc(NCc3c(F)cccc3F)c(=O)n21] | 2TFA | 441.20 | 440.18 |
| 33 | 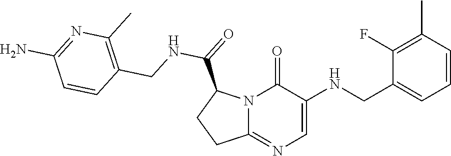 [SMILES: Cc1cccc(CNc2cnc3n(c2=O)[C@H](C(=O)NCc2ccc(N)nc2C)CC3)c1F] | 2TFA | 437.23 | 436.20 |
| 34 | 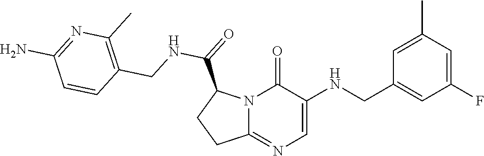 [SMILES: Cc1cc(F)cc(CNc2cnc3n(c2=O)[C@H](C(=O)NCc2ccc(N)nc2C)CC3)c1] | 2TFA | 437.21 | 436.20 |
| 35 | 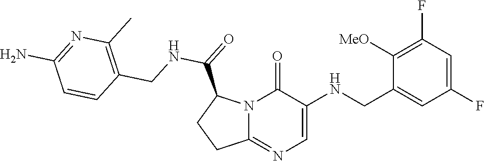 [SMILES: COc1c(F)cc(F)cc1CNc1cnc2n(c1=O)[C@H](C(=O)NCc1ccc(N)nc1C)CC2] | TFA | 471.18 | 470.19 |
| 36 | 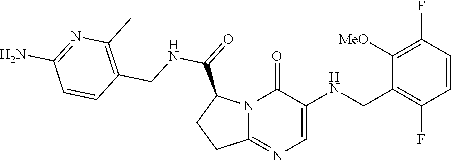 [SMILES: COc1c(F)ccc(F)c1CNc1cnc2n(c1=O)[C@H](C(=O)NCc1ccc(N)nc1C)CC2] | 2TFA | 471.19 | 470.19 |
| 37 | 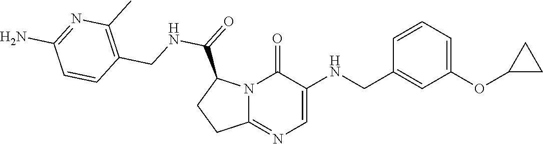 [SMILES: Cc1nc(N)ccc1CNC(=O)[C@@H]1CCc2ncc(NCc3cccc(OC4CC4)c3)c(=O)n21] | TFA | 461.28 | 460.22 |
| 38 | 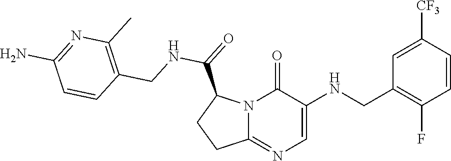 [SMILES: Cc1nc(N)ccc1CNC(=O)[C@@H]1CCc2ncc(NCc3cc(C(F)(F)F)ccc3F)c(=O)n21] | 2TFA | 491.22 | 490.17 |
| 39 | 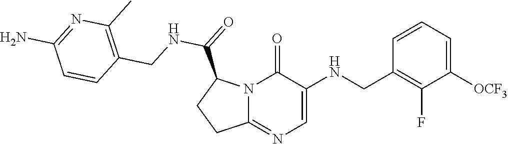 [SMILES: Cc1nc(N)ccc1CNC(=O)[C@@H]1CCc2ncc(NCc3cccc(OC(F)(F)F)c3F)c(=O)n21] | 2TFA | 507.20 | 506.17 |
| 40 | 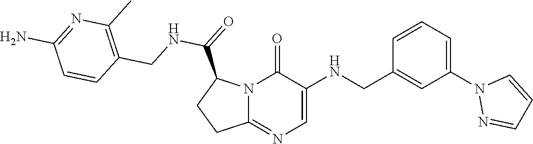 [SMILES: Cc1nc(N)ccc1CNC(=O)[C@@H]1CCc2ncc(NCc3cccc(-n4cccn4)c3)c(=O)n21] | TFA | 471.27 | 470.22 |
| 41 | 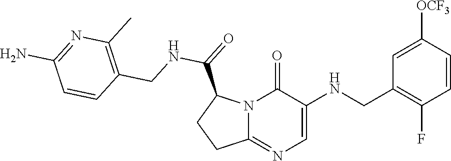 [SMILES: Cc1nc(N)ccc1CNC(=O)[C@@H]1CCc2ncc(NCc3cc(OC(F)(F)F)ccc3F)c(=O)n21] | 2TFA | 507.21 | 506.17 |
| 42 | 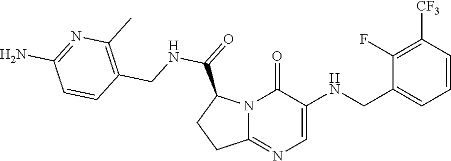 [SMILES: Cc1nc(N)ccc1CNC(=O)[C@@H]1CCc2ncc(NCc3cccc(C(F)(F)F)c3F)c(=O)n21] | 2TFA | 491.18 | 490.17 |
| 43 | 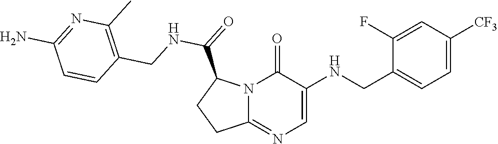 [SMILES: Cc1nc(N)ccc1CNC(=O)[C@@H]1CCc2ncc(NCc3ccc(C(F)(F)F)cc3F)c(=O)n21] | 2TFA | 491.20 | 490.17 |
| 44 | 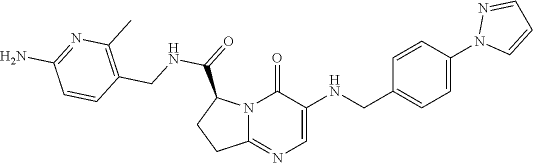 [SMILES: Cc1nc(N)ccc1CNC(=O)[C@@H]1CCc2ncc(NCc3ccc(-n4cccn4)cc3)c(=O)n21] | TFA | 471.25 | 470.22 |
| 45 | 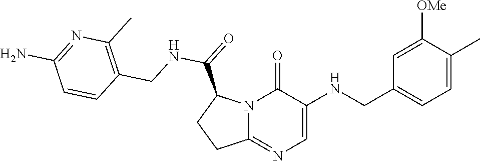 [SMILES: COc1cc(CNc2cnc3n(c2=O)[C@H](C(=O)NCc2ccc(N)nc2C)CC3)ccc1C] | TFA | 449.23 | 448.22 |
| 46 | 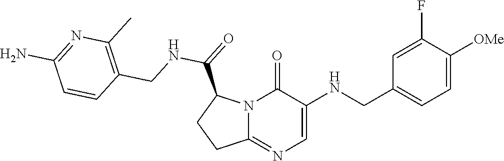 [SMILES: COc1ccc(CNc2cnc3n(c2=O)[C@H](C(=O)NCc2ccc(N)nc2C)CC3)cc1F] | 2TFA | 453.21 | 452.20 |
| 47 | 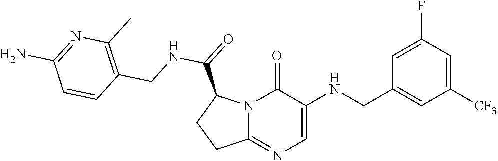 [SMILES: Cc1nc(N)ccc1CNC(=O)[C@@H]1CCc2ncc(NCc3cc(F)cc(C(F)(F)F)c3)c(=O)n21] | 2TFA | 491.21 | 490.17 |
| 48 | 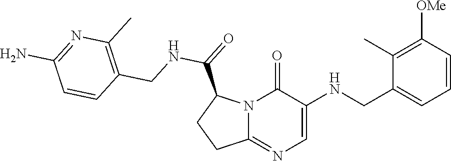 [SMILES: COc1cccc(CNc2cnc3n(c2=O)[C@H](C(=O)NCc2ccc(N)nc2C)CC3)c1C] | TFA | 449.26 | 448.22 |
| 49 | 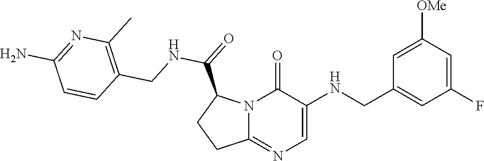 [SMILES: COc1cc(F)cc(CNc2cnc3n(c2=O)[C@H](C(=O)NCc2ccc(N)nc2C)CC3)c1] | 2TFA | 453.25 | 452.20 |
| 50 | 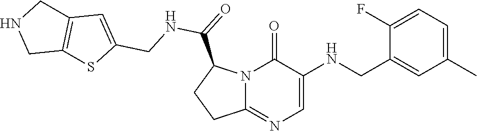 [SMILES: Cc1ccc(F)c(CNc2cnc3n(c2=O)[C@H](C(=O)NCc2cc4c(s2)CNC4)CC3)c1] | 2TFA | 454.16 | 453.16 |
| 51 | 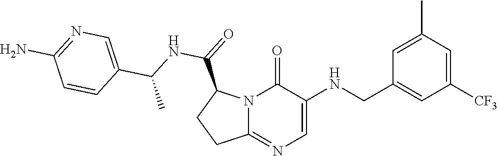 [SMILES: Cc1cc(CNc2cnc3n(c2=O)[C@H](C(=O)N[C@H](C)c2ccc(N)nc2)CC3)cc(C(F)(F)F)c1] | 2TFA | 487.23 | 486.20 |
| 52 | 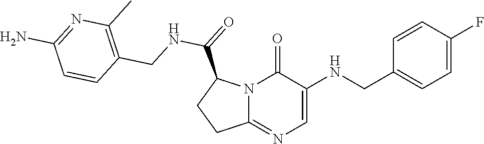 [SMILES: Cc1nc(N)ccc1CNC(=O)[C@@H]1CCc2ncc(NCc3ccc(F)cc3)c(=O)n21] | | 423.19 | 422.19 |
| 53 | 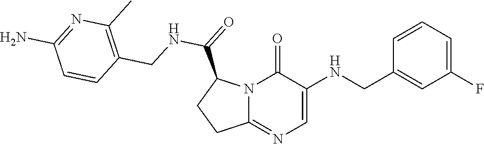 [SMILES: Cc1nc(N)ccc1CNC(=O)[C@@H]1CCc2ncc(NCc3cccc(F)c3)c(=O)n21] | | 423.06 | 422.19 |
| 54 | 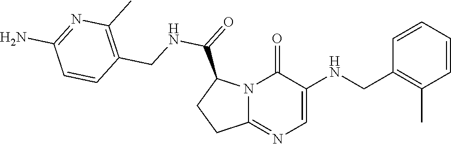 [SMILES: Cc1ccccc1CNc1cnc2n(c1=O)[C@H](C(=O)NCc1ccc(N)nc1C)CC2] | | 419.47 | 418.21 |
| 55 | 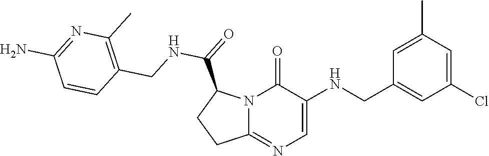 [SMILES: Cc1cc(Cl)cc(CNc2cnc3n(c2=O)[C@H](C(=O)NCc2ccc(N)nc2C)CC3)c1] | | 453.33 | 452.17 |
| 56 | 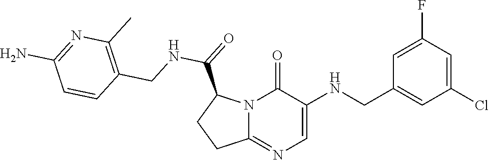 [SMILES: Cc1nc(N)ccc1CNC(=O)[C@@H]1CCc2ncc(NCc3cc(F)cc(Cl)c3)c(=O)n21] | | 457.35 | 456.15 |
| 57 | 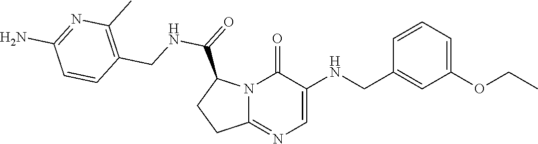 [SMILES: CCOc1cccc(CNc2cnc3n(c2=O)[C@H](C(=O)NCc2ccc(N)nc2C)CC3)c1] | | 449.39 | 448.22 |
| 58 | 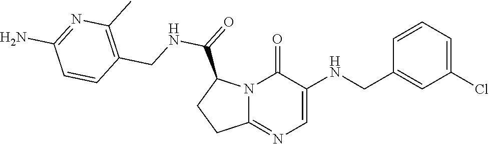 [SMILES: Cc1nc(N)ccc1CNC(=O)[C@@H]1CCc2ncc(NCc3cccc(Cl)c3)c(=O)n21] | | 439.42 | 438.16 |
| 59 | 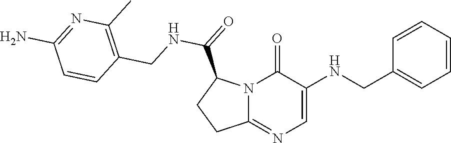 [SMILES: Cc1nc(N)ccc1CNC(=O)[C@@H]1CCc2ncc(NCc3ccccc3)c(=O)n21] | | 405.38 | 404.20 |
| 60 | 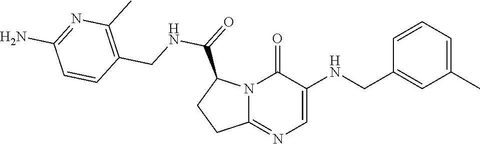 [SMILES: Cc1cccc(CNc2cnc3n(c2=O)[C@H](C(=O)NCc2ccc(N)nc2C)CC3)c1] | | 419.47 | 418.21 |
| 61 | 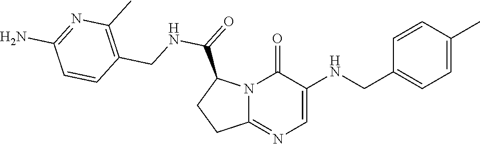 [SMILES: Cc1ccc(CNc2cnc3n(c2=O)[C@H](C(=O)NCc2ccc(N)nc2C)CC3)cc1] | | 419.47 | 418.21 |
| 62 | 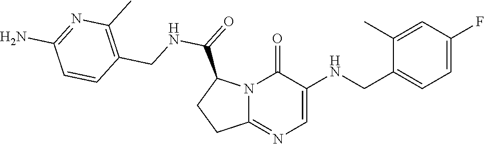 [SMILES: Cc1cc(F)ccc1CNc1cnc2n(c1=O)[C@H](C(=O)NCc1ccc(N)nc1C)CC2] | 2TFA | 437.25 | 436.20 |
| 63 | 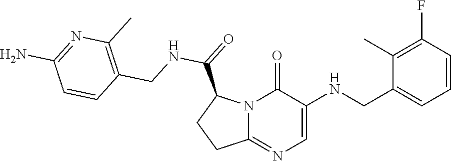 [SMILES: Cc1nc(N)ccc1CNC(=O)[C@@H]1CCc2ncc(NCc3cccc(F)c3C)c(=O)n21] | 2TFA | 437.24 | 436.20 |
| 64 | 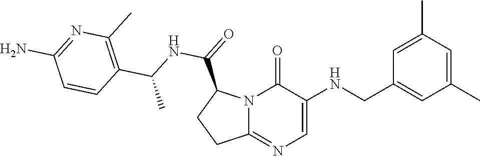 [SMILES: Cc1cc(C)cc(CNc2cnc3n(c2=O)[C@H](C(=O)N[C@H](C)c2ccc(N)nc2C)CC3)c1] | | 447.23 | 446.24 |
| 65 | 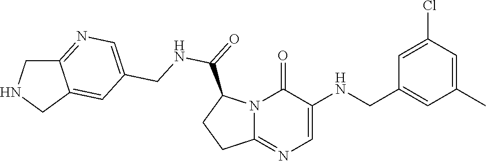 [SMILES: Cc1cc(Cl)cc(CNc2cnc3n(c2=O)[C@H](C(=O)NCc2cnc4c(c2)CNC4)CC3)c1] | 2TFA | 465.18 | 464.17 |
| 66 | 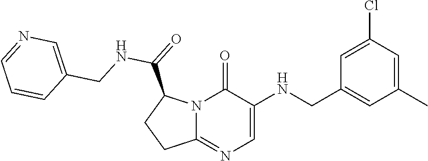 [SMILES: Cc1cc(Cl)cc(CNc2cnc3n(c2=O)[C@H](C(=O)NCc2cccnc2)CC3)c1] | TFA | 424.15 | 423.15 |
| 67 | 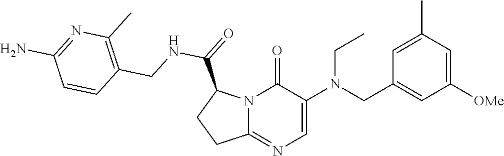 [SMILES: CCN(Cc1cc(C)cc(OC)c1)c1cnc2n(c1=O)[C@H](C(=O)NCc1ccc(N)nc1C)CC2] | | 477.24 | 476.25 |
| 68 | 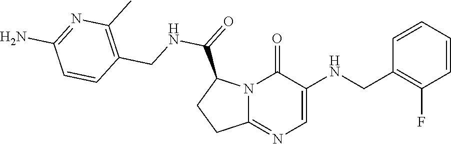 [SMILES: Cc1nc(N)ccc1CNC(=O)[C@@H]1CCc2ncc(NCc3ccccc3F)c(=O)n21] | 2TFA | 423.19 | 422.19 |
| 69 | 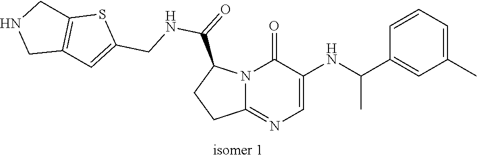 [SMILES: Cc1cccc(C(C)Nc2cnc3n(c2=O)[C@H](C(=O)NCc2cc4c(s2)CNC4)CC3)c1] | 2TFA | 450.25 | 449.19 |
| 70 | 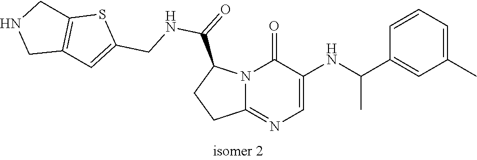 [SMILES: Cc1cccc(C(C)Nc2cnc3n(c2=O)[C@H](C(=O)NCc2cc4c(s2)CNC4)CC3)c1] | 2TFA | 450.25 | 449.19 |
| 71 | 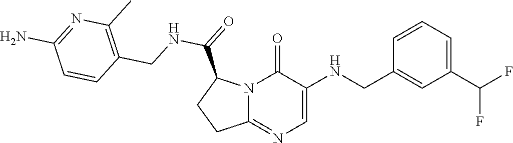 [SMILES: Cc1nc(N)ccc1CNC(=O)[C@@H]1CCc2ncc(NCc3cccc(C(F)F)c3)c(=O)n21] | | 455.34 | 454.19 |
| 72 | 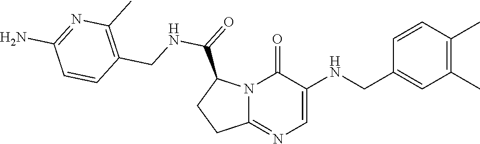 [SMILES: Cc1ccc(CNc2cnc3n(c2=O)[C@H](C(=O)NCc2ccc(N)nc2C)CC3)cc1C] | | 433.47 | 432.23 |
| 73 | 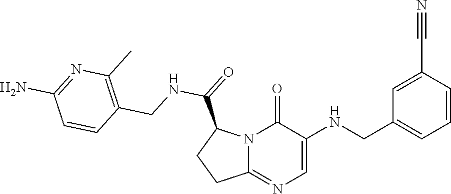 [SMILES: Cc1nc(N)ccc1CNC(=O)[C@@H]1CCc2ncc(NCc3cccc(C#N)c3)c(=O)n21] | | 430.41 | 429.19 |
| 74 | 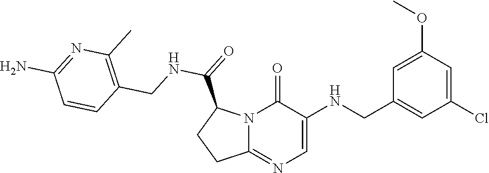 [SMILES: COc1cc(Cl)cc(CNc2cnc3n(c2=O)[C@H](C(=O)NCc2ccc(N)nc2C)CC3)c1] | | 469.34 | 468.17 |
| 75 | 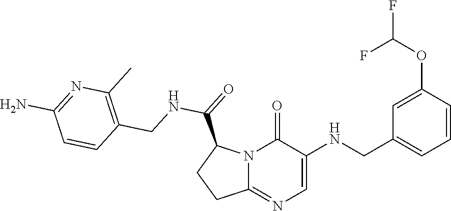 [SMILES: Cc1nc(N)ccc1CNC(=O)[C@@H]1CCc2ncc(NCc3cccc(OC(F)F)c3)c(=O)n21] | | 471.35 | 470.19 |
| 76 | 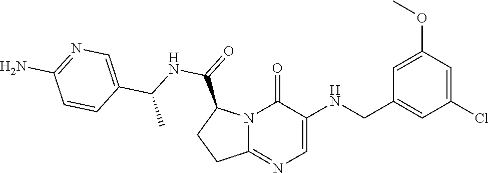 [SMILES: COc1cc(Cl)cc(CNc2cnc3n(c2=O)[C@H](C(=O)N[C@H](C)c2ccc(N)nc2)CC3)c1] | | 469.34 | 468.17 |
| 77 | 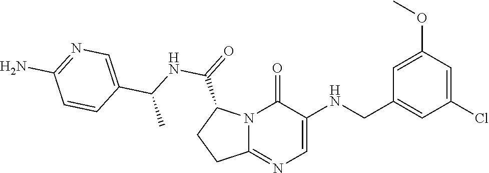 [SMILES: COc1cc(Cl)cc(CNc2cnc3n(c2=O)[C@@H](C(=O)N[C@H](C)c2ccc(N)nc2)CC3)c1] | | 469.34 | 468.17 |
| 78 | 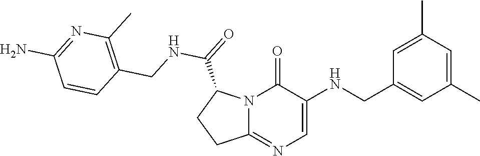 [SMILES: Cc1cc(C)cc(CNc2cnc3n(c2=O)[C@@H](C(=O)NCc2ccc(N)nc2C)CC3)c1] | | 433.27 | 432.23 |
| 79 | 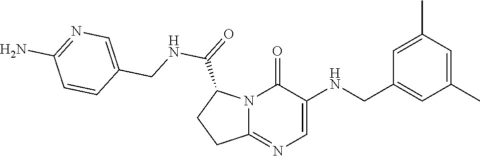 [SMILES: Cc1cc(C)cc(CNc2cnc3n(c2=O)[C@@H](C(=O)NCc2ccc(N)nc2)CC3)c1] | | 419.22 | 418.21 |
| 80 | 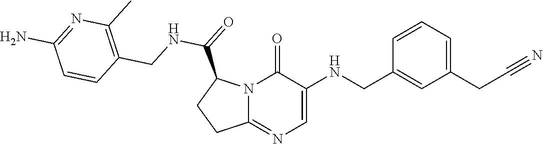 [SMILES: Cc1nc(N)ccc1CNC(=O)[C@@H]1CCc2ncc(NCc3cccc(CC#N)c3)c(=O)n21] | | 444.45 | 443.21 |
| 106 | 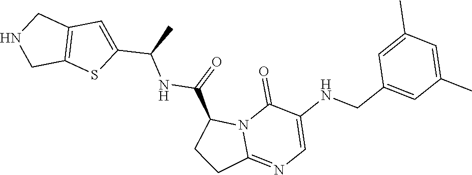 [SMILES: Cc1cc(C)cc(CNc2cnc3n(c2=O)[C@H](C(=O)N[C@H](C)c2cc4c(s2)CNC4)CC3)c1] | TFA | 464.21 | 463.20 |
| 107 | 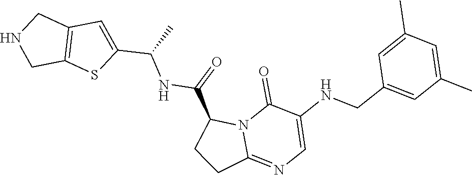 [SMILES: Cc1cc(C)cc(CNc2cnc3n(c2=O)[C@H](C(=O)N[C@@H](C)c2cc4c(s2)CNC4)CC3)c1] | TFA | 464.20 | 463.20 |

TABLE 2
Exemplary compounds of the disclosure

| | | Exact | |
|---|---|---|---|
| | | Observed | |
| | | Mass | |
| Cmp | | (ES+; | Exact Calc. |
| No. | Structure | M + H) | Mass |
| 81 | 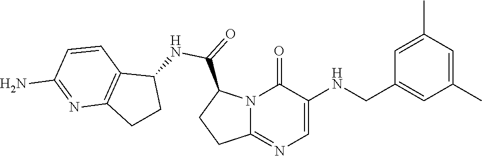 [SMILES: Cc1cc(C)cc(CNc2cnc3n(c2=O)[C@H](C(=O)N[C@@H]2CCc4nc(N)ccc42)CC3)c1] | 445.29 | 444.23 |
| 82 | 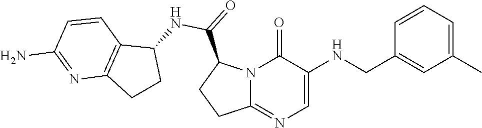 [SMILES: Cc1cccc(CNc2cnc3n(c2=O)[C@H](C(=O)N[C@@H]2CCc4nc(N)ccc42)CC3)c1] | 431.29 | 430.21 |
| 83 | 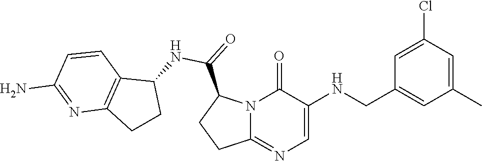 [SMILES: Cc1cc(Cl)cc(CNc2cnc3n(c2=O)[C@H](C(=O)N[C@@H]2CCc4nc(N)ccc42)CC3)c1] | 465.19 | 464.17 |
| 84 | 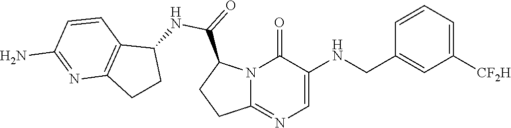 [SMILES: Nc1ccc2c(n1)CC[C@H]2NC(=O)[C@@H]1CCc2ncc(NCc3cccc(C(F)F)c3)c(=O)n21] | 467.21 | 466.19 |
| 85 | 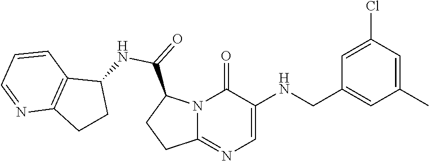 [SMILES: Cc1cc(Cl)cc(CNc2cnc3n(c2=O)[C@H](C(=O)N[C@@H]2CCc4ncccc42)CC3)c1] | 450.17 | 449.16 |
| 86 | 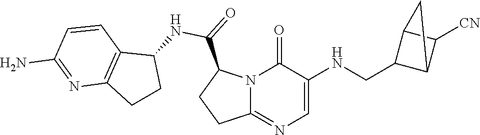 [SMILES: N#CC1C2CC1C2CNc1cnc2n(c1=O)[C@H](C(=O)N[C@@H]1CCc3nc(N)ccc31)CC2] | 432.22 | 431.21 |
| 87 | 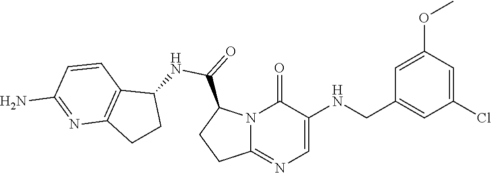 [SMILES: COc1cc(Cl)cc(CNc2cnc3n(c2=O)[C@H](C(=O)N[C@@H]2CCc4nc(N)ccc42)CC3)c1] | 481.33 | 480.17 |
| 88 | 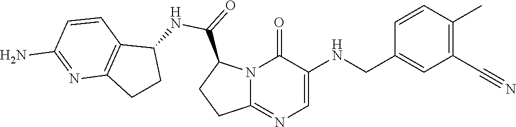 [SMILES: Cc1ccc(CNc2cnc3n(c2=O)[C@H](C(=O)N[C@@H]2CCc4nc(N)ccc42)CC3)cc1C#N] | 456.44 | 455.21 |
| 89 | 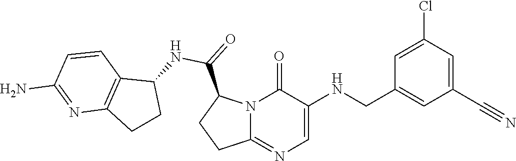 [SMILES: N#Cc1cc(Cl)cc(CNc2cnc3n(c2=O)[C@H](C(=O)N[C@@H]2CCc4nc(N)ccc42)CC3)c1] | 476.39 | 475.15 |
| 90 | 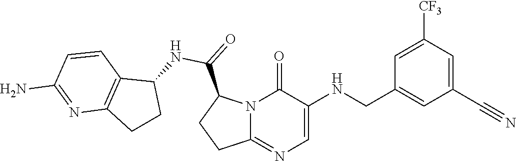 [SMILES: N#Cc1cc(CNc2cnc3n(c2=O)[C@H](C(=O)N[C@@H]2CCc4nc(N)ccc42)CC3)cc(C(F)(F)F)c1] | 510.52 | 509.18 |
| 91 | 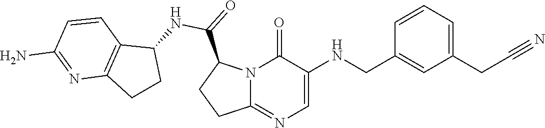 [SMILES: N#CCc1cccc(CNc2cnc3n(c2=O)[C@H](C(=O)N[C@@H]2CCc4nc(N)ccc42)CC3)c1] | 456.44 | 455.21 |
| 92 | 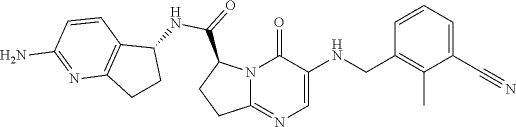 [SMILES: Cc1c(C#N)cccc1CNc1cnc2n(c1=O)[C@H](C(=O)N[C@@H]1CCc3nc(N)ccc31)CC2] | 456.44 | 455.21 |
| 93 | 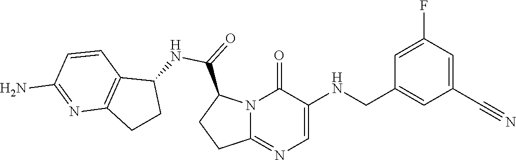 [SMILES: N#Cc1cc(F)cc(CNc2cnc3n(c2=O)[C@H](C(=O)N[C@@H]2CCc4nc(N)ccc42)CC3)c1] | 460.38 | 459.18 |
| 94 | 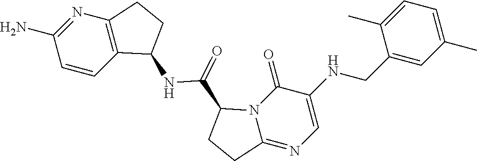 [SMILES: Cc1ccc(C)c(CNc2cnc3n(c2=O)[C@H](C(=O)N[C@@H]2CCc4nc(N)ccc42)CC3)c1] | 445.21 | 444.23 |
| 95 | 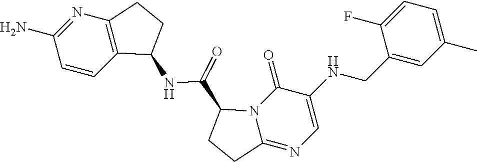 [SMILES: Cc1ccc(F)c(CNc2cnc3n(c2=O)[C@H](C(=O)N[C@@H]2CCc4nc(N)ccc42)CC3)c1] | 449.22 | 448.20 |
| 96 | 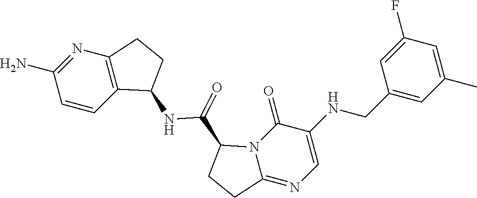 [SMILES: Cc1cc(F)cc(CNc2cnc3n(c2=O)[C@H](C(=O)N[C@@H]2CCc4nc(N)ccc42)CC3)c1] | 449.21 | 448.20 |
| 97 | 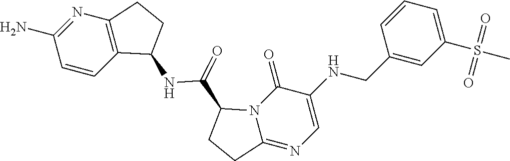 [SMILES: CS(=O)(=O)c1cccc(CNc2cnc3n(c2=O)[C@H](C(=O)N[C@@H]2CCc4nc(N)ccc42)CC3)c1] | 495.38 | 494.17 |
| 98 | 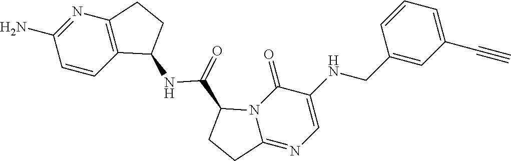 [SMILES: C#Cc1cccc(CNc2cnc3n(c2=O)[C@H](C(=O)N[C@@H]2CCc4nc(N)ccc42)CC3)c1] | 441.39 | 440.20 |
| 99 | 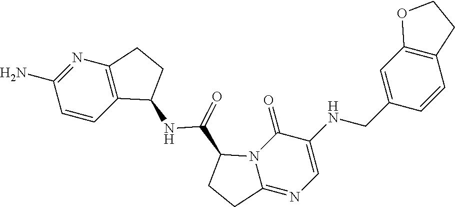 [SMILES: Nc1ccc2c(n1)CC[C@H]2NC(=O)[C@@H]1CCc2ncc(NCc3ccc4c(c3)OCC4)c(=O)n21] | 459.42 | 458.21 |
| 100 | 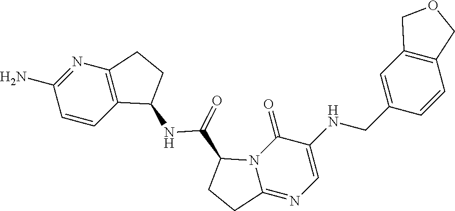 [SMILES: Nc1ccc2c(n1)CC[C@H]2NC(=O)[C@@H]1CCc2ncc(NCc3ccc4c(c3)COC4)c(=O)n21] | 459.42 | 458.21 |
| 101 | 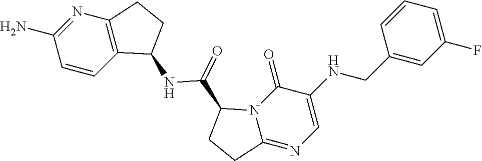 [SMILES: Nc1ccc2c(n1)CC[C@H]2NC(=O)[C@@H]1CCc2ncc(NCc3cccc(F)c3)c(=O)n21] | 435.23 | 434.19 |
| 102 | 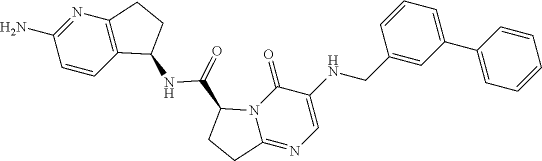 [SMILES: Nc1ccc2c(n1)CC[C@H]2NC(=O)[C@@H]1CCc2ncc(NCc3cccc(-c4ccccc4)c3)c(=O)n21] | 493.29 | 492.23 |
| 103 | 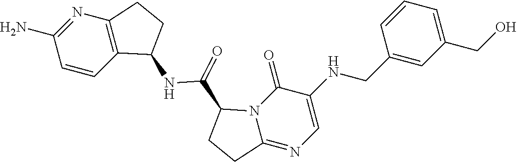 [SMILES: Nc1ccc2c(n1)CC[C@H]2NC(=O)[C@@H]1CCc2ncc(NCc3cccc(CO)c3)c(=O)n21] | 447.43 | 446.21 |
| 104 | 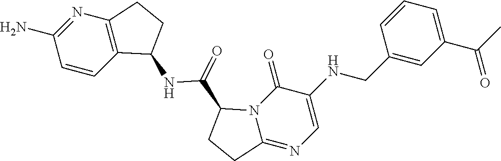 [SMILES: CC(=O)c1cccc(CNc2cnc3n(c2=O)[C@H](C(=O)N[C@@H]2CCc4nc(N)ccc42)CC3)c1] | 459.43 | 458.21 |
| 105 | 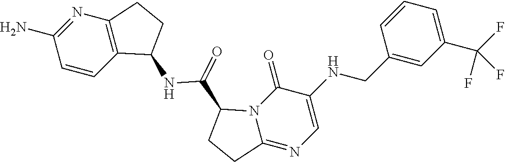 [SMILES: Nc1ccc2c(n1)CC[C@H]2NC(=O)[C@@H]1CCc2ncc(NCc3cccc(C(F)(F)F)c3)c(=O)n21] | 485.25 | 484.18 |

### Example 40

### Enzymatic Assay for MASP-2 Inhibitory Activity

The MASP-2 assay utilizes a fluorogenic substrate, based on the cleavage site for its natural substrate C2. The assay was run at room temperature in an assay buffer containing 20 mM HEPES, pH 7.4, 140 mM NaCl and 0.1% Tween 20. Assay parameters were adjusted such that the assay was linear with respect to time, enzyme, and substrate concentrations. Under the optimized assays conditions, IC50 values were equivalent to Ki values, except in the few cases of “tight binding” inhibitors. Cases of “tight binding” or possible “slow binding” inhibitors were handled by the methods described in Copeland R. A. (2013) Evaluation of Enzyme Inhibitors in Drug Discovery. 2nd Ed., John Wiley and Sons, Inc., Chapters 5-7.

The MASP-2 assay protocol was carried out as follows. Test compounds were serially diluted in DMSO and then 100 nL of each dilution was transferred to the assay plate(s). 10 μL of Assay Buffer was added, followed by 15 μL of Enzyme MASP-2 (CCP1-CCP2-SP) in Assay Buffer. 15 μL of Substrate in Assay Buffer was then added and the mixture was mixed to start the reaction. After 20 min at room temperature, 15 μL of a stop solution (0.1 M acetic acid) was added, the sample was mixed, and the plates were read on a SpectraMax i3× Microplate Reader with the data exported as an Excel file. Each assay plate included a “no inhibitor” (DMSO only) control, a “no enzyme” control, and a reference inhibitor control. % Activity values=100*(average test comp. fluorescence−average “no enzyme” fluorescence)/(average “DMSO only” fluorescence−average “no enzyme” fluorescence). IC50 and Ki replicate values were very reproducible, with values well within +2-fold.

The biological assay results for the compounds listed in Table 1 are shown in Table 3 below. The biological assay results for the compounds listed in Table 2 are shown in Table 4 below.

### Example 41

### Enzymatic Assay for Thrombin

The thrombin assay utilizes a fluorogenic peptide substrate (Boc-VPR-AMC (R&D Systems) and was run at room temperature in an assay buffer containing 20 mM Hepes, pH 7.4, 140 mM NaCl and 0.1% Tween 20. Assay parameters were adjusted such that the assay was linear with respect to time, enzyme, and substrate concentrations. Under these optimized assays conditions, IC₅₀ values were equivalent to Kᵢ values, except in a few cases of “tight binding” inhibitors. Cases of “tight binding” or possible “slow binding” inhibitors were handled by the methods described in Copeland R.A. (2013) Evaluation of Enzyme Inhibitors in Drug Discovery. 2^{nd} Ed. John Wiley and Sons, Inc., Chapters 5-7.

The thrombin assay protocol was carried out as follows. Test compounds were serially diluted in DMSO and then 100 nL of each dilution was transferred to the assay plate(s). 10 μL of Assay Buffer was added, followed by 15 μL of enzyme (human a-thrombin (BioPharm® Lab.)) in assay buffer. 15 μL of substrate in assay buffer was then added and the sample mixed to start the reaction. After 20 min at room temperature, 15 μL of a stop solution (0.1 M acetic acid) was added, the sample was mixed, and the plates were read on a SpectraMax® i3× Microplate Reader with the data exported as Excel files. Each assay plate included a “no inhibitor” (DMSO only) control, a “no enzyme” control, and a reference inhibitor control. % Activity values=100*(ave. test comp. fluorescence−ave. “no enz” fluorescence)/(ave. “DMSO only” fluorescence−ave. “no enz” fluorescence). IC₅₀ and Kᵢ values were very reproducible, with values falling well within +2-fold.

The biological assay results for the compounds listed in Table 1 are shown in Table 3 below. The biological assay results for the compounds listed in Table 2 are shown in Table 4 below.

### Example 42

### Lectin Pathway Activation (LPA) Assay in Human Serum Treated with Small Compounds

A Microtiter ELISA plate (Nunc-Maxi Sorp; Sigma-Aldrich®, USA) was coated with mannan sourced from Saccharomyces cerevisiae (Sigma-Aldrich®, USA) in a coating buffer comprising 33.3 mM Na₂CO₃ and 166.67 mM NaHCO₃. The plates were incubated at room temperature overnight. Subsequently, the plates were blocked with a 5% bovine serum albumin solution (Sigma-Aldrich®, USA) prepared in PBST for 2 hours at room temperature with agitation. Varying concentrations of MASP-2 inhibitors were dispensed onto the empty plate.

To perform the assay, pooled human serum was diluted to 1% in a solution of 20 mM HEPES (pH=7.4), 140 mM NaCl, and 1 mM MgCl₂. Then, 40 μl of the diluted human serum was dispensed into each well of the Microtiter ELISA plate and incubated for 55 minutes at room temperature. The C4 deposition on the mannan-coated surface was stopped using an EDTA stop solution (20 mM HEPES, pH=7.4, 140 mM NaCl, and 150 mM EDTA). Subsequently, rabbit anti-Human C4c antibody (Agilent Technologies, USA) prepared in PBST was added, and the plates were incubated at room temperature for 30 minutes with agitation, then washed with PBST, and Goat anti-rabbit IgG(H+L) HRP-conjugated (Southern Biotech®, USA) was added. The plates were again incubated at room temperature for 30 minutes with agitation, and then were washed with PBST. Following this, the Amplex® UltraRed® (Thermo Fischer Sci, USA) substrate solution was added for 16 minutes, followed by the addition of the Amplex® UltraRed® stop solution. The plates' fluorescence was measured using an i3× microplate reader (Molecular Devices, USA) with excitation at 550 nm and emission at 580 nm. The raw data generated from the plate reader was exported to GraphPad Prism® (version 10.0.3), and the non-linear regression curve was generated using the 4-parameter logistic fit equation. All data are reported as pIC₅₀ with a 95% confidence interval. Data for select exemplary compounds is shown in Table 5.

### Example 43

### Biological Data for Exemplary Compounds

Biological assay results for the compounds listed in Table 1 are shown in Table 3.

TABLE 3
MASP-2 and Thrombin Inhibition by Exemplary Compounds

| Cmp | MASP-2 | Thrombin |
|---|---|---|
| No. | Kᵢ (μM) | Kᵢ (μM) |
| 1 | **** | *** |
| 2 | **** | **** |
| 3 | **** | *** |
| 4 | **** | *** |
| 5 | **** | **** |
| 6 | **** | ** |
| 7 | **** | — |
| 8 | **** | *** |
| 9 | **** | ** |
| 10 | **** | *** |
| 11 | **** | *** |
| 12 | **** | ** |
| 13 | **** | * |
| 14 | **** | ** |
| 15 | **** | — |
| 16 | **** | — |
| 17 | **** | **** |
| 18 | **** | ** |
| 19 | *** | — |
| 20 | **** | — |
| 21 | **** | ** |
| 22 | **** | — |
| 23 | ** | — |
| 24 | **** | * |
| 25 | **** | — |
| 26 | **** | *** |
| 27 | **** | *** |
| 28 | **** | *** |
| 29 | **** | *** |
| 30 | **** | *** |
| 31 | **** | *** |
| 32 | **** | * |
| 33 | **** | *** |
| 34 | **** | **** |
| 35 | **** | *** |
| 36 | **** | ** |
| 37 | **** | **** |
| 38 | **** | ** |
| 39 | **** | *** |
| 40 | **** | *** |
| 41 | **** | *** |
| 42 | **** | *** |
| 43 | **** | ** |
| 44 | **** | * |
| 45 | **** | *** |
| 46 | **** | *** |
| 47 | **** | *** |
| 48 | **** | *** |
| 49 | **** | **** |
| 50 | **** | ** |
| 51 | **** | — |
| 52 | **** | ** |
| 53 | **** | *** |
| 54 | **** | *** |
| 55 | **** | **** |
| 56 | **** | *** |
| 57 | **** | *** |
| 58 | **** | *** |
| 59 | **** | ** |
| 60 | **** | *** |
| 61 | **** | ** |
| 62 | **** | ** |
| 63 | **** | *** |
| 64 | **** | * |
| 65 | **** | — |
| 66 | *** | — |
| 67 | *** | * |
| 68 | **** | ** |
| 69 | **** | *** |
| 70 | **** | * |
| 71 | **** | *** |
| 72 | **** | *** |
| 73 | **** | *** |
| 74 | **** | **** |
| 75 | **** | *** |
| 76 | **** | * |
| 77 | **** | — |
| 78 | ** | — |
| 79 | **** | — |
| 80 | **** | *** |
| 106 | **** | — |
| 107 | **** | — |

Biological assay results for the compounds listed in Table 2 are shown in Table 4.

TABLE 4
MASP-2 and Thrombin Inhibition by Exemplary Compounds

| Cmp | MASP-2 | Thrombin |
|---|---|---|
| No. | Kᵢ (μM) | Kᵢ (μM) |
| 81 | **** | *** |
| 82 | **** | *** |
| 83 | **** | **** |
| 84 | **** | *** |
| 85 | **** | — |
| 86 | **** | — |
| 87 | **** | **** |
| 88 | **** | *** |
| 89 | **** | **** |
| 90 | **** | *** |
| 91 | **** | *** |
| 92 | **** | ** |
| 93 | **** | *** |
| 94 | **** | *** |
| 95 | **** | *** |
| 96 | **** | *** |
| 97 | **** | *** |
| 98 | **** | **** |
| 99 | **** | ** |
| 100 | **** | ** |
| 101 | **** | *** |
| 102 | **** | * |
| 103 | **** | ** |
| 104 | **** | *** |
| 105 | **** | ** |

MASP-2 and Thrombin Inhibition:
* Ki between 10-25 μM
** Ki between 2.5-10 μM
*** Ki between 0.5-2.5 μM
**** Ki less than 0.5 μM
— Ki greater than 25 μM

Biological LPA assay results for selected compounds are shown in Table 5.

TABLE 5
LPA Inhibition by Exemplary Compounds

| Cmp | LPA |
|---|---|
| No. | IC₅₀ (μM) |
| 18 | +++ |
| 20 | +++ |
| 22 | +++ |
| 25 | +++ |
| 44 | +++ |
| 46 | +++ |
| 50 | +++ |
| 53 | +++ |
| 54 | +++ |
| 62 | +++ |
| 64 | +++ |
| 65 | +++ |
| 72 | +++ |
| 75 | +++ |
| 76 | +++ |
| 81 | +++ |
| 82 | +++ |
| 86 | +++ |
| 90 | +++ |
| 94 | +++ |
| 95 | +++ |
| 97 | +++ |
| 100 | +++ |
| 105 | +++ |
| 106 | +++ |

LPA Inhibition: +++ IC₅₀ less than 0.5 μM

It is understood that the examples and embodiments described herein are for illustrative purposes only and that various modifications or changes in light thereof will be suggested to persons skilled in the art and are to be included within the spirit and purview of this application and scope of the appended claims. Each reference, including without limitation all patent, patent applications, and publications, cited in the present application is incorporated herein by reference in its entirety for all purposes.

1. A compound having Structure (I): 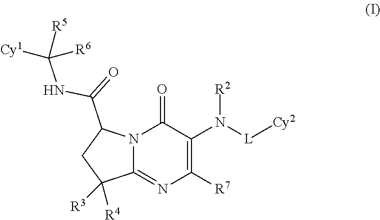
or a stereoisomer, tautomer, or pharmaceutically acceptable salt thereof, wherein:
Cy¹ is a substituted aryl or a substituted or unsubstituted 5-10-membered heteroaryl, or
Cy¹, together with one of R⁵ or R⁶ and the carbon to which they are attached, forms a substituted or unsubstituted C₃-C₆ cycloalkyl fused to a substituted or unsubstituted 5-10-membered heteroaryl, or a substituted or unsubstituted phenyl;
Cy² is a substituted aryl, a substituted or unsubstituted C₃-C₆ cycloalkyl, a substituted or unsubstituted 5-10-membered heteroaryl, or is hydrogen;
R² is hydrogen, a substituted or unsubstituted C₁-C₃ alkyl, or a substituted or unsubstituted cycloalkyl;
R³ and R⁴ are each independently hydrogen, halogen, substituted or unsubstituted C₁-C₃ alkyl, or substituted or unsubstituted cycloalkyl, or R³ and R⁴, together with the carbon to which they are attached, form a substituted or unsubstituted C₃-C₆ cycloalkyl or a substituted or unsubstituted C₅-C₆ cycloalkenyl;
R⁵ and R⁶ are each independently hydrogen, C₁-C₃ alkyl, alkoxy, haloalkyl, hydroxyalkyl, haloalkoxy, or C₃-C₆ cycloalkyl;
R⁷ is selected from the group consisting of hydrogen, halogen, substituted or unsubstituted C₁-C₃ alkyl, and substituted or unsubstituted C₃-C₆ cycloalkyl;
L is hydrogen or —(CR^{8a}R^{8b})ₙ—, wherein each —(CR^{8a}R^{8b})— is independently the same or different;
R^{8a} and R^{8b} are each independently hydrogen, substituted or unsubstituted linear or branched C₁-C₃ alkyl, or R^{8a} and R^{8b}, together with the carbon to which they are attached, form a substituted or unsubstituted C₃-C₆ cycloalkyl; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms in Structure (I) is optionally replaced with a deuterium atom; and
provided that the compound of Structure (I) does not have the structure: 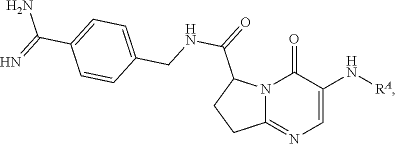
wherein R^{A} is benzyl, phenethyl, or 3-CF₃-benzyl.

2. The compound of claim 1, wherein R² is hydrogen, methyl,
or ethyl.

3. (canceled)

4. The compound of claim 1, wherein R³ and R⁴ are hydrogen.

5. The compound of claim 1, wherein R⁵ is hydrogen.

6. The compound of claim 1, wherein R⁷ is hydrogen, deuterium, C₁-C₃ alkyl, or C₁-C₃ haloalkyl.

7-8. (canceled)

9. The compound of claim 1, wherein the compound has Structure (I-A-1), (I-A-2), (I-A-3) or (I-A-4):

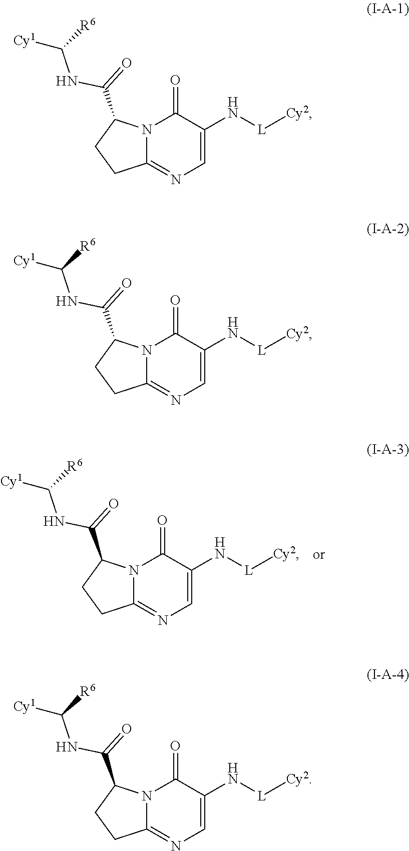

10. The compound of claim 1, wherein R⁶ is hydrogen or methyl.

11. (canceled)

12. The compound of claim 1, wherein the compound has Structure (I-B): 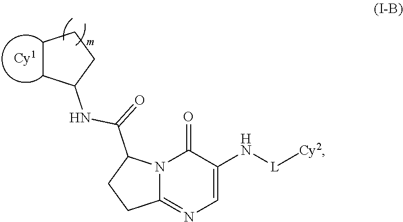
wherein m is 1 or 2.

13-15. (canceled)

16. The compound of claim 1, wherein Cy¹ is a C₆-C₁₀ aryl substituted with one or more substituents independently selected from the group consisting of hydrogen, C(═NH)NHC(═O)OR⁸, C(═NOC(═O)R⁸)NH₂, C(═NOC(═O)OR⁸)NH₂, C(═NOH)NH₂, C(═NH)NHC(═O)NHC(═O)N(CH₃)R¹⁷, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl,
wherein R⁸, R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

17. (canceled)

18. The compound of claim 1, wherein Cy¹ is selected from the group consisting of:

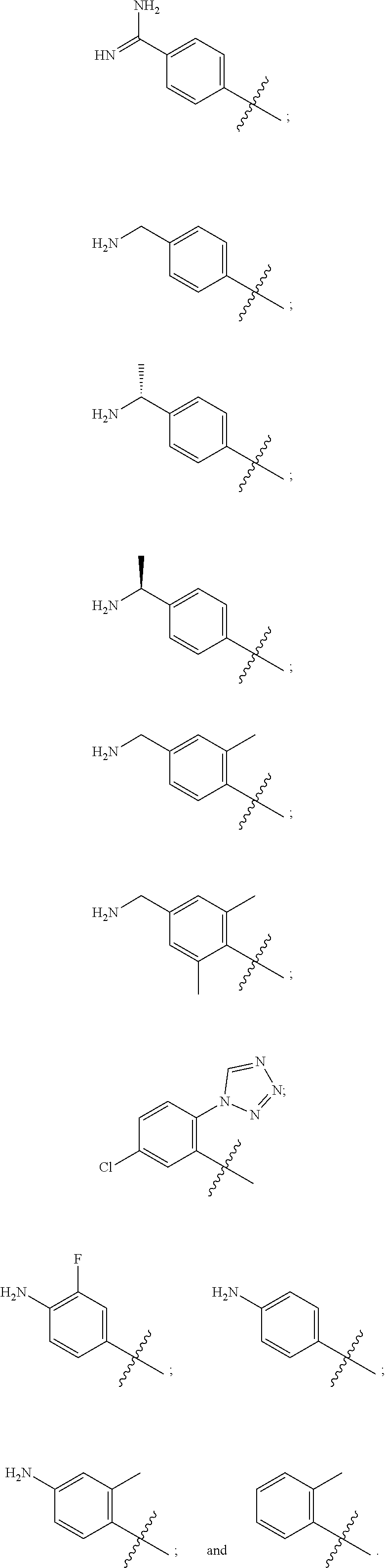

19-21. (canceled)

22. The compound of claim 1, wherein Cy¹ is a substituted or unsubstituted 5-10-membered heteroaryl, and the substituted 5-10-membered heteroaryl of Cy¹ is a 5-10-membered heteroaryl substituted with one or more substituents independently selected from the group consisting of hydrogen, C₁₋₆ deuterated alkyl, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NH)NHC(═O)NHC(═O)N(CH₃)R¹⁷, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the heteroaryl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl,
wherein the one or more substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl, and
wherein R¹⁷ is a C₆-C₁₀ aryl or 5-10-membered heteroaryl, optionally substituted with CH₂OC(═O)CH₃.

23-35. (canceled)

36. The compound of claim 1, wherein Cy¹ is selected from the group consisting of:

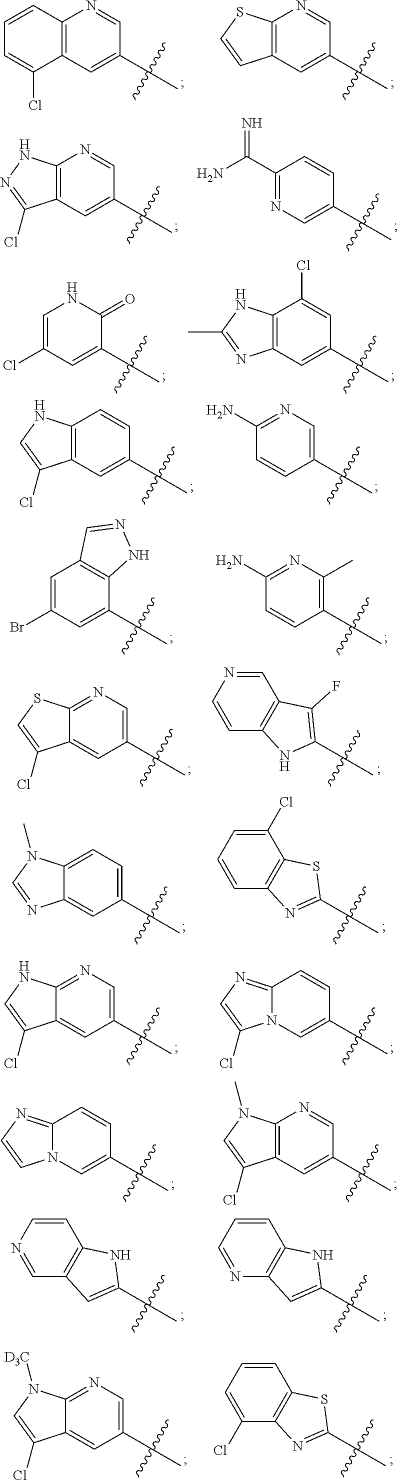

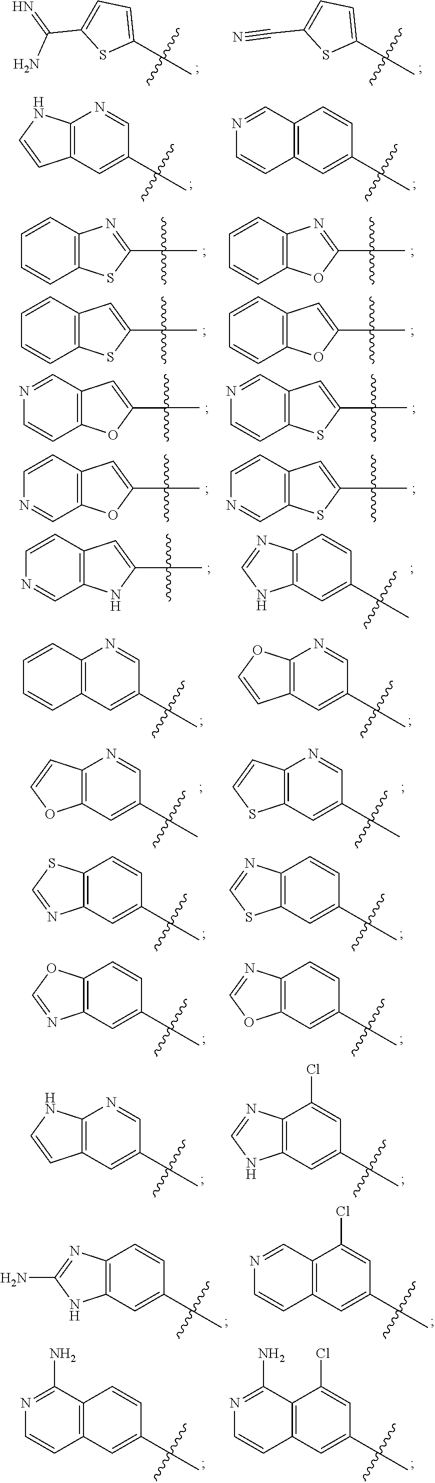

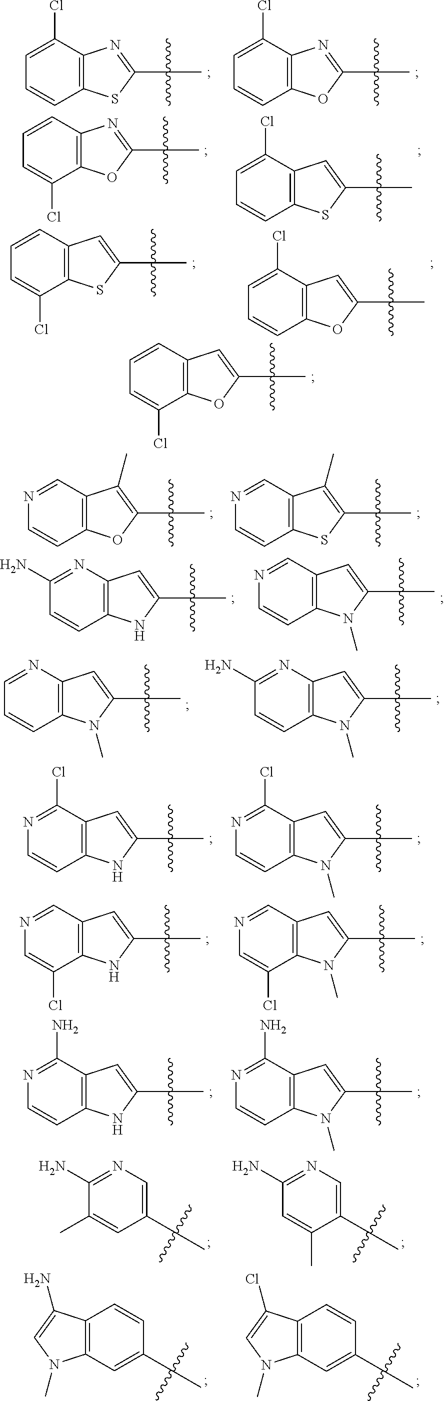

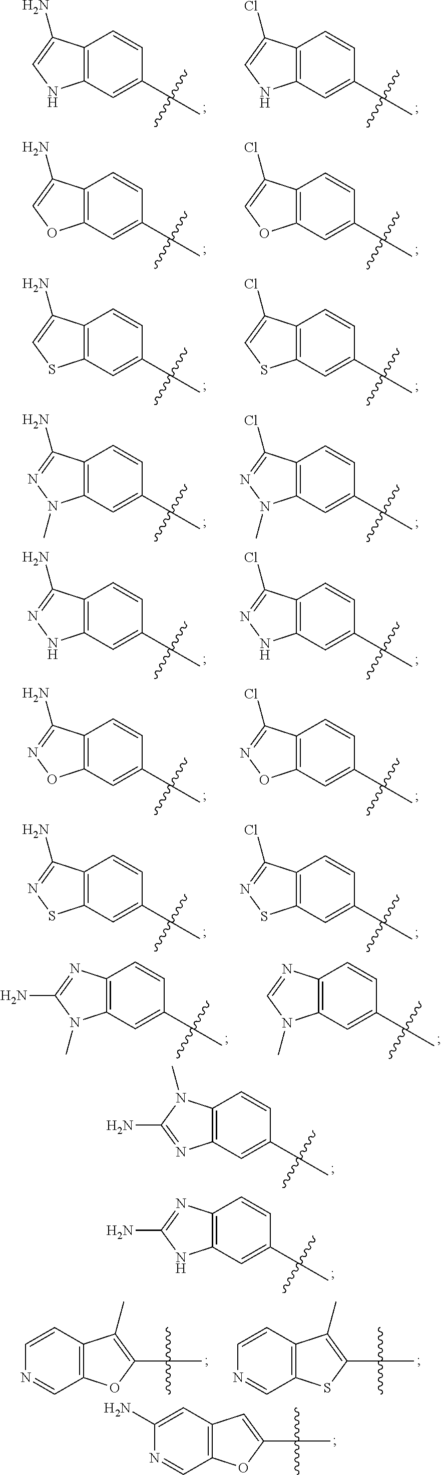

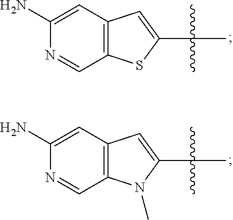 [SMILES: *c1cc2cc(N)ncc2s1 ; *c1cc2cc(N)ncc2n1C]

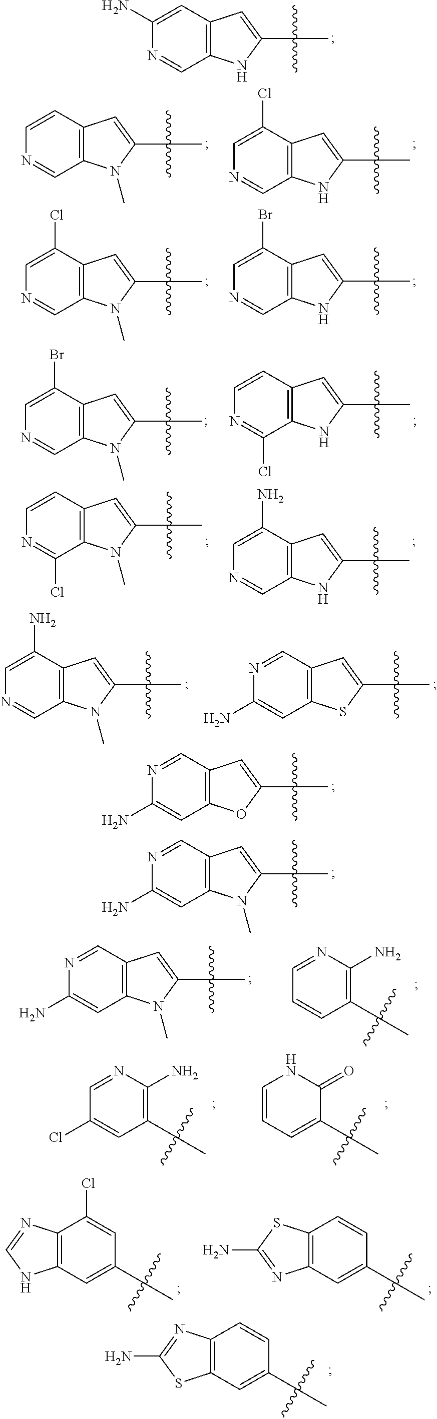

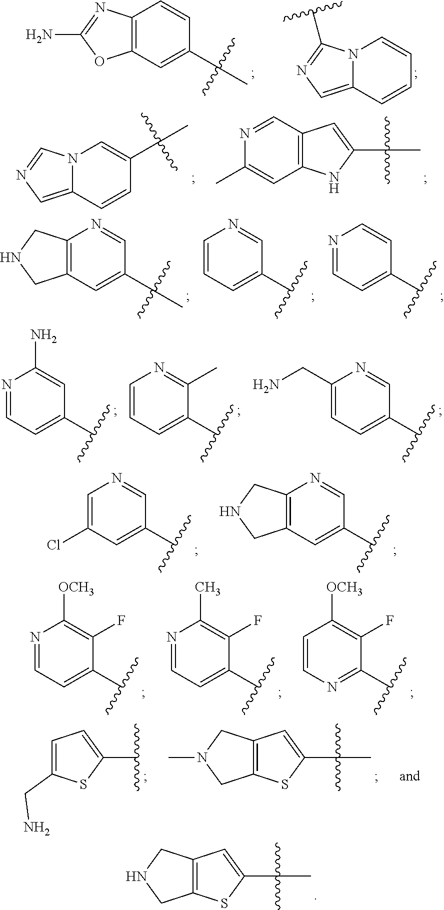

37-39. (canceled)

40. The compound of claim 1, wherein Cy¹ is selected from the group consisting of:

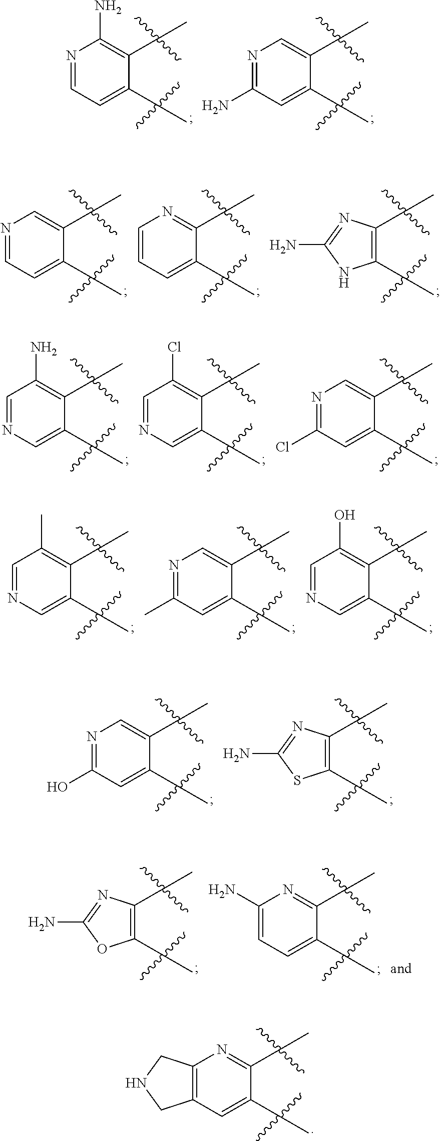

41-44. (canceled)

45. The compound of claim 1, wherein L is —CH₂—, —CH₂CH₂—, —CH(CH₃)—, or —CH₂CH₂CH₂—.

46-47. (canceled)

48. The compound of claim 1, wherein Cy² is a C₆-C₁₀ aryl substituted with one or more substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, C(═NR⁹)NR¹⁰R¹¹, C(═NOR⁹)NR¹⁰R¹¹, C(═NOC(O)R⁹)NR¹⁰R¹¹, C(═NR⁹)N(R¹⁰)C(O)OR¹¹, N(R⁹)C(═NR¹⁰)NR¹¹R¹², S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the C₆-C₁₀ aryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the C₆-C₁₀ aryl,
wherein R⁹, R¹⁰, R¹¹, and R¹², are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the one or more substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the one or more substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₄ haloalkoxy, C₁₋₆ hydroxyalkyl, cycloalkyl, heterocyclyl, and heteroaryl.

49-51. (canceled)

52. The compound of claim 1, wherein Cy¹ is selected from the group consisting of:

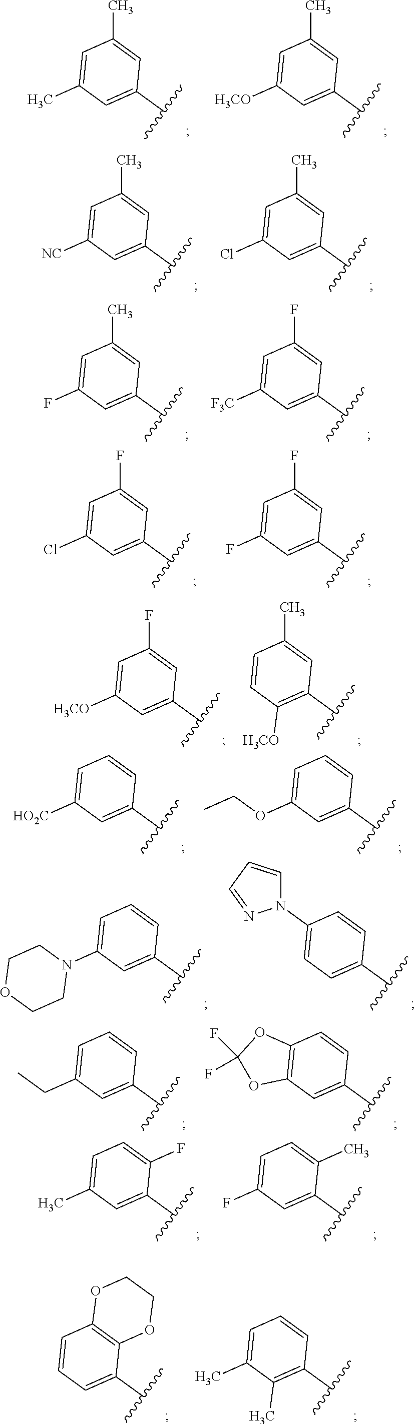

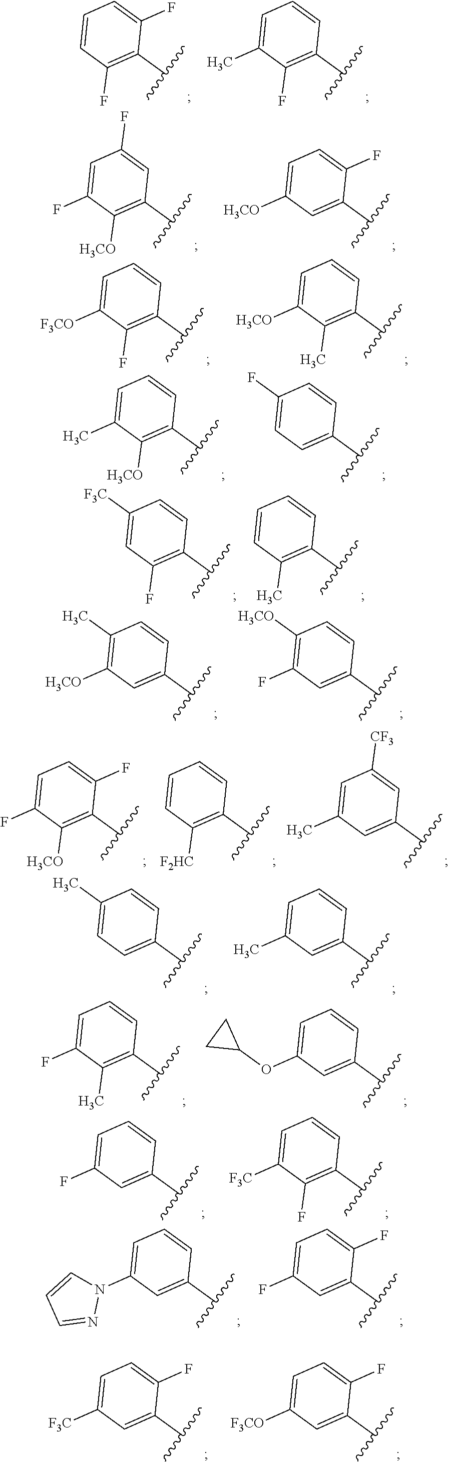

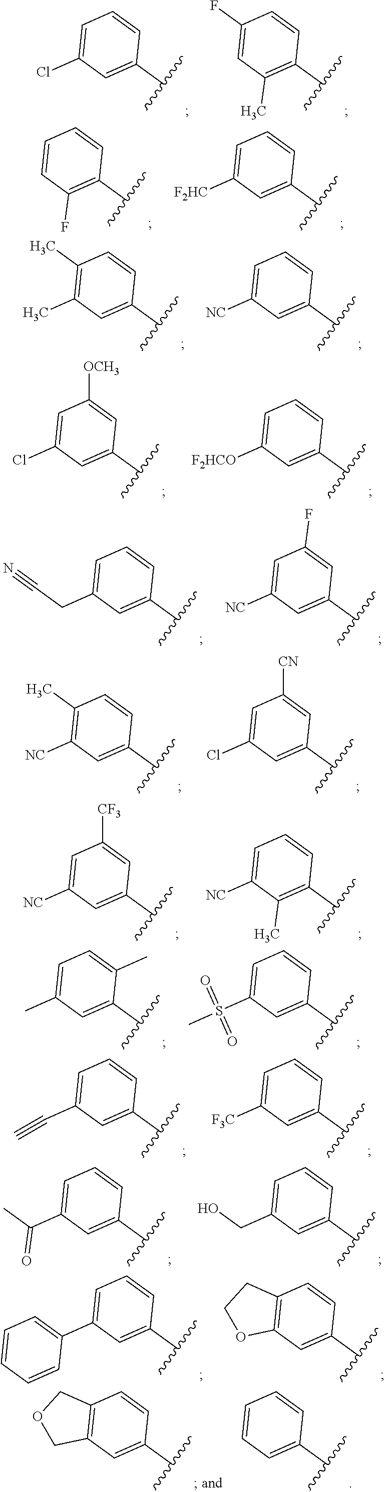

53. The compound of claim 1, wherein Cy² is a substituted or unsubstituted C₃-C₆ cycloalkyl, wherein the substituted C₃-C₆ cycloalkyl is substituted with 1-5 substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the 1-5 substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the 1-5 substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

54-55. (canceled)

56. The compound of claim 1, wherein Cy² is a substituted or unsubstituted 5-10 membered heteroaryl, wherein the substituted 5-10 membered heteroaryl is substituted with 1-4 substituents independently selected from the group consisting of hydrogen, C₂₋₆ alkenyl, C₂₋₆ alkynyl, halogen, C₁₋₆ haloalkyl, aminylalkyl, hydroxyalkyl, cyano, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, oxo, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or wherein when the heteroaryl is substituted at two adjacent atoms, the two substituents are connected, together with the atoms to which they are attached, to form a substituted or unsubstituted C₅-C₆ cycloalkyl, or a substituted or unsubstituted 5- or 6-membered heterocyclic ring comprising 1-4 heteroatoms selected from the group consisting of N, S, and O, fused to the heteroaryl,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the 1-4 substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the 1-4 substituents are a substituted C₁₋₆ alkyl, a substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl.

57-60. (canceled)

61. The compound of claim 1, having a structure selected from the group consisting of: 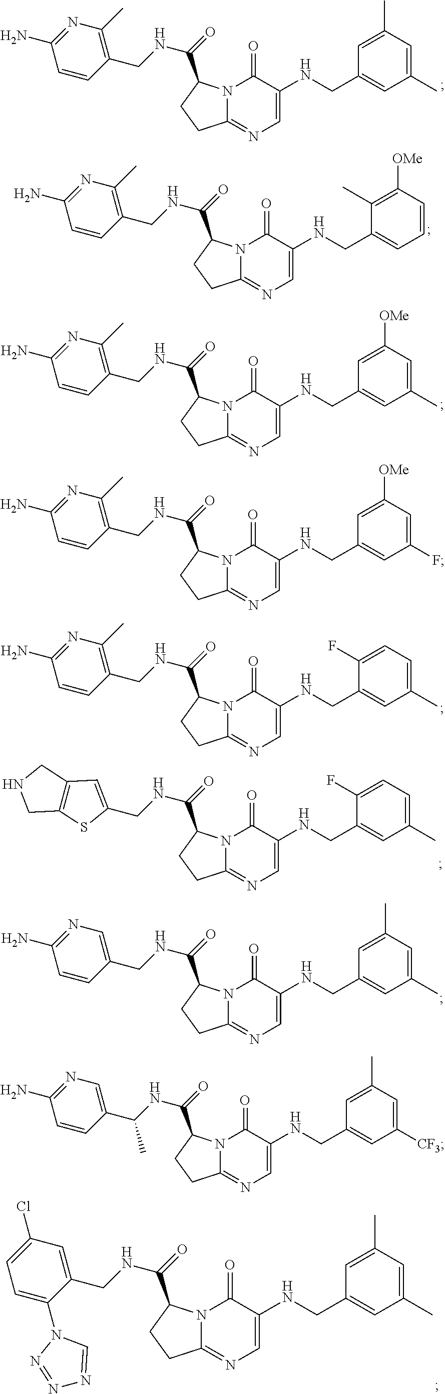 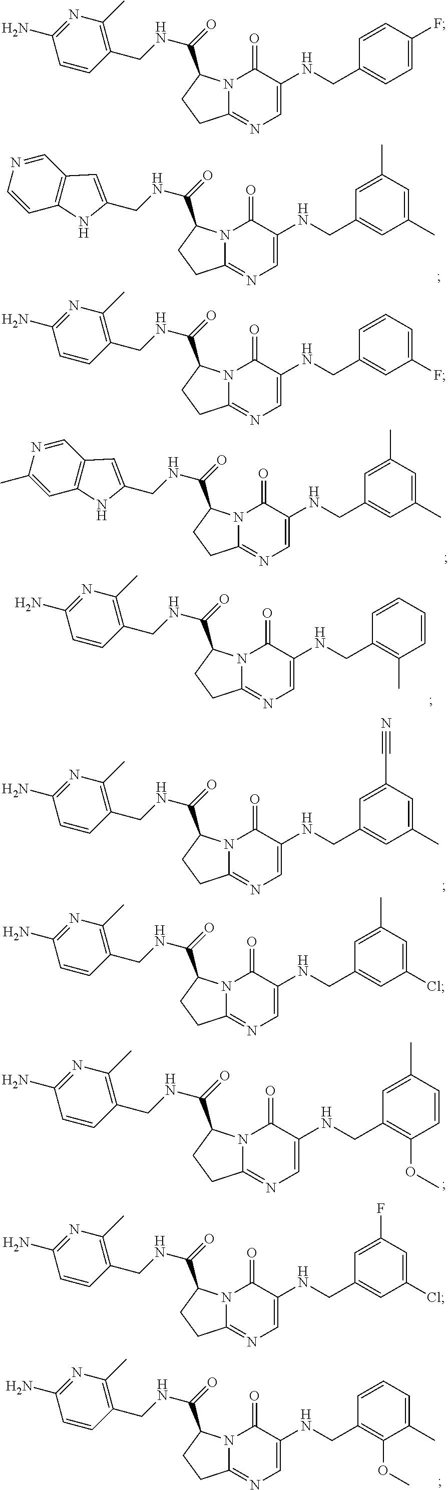 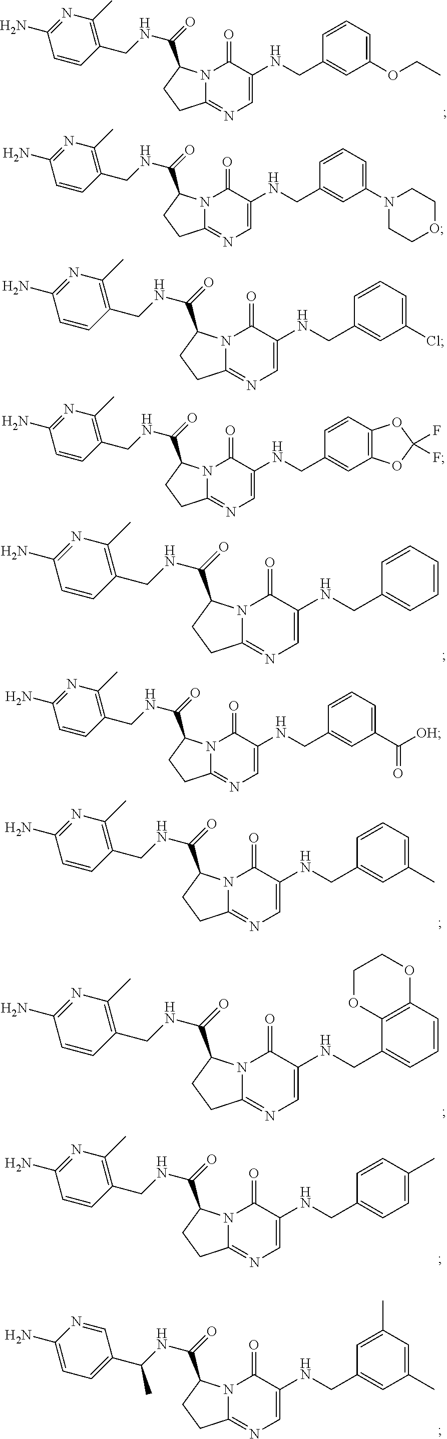 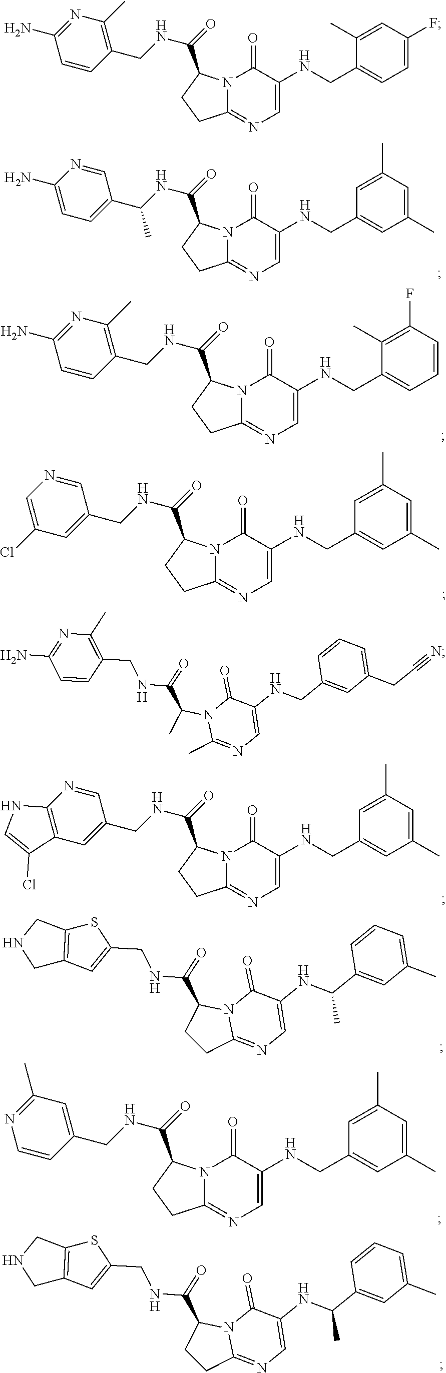 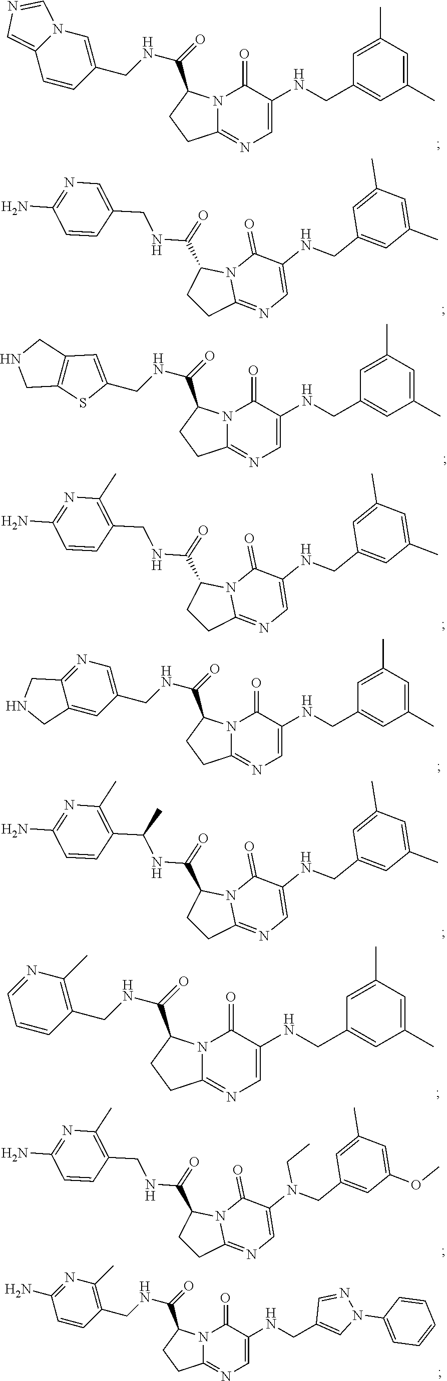 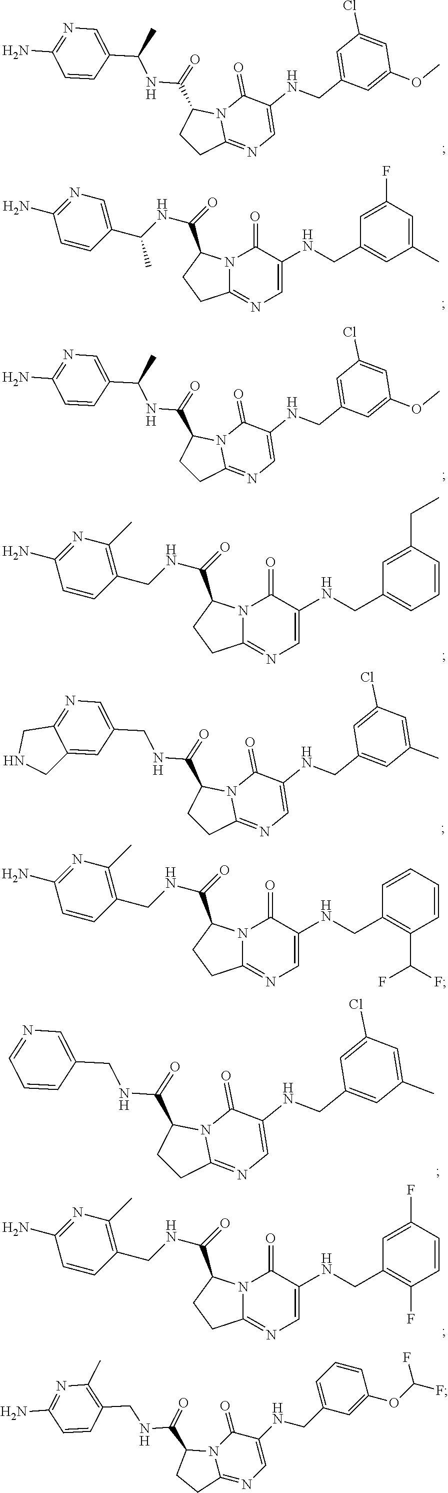 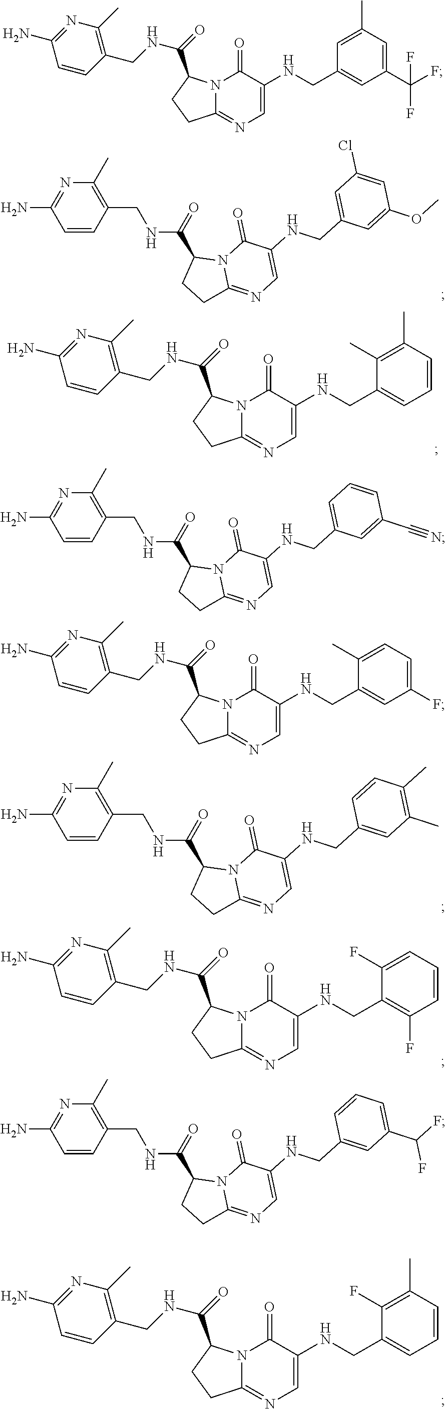 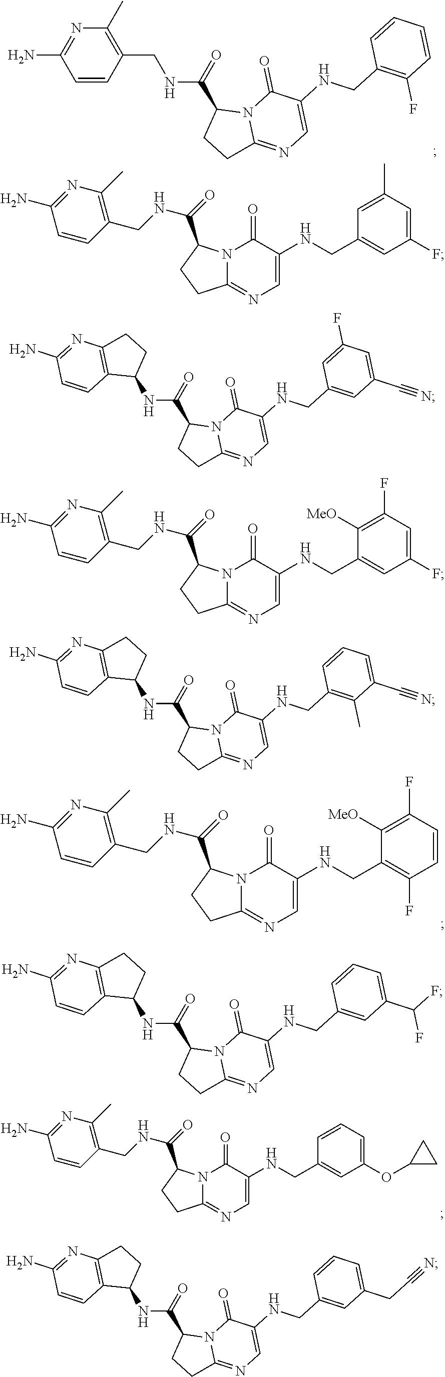 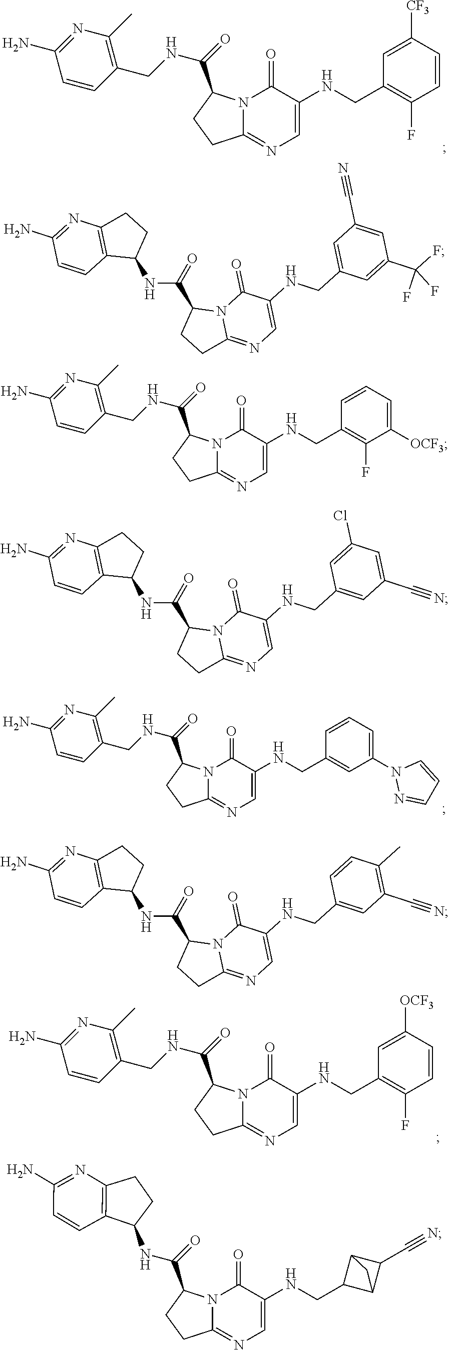 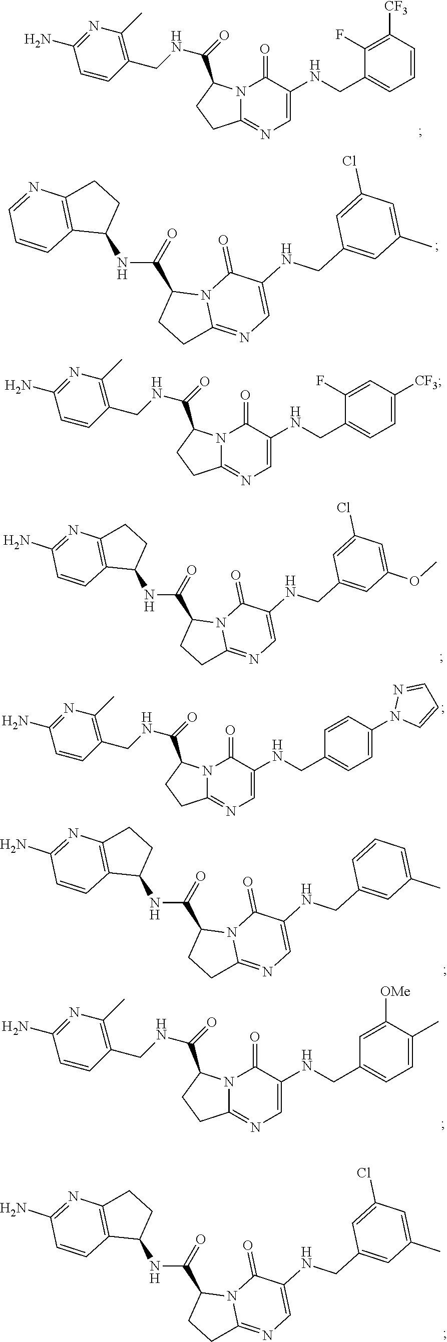 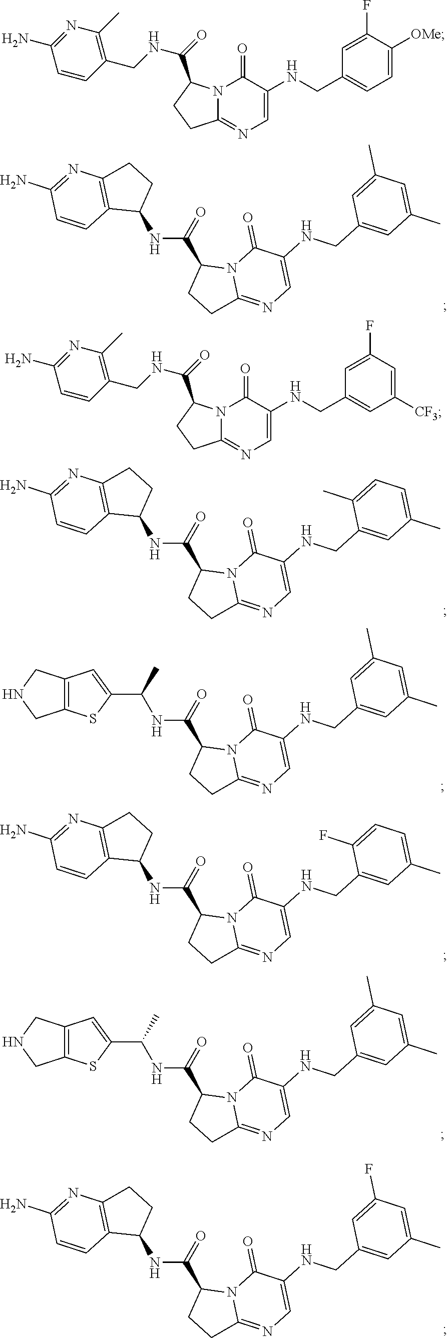 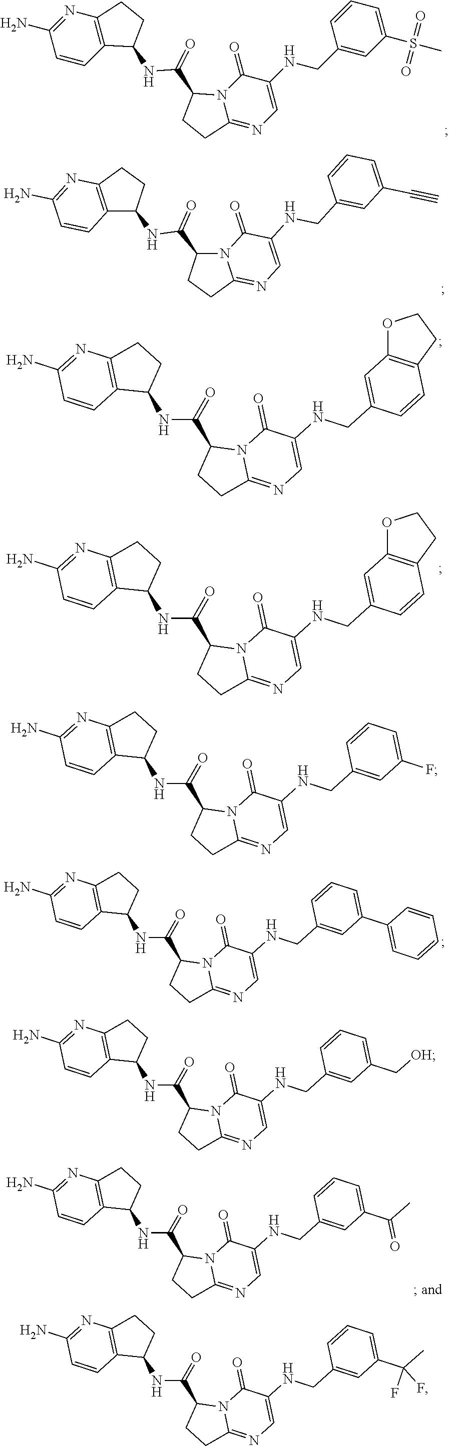
or a stereoisomer, tautomer, or pharmaceutically acceptable salt thereof.

62. A compound having Structure (LI): 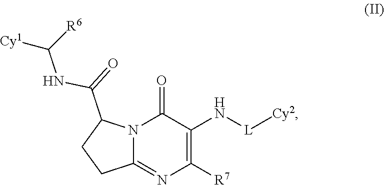
or a stereoisomer, tautomer, or pharmaceutically acceptable salt thereof, wherein:
Cy¹ is selected from the group consisting of: 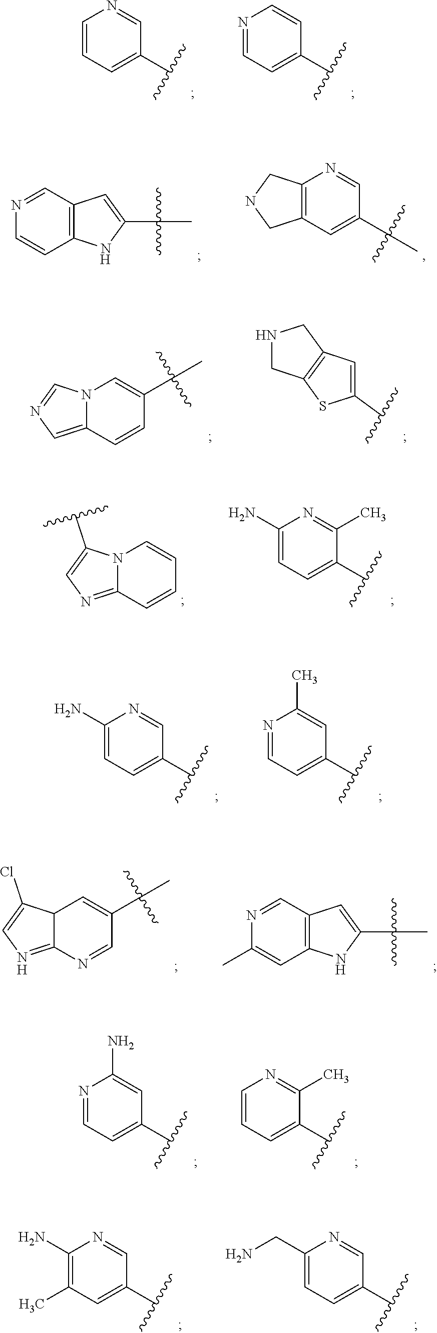 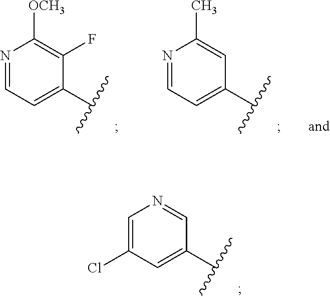
Cy² is a phenyl substituted with 1-5 substituents independently selected from the group consisting of hydrogen, C₂₋₆ alkenyl, C₂₋₆ alkynyl, halogen, C₁₋₆ haloalkyl, amino C₁₋₆ alkyl, C₁₋₆ alkyloxy, cyano, cyanomethyl, OR⁹, SR⁹, C(O)R⁹, C(O)NR⁹R¹⁰, C(O)OR⁹, OC(O)R⁹, OC(O)OR⁹, OC(O)NR⁹R¹⁰, NR⁹R¹⁰, N(R⁹)C(O)R¹⁰, N(R⁹)C(O)NR¹⁰R¹¹, N(R⁹)C(O)OR¹⁰, S(O)R⁹, S(O)NR⁹R¹⁰, S(O)₂R⁹, N(R⁹)S(O)₂R¹⁰, S(O)₂NR⁹R¹⁰, substituted or unsubstituted C₁₋₆ alkyl, substituted or unsubstituted C₆₋₁₀ aryl, substituted or unsubstituted C₆₋₁₀ arylalkyl, substituted or unsubstituted C₆₋₁₀ aryloxy, substituted or unsubstituted C₆₋₁₀ arylalkoxy, substituted or unsubstituted 5-10 membered heteroaryl, substituted or unsubstituted C₃₋₁₀ cycloalkyl, and substituted or unsubstituted 4-10 membered heterocyclyl, or the phenyl is substituted at two adjacent carbon atoms and the substituents, together with the carbon atoms to which they are attached, form a C₅-C₆ cycloalkyl or a 5-6 membered heterocycle fused to the phenyl, each of which is optionally substituted with 1-8 substituents independently selected from the group consisting of C₁₋₆ alkyl, C₃₋₆ cycloalkyl, C₁₋₆ haloalkyl, C₃₋₆ halocycloalkyl, and halogen,
wherein R⁹ and R¹⁰ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, heteroarylalkyl, and heteroaryl,
wherein the 1-5 substituents are optionally substituted with one or more substituents independently selected from the group consisting of halogen, CN, OR¹³, SR¹³, C(O)R¹³, C(O)NR¹³R¹⁴, C(O)OR¹³, OC(O)R¹³, OC(O)NR¹³R¹⁴, NR¹³R¹⁴, NR¹³C(O)R¹⁴, NR¹³C(O)NR¹⁴R¹⁵, NR¹³C(O)OR¹⁴, C(═NR¹³)NR¹⁴R¹⁵, NR¹³C(═NR¹⁴)NR¹⁵R¹⁶, S(O)R¹³, S(O)NR¹³R¹⁴, S(O)₂R¹³, NR¹³S(O)₂R¹⁴, S(O)₂NR¹³R¹⁴ and oxo when the 1-5 substituents are a substituted C₁₋₆ alkyl, substituted C₆₋₁₀ aryl, a substituted C₆₋₁₀ arylalkyl, a substituted C₆₋₁₀ aryloxy, a substituted C₆₋₁₀ arylalkoxy, a substituted 5-10 membered heteroaryl, a substituted C₃₋₁₀ cycloalkyl, or a substituted 4-10 membered heterocyclyl,
wherein R¹³, R¹⁴, R¹⁵, and R¹⁶ are, at each occurrence, independently selected from the group consisting of hydrogen, C₁₋₆ alkyl, C₂₋₆ alkenyl, C₂₋₆ alkynyl, hydroxyl, C₁₋₆ alkoxy, aryl, arylalkyl, C₁₋₆ haloalkyl, C₁₋₆ haloalkoxy, C₁₋₆ hydroxyalkyl, cycloalkyl, heterocyclyl, and heteroaryl;
R⁶ is hydrogen, C₁₋₃ alkyl, or C₁₋₃ haloalkyl;
R⁷ is hydrogen, deuterium, C₁₋₆ alkyl, C₃₋₆ cycloalkyl, C₁₋₆ haloalkyl, C₃₋₆ halocycloalkyl, or halogen;
L is —CH₂—, —CH₂CH₂—, —CH(CH₃)—, or —CH₂CH₂CH₂—, and
wherein one or more hydrogen atoms are optionally replaced with one or more deuterium atoms.

63-73. (canceled)

74. A compound having Structure (III): 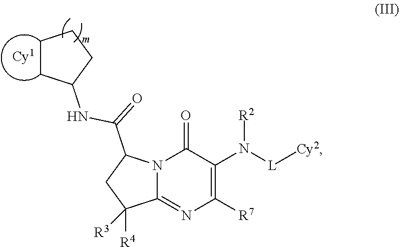
or a stereoisomer, tautomer, or pharmaceutically acceptable salt thereof, wherein:
Cy¹ is a substituted or unsubstituted aryl, or a substituted or unsubstituted 5-10-membered heteroaryl;
Cy² is a substituted aryl, a substituted or unsubstituted C₃-C₆ cycloalkyl, or a substituted or unsubstituted 5-10-membered heteroaryl;
R² is hydrogen, a substituted or unsubstituted C₁-C₃ alkyl, or a substituted or unsubstituted cycloalkyl;
R³ and R⁴ are each independently hydrogen, halogen, substituted or unsubstituted C₁-C₃ alkyl, or substituted or unsubstituted cycloalkyl, or R³ and R⁴, together with the carbon to which they are attached, form a substituted or unsubstituted C₃-C₆ cycloalkyl or a substituted or unsubstituted 5-6-membered cycloalkenyl;
R⁷ is selected from the group consisting of hydrogen, halogen, substituted or unsubstituted C₁-C₃ alkyl, and substituted or unsubstituted C₃-C₆ cycloalkyl;
L is absent or —(CR^{8a}R^{8b})ₙ—, wherein each (CR^{8a}R^{8b})— is independently the same or different;
R^{8a} and R^{8b} are each independently hydrogen, substituted or unsubstituted C₁-C₃ alkyl, or R^{8a} and R^{8b}, together with the carbon to which they are attached, form a substituted or unsubstituted C₃-C₆ cycloalkyl;
m is 1 or 2; and
n is 1, 2, or 3, and
wherein the C₃-C₆ cycloalkyl consists of a monocyclic or bicyclic ring system which comprises a fused or bridged ring system,
wherein the 5-10-membered heteroaryl consists of a monocyclic or bicyclic ring system comprising at least one aromatic ring and one to six heteroatoms selected from the group consisting of nitrogen, oxygen, and sulfur, and
wherein one or more hydrogen atoms is optionally replaced with a deuterium atom.

75-116. (canceled)

117. The compound of claim 1, wherein the salt of the pharmaceutically acceptable salt is trifluoroacetic acid, hydrogen chloride, acetic acid, hydrogen bromide, sulfuric acid, phosphoric acid, maleic acid, fumaric acid, lactic acid, tartric acid, citric acid, and gluconic acid.

118. (canceled)

119. A pharmaceutical composition, comprising a compound of claim 1, or a stereoisomer, tautomer, or pharmaceutically acceptable salt thereof, and a pharmaceutically acceptable carrier or excipient.

120. A method for inhibiting MASP-2 in a subject, comprising administering to the subject a compound of claim 1, or a stereoisomer, tautomer, or pharmaceutically acceptable salt thereof, in an amount effective to inhibit MASP-2.

121-123. (canceled)

124. A method for treating or preventing a disease or disorder treatable by inhibiting MASP-2, comprising administering to a subject in need thereof a therapeutically effective amount of a compound of claim 1, or a stereoisomer, tautomer, or pharmaceutically acceptable salt thereof.

125-161. (canceled)


SEQUENCE LISTING (1 sequence)

SEQ ID NO: 1 - protein, 686 aa, Homo sapiens
MRLLTLLGLLCGSVATPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF